(19) United States
(12) Patent Application Publication
(10) Pub. No.: US 2010/0021420 A1
(43) Pub. Date: Jan. 28, 2010

(54) COMBINATIONS OF PYRAZOLE DERIVATIVES FOR THE INHIBITION OF CDKS AND GSK'S

(75) Inventors: John Francis Lyons, London (GB); Matthew Simon Squires, Cambridge (GB); Neil Thomas Thompson, Cambridge (GB); Neil James Gallagher, Basel (CH)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (GB)

(21) Appl. No.: 12/373,827

(22) PCT Filed: Jul. 13, 2007
(86) PCT No.: PCT/GB07/02654

§ 371 Date: Jun. 22, 2009

Related U.S. Application Data
(60) Provisional application No. 60/830,968, filed on Jul. 14, 2006.

(30) Foreign Application Priority Data
Jul. 20, 2006 (GB) 0614457.0

(51) Int. Cl. A61K 31/454; A61K 31/496; A61K 31/5377; A61K 31/541; A61K 39/395; A61K 38/21; A61K 38/20; A61K 33/24

(52) U.S. Cl. 424/85.2; 514/326; 514/254.05; 514/235.8; 514/227.8; 514/299; 424/133.1; 424/85.4; 424/649; 424/143.1

(57) ABSTRACT

A combination comprising (a) a compound of formula (0): or salts or tautomers or N-oxides or solvates thereof; wherein X is R¹-A-NR⁴— or a 5- or 6-membered carbocyclic or heterocyclic ring; A is a bond, SO₂, C═O, NR⁹(C═O) or 0(C═O) wherein R⁹ is hydrogen or C₁₋₄ hydrocarbyl optionally substituted by hydroxy or C₁₋₄ alkoxy; Y is a bond or an alkylene chain of 1 to 3 carbon atoms; R¹ is hydrogen; a carbocyclic or heterocyclic group having from 3 to 12 ring members; or an optionally substituted C₁₋₈ hydrocarbyl group wherein 1 or 2 of the carbon atoms of the hydrocarbyl group may optionally be replaced by an atom or group selected from O, S, NH, SO, SO₂; R² is hydrogen; halogen; C₁₋₄ alkoxy; or a C₁₋₄ hydrocarbyl group optionally substituted by halogen, hydroxyl or C₁₋₄ alkoxy; R³ is selected from hydrogen and carbocyclic and heterocyclic groups having from 3 to 12 ring members; and R⁴ is hydrogen or a C₁₋₄ hydrocarbyl group optionally substituted by halogen, hydroxyl or C₁₋₄ alkoxy; and (b) a compound of formula (I′″) or salts, tautomers, solvates and N-oxides thereof: wherein R¹ is 2,6-dichlorophenyl; R^{2a} and R^{2b} are both hydrogen; and R³ is a group: formula (A) where R⁴ is C₁₋₄ alkyl.

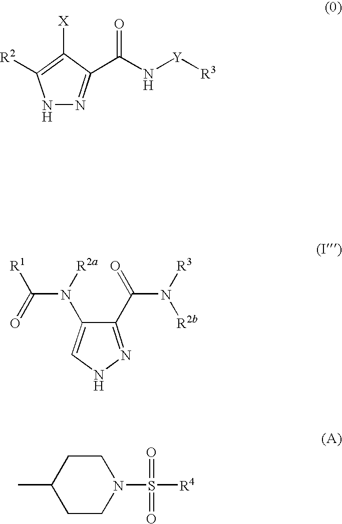

### TECHNICAL FIELD

This invention relates to combinations comprising (or consisting essentially of) one or more compounds of the formula (0) as defined herein, with one or more compounds of the formula (I′″) as herein defined (optionally further combined with one or more of the auxiliary compounds described herein), to processes for preparing the combinations, and to various therapeutic uses of the combinations. Also provided are pharmaceutical compositions containing the combinations.

### BACKGROUND OF THE INVENTION

The combinations of the invention comprise pyrazole compounds of formula (0) and compounds of formula (I′″) that inhibit or modulate the activity of Cyclin Dependent Kinases (CDK) and/or Glycogen Synthase Kinases (GSK, e.g. GSK-3) and optionally one or more auxilliary compounds. The auxilliary compounds may themselves exhibit protein kinase modulatory or inhibitory activity and such activity may be quite distinct from that of the pyrazole component of the combinations (as described infra). Thus, depending on the identity of the auxilliary compound(s) present, the combination as a whole may inhibit or modulate the activity of one or more of a range of different protein kinases, including those described below.

### Protein Kinases

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

### Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale. Progression from the G1 phase to the S phase of the cell cycle is primarily regulated by cdk2, cdk3, cdk4 and cdk6 via association with members of the D and E type cyclins. The D-type cyclins appear instrumental in enabling passage beyond the G1 restriction point, where as the cdk2/cyclin E complex is key to the transition from the G1 to S phase. Subsequent progression through S phase and entry into G2 is thought to require the cdk2/cyclin A complex. Both mitosis, and the G2 to M phase transition which triggers it, are regulated by complexes of cdk1 and the A and B type cyclins.

During G1 phase Retinoblastoma protein (Rb), and related pocket proteins such as p130, are substrates for cdk(2, 4, & 6)/cyclin complexes. Progression through G1 is in part facilitated by hyperphosphorylation, and thus inactivation, of Rb and p130 by the cdk(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the cdk2/cyclin E complex which amplifies, or maintains, E2F levels via further phosphorylation of Rb. The cdk2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the cdk2/cyclin E pathway. Cdk2 is also connected to the p53 mediated DNA damage response pathway via p53 regulation of p21 levels. p21 is a protein inhibitor of cdk2/cyclin E and is thus capable of blocking, or delaying, the G1/S transition. The cdk2/cyclin E complex may thus represent a point at which biochemical stimuli from the Rb, Myc and p53 pathways are to some degree integrated. Cdk2 and/or the cdk2/cyclin E complex therefore represent good targets for therapeutics designed at arresting, or recovering control of, the cell cycle in aberrantly dividing cells.

The exact role of cdk3 in the cell cycle is not clear. As yet no cognate cyclin partner has been identified, but a dominant negative form of cdk3 delayed cells in G1, thereby suggesting that cdk3 has a role in regulating the G1/S transition.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-1, synapsin1, DARPP32 and the Muncl8/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk7 is a nuclear protein that has cdc2 CAK activity and binds to cyclin H. Cdk7 has been identified as component of the TFIIH transcriptional complex which has RNA polymerase II C-terminal domain (CTD) activity. This has been associated with the regulation of HIV-1 transcription via a Tat-mediated biochemical pathway. Cdk8 binds cyclin C and has been implicated in the phosphorylation of the CTD of RNA polymerase II. Similarly the cdk9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. Cdk7, cdk8, cdk9 and the P-TEFb complex are therefore potential targets for anti-viral therapeutics.

At a molecular level mediation of cdk/cyclin complex activity requires a series of stimulatory and inhibitory phosphorylation, or dephosphorylation, events. Cdk phosphorylation is performed by a group of cdk activating kinases (CAKs) and/or kinases such as wee1, Myt1 and Mik1. Dephosphorylation is performed by phosphatases such as cdc25(a & c), pp 2a, or KAP.

Cdk/cyclin complex activity may be further regulated by two families of endogenous cellular proteinaceous inhibitors: the Kip/Cip family, or the INK family. The INK proteins specifically bind cdk4 and cdk6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{kip2}. As discussed previously p21 is induced by p53 and is able to inactivate the cdk2/cyclin(E/A) and cdk4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of cdks, and their associated proteins, in co-ordinating and driving the cell cycle in proliferating cells have been outlined above. Some of the biochemical pathways in which cdks play a key role have also been described. The development of monotherapies for the treatment of proliferative disorders, such as cancers, using therapeutics targeted generically at cdks, or at specific cdks, is therefore potentially highly desirable. Cdk inhibitors could conceivably also be used to treat other conditions such as viral infections, autoimmune diseases and neuro-degenerative diseases, amongst others. Cdk targeted therapeutics may also provide clinical benefits in the treatment of the previously described diseases when used in combination therapy with either existing, or new, therapeutic agents. Cdk targeted anticancer therapies could potentially have advantages over many current antitumour agents as they would not directly interact with DNA and should therefore reduce the risk of secondary tumour development.

### Glycogen Synthase Kinase

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3p at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimulii.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimulii. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

### Diffuse Large B-cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. 1999 July; 80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris M A. Department of Pathology, Virgen de la Salud Hospital, Toledo, Spain.)

### Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% v 3%) and duration of progression-free survival (33 v 17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed in B-CLL (Blood. 1998 Nov. 15; 92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Grever M R).

### Aurora Kinases

Relatively recently, a new family of serine/threonine kinases known as the Aurora kinases has been discovered that are involved in the G2 and M phases of the cell cycle, and which are important regulators of mitosis.

The precise role of Aurora kinases has yet to be elucidated but that they play a part in mitotic checkpoint control, chromosome dynamics and cytokinesis (Adams et al., Trends Cell Biol., 11: 49-54 (2001). Aurora kinases are located at the centrosomes of interphase cells, at the poles of the bipolar spindle and in the mid-body of the mitotic apparatus.

Three members of the Aurora kinase family have been found in mammals so far (E. A. Nigg, Nat. Rev. Mol. Cell. Biol. 2: 21-32, (2001)). These are:
Aurora A (also referred to in the literature as Aurora 2);
Aurora B (also referred to in the literature as Aurora 1); and
Aurora C (also referred to in the literature as Aurora 3).

The Aurora kinases have highly homologous catalytic domains but differ considerably in their N-terminal portions (Katayama H, Brinkley W R, Sen S.; The Aurora kinases: role in cell transformation and tumorigenesis; Cancer Metastasis Rev. 2003 December; 22(4):451-64).

The substrates of the Aurora kinases A and B have been identified as including a kinesin-like motor protein, spindle apparatus proteins, histone H3 protein, kinetochore protein and the tumour suppressor protein p53.

Aurora A kinases are believed to be involved in spindle formation and become localised on the centrosome during the early G2 phase where they phosphorylate spindle-associated proteins (Prigent et al., Cell, 114: 531-535 (2003). Hirota et al, Cell, 114:585-598, (2003) found that cells depleted of Aurora A protein kinase were unable to enter mitosis. Furthermore, it has been found (Adams, 2001) that mutation or disruption of the Aurora A gene in various species leads to mitotic abnormalities, including centrosome separation and maturation defects, spindle aberrations and chromosome segregation defects.

The Aurora kinases are generally expressed at a low level in the majority of normal tissues, the exceptions being tissues with a high proportion of dividing cells such as the thymus and testis. However, elevated levels of Aurora kinases have been found in many human cancers (Giet et al., J. Cell. Sci. 112: 3591-361, (1999) and Katayama (2003). Furthermore, Aurora A kinase maps to the chromosome 20q13 region that has frequently been found to be amplified in many human cancers.

Thus, for example, significant Aurora A over-expression has been detected in human breast, ovarian and pancreatic cancers (see Zhou et al., Nat. Genet. 20: 189-193, (1998), Tanaka et al., Cancer Res., 59: 2041-2044, (1999) and Han et al., cancer Res., 62: 2890-2896, (2002).

Moreover, Isola, American Journal of Pathology 147, 905-911 (1995) has reported that amplification of the Aurora A locus (20q13) correlates with poor prognosis for patients with node-negative breast cancer.

Amplification and/or over-expression of Aurora-A is observed in human bladder cancers and amplification of Aurora-A is associated with aneuploidy and aggressive clinical behaviour, see Sen et al., J. Natl. Cancer Inst, 94: 1320-1329 (2002).

Elevated expression of Aurora-A has been detected in over 50% of colorectal cancers, (see Bischoff et al., EMBO J., 17: 3052-3065, (1998) and Takahashi et al., Jpn. J. Cancer Res., 91: 1007-1014 (2000)) ovarian cancers (see Gritsko et al. Clin. Cancer Res., 9: 1420-1426 (2003), and gastric tumours Sakakura et al., British Journal of Cancer, 84: 824-831 (2001).

Tanaka et al. Cancer Research, 59: 2041-2044 (1999) found evidence of over-expression of Aurora A in 94% of invasive duct adenocarcinomas of the breast.

High levels of Aurora A kinase have also been found in renal, cervical, neuroblastoma, melanoma, lymphoma, pancreatic and prostate tumour cell lines Bischoff et al. (1998), EMBO J., 17: 3052-3065 (1998); Kimura et al. J. Biol. Chem., 274: 7334-7340 (1999); Zhou et al., Nature Genetics, 20: 189-193 (1998); Li et al., Clin Cancer Res. 9 (3): 991-7 (2003)].

Aurora-B is highly expressed in multiple human tumour cell lines, including leukemic cells [Katayama et al., Gene 244: 1-7)]. Levels of this enzyme increase as a function of Duke's stage in primary colorectal cancers [Katayama et al., J. Natl Cancer Inst., 91: 1160-1162 (1999)].

High levels of Aurora-3 (Aurora-C) have been detected in several tumour cell lines, even though this kinase tends to be restricted to germ cells in normal tissues (see Kimura et al. Journal of Biological Chemistry, 274: 7334-7340 (1999)). Over-expression of Aurora-3 in approximately 50% of colorectal cancers has also been reported in the article by Takahashi et al., Jpn J. Cancer Res. 91: 1007-1014 (2001)].

Other reports of the role of Aurora kinases in proliferative disorders may be found in Bischoff et al., Trends in Cell Biology 9: 454-459 (1999); Giet et al. Journal of Cell Science, 112: 3591-3601 (1999) and Dutertre, et al. Oncogene, 21: 6175-6183 (2002).

Royce et al report that the expression of the Aurora 2 gene (known as STK15 or BTAK) has been noted in approximately one-fourth of primary breast tumours. (Royce M E, Xia W, Sahin A A, Katayama H, Johnston D A, Hortobagyi G, Sen S, Hung M C; STK15/Aurora-A expression in primary breast tumours is correlated with nuclear grade but not with prognosis; Cancer. 2004 Jan. 1; 100(1):12-9).

Endometrial carcinoma (EC) comprises at least two types of cancer: endometrioid carcinomas (EECs) are estrogen-related tumours, which are frequently euploid and have a good prognosis. Nonendometrioid carcinomas (NEECs; serous and clear cell forms) are not estrogen related, are frequently aneuploid, and are clinically aggressive. It has also been found that Aurora was amplified in 55.5% of NEECs but not in any EECs (P<or =0.001) (Moreno-Bueno G, Sanchez-Estevez C, Cassia R, Rodriguez-Perales S, Diaz-Uriarte R, Dominguez O, Hardisson D, Andujar M, Prat J, Matias-Guiu X, Cigudosa J C, Palacios J. Cancer Res. 2003 Sep. 15; 63(18):5697-702).

Reichardt et al (Oncol Rep. 2003 September-October; 10(5):1275-9) have reported that quantitative DNA analysis by PCR to search for Aurora amplification in gliomas revealed that five out of 16 tumours (31%) of different WHO grade (1× grade II, 1× grade III, 3× grade IV) showed DNA amplification of the Aurora 2 gene. It was hypothesized that amplification of the Aurora 2 gene may be a non-random genetic alteration in human gliomas playing a role in the genetic pathways of tumourigenesis.

Results by Hamada et al (Br. J. Haematol. 2003 May; 121(3):439-47) also suggest that Aurora 2 is an effective candidate to indicate not only disease activity but also tumourigenesis of non-Hodgkin's lymphoma. Retardation of tumour cell growth resulting from the restriction of this gene's functions could be a therapeutic approach for non-Hodgkin's lymphoma.

In a study by Gritsko et al (Clin Cancer Res. 2003 April; 9(4):1420-6)), the kinase activity and protein levels of Aurora A were examined in 92 patients with primary ovarian tumours. In vitro kinase analyses revealed elevated Aurora A kinase activity in 44 cases (48%). Increased Aurora A protein levels were detected in 52 (57%) specimens. High protein levels of Aurora A correlated well with elevated kinase activity.

Results obtained by Li et al (Clin. Cancer Res. 2003 March; 9(3):991-7) showed that the Aurora A gene is overexpressed in pancreatic tumours and carcinoma cell lines and suggest that overexpression of Aurora A may play a role in pancreatic carcinogenesis.

Similarly, it has been shown that Aurora A gene amplification and associated increased expression of the mitotic kinase it encodes are associated with aneuploidy and aggressive clinical behaviour in human bladder cancer. (J. Natl. Cancer Inst. 2002 Sep. 4; 94(17):1320-9).

Investigation by several groups (Dutertre S, Prigent C.,Aurora-A overexpression leads to override of the microtubule-kinetochore attachment checkpoint; Mol. Interv. 2003 May; 3(3):127-30 and Anand S, Penrhyn-Lowe S, Venkitaraman A R., Aurora-A amplification overrides the mitotic spindle assembly checkpoint, inducing resistance to Taxol, Cancer Cell. 2003 January;3(1):51-62) suggests that overexpression of Aurora kinase activity is associated with resistance to some current cancer therapies. For example overexpression of Aurora A in mouse embryo fibroblasts can reduce the sensitivity of these cells to the cytotoxic effects of taxane derivatives. Therefore Aurora kinase inhibitors may find particular use in patients who have developed reistance to existing therapies.

On the basis of work carried out to date, it is apparent that inhibition of Aurora kinases, particularly Aurora kinase A and Aurora kinase B, will prove an effective means of arresting tumour development.

Harrington et al (Nat. Med. 2004 March; 10(3):262-7) have demonstrated that an inhibitor of the Aurora kinases suppresses tumour growth and induces tumour regression in vivo. In the study, the Aurora kinase inhibitor blocked cancer cell proliferation, and also triggered cell death in a range of cancer cell lines including leukaemic, colorectal and breast cell lines. In addition, it has shown potential for the treatment of leukemia by inducing apoptosis in leukemia cells. VX-680 potently killed treatment-refractory primary Acute Myelogenous Leukemia (AML) cells from patients (Andrews, Oncogene, 2005, 24, 5005-5015).

Recent reports indicate that Aurora kinases A and B are overexpressed in human leukaemia cells and that a small molecule Aurora kinase inhibitor is active against the growth of primary acute myeloid cells in vitro (Harrington et al, 2004). Moreover it has recently been reported that the product of the PML gene that is disrupted in acute promyelocytic leukaemia by a t(15:17) translocation (PML3), interacts with Aurora A and suppresses its kinase activity. Further evidence is emerging that PML is a tumor suppressor and that its disruption is not limited to leukaemias but may also be common in lymphomas and some solid tumors (Xu et al, Molecular Cell 17: 721-732, 2005).

Cancers which may be particularly amenable to Aurora inhibitors include breast, bladder, colorectal, pancreatic, ovarian, non-Hodgkin's lymphoma, gliomas and nonendometrioid endometrial carcinomas. Leukemias particularly amenable to Aurora inhibitors include Acute Myelogenous Leukemia (AML), chronic myelogenous leukaemia (CML), B-cell lymphoma (Mantle cell), and Acute Lymphoblastic Leukemia (ALL). Further leukemias include acute promyelocytic leukaemia.

### C-Abl

A chromosomal translocation event which fuses a BCR encoded sequence to a truncated c-abl gene greatly increases c-abl's tyrosine kinase activity and is the transforming agent in 95% of all Chronic Myeloid Leukaemia (CML) patients. This translocation occurs between chromosomes 9 and 22 resulting in an altered chromosome 22, the Philadelphia (Ph+) chromosome, which can be distinguished by cytogenetic methods. The fusion of BCR and Abl gene sequences results in the oligomerization of the Bcr-Abl gene product, increased trans-autophosphorylation and activation. An auto-inhibitory domain of the c-abl protein is also deleted as a result of the gene fusion. The sub-cellular localization of c-abl is also affected as a result of the gene fusion. The oncogenic effects of Bcr-Abl are complicated, but are believed to involve induction of G1 to S phase transition through activation of Ras, Erk and Jun pathways. Bcr-Abl also affects cell survival through the PI3K/Akt pathway. The oncogenic effects of Bcr-Abl have been demonstrated in animal models which indicate that the Bcr-Abl protein is able to establish CML symptoms in mice.

CML is a fatal disease, which progresses through three stages: chronic phase, accelerated phase, and blast crisis. CML is characterized in early stages by the proliferation of terminally differentiated neutrophils. As the disease progresses an excessive number of myeloid or lymphoid progenitor cells are produced. This chronic phase of the disease may last for years before advancing to an acute blast stage, characterized by multiple additional genetic mutations. CML primarily affects adults who have a mean survival of 5 years after the disease is manifested. CML has been successfully treated in early phases by an ATP competitive inhibitor of c-abl, imatinib (Gleevec). A 95% remission rate was demonstrated for this drug in a phase I clinical trial. Durable responses to imatinib have been observed for CML patients in the chronic phase, however remissions in blast phase only last 2-6 months. Unfortunately the development of acquired resistance to imatinib in CML patients is estimated to be as high as 15%/year.

Kinase domain mutations in BCR-ABL represent the most common mechanism of acquired resistance to imatinib, occurring in 50%-90% of cases. The most common cause of imatinib resistance is through the development of point mutations in the c-abl kinase domain, which directly or indirectly affect imatinib binding. More than 25 distinct Abl kinase domain mutations have been identified in imatinib treated CML patients and are associated with clinical resistance to imatinib (Hematology Shah 2005 (1): 183). These mutations have varying degrees of sensitivity to imatinib. Imatinib has been shown to bind to the ABL kinase domain in the inactive, or closed, conformation and to induce a variety of conformational changes to the protein upon binding. While some resistance-associated mutations occur at amino acid positions implicated in directly contacting imatinib, the majority are felt to prevent the kinase domain from adopting the specific conformation to which imatinib binds. Studies have shown that some mutations confer only a moderate degree of resistance, and as a result, dose escalation is predicted to recapture responses in some cases. Co-administration of second generation BCR-ABL inhibitors (e.g. BMS354825, AMN-107) have been shown to effectively inhibit many imatinib resistant c-abl mutants. However there are no drugs in the clinic which have been shown to be efficacious against the most imatinib resistant c-abl mutation, T3151.

### FMS-Like Tyrosine-kinase 3 (FLT3)

FLT3 (short for fms-like tyrosine-kinase 3) is a class III receptor tyrosine kinase (RTK) structurally related to the receptors for platelet derived growth factor (PDGF), colony stimulating factor 1 (CSF1), and KIT ligand (KL). FLT3 contains an intracellular tyrosine kinase domain split in two by a specific hydrophilic insertion termed a kinase insert.

FLT3 and its specific ligand FLT3-ligand (FL) plays a role in regulation of haematopoietic progenitor cells and is expressed on haematopoietic cells including CD34-positive bone marrow cells, corresponding to multipotential, myeloid and B-lymphoid progenitor cells, and on monocytic cells.

Activating mutations of FLT3 are one of the most frequent mutations observed in acute myeloid leukaemia. The most frequent mutations are referred to as length mutations (LM) or internal tandem duplications (ITD) and consist of a duplicated sequence or insert belonging to exon 11 and sometimes involving intron 11 and exon 12.

Internal tandem duplications and/or insertions and, rarely, deletions in the FLT3-gene are implicated in 20-25% of all acute myeloid leukemias (AML) and 5-10% myelodysplastic syndromes (MDS) and some cases with acute lymphoblastic leukemia (ALL).

The mutation of the FLT3 protein causes constitutive activation of the tyrosine kinase activity due to disruption of a negative regulatory domain. This activation results in stimulation of several growth factor dependent pathways including the raf-MEK-ERK pathway and contributes to the growth and survival of the leukaemic cells. Thus inhibition of the kinase activity of FLT3 would be an effective treatment for diseases such as those described above which are dependent upon the FLT3 activity.

### 3-Phosphoinositide-Dependent Protein Kinase-1 (PDK1)

The 3-phosphoinositide-dependent protein kinase-1 (PDK1) plays a key role in regulating the activity of a number of kinases belonging to the AGC subfamily of protein kinases (Alessi, D. et al., Biochem. Soc. Trans, 29, p1-14, 2001). These include protein kinase B (PKB/AKT), p70 ribosomal S6 kinase (S6K) (Avruch, J. et al., Prog. Mol. Subcell. Biol., 2001, p115-154, 2001) and p90 ribosomal S6 kinase (Frodin, M. et al., EMBO J., 19, p2924-2934, 2000). Kinase activity of serum and glucocordicoid regulated kinase (SGK) can also be phosphorylated and activated by PDK-1. Other potential substrates include protein kinase C, cAMP-dependent protein kinase (PKA), PRK1 and Protein kinase G.

PDK1 mediated signalling is activated in response to insulin and growth factors and as a consequence of attachment of the cell to the extracellular matrix (integrin signalling). Once activated these enzymes mediate many diverse cellular events by phosphorylating key regulatory proteins that play important roles controlling processes such as cell survival, growth, proliferation and glucose regulation (Lawlor, M. A. et al., J. Cell Sci., 114, p2903-2910, 2001), (Lawlor, M. A. et al., EMBO J., 21, p3728-3738, 2002). PDK-1 inhibitors therefore may provide novel therapeutic treatment for diseases such as diabetes and cancer.

PDK1 is a 556 amino acid protein, with an N-terminal catalytic domain and a C-terminal pleckstrin homology (PH) domain, which activates its substrates by phosphorylating these kinases at their activation loop (Belham, C. et al., Curr. Biol., 9, pR93-96, 1999). Many human cancers including prostate and NSCL have elevated PDK1 signalling pathway function resulting from a number of distinct genetic events such as PTEN mutations or over-expression of certain key regulatory proteins [(Graff, J. R., Expert Opin. Ther. Targets, 6, p103-13, 2002), (Brognard, J., et al., Cancer Res., 61 p3986-97, 2001)]. Inhibition of PDK1 as a potential mechanism to treat cancer was demonstrated by transfection of a PTEN negative human cancer cell line (IJ87MG) with antisense oligonucleotides directed against PDK1. The resulting decrease in PDK1 protein levels led to a reduction in cellular proliferation and survival (Flynn, P., et al., Curr. Biol., 10, p1439-42, 2000). Therefore inhibition of PDK-1 could offer an attractive target for cancer therapy.

PDK-1-mediated phosphorylation of PKB/AKT, which is largely present in an inactive form in unstimulated cells, converts the enzyme to a catalytically active form. This occurs through the phosphorylation of the activation loop domain of AKT at threonine-309 in AKT2 and theonine-308 in AKT1. Although AKT displays low, basal levels of activation in normal, unstimulated cells, AKT often becomes constitutively activated in tumor cells. This occurs through the up-regulation of a variety of different signalling molecules or the presence of oncogenenic mutations commonly found in cancer cells that can promote the activation of AKT, such as PI-3 kinase, growth factor receptors (e.g., EGFR family members), Ras, Src, and BCR-ABL activation. Loss of the tumor suppressor PTEN is another means of greatly increasing AKT activity in cancer cells (Besson, A. et al., Eur. J. Biochem. (1999), Vol. 263, No. 3, pp. 605-611). PTEN mutation or down regulation of PTEN protein is found in a large number of tumors and cancer cell lines. PTEN is a phosphatase that removes the D-3 phosphate from the products of PI-3 kinase such as phosphatidylinositol 3,4,5-trisphosphate and phosphatidylinositol-3,4-bisphosphate (Myers, M. P. et al., Proc. Natl. Acad. Sci. USA (1998), Vol. 95, No. 23, pp. 13513-13518; Stambolic, V. et al., Cell (1998), Vol. 95 p29-39). Loss of PTEN, therefore has the effect of increasing products of PI-3 kinase and promoting constitutive activation of AKT. Cancers with highly upregulated levels of AKT may be especially sensitive to the effects of PDK-1/AKT pathway inhibitors.

Therefore PDK1 is a critical mediator of the PI3K signalling pathway, which regulates a multitude of cellular function including growth, proliferation and survival. Consequently, inhibition of this pathway affects many defining requirements for cancer progression, so that a PDK1 inhibitor has an effect on the growth of a very wide range of human cancers.

### Vascular Endothelial Growth Factor (VEGFR)

Chronic proliferative diseases are often accompanied by profound angiogenesis, which can contribute to or maintain an inflammatory and/or proliferative state, or which leads to tissue destruction through the invasive proliferation of blood vessels. (Folkman, EXS, 79, 1-81 (1997); Folkman, Nature Medicine, 1, 27-31 (1995); Folkman and Shing, J. Biol. Chem., 267, 10931 (1992)).

Angiogenesis is generally used to describe the development of new or replacement blood vessels, or neovascularisation. It is a necessary and physiological normal process by which vasculature is established in the embryo. Angiogenesis does not occur, in general, in most normal adult tissues, exceptions being sites of ovulation, menses and wound healing. Many diseases, however, are characterized by persistent and unregulated angiogenesis. For instance, in arthritis, new capillary blood vessels invade the joint and destroy cartilage (Colville-Nash and Scott, Ann. Rhum. Dis., 51, 919 (1992)). In diabetes (and in many different eye diseases), new vessels invade the macula or retina or other ocular structures, and may cause blindness (Brooks, et al., Cell, 79, 1157 (1994)). The process of atherosclerosis has been linked to angiogenesis (Kahlon, et al., Can. J. Cardiol., 8, 60 (1992)). Tumor growth and metastasis have been found to be angiogenesis-dependent (Folkman, Cancer Biol, 3, 65 (1992); Denekamp, Br. J. Rad., 66, 181 (1993); Fidler and Ellis, Cell, 79, 185 (1994)).

The recognition of the involvement of angiogenesis in major diseases has been accompanied by research to identify and develop inhibitors of angiogenesis. These inhibitors are generally classified in response to discrete targets in the angiogenesis cascade, such as activation of endothelial cells by an angiogenic signal; synthesis and release of degradative enzymes; endothelial cell migration; proliferation of endothelial cells; and formation of capillary tubules. Therefore, angiogenesis occurs in many stages and attempts are underway to discover and develop compounds that work to block angiogenesis at these various stages.

There are publications that teach that inhibitors of angiogenesis, working by diverse mechanisms, are beneficial in diseases such as cancer and metastasis (O'Reilly, et al., Cell, 79, 315 (1994); Ingber, et al., Nature, 348, 555 (1990)), ocular diseases (Friedlander, et al., Science, 270, 1500 (1995)), arthritis (Peacock, et al., J. Exp. Med., 175, 1135 (1992); Peacock et al., Cell. Immun., 160, 178 (1995)) and hemangioma (Taraboletti, et al., J. Natl. Cancer Inst., 87, 293 (1995)).

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity that leads to phosphorylation of tyrosine residues on both the receptor and other intracellular proteins, leading to a variety of cellular responses. To date, at least nineteen distinct RTK subfamilies, defined by amino acid sequence homology, have been identified.

Vascular endothelial growth factor (VEGF), a polypeptide, is mitogenic for endothelial cells in vitro and stimulates angiogenic responses in vivo. VEGF has also been linked to inappropriate angiogenesis (Pinedo, H. M., et al., The Oncologist, 5(90001), 1-2 (2000)). VEGFR(s) are protein tyrosine kinases (PTKs). PTKs catalyze the phosphorylation of specific tyrosine residues in proteins involved in cell function thus regulating cell growth, survival and differentiation. (Wilks, A. F., Progress in Growth Factor Research, 2, 97-111 (1990); Courtneidge, S. A., Dev. Supp.I, 57-64 (1993); Cooper, J. A., Semin. Cell Biol., 5(6), 377-387 (1994); Paulson, R. F., Semin. Immunol., 7(4), 267-277 (1995); Chan, A. C., Curr. Opin. Immunol., 8(3), 394-401 (1996)).

Three PTK receptors for VEGF have been identified: VEGFR-1 (Flt-1); VEGFR-2 (Flk-1 or KDR) and VEGFR-3 (Flt-4). These receptors are involved in angiogenesis and participate in signal transduction (Mustonen, T., et al., J. Cell Biol., 129, 895-898 (1995)).

Of particular interest is VEGFR-2, which is a transmembrane receptor PTK expressed primarily in endothelial cells. Activation of VEGFR-2 by VEGF is a critical step in the signal transduction pathway that initiates tumour angiogenesis. VEGF expression may be constitutive to tumour cells and can also be upregulated in response to certain stimuli. One such stimuli is hypoxia, where VEGF expression is upregulated in both tumour and associated host tissues. The VEGF ligand activates VEGFR-2 by binding with its extracellular VEGF binding site. This leads to receptor dimerization of VEGFRs and autophosphorylation of tyrosine residues at the intracellular kinase domain of VEGFR-2. The kinase domain operates to transfer a phosphate from ATP to the tyrosine residues, thus providing binding sites for signalling proteins downstream of VEGFR-2 leading ultimately to initiation of angiogenesis (McMahon, G., The Oncologist, 5(90001), 3-10 (2000)).

Inhibition at the kinase domain binding site of VEGFR-2 would block phosphorylation of tyrosine residues and serve to disrupt initiation of angiogenesis.

### FGFR

The fibroblast growth factor (FGF) family of protein tyrosine kinase (PTK) receptors regulates a diverse array of physiologic functions including mitogenesis, wound healing, cell differentiation and angiogenesis, and development. Both normal and malignant cell growth as well as proliferation are affected by changes in local concentration of these extracellular signaling molecules, which act as autocrine as well as paracrine factors. Autocrine FGF signaling may be particularly important in the progression of steroid hormone-dependent cancers and to a hormone independent state (Powers, et al., Endocr. Relat. Cancer, 7, 165-197 (2000)).

FGFs and their receptors are expressed at increased levels in several tissues and cell lines and overexpression is believed to contribute to the malignant phenotype. Furthermore, a number of oncogenes are homologues of genes encoding growth factor receptors, and there is a potential for aberrant activation of FGF-dependent signaling in human pancreatic cancer (Ozawa, et al., Teratog. Carcinog. Mutagen., 21, 27-44 (2001)).

The two prototypic members are acidic fibroblast growth factor (aFGF or FGF1) and basic fibroblast growth factor (bFGF or FGF2), and to date, at least twenty distinct FGF family members have been identified. The cellular response to FGFs is transmitted via four types of high affinity transmembrane protein tyrosine-kinase fibroblast growth factor receptors numbered 1 to 4 (FGFR1 to FGFR4). Upon ligand binding, the receptors dimerize and auto- or trans-phosphorylate specific cytoplasmic tyrosine residues to transmit an intracellular signal that ultimately regulates nuclear transcription factor effectors.

Disruption of the FGFR1 pathway should affect tumor cell proliferation since this kinase is activated in many tumor types in addition to proliferating endothelial cells. The over-expression and activation of FGFR1 in tumor-associated vasculature has suggested a role for these molecules in tumor angiogenesis.

Fibroblast growth factor receptor 2 has high affinity for the acidic and/or basic fibroblast growth factors, as well as the keratinocyte growth factor ligands. Fibroblast growth factor receptor 2 also propagates the potent osteogenic effects of FGFs during osteoblast growth and differentiation. Mutations in fibroblast growth factor receptor 2, leading to complex functional alterations, were shown to induce abnormal ossification of cranial sutures(craniosynostosis), implying a major role of FGFR signaling in intramembranous bone formation. For example, in Apert (AP) syndrome, characterized by premature cranial suture ossification, most cases are associated with point mutations engendering gain-of-function in fibroblast growth factor receptor 2 (Lemonnier, et al., J. Bone Miner. Res., 16, 832-845 (2001)).

Several severe abnormalities in human skeletal development, including Apert, Crouzon, Jackson-Weiss, Beare-Stevenson cutis gyrata, and Pfeiffer syndromes are associated with the occurrence of mutations in fibroblast growth factor receptor 2. Most, if not all, cases of Pfeiffer Syndrome (PS) are also caused by de novo mutation of the fibroblast growth factor receptor 2 gene (Meyers, et al., Am. J. Hum. Genet., 58, 491-498 (1996); Plomp, et al., Am. J. Med. Genet., 75, 245-251 (1998)), and it was recently shown that mutations in fibroblast growth factor receptor 2 break one of the cardinal rules governing ligand specificity. Namely, two mutant splice forms of fibroblast growth factor receptor, FGFR2c and FGFR2b, have acquired the ability to bind to and be activated by atypical FGF ligands. This loss of ligand specificity leads to aberrant signaling and suggests that the severe phenotypes of these disease syndromes result from ectopic ligand-dependent activation of fibroblast growth factor receptor 2 (Yu, et al., Proc. Natl. Acad. Sci. U.S.A., 97, 14536-14541 (2000)).

Genetic aberrations of the FGFR3 receptor tyrosine kinase such as chromosomal translocations or point mutations result in ectopically expressed or deregulated, constitutively active, FGFR3 receptors. Such abnormalities are linked to a subset of multiple myelomas and in bladder, hepatocellular, oral squamous cell carcinoma and cervical carcinomas (Powers, C. J., et al., Endocr. Rel. Cancer, 7, 165 (2000), Qiu, W., et. al., World Journal Gastroenterol, 11 (34) 2005). Accordingly, FGFR3 inhibitors would be useful in the treatment of multiple myeloma, bladder and cervical carcinomas.

As such, the compounds are useful in providing a means of preventing the growth or inducing apoptosis of neoplasias and in tumours, particularly by inhibiting angiogenesis. The compounds are useful in treating or preventing proliferative disorders such as cancers. In particular tumours with activating mutants of receptor tyrosine kinases or upregulation of receptor tyrosine kinases may be particularly sensitive to the inhibitors. Patients with activating mutants of any of the isoforms of the specific RTKs discussed herein may also find treatment with RTK inhibitors particularly beneficial.

Over expression of FGFR4 has been linked to poor prognosis in both prostate and thyroid carcinomas (Ezzat, S., et al. The Journal of Clinical Investigation, 109, 1 (2002), Wang et al. Clinical Cancer Research, 10 (2004)). In addition a germline polymorphism (Gly388Arg) is associated with increased incidence of lung, breast, colon and prostate cancers (Wang et al. Clinical Cancer Research, 10 (2004)).

### RET

The Ret proto-oncogene encodes a receptor tyrosine kinase that is expressed during development in a variety of tissues, including the peripheral and central nervous systems and the kidney. The abnormalities present in ret null mice suggest that Ret is critical for the migration and innervation of enteric neurons to the hindgut, and for proliferation and branching of the ureteric bud epithelium during kidney development (Nature 367, 380-383, 1994).

Mutations in the RET receptor tyrosine kinase provides a classic example of phenotypic heterogeneity in a variety of diseases. Gain-of-function mutations of RET are associated with human cancer and in particular cause inherited and non-inherited thyroid cancer. Gene rearrangements juxtaposing the tyrosine kinase domain of RET to heterologous gene partners have been found in sporadic papillary carcinomas of the thyroid (PTC). These rearrangements generate chimeric RET/PTC oncogenes. In germline cancers, point mutations of RET are responsible for multiple endocrine neoplasia type 2 (MEN 2A and 2B) and familial medullary thyroid carcinoma (FMTC). Both MEN 2 mutations and PTC gene rearrangements potentiate the intrinsic tyrosine kinase activity of RET and, ultimately, activate targets downstream of RET.

Thus somatic gene rearrangements of RET have been found in papillary thyroid carcinoma (PTC) and germline point mutations in multiple endocrine neoplasia (MEN) types 2A and 2B and familial medullary thyroid carcinoma (FMTC). Conversely, loss-of-function mutations are responsible for the development of Hirschsprung's disease, a congenital malformation of the enteric nervous system. (Naoya Asai et al, Pathology International, Volume 56 Page 164, April 2006)

### SRC

The Src family kinases (SFK) comprises nine members of which three (Src, Fyn Yes) are ubiquitously expressed. Src itself is implicated in the pathogenesis of human malignancies. Activated mutants of c-Src can transform human cells in culture and Src protein expression and/or activity is increased in epithelial cancers. In colon cancer there is frequent elevation of Src activity compared to adjacent normal mucosa. Furthermore the Src activation is often elevated in metastases compared to the primary tumour implying a possible role for the protein in invasion and metastasis. Moreover Src expression is strongly correlated with disease progression. Similarly Src expression and activation are also elevated in breast, pancreatic, oesophageal, ovarian, lung, head and neck and gastric cancers compared to normal tissues.

### EGFR and PDGFR

A malignant tumour is the product of uncontrolled cell proliferation. Cell growth is controlled by a delicate balance between growth-promoting and growth-inhibiting factors. In normal tissue the production and activity of these factors results in differentiated cells growing in a controlled and regulated manner that maintains the normal integrity and functioning of the organ. The malignant cell has evaded this control; the natural balance is disturbed (via a variety of mechanisms) and unregulated, aberrant cell growth occurs. One driver for growth is the epidermal growth factor (EGF), and the receptor for EGF (EGFR) has been implicated in the development and progression of a number of human solid tumours including those of the lung, breast, prostate, colon, ovary, head and neck. EGFR is a member of a family of four receptors, namely EGFR (HER1 or ErbB1), ErbB2 (HER2/neu), ErbB3 (HER3), and ErbB4 (HER4). These receptors are large proteins that reside in the cell membrane, each having a specific external ligand binding domain, a transmembrane domain and an internal domain which has tyrosine kinase enzyme activity. When EGF attaches to EGFR, it activates the tyrosine kinase, triggering reactions that cause the cells to grow and multiply. EGFR is found at abnormally high levels on the surface of many types of cancer cells, which may divide excessively in the presence of EGF. Inhibition of EGFR activity has therefore been a target for chemotherapeutic research in the treatment of cancer. Such inhibition can be effected by direct interference with the target EGFR on the cell surface, for example by the use of antibodies, or by inhibiting the subsequent tyrosine kinase activity.

Examples of agents which target EGFR tyrosine kinase activity include the tyrosine kinase inhibitors gefitinib and erlotinib. Gefitinib which has the chemical name 4-(3-chloro-4-fluoroanilino)-7-methoxy-6-(3-morpholinopropoxy)quinazoline, is used for the treatment of non-small-cell lung cancer, and is also under development for other solid tumours that over-express EGF receptors such as breast and colorectal cancer. Erlotinib, which has the chemical name N-(3-ethynyl-phenyl)-6,7-bis(2-methoxyethoxy)-4-quinazoline, has also been used for the treatment of non-small-cell lung cancer, and is being developed for the treatment of various other solid tumours such as pancreatic cancer.

Another growth factor of importance in tumour development is the platelet-derived growth factor (PDGF) that comprises a family of peptide growth factors that signal through cell surface tyrosine kinase receptors (PDGFR) and stimulate various cellular functions including growth, proliferation, and differentiation. PDGF expression has been demonstrated in a number of different solid tumours including glioblastomas and prostate carcinomas. The tyrosine kinase inhibitor imatinib mesylate, which has the chemical name 4-[(4-methyl-1-piperazinyl)methyl]-N-[4-methyl-3-[[4-(3-pyridinyl)-2-ylpyridinyl]amino]-phenyl]benzamide methanesulfonate, blocks activity of the Bcr-Abl oncoprotein and the cell surface tyrosine kinase receptor c-Kit, and as such is approved for the treatment of chronic myeloid leukemia and gastrointestinal stromal tumours. Imatinib mesylate is also a potent inhibitor of PDGFR kinase and is currently being evaluated for the treatment of chronic myelomonocytic leukemia and glioblastoma multiforme, based upon evidence in these diseases of activating mutations in PDGFR. In addition, sorafenib (BAY 43-9006) which has the chemical name 4-(4-(3-(4-chloro-3 (trifluoromethyl)phenyl)ureido)phenoxy)-N-2-methylpyridine-2-carboxamide, targets both the Raf signalling pathway to inhibit cell proliferation and the VEGFR/PDGFR signalling cascades to inhibit tumour angiogenesis. Sorafenib is being investigated for the treatment of a number of cancers including liver and kidney cancer.

### Compounds of Formula (0)

A wide variety of compounds of the formula (0) find application in the combinations of the invention, as described in detail below. The compounds of formula (0) for use in the combinations of the invention correspond to those of formula (0) in WO 2005/012256 (the contents of which are incorporated herein by reference) and various possible substituents, sub-groups, embodiments and examples thereof as therein defined. The content of WO 2005/012256 describing the various possible substituents, subgroups, embodiments and examples of compounds of formula (0) is hereby incorporated herein by reference.

The formula (0) of WO 2005/012256 (PCT/GB2004/003179) is herein also referred to as formula (0) and references to formula (0) herein are to be interpreted accordingly.

Compounds of formula (I′″)

A wide variety of compounds of the formula (I′″) find application in the combinations of the invention, as described in detail below. The compounds of formula (I′″) for use in the combinations of the invention correspond to those of formula (I) described in WO 2006/077416 and include the various possible substituents, sub-groups, embodiments and examples thereof as therein defined. The content of WO 2006/077416 describing the various possible substituents, subgroups, embodiments and examples of compounds of formula (I) (i.e. formula (I′″) herein) is hereby incorporated herein by reference.

In this specification in general (and this section in particular), unless the context indicates otherwise, references to a compound of formula (I′″) includes all subgroups of formula (I′″) as defined herein and the term ‘subgroups’ includes all preferences, embodiments, examples and particular compounds defined herein. Any references to formula (I′″) herein shall also be taken to refer to and any sub-group of compounds within formula (I′″) and any preferences and examples thereof unless the context requires otherwise.

The formula (I) of WO 2006/077416 is herein referred to as formula (I′″) and references to formula (I′″) herein are to be interpreted accordingly.

### Auxiliary Compounds

A wide variety of optional auxiliary compounds may be further combined with the combinations of the invention, as described in detail below. The optional auxiliary compounds may be anti-cancer agents.

It is an object of the invention to provide therapeutic combinations comprising (or consisting essentially on one or more compounds of the formula (0) and one or more compounds of the formula (I′″) which inhibit or modulate (in particular inhibit) the activity of cyclin dependent kinases (CDK) and/or glycogen synthase kinase (e.g. GSK-3) and optionally one or more auxilliary compounds. Such combinations may have an advantageous efficacious effect against tumour cell growth, in comparison with the respective effects shown by the individual components of the combination.

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulphinyl- and sulphonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group.

WO 01/98290 (Pharmacia & Upjohn) discloses a class of 3-aminocarbonyl-2-carboxamido thiophene derivatives as protein kinase inhibitors.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 00/39108 and WO 02/00651 (both to Du Pont Pharmaceuticals) describe heterocyclic compounds that are inhibitors of trypsin-like serine protease enzymes, especially factor Xa and thrombin. The compounds are stated to be useful as anticoagulants or for the prevention of thromboembolic disorders.

US 2002/0091116 (Zhu et al.), WO 01/19798 and WO 01/64642 each disclose diverse groups of heterocyclic compounds as inhibitors of Factor Xa. Some 1-substituted pyrazole carboxamides are disclosed and exemplified.

U.S. Pat. No. 6,127,382, WO 01/70668, WO 00/68191, WO 97/48672, WO 97/19052 and WO 97/19062 (all to Allergan) each describe compounds having retinoid-like activity for use in the treatment of various hyperproliferative diseases including cancers.

WO 02/070510 (Bayer) describes a class of amino-dicarboxylic acid compounds for use in the treatment of cardiovascular diseases. Although pyrazoles are mentioned generically, there are no specific examples of pyrazoles in this document.

WO 97/03071 (Knoll AG) discloses a class of heterocyclyl-carboxamide derivatives for use in the treatment of central nervous system disorders. Pyrazoles are mentioned generally as examples of heterocyclic groups but no specific pyrazole compounds are disclosed or exemplified.

WO 97/40017 (Novo Nordisk) describes compounds that are modulators of protein tyrosine phosphatases.

WO 03/020217 (Univ. Connecticut) discloses a class of pyrazole 3-carboxamides as cannabinoid receptor modulators for treating neurological conditions. It is stated (page 15) that the compounds can be used in cancer chemotherapy but it is not made clear whether the compounds are active as anti-cancer agents or whether they are administered for other purposes.

WO 01/58869 (Bristol Myers Squibb) discloses cannabinoid receptor modulators that can be used inter alia to treat a variety of diseases. The main use envisaged is the treatment of respiratory diseases, although reference is made to the treatment of cancer.

WO 01/02385 (Aventis Crop Science) discloses 1-(quinoline-4-yl)-1H-pyrazole derivatives as fungicides. 1-Unsubstituted pyrazoles are disclosed as synthetic intermediates.

WO 2004/039795 (Fujisawa) discloses amides containing a 1-substituted pyrazole group as inhibitors of apolipoprotein B secretion. The compounds are stated to be useful in treating such conditions as hyperlipidemia.

WO 2004/000318 (Cellular Genomics) discloses various amino-substituted monocycles as kinase modulators. None of the exemplified compounds are pyrazoles.

WO 2005/012256 (Astex Technology Limited) discloses various compounds of formula (0) having activity as inhibitors of various kinases for use in the treatment of disease states and conditions such as cancer.

WO 2006/077424 (Astex Therapeutics Limited) discloses a combination of a cytotoxic compound or signalling inhibitor and a compound having the formula (0).

WO 2006/077426 (Astex Therapeutics Limited) discloses various compounds and salts of formula (0) having activity as inhibitors of cyclin dependent kinases, and glycogen synthase kinase-3.

WO 2006/077416 (Astex Therapeutics Limited) discloses various compounds of formula (I′″) having activity as inhibitors of cyclin dependent kinases, glycogen synthase kinase-3 and Aurora kinases.

### SUMMARY OF THE INVENTION

According to the present invention there is provided a combination comprising (or consisting essentially of):

(a) a compound of formula (0):

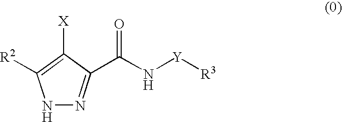

or salts or tautomers or N-oxides or solvates thereof;

wherein
X is a group R¹-A-NR⁴— or a 5- or 6-membered carbocyclic or heterocyclic ring;
A is a bond, SO₂, C═O, NR⁹(C═O) or O(C═O) wherein R⁹ is hydrogen or C₁₋₄ hydrocarbyl optionally substituted by hydroxy or C₁₋₄ alkoxy;
Y is a bond or an alkylene chain of 1, 2 or 3 carbon atoms in length;

R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);
R³ is selected from hydrogen and carbocyclic and heterocyclic groups having from 3 to 12 ring members; and
R⁴ is hydrogen or a C₁₋₄ hydrocarbyl group optionally substituted by halogen (e.g. fluorine), hydroxyl or C₁₋₄ alkoxy (e.g. methoxy); and

(b) a compound of formula (I′″):

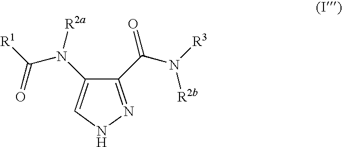

or salts, tautomers, solvates and N-oxides thereof;

wherein:

R¹ is 2,6-dichlorophenyl;

R^{2a} and R^{2b} are both hydrogen;

and R³ is a group:

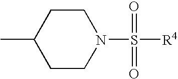

where R⁴ is C₁₋₄ alkyl.

The invention also provides the further combinations, uses, methods, compounds and processes as set out in the claims below.

### General Preferences and Definitions

In this specification, unless the context indicates otherwise, references to formula (0) include formulae (I), (I⁰), (Ia), (Ib), (II′), (III), (IV), (IVa), (Va), (Vb), (VIa), (VIb), (VII) or (VIII) as described in WO 2005/012256 and sub-groups, examples or embodiments of formulae (0), (I⁰), (Ia), (Ib), (II′), (III), (IV), (IVa), (Va), (Vb), (VIa), (VIb), (VII) or (VIII) as described in WO 2005/012256. Moreover, in this specification in general, unless the context indicates otherwise, references to a compound of formula (I′″) as described in WO 2006/077416 includes all subgroups of formula (I′″) as defined herein and the term ‘subgroups’ includes all preferences, embodiments, examples and particular compounds defined herein. Any references to formula (I′″) herein shall also be taken to refer to and any sub-group of compounds within formula (I′″) and any preferences and examples thereof unless the context requires otherwise.

As used herein, the term “modulation”, as applied to the activity of cyclin dependent kinase (CDK), Aurora kinases and glycogen synthase kinase (GSK, e.g. GSK-3) and/or any other kinase as described herein, is intended to define a change in the level of biological activity of the kinase(s). Thus, modulation encompasses physiological changes which effect an increase or decrease in the relevant kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of the kinase actitvty e.g. Aurora kinase, cyclin dependent kinase (CDK) and/or glycogen synthase kinase-3 (GSK-3) activity and/or activity of any other kinase described herein, or at the level of enzyme (e.g. cyclin dependent kinase (CDK) and/or glycogen synthase kinase-3 (GSK-3)) activity and/or any other kinase described herein (for example by allosteric mechanisms, competitive inhibition, active-site inactivation, perturbation of feedback inhibitory pathways etc.). Thus, modulation may imply elevated/suppressed expression or over- or under-expression of the kinase (e.g. cyclin dependent kinase (CDK) and/or glycogen synthase kinase-3 (GSK-3) or any other kinase described herein), including gene amplification (i.e. multiple gene copies) and/or increased or decreased expression by a transcriptional effect, as well as hyper- (or hypo-)activity and (de)activation of the protein kinase(s) (e.g. cyclin dependent kinase (CDK) and/or glycogen synthase kinase-3 (GSK-3) and/or any other protein kinase described herein) (including (de)activation) by mutation(s). The terms “modulated”, “modulating” and “modulate” are to be interpreted accordingly.

The term “upregulation of Aurora kinase” as used herein is defined as including elevated expression or over-expression of Aurora kinase, including gene amplification (i.e. multiple gene copies) and increased expression by a transcriptional effect, and hyperactivity and activation of Aurora kinase, including activation by mutations.

As used herein, the term “mediated”, as used e.g. in conjunction with a kinase as described herein (e.g. cyclin dependent kinases (CDK) and/or glycogen synthase kinase-3 (GSK-3) as described herein) (and applied for example to various physiological processes, diseases, states, conditions, therapies, treatments or interventions) is intended to operate limitatively so that the various processes, diseases, states, conditions, treatments and interventions to which the term is applied are those in which the kinase (e.g. cyclin dependent kinase (CDK) and/or glycogen synthase kinase-3 (GSK-3)) plays a biological role. In cases where the term is applied to a disease, state or condition, the biological role played by the kinase (e.g. cyclin dependent kinase (CDK) and/or glycogen synthase kinase-3 (GSK-3)) may be direct or indirect and may be necessary and/or sufficient for the manifestation of the symptoms of the disease, state or condition (or its aetiology or progression). Thus, kinase acitivty (e.g. cyclin dependent kinase (CDK) and/or glycogen synthase kinase-3 (GSK-3) activity) (and in particular aberrant levels of cyclin dependent kinase (CDK) and/or glycogen synthase kinase-3 (GSK-3)activity, e.g. cyclin dependent kinases (CDK) and/or glycogen synthase kinase-3 (GSK-3) over-expression) need not necessarily be the proximal cause of the disease, state or condition: rather, it is contemplated that the kinase mediated diseases, states or conditions (e.g. CDK- and/or GSK- (e.g. GSK-3-) mediated diseases, states or conditions) include those having multifactorial aetiologies and complex progressions in which the kinase (e.g. CDK and/or GSK-3) is only partially involved. In cases where the term is applied to treatment, prophylaxis or intervention (e.g. in the “CDK-mediated treatments” and “GSK-3-mediated prophylaxis” of the invention), the role played by the kinase (e.g. CDK and/or GSK-3) may be direct or indirect and may be necessary and/or sufficient for the operation of the treatment, prophylaxis or outcome of the intervention. Thus, a disease state or condition mediated by the kinase (e.g. cyclin dependent kinases (CDK) and/or glycogen synthase kinase-3 (GSK-3) and/or Aurora kinase and/or any other kinase as described herein) includes a disease state or condition which has arisen as a consequence of the development of resistance to any particular cancer drug or treatment (including in particular resistance to one or more of the auxiliary compounds described herein).

The term “intervention” is a term of art used herein to define any agency which effects a physiological change at any level. Thus, the intervention may comprise the induction or repression of any physiological process, event, biochemical pathway or cellular/biochemical event. The interventions of the invention typically effect (or contribute to) the therapy, treatment or prophylaxis of a disease or condition.

The combinations of the invention may produce a therapeutically efficacious effect relative to the therapeutic effect of the individual compounds when administered separately.

The term ‘efficacious’ includes advantageous effects such as additivity, synergism, reduced side effects, reduced toxicity, increased time to disease progression, increased time of survival, sensitization or resensitization of one agent to another, or improved response rate. Advantageously, an efficacious effect may allow for lower doses of each or either component to be administered to a patient, thereby decreasing the toxicity of chemotherapy, whilst producing and/or maintaining the same therapeutic effect.

A “synergistic” effect in the present context refers to a therapeutic effect produced by the combination which is larger than the sum of the therapeutic effects of the components of the combination when presented individually.

An “additive” effect in the present context refers to a therapeutic effect produced by the combination which is larger than the therapeutic effect of any of the components of the combination when presented individually.

The term “response rate” as used herein refers, in the case of a solid tumour, to the extent of reduction in the size of the tumour at a given time point, for example 12 weeks. Thus, for example, a 50% response rate means a reduction in tumour size of 50%. References herein to a “clinical response” refer to response rates of 50% or greater. A “partial response” is defined herein as being a response rate of less than 50%.

As used herein, the term “combination”, as applied to two or more compounds and/or agents (also referred to herein as the components), is intended tomay define material in which the two or more compounds/agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The combinations of the invention may produce a therapeutically efficacious effect relative to the therapeutic effect of the individual compounds/agents when administered separately.

The term “auxiliary compound” as used herein may define a compound which yields an efficacious combination (as herein defined) when combined with the combinations (or compounds of the formulae (0) and/or (I′″) as defined herein). The auxiliary compound may therefore act as an adjunct to the constituents of the combinations of the invention, or may otherwise contribute to the efficacy of the combination (for example, by producing a synergistic or additive effect or improving the response rate, as herein defined).

The term “checkpoint targeting agent” is used herein to define a functional class of agents which act to initiate activation of a cell cycle checkpoint or agents that interfere with or modulate the normal action of the cell cycle checkpoint in replicating tumour cells. The term therefore covers various agents (including, for example, platinum compounds, nucleoside analogues, CDK inhibitors, taxanes, epothilones, vinca alkaloids, polo-like kinase inhibitors, CHK kinase inhibitors, inhibitors of the BUB kinase family and kinesin inhibitors) that target the cell cycle checkpoint.

The targeting of the checkpoint may be mediated by any mechanism, including for example via stabilisation of spindle microtubules (so preventing spindle contraction, as mediated e.g. by various taxanes) or by prevention of spindle formation (as mediated e.g. by various vinca alkaloids) or by agents which cause damage to cellular components (e.g. DNA as caused by the platinum compounds or nucleoside analogues) thus causing activation of the checkpoint during cell proliferation. Thus, the checkpoint targeting agents typically cause chromosome mis-alignment or premature cytokinesis leading to death of the tumour cell. Checkpoint targeting agents may be identified by various techniques known to those skilled in the art for assessing cell cycle dynamics (e.g. for detecting multinucleation events), including for example flow cytometry, DNA staining, Western blot analysis for cell cycle markers (e.g. cyclins) and direct visualization by various microscopic techniques (e.g. focal microscopy).

General Preferences and Definitions for compounds of formula (0)

A wide variety of compounds of the formula (0) find application in the combinations of the invention, as described in detail below. The compounds of formula (0) for use in the combinations of the invention correspond to those of formula (0) in WO 2005/012256 (the contents of which are incorporated herein by reference) and various possible substituents, sub-groups, embodiments and examples thereof as therein defined. The content of WO 2005/012256 describing the various possible substituents, subgroups, embodiments and examples of compounds of formula (0) is hereby incorporated herein by reference.

The formula (0) of WO 2005/012256 (PCT/GB2004/003179) is herein also referred to as formula (0) and references to formula (0) herein are to be interpreted accordingly.

Thus, the compound of formula (0) for use in the combinations of the invention has the formula:

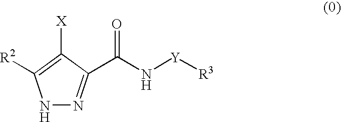

or salts or tautomers or N-oxides or solvates thereof;
wherein

X is a group R¹-A-NR⁴— or a 5- or 6-membered carbocyclic or heterocyclic ring;

A is a bond, SO₂, C═O, NR^{g}(C═O) or O(C═O) wherein R^{g} is hydrogen or C₁₋₄ hydrocarbyl optionally substituted by hydroxy or C₁₋₄ alkoxy;

Y is a bond or an alkylene chain of 1, 2 or 3 carbon atoms in length;

R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;

R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);

R³ is selected from hydrogen and carbocyclic and heterocyclic groups having from 3 to 12 ring members; and

R⁴ is hydrogen or a C₁₋₄ hydrocarbyl group optionally substituted by halogen (e.g. fluorine), hydroxyl or C₁₋₄ alkoxy (e.g. methoxy),

so corresponding to formula (0) in WO 2005/012256 (PCT/GB2004/003179) and including the various possible substituents, subgroups, embodiments and examples thereof as defined in WO 2005/012256 (PCT/GB2004/003179), so that the general preferences and definitions defined in WO 2005/012256 (PCT/GB2004/003179) shall apply to each of the moieties X, Y, R^{g}, R¹ to R⁴ and any substituent, moieties, sub-definition, sub-group or embodiment thereof, unless the context indicates otherwise.

In particular the carbocyclic and heterocyclic groups forming part of X, R¹ and R³ may be optionally substituted as defined in WO 2005/012256.

Particular compounds of the formula (0) are those defined in, for example, the compounds of formulae (I⁰), (I), (Ia), (Ib), (II), (III), (IV), (IVa), (Va), (Vb), (VIa), (VIb), (VII) or (VIII), and any sub-groups thereof in PCT/GB2004/003179 (WO 2005/012256), the compounds listed in PCT/GB2004/003179 (WO 2005/012256) and the compounds exemplified in the Examples section of PCT/GB2004/003179 (WO 2005/012256), the aforementioned sections of PCT/GB2004/003179 (WO 2005/012256) being hereby incorporated by reference.

A preferred sub-group of CDK inhibitor compounds within WO 2005/012256 is represented by the formula (Va):

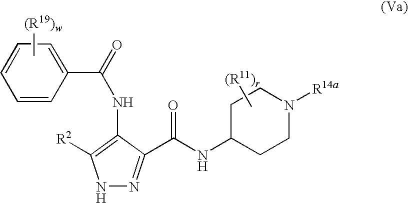

or salts or tautomers or N-oxides or solvates thereof;
wherein R^{14a} is selected from hydrogen, C₁₋₄ alkyl optionally substituted by fluoro (e.g. methyl, ethyl, n-propyl, i-propyl, butyl and 2,2,2-trifluoroethyl), cyclopropylmethyl, phenyl-C₁₋₂ alkyl (e.g. benzyl), C₁₋₄ alkoxycarbonyl (e.g. ethoxycarbonyl and t-butyloxycarbonyl), phenyl-C₁₋₂ alkoxycarbonyl (e.g. benzyloxycarbonyl), C₁₋₂-alkoxy-C₁₋₂ alkyl (e.g. methoxymethyl and methoxyethyl), and C₁₋₄ alkylsulphonyl (e.g. methanesulphonyl), wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl, most preferably hydrogen;
r is 0, 1 or 2;
R¹¹ is selected from hydrogen and C₁₋₃ alkyl (and more preferably is selected from hydrogen and methyl and most preferably is hydrogen); and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

Particular compounds within formula (VIb) of WO 2005/012256 include:
4-(2,6-difluoro-benzoylamino)-1H-pyrazole-3-carboxylic acid piperidin-4-ylamide;
4-(2,6-difluoro-benzoylamino)-1H-pyrazole-3-carboxylic acid (1-methyl-piperidin-4-yl)-amide;
4-(2,6-dichloro-benzoylamino)-1H-pyrazole-3-carboxylic acid piperidin-4-ylamide; and
4-(2-fluoro-6-methoxy-benzoylamino)-1H-pyrazole-3-carboxylic acid piperidin-4-ylamide; or salts or tautomers or N-oxides or solvates thereof.

The compound 4-(2,6-dichloro-benzoylamino)-1H-pyrazole-3-carboxylic acid piperidin-4-ylamide may be present in the form of an acid addition salt which may be a salt formed with hydrochloric acid or a salt as described in WO 2006/077426, the contents of which are incorporated herein by reference.

For example, the acid addition salt may be selected from salts formed with an acid selected from the group consisting of acetic, adipic, alginic, ascorbic (e.g. L-ascorbic), aspartic (e.g. L-aspartic), benzenesulphonic, benzoic, camphoric (e.g. (+) camphoric), capric, caprylic, carbonic, citric, cyclamic, dodecanoate, dodecylsulphuric, ethane-1,2-disulphonic, ethanesulphonic, fumaric, galactaric, gentisic, glucoheptonic, D-gluconic, glucuronic (e.g. D-glucuronic), glutamic (e.g. L-glutamic), α-oxoglutaric, glycolic, hippuric, isethionic, isobutyric, lactic (e.g. (+)-L-lactic and (±)-DL-lactic), lactobionic, laurylsulphonic, maleic, malic, (−)-L-malic, malonic, methanesulphonic, mucic, naphthalenesulphonic (e.g. naphthalene-2-sulphonic), naphthalene-1,5-disulphonic, nicotinic, oleic, orotic, oxalic, palmitic, pamoic, phosphoric, propionic, sebacic, stearic, succinic, sulphuric, tartaric (e.g. (+)-L-tartaric), thiocyanic, toluenesulphonic (e.g. p-toluenesulphonic), valeric and xinafoic acids.

One sub-group of acid addition salts includes salts formed with an acid selected from the group consisting of acetic, adipic, ascorbic (e.g. L-ascorbic), aspartic (e.g. L-aspartic), caproic, carbonic, citric, dodecanoic, fumaric, galactaric, glucoheptonic, gluconic (e.g. D-gluconic), glucuronic (e.g. D-glucuronic), glutamic (e.g. L-glutamic), glycolic, hippuric, lactic (e.g. (+)-L-lactic and (±)-DL-lactic), maleic, palmitic, phosphoric, sebacic, stearic, succinic, sulphuric, tartaric (e.g. (+)-L-tartaric) and thiocyanic acids.

More particularly the salts are acid addition salts formed with an acid selected from methanesulphonic acid and acetic acid, and mixtures thereof.

In one embodiment, the salt is an acid addition salt formed with methanesulphonic acid.

In another embodiment, the salt is an acid addition salt formed with acetic acid.

The salts may be prepared from 4-(2,6-dichloro-benzoylamino)-1H-pyrazole-3-carboxylic acid piperidin-4-ylamide by the methods described in WO 2006/077426.

The salts may be substantially crystalline; i.e. they are from 50% to 100% crystalline, and more particularly they may be at least 50% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

More preferably the salts may be those (or may be selected from the group consisting of those) that are 95% to 100% crystalline, for example at least 98% crystalline, or at least 99% crystalline, or at least 99.5% crystalline, or at least 99.6% crystalline or at least 99.7% crystalline or at least 99.8% crystalline or at least 99.9% crystalline, for example 100% crystalline.

One example of a substantially crystalline salt is a crystalline salt formed with methanesulphonic acid. Another example of a substantially crystalline salt is a crystalline salt formed with acetic acid.

The salts of the invention, in the solid state, can be solvated (e.g. hydrated) or non-solvated (e.g. anhydrous).

In one embodiment, the salts are non-solvated (e.g. anhydrous). An example of a non-solvated salt is the crystalline salt formed with methanesulphonic acid.

In one embodiment, the salt is a methanesulphonic acid salt of 4-(2,6-dichlorobenzoylamino)-1H-pyrazole-3-carboxylic acid piperidin-4-ylamide mesylate salt which is crystalline and is characterised by any one or more (in any combination) or all of the following parameters, namely that the salt:

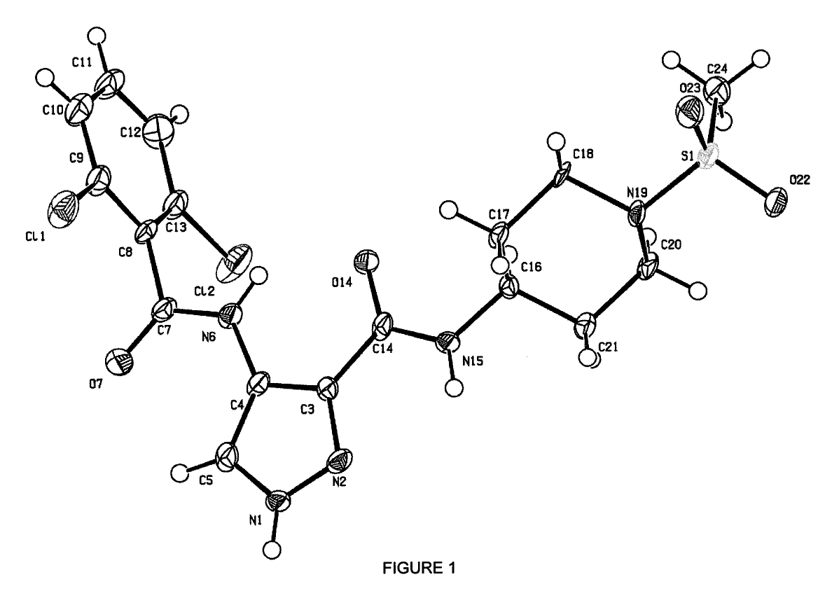

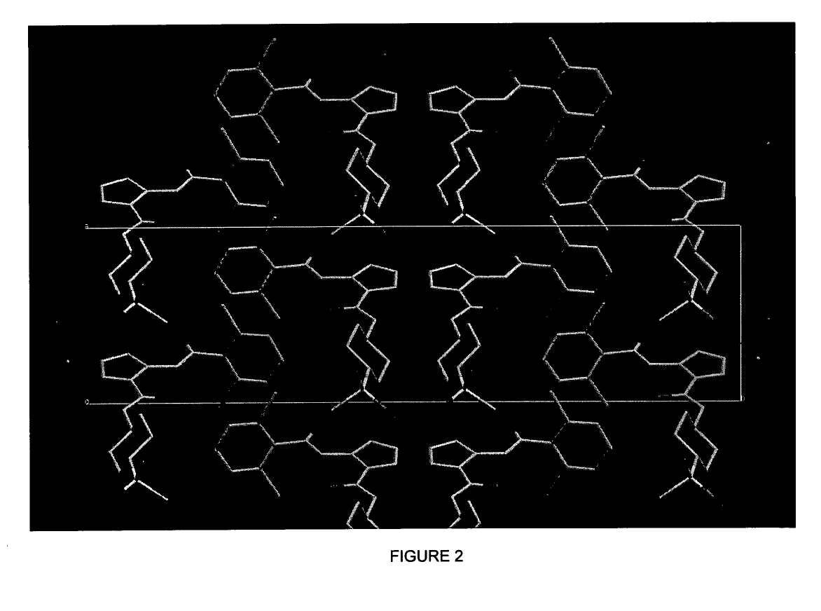

(a) has a crystal structure as set out in FIGS. 1 and 2 of WO 2006/077426; and/or
(b) has a crystal structure as defined by the coordinates in Example 2 of WO 2006/077426; and/or
(c) has crystal lattice parameters at 93 K a=8.90(10), b=12.44(10), c=38.49(4) Å, α=β=γ=90°; and/or
(d) has a crystal structure that belongs belong to an orthorhombic space group such as Pbca (# 61); and/or
(e) has an X-ray powder diffraction pattern characterised by the presence of major peaks at the diffraction angles (2θ) and interplanar spacings (d) set forth in Table A of WO 2006/077426, and optionally Table B of WO 2006/077426; for example wherein the X-ray powder diffraction pattern is characterised by the presence of major peaks at the diffraction angles (2E), interplanar spacings (d) and intensities set forth in Table C of WO 2006/077426; and/or
(f) exhibits peaks at the same diffraction angles as those of the X-ray powder diffraction pattern shown in FIG. 3 of WO 2006/077426 and optionally wherein the peaks have the same relative intensity as the peaks in FIG. 3 of WO 2006/077426; and/or
(g) has an X-ray powder diffraction pattern substantially as shown in FIG. 3 of WO 2006/077426; and/or
(h) is anhydrous and exhibits an endothermic peak at 379-380° C. e.g. 379.8° C. when subjected to DSC; and/or
(i) exhibits an infra-red spectrum, when analysed using the KBr disc method, that contains characteristic peaks at 3233, 3002, 2829, 1679, 1632, 1560, 1430, 1198, 1037, 909 and 784 cm⁻¹.

Particular pharmaceutical compositions comprising an aqueous solution containing an acid addition salt of 4-(2,6-dichloro-benzoylamino)-1H-pyrazole-3-carboxylic acid piperidin-4-ylamide (such as the mesylate and acetate and mixtures thereof, and preferably the mesylate) are also described in WO 2006/077426.

Methods of Treatment using the compounds of Fomrula (0) are described in WO 2005/012256 pages 105 to 107, and WO 2006/077426 pages 58 to 61, and are further described herein. Methods of Diagnosis of a patient to determine whether a disease or condition from which the patient is or may be suffering is one which would be susceptible to treatment with a compound having activity against CDK are described in WO 2005/012256 pages 107 to 111, and WO 2006/077426 pages 62 to 65, and are further described herein.

A preferred compound of the formula (0) is 4-(2,6-dichloro-benzoylamino)-1H-pyrazole-3-carboxylic acid piperidin-4-ylamide.

Accordingly, a preferred combination comprises (or consists essentially of) 4-(2,6-dichloro-benzoylamino)-1H-pyrazole-3-carboxylic acid piperidin-4-ylamide.

A further combination of the invention comprises (or consists essentially of) 4-(2,6-dichloro-benzoylamino)-1H-pyrazole-3-carboxylic acid piperidin-4-ylamide in the form of a salt selected from the acid addition salts formed with hydrochloric acid, methanesulphonic acid and/or acetic acid.

In addition combination of the invention comprises (or consists essentially of) the methane sulphonic acid salt of 4-(2,6-dichloro-benzoylamino)-1H-pyrazole-3-carboxylic acid piperidin-4-ylamide.

In addition combination of the invention comprises (or consists essentially of) methane sulphonic acid salt of 4-(2,6-dichloro-benzoylamino)-1H-pyrazole-3-carboxylic acid piperidin-4-ylamide is in crystalline form.

Methods for the Preparation of Compounds of the Formula (0) are as described in WO 2005/012256, WO 2006/077416 and WO 2006/077426, the contents of which are incorporated herein by reference. In particular, the contents of WO 2005/012556 which relate to the relevant processes at pages 91 to 101 are hereby incorporated herein by reference. In particular, the contents of WO 2006/074416 which relate to the relevant processes at pages 33 to 39 are hereby incorporated herein by reference. In particular, the contents of WO 2006/077426 which relate to the relevant processes at pages 30 to 36 are hereby incorporated herein by reference.

The Biological Activity of the compounds of formula (0) are described at pages 88 to 91 of WO 2005/012556 and at pages 39 to 49 of WO 2006/077426, which disclosure is hereby incorporated herein by reference, and further described herein.

### General Preferences and Definitions for Compounds of Formula (I′″)

A wide variety of compounds of the formula (I′″) find application in the combinations of the invention, as described in detail below. The compounds of formula (I′″) for use in the combinations of the invention correspond to those of formula (I) described in WO 2006/077416 and include the various possible substituents, sub-groups, embodiments and examples thereof as therein defined. The content of WO 2006/077416 describing the various possible substituents, subgroups, embodiments and examples of compounds of formula (I) (i.e. formula (I′″) herein) is hereby incorporated herein by reference.

In this specification in general (and this section in particular), unless the context indicates otherwise, references to a compound of formula (I′″) includes all subgroups of formula (I′″) as defined herein and the term ‘subgroups’ includes all preferences, embodiments, examples and particular compounds defined herein. Any references to formula (I′″) herein shall also be taken to refer to and any sub-group of compounds within formula (I′″) and any preferences and examples thereof unless the context requires otherwise.

The formula (I) of WO 2006/077416 is herein referred to as formula (I′″) and references to formula (I′″) herein are to be interpreted accordingly.

As described infra, compounds of the formula (I′″) have the formula:

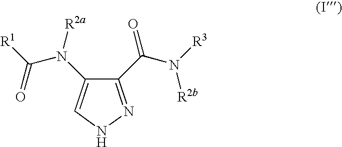

or salts, tautomers, solvates and N-oxides thereof;
wherein:
R¹ is 2,6-dichlorophenyl;
R^{2a} and R^{2b} are both hydrogen;
and R³ is a group:

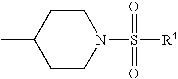

where R⁴ is C₁₋₄ alkyl.

Within this genus are many ‘subgroups’, preferences, embodiments, examples and particular compounds and while any references to formula (I′″) herein shall also be taken to refer to and any sub-group of compounds within formula (I′″) and any preferences and examples thereof unless the context requires otherwise, the following sections describe certain general prefernces and definitions in relation to compounds of the formula (I′″) for use in the combinations of the invention.

The C₁₋₄ alkyl group can be a C₁, C₂, C₃ or C₄ alkyl group.

Within the group of C₁₋₄ alkyl groups are the sub-groups of:
C₁₋₃ alkyl groups;
C₁₋₂ alkyl groups;
C₂₋₃ alkyl groups; and
C₂₋₄ alkyl groups.

One particular sub-group is C₁₋₃ alkyl.

Particular C₁₋₄ alkyl groups are methyl, ethyl, i-propyl, n-butyl, i-butyl and tert-butyl groups.

Another sub-group of C₁₋₄ alkyl groups consists of methyl, ethyl, i-propyl and n-propyl groups.

One preferred group is a methyl group.

Other particular groups R⁴ are ethyl and isopropyl.

Accordingly, a preferred combination comprises (or consists essentially of) 4-(2,6-dichloro-benzoylamino)-1H-pyrazole-3-carboxylic acid (1-methanesulphonyl-piperidin-4-yl)-amide.

A further combination comprises (or consists essentially of) substantially crystalline 4-(2,6-dichloro-benzoylamino)-1H-pyrazole-3-carboxylic acid (1-methanesulphonyl-piperidin-4-yl)-amide or crystal form thereof.

A further combination comprises (or consists essentially of) formulations comprising 4-(2,6-dichloro-benzoylamino)-1H-pyrazole-3-carboxylic acid (1-methanesulphonyl-piperidin-4-yl)-amide.

In such embodiments, the compound may be 4-(2,6-dichloro-benzoylamino)-1H-pyrazole-3-carboxylic acid (1-methanesulphonyl-piperidin-4-yl)-amide (for example 4-(2,6-dichloro-benzoylamino)-1H-pyrazole-3-carboxylic acid (1-methanesulphonyl-piperidin-4-yl)-amide in crystalline form).

A preferred compound within formula (I′) is 4-(2,6-dichloro-benzoylamino)-1H-pyrazole-3-carboxylic acid (1-methanesulphonyl-piperidin-4-yl)-amide.

The compound 4-(2,6-dichloro-benzoylamino)-1H-pyrazole-3-carboxylic acid (1-methanesulphonyl-piperidin-4-yl)-amide may be substantially crystalline; i.e. it is from 50% to 100% crystalline. Crystalline forms of 4-(2,6-dichloro-benzoylamino)-1H-pyrazole-3-carboxylic acid (1-methanesulphonyl-piperidin-4-yl)-amide are disclosed in our applications U.S. 60/746,541 and U.S. 60/830,967, the contents of each of which are incorporated herein by reference.

More particularly, the compound 4-(2,6-dichloro-benzoylamino)-1H-pyrazole-3-carboxylic acid (1-methanesulphonyl-piperidin-4-yl)-amide may be at least 55% crystalline, or at least 60% crystalline, or at least 65% crystalline, or at least 70% crystalline, or at least 75% crystalline, or at least 80% crystalline, or at least 85% crystalline, or at least 90% crystalline, or at least 95% crystalline, or at least 98% crystalline, or at least 99% crystalline, or at least 99.5% crystalline, or at least 99.9% crystalline, for example 100% crystalline.

The crystalline forms of the compound may be solvated (e.g. hydrated) or non-solvated (e.g. anhydrous). In one embodiment, the crystalline forms are solvated (e.g. hydrated). In another embodiment, the crystalline forms are non-solvated (e.g. anhydrous).

In one embodiment, the crystalline form of 4-(2,6-dichloro-benzoylamino)-1H-pyrazole-3-carboxylic acid (1-methanesulphonyl-piperidin-4-yl)-amide is a crystalline form which is characterised by any one or more (in any combination) or all of the following parameters, namely that the crystalline form:
has a crystal structure as set out in FIGS. 1 and 2 herein (and as set forth in U.S. 60/746,541 and U.S. 60/830,967); and/or
has a crystal structure as defined by the coordinates in Table 1 herein (and as set forth in U.S. 60/746,541 and U.S. 60/830,967); and/or
has crystal lattice parameters at a=9.15, b=31.32, c=7.93 Å, β=113.3°, α=γ=90°; and/or
has a crystal structure that belongs belong to a monoclinic space group such as C2/c (# 15); and/or
has an X-ray powder diffraction pattern characterised by the presence of major peaks at the diffraction angles (2θ) and interplanar spacings (d) set forth in Table A:

TABLE A

| 2θ/° | d/Å | I |
|---|---|---|
| 16.57 | 5.35 | 59 |
| 16.95 | 5.23 | 62 |
| 20.42 | 4.35 | 76 |
| 22.66 | 3.92 | 100 |
| 24.33 | 3.66 | 40 |

and optionally Table B:

TABLE B

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 19.53 | 4.55 | 37 |
| 20.88 | 4.25 | 23 |
| 24.99 | 3.56 | 16 |

and/or
exhibits peaks at the same diffraction angles as those of the X-ray powder diffraction pattern shown in FIG. 3 herein (and as set forth in U.S. 60/746,541 and U.S. 60/830,967) and optionally wherein the peaks have the same relative intensity as the peaks in FIG. 3 herein (and as set forth in U.S. 60/746,541 and U.S. 60/830,967); and/or
has an X-ray powder diffraction pattern substantially as shown in FIG. 3 herein (and as set forth in U.S. 60/746,541 and U.S. 60/830,967); and/or
is anhydrous and exhibits an endothermic peak at 293-296° C., for example 294.5-295° C., when subjected to DSC; and/or
exhibits an infra-red spectrum, when analysed using the Universal Attenuated Total Reflectance (UATR) method, that contains characteristic peaks at containing characteristic peaks at 3362, 3019, 2843, 1677, 1577, 1547, 1533, 1326, 1150, 926, 781, 667 cm⁻¹.

TABLE 1

space group: C2/c (# 15)
unit cell at 104K with a, b & c having 5% s.u.:
a = 9.150
b = 31.320
c = 7.930
alpha = gamma = 90.00
beta = 113.30
loop_{—}
_atom_site_label
_atom_site_type_symbol
_atom_site_fract_x
_atom_site_fract_y
_atom_site_fract_z
_atom_site_U_iso_or_equiv
_atom_site_adp_type
_atom_site_occupancy
_atom_site_symmetry_multiplicity
_atom_site_calc_flag
_atom_site_refinement_flags
_atom_site_disorder_assembly
_atom_site_disorder_group
C11 Cl 1.55055(16) 0.20997(4) 1.6202(2) 0.0376(4) Uani 1 1 d . . .
C12 Cl 0.97743(17) 0.20548(4) 1.6837(3) 0.0447(5) Uani 1 1 d . . .
S1 S 0.57041(12) 0.07771(3) 0.25572(15) 0.0212(3) Uani 1 1 d . . .
O7 O 1.3597(5) 0.14890(12) 1.8380(5) 0.0376(10) Uani 1 1 d . . .
O14 O 1.0227(4) 0.12633(10) 1.1610(5) 0.0266(8) Uani 1 1 d . . .
O22 O 0.4600(4) 0.04232(10) 0.1911(5) 0.0285(9) Uani 1 1 d . . .
O23 O 0.6695(4) 0.08741(13) 0.1578(5) 0.0282(9) Uani 1 1 d . . .
N1 N 1.2370(5) 0.02604(12) 1.5929(6) 0.0215(9) Uani 1 1 d . . .
H1 H 1.2665 0.0019 1.6538 0.026 Uiso 1 1 calc . . .
N2 N 1.1481(5) 0.02788(12) 1.4095(6) 0.0241(10) Uani 1 1 d . . .
N6 N 1.2053(5) 0.13987(12) 1.5365(6) 0.0226(9) Uani 1 1 d . . .
H6 H 1.1513 0.1533 1.4330 0.027 Uiso 1 1 calc . . .
N15 N 0.9606(5) 0.05870(11) 1.0508(6) 0.0192(9) Uani 1 1 d . . .
H15 H 0.9804 0.0313 1.0720 0.023 Uiso 1 1 calc . . .
N19 N 0.6881(4) 0.06785(12) 0.4705(5) 0.0185(9) Uani 1 1 d . . .
C3 C 1.1279(5) 0.06988(14) 1.3718(7) 0.0196(10) Uani 1 1 d . . .
C4 C 1.2051(5) 0.09437(14) 1.5332(7) 0.0210(10) Uani 1 1 d . . .
C5 C 1.2765(6) 0.06537(16) 1.6738(8) 0.0240(11) Uani 1 1 d . . .
H5 H 1.3393 0.0714 1.7992 0.029 Uiso 1 1 calc . . .
C7 C 1.2811(6) 0.16340(14) 1.6846(7) 0.0243(11) Uani 1 1 d . . .
C8 C 1.2638(7) 0.21135(14) 1.6550(8) 0.0239(11) Uani 1 1 d . . .
C9 C 1.3834(6) 0.23627(16) 1.6278(7) 0.0260(11) Uani 1 1 d . . .
C10 C 1.3723(7) 0.27967(18) 1.6094(8) 0.0331(13) Uani 1 1 d . . .
H10 H 1.4564 0.2955 1.5978 0.040 Uiso 1 1 calc . . .
C11 C 1.2352(7) 0.30098(16) 1.6076(8) 0.0333(14) Uani 1 1 d . . .
H11 H 1.2266 0.3311 1.5928 0.040 Uiso 1 1 calc . . .
C12 C 1.1136(7) 0.27794(18) 1.6273(8) 0.0354(14) Uani 1 1 d . . .
H12 H 1.0207 0.2921 1.6242 0.043 Uiso 1 1 calc . . .
C13 C 1.1291(6) 0.23383(16) 1.6518(8) 0.0321(14) Uani 1 1 d . . .
C14 C 1.0327(5) 0.08684(14) 1.1863(7) 0.0218(11) Uani 1 1 d . . .
C16 C 0.8492(5) 0.07270(14) 0.8678(7) 0.0184(10) Uani 1 1 d . . .
H16 H 0.7916 0.0985 0.8838 0.022 Uiso 1 1 calc . . .
C17 C 0.9342(5) 0.08479(14) 0.7426(7) 0.0211(11) Uani 1 1 d . . .
H17A H 0.9903 0.0595 0.7223 0.025 Uiso 1 1 calc . . .
H17B H 1.0142 0.1073 0.8019 0.025 Uiso 1 1 calc . . .
C18 C 0.8119(5) 0.10120(15) 0.5567(7) 0.0225(10) Uani 1 1 d . . .
H18A H 0.7612 0.1276 0.5760 0.027 Uiso 1 1 calc . . .
H18B H 0.8665 0.1080 0.4743 0.027 Uiso 1 1 calc . . .
C20 C 0.6048(5) 0.05454(15) 0.5920(7) 0.0242(11) Uani 1 1 d . . .
H20A H 0.5265 0.0319 0.5305 0.029 Uiso 1 1 calc . . .
H20B H 0.5466 0.0792 0.6132 0.029 Uiso 1 1 calc . . .
C21 C 0.7264(6) 0.03785(14) 0.7776(7) 0.0234(11) Uani 1 1 d . . .
H21A H 0.6712 0.0302 0.8584 0.028 Uiso 1 1 calc . . .
H21B H 0.7798 0.0120 0.7578 0.028 Uiso 1 1 calc . . .
C24 C 0.4560(6) 0.12321(16) 0.2544(8) 0.0279(12) Uani 1 1 d . . .
H24A H 0.5263 0.1479 0.2999 0.042 Uiso 1 1 calc . . .
H24B H 0.3984 0.1181 0.3338 0.042 Uiso 1 1 calc . . .
H24C H 0.3796 0.1288 0.1288 0.042 Uiso 1 1 calc . . .

Pharmaceutical compositions comprising a compound of formula (I′″) and a pharmaceutically acceptable carrier in a form suitable for oral administration are described in WO 2006/077416 at pages 37 to 48.

In particular a pharmaceutical composition comprising a substantially amorphous solid solution, said solid solution comprising (a) a compound of the formula (I′″), for example the compound of Example 1; and (b) a polymer selected from the group consisting of: polyvinylpyrrolidone (povidone), crosslinked polyvinylpyrrolidone (crospovidone), hydroxypropyl methylcellulose, hydroxypropylcellulose, polyethylene oxide, gelatin, crosslinked polyacrylic acid (carbomer), carboxymethylcellulose, crosslinked carboxymethylcellulose (croscarmellose), methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;

wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

Further compositions are disclosed in our applications U.S. 60/746,541 and U.S. 60/830,967, the contents of each of which are incorporated herein by reference.

Methods of Treatment using this compound are described in WO 2006/077416 at pages 48 to 52 and further described herein. Methods of Diagnosis of a patient to determine whether a disease or condition from which the patient is or may be suffering is one which would be susceptible to treatment with a compound having activity against CDK are described at pages 52 to 56 and further described herein.

The Biological Activity of the compounds of formula (I′″) are described at pages 25 to 30 of WO 2006/074416, which disclosure is hereby incorporated herein by reference and further described herein.

### Salts, Solvates, Tautomers, Isomers, N-Oxides, Esters, Prodrugs and Isotopes

A reference to a particular compound (including inter alia any of the compounds of formula (0) and (I′″) or the auxiliary compounds described herein) also includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

Such salts, solvates, tautomers, isomers, N-oxides, esters, prodrugs and isotopes are described in WO 2005/012256 at pages 81 to 88 in relation to compounds of “formula (0)” therein (which correspond to the compounds of formula (0) herein, as explained above) and in WO 2006/077426. This disclosures in WO 2005/012256 and WO 2006/077426 are hereby incorporated herein by reference, and the teachings relating to salts, solvates, tautomers, isomers, N-oxides, esters, prodrugs and isotopes of compounds of formula (0) are to be extended to the various compounds (including inter alia any of the compounds of formula (0), (I′″) or the auxiliary compounds described herein).

Such salts, solvates, tautomers, isomers, N-oxides, esters, prodrugs and isotopes are described in WO 2006/077416 at pages 18 to 25 in relation to compounds of “formula (I)” therein (which correspond to the compounds of formula (I′″) herein, as explained above). This disclosure in WO 2006/077416 is hereby incorporated herein by reference, and the teachings relating to salts, solvates, tautomers, isomers, N-oxides, esters, prodrugs and isotopes of compounds of formula (I) are to be extended to the various compounds (including inter alia any of the compounds of formula (0), (I′″) or the auxiliary compounds described herein).

### Advantages of the Compounds of Formula (I′″) as Components of the Combinations of the Invention

These are described at pages 28 to 30 of WO 2006/077416, which disclosure is hereby incorporated herein by reference.

### Methods for the Preparation of Compounds of the Formula (I′″)

These are described at pages 30 to 36 of WO 2006/077416, which disclosure is hereby incorporated herein by reference.

Methods for the Preparation of Compounds of the Formula (I′″) are as described in WO 2005/012256, WO 2006/077416 and WO 2006/077426, the contents of which are incorporated herein by reference. In particular, the contents of WO 2006/074416 which relate to the relevant processes at pages 33 to 39 are hereby incorporated herein by reference.

### Methods of Purification of the Compounds of Formula (I′″)

These are described at pages 36 to 37 of WO 2006/077416, which disclosure is hereby incorporated herein by reference.

### Biological Activity

Biological activity of the compounds of formula (I′″) are described at pages 25 to 28 of WO 2006/077416, which disclosure is hereby incorporated herein by reference. The biological activity of the compounds of formula (0) are described at pages 88 to 91 of WO 2005/012556 and at pages 39 to 49 of WO 2006/077426, which disclosure is hereby incorporated herein by reference.

The compounds of the formula (0) and (I′″) are inhibitors of cyclin dependent kinases. For example, the compounds of the formula (0) and (I′″) are inhibitors of cyclin dependent kinases selected from CDK1, CDK2, CDK3, CDK4, CDK5, CDK6 and CDK9, and more particularly selected from CDK1, CDK2, CDK3, CDK4, CDK5 and CDK9.

The compounds of the formula (0) and (I′″) also have activity against glycogen synthase kinase-3 (GSK-3).

As a consequence of their activity in modulating or inhibiting CDK and glycogen synthase kinase, the compounds of the formula (0) and (I′″) will be useful in providing a means of arresting, or recovering control of, the cell cycle in abnormally dividing cells. The compounds will therefore prove useful in treating or preventing proliferative disorders such as cancers. The compounds of the formula (0) and (I′″) will also be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease, for example autoimmune diseases and neurodegenerative diseases.

One sub-group of disease states and conditions where the compounds of the formula (0) and (I′″) will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, chronic lymphocytic leukaemia, B-cell lymphoma (such as diffuse large B cell lymphoma), T-cell lymphoma, Hodgkin's lymphoma, non-Hodgkin's lymphoma, hairy cell lymphoma, or Burkett's lymphoma; a hematopoietic tumour of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumour of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; or Kaposi's sarcoma.

The cancers may be cancers which are sensitive to inhibition of any one or more cyclin dependent kinases selected from CDK1, CDK2, CDK3, CDK4, CDK5 and CDK6, for example, one or more CDK kinases selected from CDK1, CDK2, CDK4 and CDK5, e.g. CDK1 and/or CDK2.

Whether or not a particular cancer is one which is sensitive to inhibition by a cyclin dependent kinase may be determined by means of a cell growth assay as set out in the examples below or by a method as set out in the section headed “Methods of Diagnosis”.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-senstive rhinosinusitis, cystic fibrosis, multiple sclerosis, kidney diseases and cancer pain.

Thus, in the pharmaceutical compositions, uses or methods of this invention for treating a disease or condition comprising abnormal cell growth, the disease or condition comprising abnormal cell growth in one embodiment is a cancer.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma

Another sub-set of cancers includes breast cancer, ovarian cancer, colon cancer, prostate cancer, oesophageal cancer, squamous cancer and non-small cell lung carcinomas.

The activity of the compounds of the formula (0) and (I′″) as inhibitors of cyclin dependent kinases and glycogen synthase kinase-3 can be measured using the assays set forth herein and the level of activity exhibited by a given compound can be defined in terms of the IC₅₀ value.

### Optional Auxiliary Compounds for Use According to the Invention

Any of a wide variety of auxiliary compounds may optionally be used as further constituents of the combinations of the invention. Such optional auxiliary compounds may be anti-cancer agents. A particular combination comprises (or consists essentially of) 4-(2,6-dichloro-benzoylamino)-1H-pyrazole-3-carboxylic acid piperidin-4-ylamide and salts thereof, and 4-(2,6-dichloro-benzoylamino)-1H-pyrazole-3-carboxylic acid (1-methanesulphonyl-piperidin-4-yl)-amide and one or more optional auxiliary compounds as described herein.

In this section, and in all other sections of this specification, unless the context indicates otherwise, references to formula (0) include formulae (I), (I⁰), (Ia), (Ib), (II′), (III), (IV), (IVa), (Va), (Vb), (VIa), (VIb), (VII) or (VIII) as described in WO 2005/012256 and sub-groups, examples or embodiments of formulae (0), (I⁰), (Ia), (Ib), (II′), (III), (IV), (IVa), (Va), (Vb), (VIa), (VIb), (VII) or (VIII) as described in WO 2005/012256. Moreover, in this specification in general, unless the context indicates otherwise, references to a compound of formula (I′″) includes all subgroups of formula (I′″) as defined herein and as described in WO 2006/077416 and the term ‘subgroups’ includes all preferences, embodiments, examples and particular compounds defined herein and as described in WO 2006/077416. Any references to formula (I′″) herein shall also be taken to refer to and any sub-group of compounds within formula (I′″) and any preferences and examples thereof unless the context requires otherwise.

Preferably, the optional auxiliary compounds for use in the combinations of the invention are selected from the following class lists:

### List A

1. hormones, hormone agonists, hormone antagonists and hormone modulating agents (including corticosteroids, antiandrogens, antiestrogens and GNRAs);
2. cytokines and cytokine activating agents;
3. retinoids and rexinoids
4. monoclonal antibodies (including monoclonal antibodies to cell surface antigen(s));
5. camptothecin compounds and other topoisomerase I inhibitors;
6. antimetabolites;
7. vinca alkaloids and other tubulin targeting agents;
8. taxanes;
9. epothilones;
10. platinum compounds;
11. DNA binders and Topo II inhibitors (including anthracycline derivatives);
12. alkylating agents (including aziridine, nitrogen mustard and nitrosourea alkylating agents);
13. signalling inhibitors (including PKA/B inhibitors and PKB pathway inhibitors);
14. CDK inhibitors (including ancilliary CDK inhibitors);
15. COX-2 inhibitors;
16. HDAC inhibitors;
17. Selective immunoresponse modulators;
18. DNA methyl transferase inhibitors;
19. proteasome inhibitors;
20. Aurora inhibitors;
21. Hsp90 inhibitors;
22. Checkpoint targeting agents;
23. DNA repair inhibitors;
24. Inhibitors of G-protein coupled receptor inhibitors.

In embodiments where the combination of the invention comprises one or more auxiliary compounds, the auxiliary compound(s) are preferably independently selected from the classes (1) (in particular corticosteroids), (4), (6), (7), (8), (10), (11), (12), (13), (17), (18), (19), (23) and (24) of list A (above). Most preferably, the one or more auxiliary compounds are independently selected from classes (1) in particular corticosteroids, (4), (6), (8), (10), (11), (12), (13), (18), (19), and (24) of list A (above).

In embodiments where the combination of the invention comprises two or more auxiliary compounds, then the two or more auxiliary compounds are preferably independently selected from the classes (1) to (24) of list A set out above.

In embodiments where the combination of the invention comprises two or more auxiliary compounds, then the two or more auxiliary compounds are preferably independently selected from the classes (1) (in particular corticosteroids), (2), (3), (17), (22), (23) and (24) of list A set out above.

### List B

In some embodiments the optional auxiliary compounds for use in the combination with the compounds of formula (I) are selected from the following classes:
1. hormones, hormone agonists, hormone antagonists and hormone modulating agents (including antiandrogens, antiestrogens and GNRAs);
2. monoclonal antibodies (e.g. monoclonal antibodies to cell surface antigen(s));
3. camptothecin compounds and other topoisomerase I inhibitors;
4. antimetabolites;
5. vinca alkaloids and other tubulin targeting agents;
6. taxanes;
7. epothilones;
8. platinum compounds;
9. DNA binders and Topo II inhibitors (including anthracycline derivatives);
10. alkylating agents (including aziridine, nitrogen mustard and nitrosourea alkylating agents);
11. signalling inhibitors (including PKA/B inhibitors and PKB pathway inhibitors);
12. CDK inhibitors (including ancillary CDK inhibitors);
13. COX-2 inhibitors;
14. HDAC inhibitors;
15. DNA methylase inhibitors;
16. proteasome inhibitors;
17. Aurora inhibitors;
18. Hsp90 inhibitors;
19. a combination of two or more of the foregoing classes (4), (6) and/or (11);
20. a combination of two or more of the foregoing classes (3)-(6), (8), (9) and/or (11);
21. a combination of two or more of the foregoing classes (10) and/or (12)-(16);
22. a combination of two or more of the foregoing classes (1-6)-(8-16);
23. a combination of two or more of the foregoing classes (1), (2), (3), (4), (5), (6), (8), (9), (10), (11) and (16);
24. a combination of two or more of the foregoing classes (1), (2), (3), (4), (5), (6), (8) and (10);
25. a combination of two or more of the foregoing classes (3), (4), (5), (6), (8), (9), (10);
26. a combination of two or more of the foregoing classes (4), (6) and (8);
27. a combination of two or more of the foregoing classes (7) and (1)-(6) and/or (8)-(18)
28. a combination of two or more of the foregoing classes (7), (17), and (18)

In embodiments where the combination of the invention comprises two or more auxiliary compounds, then the two or more auxiliary compounds are preferably independently selected from the classes 1 to 18 set out above.

A reference to a particular auxiliary compound herein is intended to include ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof.

The various compounds/compound classes described above are now described in more detail, wherein the numbering of the compound classes corresponds to that used in list A (above).

### 1. Hormones, Hormone Agonists, Hormone Antagonists and Hormone Modulating Agents

Definition: The terms “corticosteroid”, “antiandrogen”, “antiestrogen”, “antiandrogen agent” and “antiestrogen agent” as used herein refers to those described herein and analogues thereof, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Biological activity: The hormones, hormone agonists, hormone antagonists and hormone modulating agents (including the antiandrogens and antiestrogen agents) working via one or more pharmacological actions as described herein have been identified as suitable anti-cancer agents. The term ‘hormonal therapies’ is used to collectively to refer to hormones, hormone agonists, hormone antagonists and hormone modulating agents.

Technical background: Hormonal therapy plays an important role in the treatment of certain types of cancer where tumours are formed in tissues that are sensitive to hormonal growth control such as the breast and prostate. Thus, for example, estrogen promotes growth of certain breast cancers and testosterone promotes growth of prostate cancers. Since the growth of such tumours is dependent on specific hormones, considerable research has been carried out to investigate whether it is possible to affect tumour growth by increasing or decreasing the levels of certain hormones in the body. Hormonal therapy attempts to control tumour growth in these hormone-sensitive tissues by manipulating the activity of the hormones.

Cancers which are derived from either lymphocyte precursors or mature lymphocytes such as certain types of leukemia, Hodgkin's disease and non-Hodgkin's lymphoma often retain the sensitivity to treatment with corticosteroids including prednisolone, predisone and dexamethasone exhibited by mature lymphophocytes. As a consequence treatment with one or more corticosteroids is often incorporated into the treatment of these diseases. Thus contemplated for use with the invention are corticosteroids.

With regard to breast cancer, tumour growth is stimulated by estrogen, and antiestrogen agents have therefore been proposed and widely used for the treatment of this type of cancer. One of the most widely used of such agents is tamoxifen which is a competitive inhibitor of estradiol binding to the estrogen receptor (ER). When bound to the ER, tamoxifen induces a change in the three-dimensional shape of the receptor, inhibiting its binding to the estrogen responsive element on DNA. Under normal physiological conditions, estrogen stimulation increases tumour cell production of transforming growth cell b (TGF-b), an autocrine inhibitor of tumour cell growth. By blocking these pathways, the net effect of tamoxifen treatment is to decrease the autocrine stimulation of breast cancer growth. In addition, tamoxifen decreases the local production of insulin-like growth factor (IGF-1) by surrounding tissues: IGF-I is a paracrine growth factor for the breast cancer cell (Jordan and Murphy, Endocr. Rev., 1990, 11; 578-610). An alternative approach to disease control is to reduce circulating levels of estradiol by inhibition of aromatase—an enzyme which is critical for its production. Both Tamoxifen and aromatase inhibitors including anastrazole, letrozole and examestane are widely used in the treatment of post-menopausal women with breast cancer both in the adjuvant and metatsatic setting (e.g. metastatic breast cancer). Tamoxifen is also used in pre-menopausal women with ER-positive tumours. There are various potential side-effects of long-term tamoxifen treatment, for example the possibility of endometrial cancer and the occurrence of thrombo-embolic events. Although aromatase inhibitors are generally better tolerated than tamoxifen patients often experience musculo-skeletal pain and significant bone loss leading to osteoporosis.

Other estrogen receptor antagonists (or selective estrogen receptor modulators (SERMs)) with broadly similar action to tamoxifen include toremifene and raloxifene. Toremifene is a non-steroidal SERM, which has the chemical name 2-(4-[(Z)-4-chloro-1,2-diphenyl-1-butenyl]-phenoxy)-N,N-dimethylethylamine, and is used for the treatment of metastatic breast cancer, side-effects including hot flushes, nausea and dizziness. Raloxifene is a benzothiophene SERM, which has the chemical name [6-hydroxy-2-(4-hydroxyphenyl)benzo[b]thien-3-yl]-[4-[2-(1-piperidinyl)ethoxy]-phenyl]-methanone hydrochloride, and is being investigated for the treatment of breast cancer, side-effects including hot flushes and leg cramps.

Fulvestrant, which acts by reducing the expression of the ER in tumour tissue has the chemical name 7-a-[9-(4,4,5,5,5-pentafluoropentylsulphinyl)-nonyl]estra-1,3,5-(10)-triene-3,17-beta-diol, is often used following treatment with tamoxifen and an aromatase inhibitor (e.g. as a second line treatment of advanced breast cancer). Treatment may be accompanied by hot flushes and endometrial stimulation.

Prostate cancer cells almost invariably overexpress the androgen receptor, and thus antiandrogens are widely used in the treatment of the disease. Antiandrogens are androgen receptor antagonists which bind to the androgen receptor and prevent dihydrotestosterone from binding. Dihydrotestosterone stimulates new growth of prostate cells, including cancerous prostate cells. An example of an antiadrogen is bicalutamide, which has the chemical name (R,S)—N-(4-cyano-3-(4-fluorophenylsulfonyl)-2-hydroxy-2-methyl-3-(trifluoromethyl)propanamide, and has been approved for use in combination with luteinizing hormone-releasing hormone (LHRH) analogs for the treatment of advanced prostate cancer, side effects including hot flushes, bone pain, hematuria and gastro-intestinal symptoms. An alternative means of reducing circulating levels of dihydrotestosterone is to directly inhibit its production from testosterone using flutamide.

In one emboiment the hormonal therapies include fulvestrant, toremifene and raloxifene.

A further type of hormonal cancer treatment comprises the use of progestin analogs. Progestin is the synthetic form of progesterone, a hormone secreted by the ovaries and endometrial lining of the uterus. Acting with estrogen, progesterone promotes breast development and growth of endometrial cells during the menstrual cycle. It is believed that progestins may act by suppressing the production of estrogen from the adrenal glands (an alternate source particularly in post-menopausal women), lowering estrogen receptor levels, or altering tumour hormone metabolism.

Progestin analogs are used in the management of uterine cancer (e.g. advanced uterine cancer) or renal cancer. They can also be used for treating advanced breast cancer, although this use is less common, due to the numerous anti-estrogen treatment options available. Occasionally, progestin analogs are used as hormonal therapy for prostate cancer. An example of a progestin analog is megestrol acetate (a.k.a. megestrel acetate), which has the chemical name 17a-acetyloxy-6-methylpregna-4,6-diene-3,20-dione, and is a putative inhibitor of pituitary gonadotrophin production with a resultant decrease in estrogen secretion, The drug is used for the palliative treatment of advanced carcinoma of the breast or endometrium (i.e., recurrent, inoperable, or metastatic disease), side-effects including oedema and thromoembolic episodes.

Preferences and specific embodiments: A particularly preferred antiestrogen agent for use in accordance with the invention is tamoxifen. Tamoxifen is commercially available for example from AstraZeneca pic under the trade name Nolvadex, or may be prepared for example as described in U.K. patent specifications 1064629 and 1354939, or by processes analogous thereto.

Yet another preferred antiestrogen agent is droloxifene. Fulvestrant is commercially available for example from AstraZeneca plc under the trade name Faslodex, or may be prepared for example as described in European patent specification No. 138504, or by processes analogous thereto. Raloxifene is commercially available for example from Eli Lilly and Company under the trade name Evista, or may be prepared for example as described in U.S. patent specification No. 4418068, or by processes analogous thereto. Toremifene is commercially available for example from Schering Corporation under the trade name Fareston, or may be prepared for example as described in U.S. patent specification No. 4696949, or by processes analogous thereto. The antiestrogen agent droloxifene, which may be prepared for example as described in U.S. patent specification No. 5047431, or by processes analogous thereto, can also be used in accordance with the invention.

A preferred antiandrogen for use in accordance with the invention is bicalutamide which is commercially available for example from AstraZeneca pic under the trade name Casodex, or may be prepared for example as described in European patent specification No. 100172, or by processes analogous thereto. Other preferred hormonal therapies for use in accordance with the invention include tamoxifen, fulvestrant, raloxifene, toremifene, droloxifene, letrazole, anastrazole, exemestane, bicalutamide, luprolide, megestrol/megestrel acetate, aminoglutethimide (alternatively spelt aminoglutethamide) and flutamide.

Other preferred hormonal therapies for use in accordance with the invention include tamoxifen, fulvestrant, raloxifene, toremifene, droloxifene, letrazole, anastrazole, exemestane, bicalutamide, luprolide, megestrol/megestrel acetate, aminoglutethimide and bexarotene.

A preferred progestin analog is megestrol/megestrel acetate which is commercially available for example from Bristol-Myers Squibb Corporation under the trade name Megace, or may be prepared for example as described in US Patent Specification No. 2891079, or by processes analogous thereto.

Thus, specific embodiments of these anti-cancer agents for use in the combinations of the invention include: tamoxifen; toremifene; raloxifene; medroxyprogesterone; megestrol/megestrel; aminoglutethimide; letrozole; anastrozole; exemestane; goserelin; leuprolide; abarelix; fluoxymestrone; diethylstilbestrol; ketoconazole; fulvestrant; flutamide; bicalutimide; nilutamide; cyproterone and buserelin.

Thus, contemplated for use in the combinations of the invention are antiandrogens and antiestrogens.

In other embodiments, the hormone, hormone agonist, hormone antagonist or hormone modulating agent is fulvestrant, raloxifene, droloxifene, toremifene, megestrol/megestrel and flutamide.

In other embodiments, the hormone, hormone agonist, hormone antagonist or hormone modulating agent is fulvestrant, raloxifene, droloxifene, toremifene, megestrol/megestrel and bexarotene.

In one embodiment the hormones, hormone agonists, hormone antagonists and hormone modulating agents include corticosteroids, antiandrogens, antiestrogens and GNRAs. In another embodiment the hormones, hormone agonists, hormone antagonists and hormone modulating agents include antiandrogens, antiestrogens and GNRAs.

Posology: The antiandrogen or antiestrogen agent is advantageously administered in a dosage of about 1 to 100 mg daily depending on the particular agent and the condition being treated. Tamoxifen is advantageously administered orally in a dosage of 5 to 50 mg, preferably 10 to 20 mg twice a day (or 20 mg once a day), continuing the therapy for sufficient time to achieve and maintain a therapeutic effect.

With regard to the other preferred antiestrogen agents: fulvestrant is advantageously administered in the form of a 250 mg monthly injection (though doses of 250-750 mg per month may also be employed); toremifene is advantageously administered orally in a dosage of about 60 mg once a day, continuing the therapy for sufficient time to achieve and maintain a therapeutic effect; droloxifene is advantageously administered orally in a dosage of about 20-100 mg once a day; and raloxifene is advantageously administered orally in a dosage of about 60 mg once a day.

With regard to the preferred antiandrogen bicalutamide, this is generally administered in an oral dosage of 50 mg daily.

With regard to the preferred progestin analog megestrol/megestrel acetate, this is generally administered in an oral dosage of 40 mg four times daily.

The dosages noted above may generally be administered for example once, twice or more per course of treatment, which may be repeated for example daily or every 7, 14, 21 or 28 days in particular every 7, 14, 21 or 28 days.

### Aromatase Inhibitors

Of the hormones, hormone agonists, hormone antagonists and hormone modulating agents for use in the combinations of the invention, preferred are aromatase inhibitors.

In post-menopausal women, the principal source of circulating estrogen is from conversion of adrenal androgens (androstenedione and testosterone) to estrogens (estrone and estradiol) by the aromatase enzyme in peripheral tissues. Estrogen deprivation through aromatase inhibition or inactivation is an effective and selective treatment for some post-menopausal patients with hormone-dependent breast cancer. Examples of such hormone modulating agents include aromatase inhibitors or inactivators, such as exemestane, anastrozole, letrozole and aminoglutethimide.

Exemestane, which has the chemical name 6-methylenandrosta-1,4-diene-3,17-dione, is used for the treatment of advanced breast cancer in post-menopausal women whose disease has progressed following tamoxifen therapy, side effects including hot flashes and nausea. Anastrozole, which has the chemical name, α,α,α′,α′-tetramethyl-5-(1H-1,2,4-triazol-1-ylmethyl)-1,3-benzenediacetonitrile, is used for adjuvant treatment of post-menopausal women with hormone receptor-positive early breast cancer, and also for the first-line treatment of post-menopausal women with hormone receptor-positive or hormone receptor-unknown locally advanced or metastatic breast cancer, and for the treatment of advanced breast cancer in post-menopausal women with disease progression following tamoxifen therapy. Administration of anastrozole usually results in side-effects including gastrointestinal disturbances, musculoskeletal pain, rashes and headaches. Letrozole, which has the chemical name 4,4′-(1H-1,2,4-triazol-1-ylmethylene)-dibenzonitrile, is used for the adjuvant treatment of ER positive breast cancer, for first-line treatment of post-menopausal women with hormone receptor-positive or hormone receptor-unknown locally advanced or metastatic breast cancer, and for the treatment of advanced breast cancer in post-menopausal women with disease progression following antiestrogen therapy, possible side-effects including occasional transient thrombocytopenia and elevation of liver transaminases.

Aminoglutethimide which has the chemical name 3-(4-aminophenyl)-3-ethyl-2,6-piperidinedione, is also used for treating breast cancer but suffers from the side-effects of skin rashes and less commonly thrombocytopenia and leukopenia.

Preferred aromatase inhibitors include letrozole, anastrozole, exemestane and aminoglutethimide. Letrozole is commercially available for example from Novartis A. G. under the trade name Femara, or may be prepared for example as described in U.S. patent specification No. 4978672, or by processes analogous thereto. Anastrozole is commercially available for example from AstraZeneca plc under the trade name Arimidex, or may be prepared for example as described in U.S. Patent Specification No. 4935437, or by processes analogous thereto. Exemestane is commercially available for example from Pharmacia Corporation under the trade name Aromasin, or may be prepared for example as described in U.S. patent specification No. 4978672, or by processes analogous thereto. Aminoglutethimide is commercially available for example from Novartis A. G. under the trade name Cytadren, or may be prepared for example as described in U.S. patent specification No 2848455, or by processes analogous thereto. The aromatase inhibitor vorozole, which may be prepared for example as described in European patent specification No. 293978, or by processes analogous thereto, can also be used in accordance with the invention.

With regard to the preferred aromatase inhihibitors, these are generally administered in an oral daily dosage in the range 1 to 1000 mg, for example letrozole in a dosage of about 2.5 mg once a day; anastrozole in a dosage of about 1 mg once a day; exemestane in a dosage of about 25 mg once a day; and aminoglutethimide in a dosage of 250 mg 2-4 times daily.

Particularly preferred are aromatase inhibitors selected from the agents described herein, for example, letrozole, anastrozole, exemestane and aminoglutethimide.

### GNRAs

Of the hormones, hormone agonists, hormone antagonists and hormone modulating agents for use in the combinations of the invention, preferred are agents of the GNRA class.

Definition: As used herein the term GNRA is intended to define gonadotropin-releasing hormone (GnRH) agonists and antagonists (including those described below), together with the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Technical background: When released from the hypothalamus in the brain, gonadotropin-releasing hormone (GnRH) agonists stimulate the pituitary gland to produce gonadotropins. Gonadotropins are hormones that stimulate androgen synthesis in the testes and estrogen synthesis in the ovaries. When GnRH agonists are first administered, they can cause an increase in gonadotropin release, but with continued administration, GnRH will block gonadotropin release, and therefore decrease the synthesis of androgen and estrogen. GnRH analogs are used to treat metastatic prostate cancer. They have also been approved for treatment of metastatic breast cancer in pre-menopausal women. Examples of GnRH analogs include goserelin acetate and leuprolide acetate. In contrast GnRH antagonists such as aberelix cause no initial GnRH surge since they have no agonist effects. However, due to their narrow therapeutic index, their use is currently limited to advanced prostate cancer that is refractory to other hormonal treatment such as GnRH agonists and anti-androgens.

Goserelin acetate is a synthetic decapeptide analog of LHRH or GnRH, and has the chemical structure of pyro-Glu-His-Trp-Ser-Tyr-D-Ser(Bu)-Leu-Arg-Pro-Azgly-NH₂ acetate, and is used for the treatment of breast and prostate cancers and also endometriosis, side effects include hot flashes, bronchitis, arrhythmias, hypertension, anxiety and headaches. Leuprolide acetate is a synthetic nonapeptide analog of GnRH or LHRH, and has the chemical name 5-oxo-L-prolyl-L-histidyl-L-tryptophyl-L-seryl-L-tyrosyl-D-leucyl-L-leucyl-L-arginyl-N-ethyl-L-prolinamide acetate. Leuprolide acetate is used for the treatment of prostate cancer, endometriosis, and also breast cancer, side effects being similar to those of goserelin acetate.

Abarelix is a synthetic decapeptide Ala-Phe-Ala-Ser-Tyr-Asn-Leu-Lys-Pro-Ala, and has the chemical name N-Acetyl-3-(2-naphthalenyl)-D-alanyl-4-chloro-D-phenylalanyl-3-(3-pyridinyl)-D-alanyl-L-seryl-N-methyl-L-tyrosyl-D-asparaginyl-L-leucyl-N6-(1-methylethyl)-L-lysyl-L-prolyl-D-alaninamide. Abarelix can be prepared according to R. W. Roeske, WO9640757 (1996 to Indiana Univ. Found.).

Preferences and specific embodiments: Preferred GnRH agonists and antagonists for use in accordance with the invention include any of the GNRAs described herein, including in particular goserelin, leuprolide/leuporelin, triptorelin, buserelin, abarelix, goserelin acetate and leuprolide acetate. Particularly preferred are goserelin and leuprolide. Goserelin acetate is commercially available for example from AstraZeneca plc under the trade name Zoladex, or may be prepared for example as described in U.S. Patent Specification No. 5510460, or by processes analogous thereto. Leuprolide acetate is commercially available for example from TAP Pharmaceuticals Inc. under the trade name Lupron, or may be prepared for example as described in U.S. Patent Specification No. 3914412, or by processes analogous thereto. Goserelin is commercially available from AstraZeneca under the trade name Zoladex and may be prepared for example as described in ICI patent publication U.S. Pat. No. 4,100,274 or Hoechst patent publication EP475184 or by processes analagous thereto. Leuprolide is commercially available in the USA from TAP Pharmaceuticals Inc. under the trade name Lupron and in Europe from Wyeth under the trade name Prostap and may be prepared for example as described in Abbott patent publication U.S. Pat. No. 4,005,063 or by processes analogous thereto. Triptorelin is commercially available from Watson Pharma under the trade name Trelstar and may be prepared for example as described in Tulane patent publication U.S. Pat. No. 5,003,011 or by processes analagous thereto. Buserelin is commercially available under the trade name Suprefact and may be prepared for example as described in Hoechst patent publication U.S. Pat. No. 4,024,248 or by processes analogous thereto. Abarelix is commercially available from Praecis Pharmaceuticals under the trade name Plenaxis and may be prepared for example as described by Jiang et al., J Med Chem (2001), 44(3), 453-467 or Polypeptide Laboratories patent publication WO2003055900 or by processes analogous thereto.

Other GnRH agonists and antagonists for use in accordance with the invention include, but are not limited to, Histrelin from Ortho Pharmaceutical Corp, Nafarelin acetate from Roche, and Deslorelin from Shire Pharmaceuticals.

Posology: The GnRH agonists and antagonists are advantageously administered in dosages of 1.8 mg to 100 mg, for example 3.6 mg monthly or 10.8 mg every three months for goserelin or 7.5 mg monthly, 22.5 mg every three months or 30 mg every four months for leuprolide.

With regard to the preferred GnRH analogs, these are generally administered in the following dosages, namely goserelin acetate as a 3.6 mg subcutaneous implant every 4 weeks, and leuprolide as a 7.5 mg intramuscular depot every month.

### 2. Cytokines and Cytokine-activating Agents

Definition: The term “cytokine” is a term of art, and references to cytokines herein is intended to cover the cytokine per se together with the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. The term “cytokine-activating agent” is intended to cover any agent which (directly or indirectly) induces, potentiates, stimulates, activates or promotes endogenous cytokine production or the activity thereof in vivo, together with the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: Cytokines are a class of proteins or polypeptides predominantly produced by cells of the immune system which have the capacity to control the function of a second cell. In relation to anticancer therapy cytokines are used to control the growth or kill the cancer cells directly and to modulate the immune system more effectively to control the growth of tumours.

Cytokines, such as interferon (IFN) alpha and Interleukin-2, induce growth arrest or tumour cell death. IFN-alpha is used the treatment of malignant melanoma, chronic myelogenous leukemia (CML), hairy cell leukemia, and Kaposi's sarcoma. Interleukin-2 is used in the treatment of malignant melanoma and renal cell cancer either alone or in combination with IFN-alpha.

Cytokines exhibit antitumour activity through a variety of different mechanisms including the stimulation of immune cells to fight tumors For example, the T cell growth factor, IL-2 promotes T-cell and natural killer (NK) cell activation. Other cytokines such as the interferons and granulocyte-macrophage colony-stimulating factor (GM-CSF) act on antigen presenting cells to facilitate the activation of the key immune effector B cells and T cells.

Preferences and specific embodiments: Any of the cytokines and cytokine-modulating agents described herein may find application in the invention, including in particular interferons (such as interferon-γ and interferon α) and interleukins (e.g. interleukin 2). Interferon α-2b (recombinant) is available commercially under the trade name of INTRON® A from Schering Plough.

Other preferred interferons include Interferon α-2a which is available under the trade name of ROFERON from Roche.

A particularly preferred interleukin is PROLEUKIN® IL-2 (aldesleukin) which is available from Chiron Corp.

Posology: The interferons are administered by injection in a schedule which is dependent on tha particular indication. For IntronA treatment of malignant melanoma preferably in a schedule that includes induction treatment on 5 consecutive days per week for 4 weeks as an intravenous (IV) infusion at a dose of 20 million IU/m2, followed by maintenance treatment three times per week for 48 weeks as a subcutaneous (SC) injection, at a dose of 10 million IU/m2. For Intron A treatment of non-Hodgkin's Lymphoma preferably in a schedule of 5 million IU subcutaneously three times per week for up to 18 months in conjunction with an anthracycline-containing chemotherapy regimen.

The recommended initial dose of Roferon-A for CML is 9 MIU daily administered as a subcutaneous or intramuscular injection. Based on clinical experience short-term tolerance may be improved by gradually increasing the dose of Roferon-A over the first week of administration from 3 MIU daily for 3 days to 6 MIU daily for 3 days to the target dose of 9 MIU daily for the duration of the treatment period. The induction dose of Roferon-A for Hairy cell leukaemia is 3 MIU daily for 16 to 24 weeks, administered as a subcutaneous or intramuscular injection. Subcutaneous administration is particularly suggested for, but not limited to, thrombocytopenic patients (platelet count <50,000) or for patients at risk for bleeding. The recommended maintenance dose is 3 MIU, three times a week (tiw).

For PROLEUKIN the following schedule has been used to treat adult patients with metastatic renal cell carcinoma (metastatic RCC) or metastatic melanoma (each course of treatment consists of two 5-day treatment cycles separated by a rest period): 600,000 IU/kg (0.037 mg/kg) dose administered every 8 hours by a 15-minute IV infusion for a maximum of 14 doses. Following 9 days of rest, the schedule is repeated for another 14 doses, for a maximum of 28 doses per course, as tolerated.

Cytokine-activating agents: Preferred cytokine-activating agents include: (a) Picibanil from Chugai Pharmaceuticals, an IFN-gamma-inducing molecule for carcinoma treatment; (b) Romurtide from Daiichi which activates the cytokine network by stimulation of colony stimulating factor release; (c) Sizofuran from Kaken Pharmaceutical, a beta1-3, beta1-6 D-glucan isolated from suchirotake mushroom, which stimulates production of IFN-gamma and IL-2 by mitogen-stimulated peripheral blood mononuclear cells, and is useful in uterine cervix tumour and lung tumour treatment; (d) Virulizin from Lorus Therapeutics Inc, a NK agonist and cytokine release modulator which stimulates IL-17 synthesis and IL-12 release for the treatment of sarcoma, melanoma, pancreas tumours, breast tumours, lung tumours, and Kaposis sarcoma (e) Thymosin alpha 1, a synthetic 28-amino acid peptide with multiple biological activities primarily directed towards immune response enhancement for increased production of Th1 cytokines, which is useful in the treatment of non-small-cell lung cancer, hepatocellular carcinoma, melanoma, carcinoma, and lung brain and renal tumours.

### 3. Retinoids and Rexinoids

Definition: The term “retinoid” is a term of art used herein in a broad sense to include not only the specific retinoids disclosed herein, but also the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. The term ‘rexinoids’ refers to synthetic agents that bind specifically to retinoid X receptors.

Technical background: Tretinoin is an endogenous metabolite of retinol. It induces terminal differentiation in several hemopoietic precursor cell lines, including human myeloid cell lines. Acute Promyelocytic Leukemia (APL) is associated with a specific translocation between chromosomes 15 and 17; the retinoic acid receptor—α is located on chromosome 17. The translocation appears to inhibit differentiation and lead to carcinogenesis; tretinoin may overcome this when used in high doses. Tretinoin induces remissions in 64-100% of APL patients, with time to remission usually between 8 and 119 days of therapy. Acquired resistance during therapy is common especially with prolonged dosing (4-6 months). Alitretinoin is a 9-cis-retinoic acid derivative which appears to be selective for the RXR subfamily of retinoid receptors. This selectivity may preserve therapeutic antineoplastic effects while reducing significant side effects of retinoid therapy including birth defects at fetal exposure, irritation of skin and mucosal surfaces or skeletal abnormalities. Topical alitretinoin is approved in the US for the treatment of Kaposi's Sarcoma. Oral and gel (topical) formulations of bexarotene (Targretin; LGD-1069), a retinoid X receptor (RXR)-selective antitumor retinoid, are available for the treatment of cutaneous T-cell lymphoma (CTCL).

U.S. Pat. No. 6,127,382, WO 01/70668, WO 00/68191, WO 97/48672, WO 97/19052 and WO 97/19062 (all to Allergan) each describe compounds having retinoid-like activity for use in the treatment of various hyperproliferative diseases including cancers.

Preferences and specific embodiments: Preferred retinoids for use in accordance with the invention include any of the retinoids disclosed herein, including in particular tretinoin (all-trans retinoic acid), alitretinoin and bexarotene. Tretinoin (Retacnyl, Aknoten, Tretin M) is commercially available from Roche under the trade name Vesanoid and may be prepared for example as described in D. A. van Dorp, J. R. Arens, Rec. Trav. Chim. 65, 338 (1946); C. D. Robeson et al., J. Am. Chem. Soc. 77, 4111 (1955); R. Marbet, DE 2061507; U.S. Pat. No. 3,746,730 (1971, 1973 both to Hoffmann-La Roche), or by processes analogous thereto. Alitretinoin (9-cis-Tretinoin, Panrexin) is commercially available from Ligand Pharmaceuticals under the trade name Panretin and may be prepared for example as described in C. D. Robeson et al., J. Am. Chem. Soc. 77, 4111 (1955); M. Matsui et al., J. Vitaminol. 4, 178 (1958); M. F. Boehm et al., J. Med. Chem. 37, 408 (1994), or by processes analogous thereto. Bexarotene (Targrexin, Targret) is commercially available from Eisai Inc under the trade name Targretin and may be prepared for example as described in M. F. Boehm et al., WO 9321146 (1993 to Ligand Pharm.); M. L. Dawson et al., U.S. Pat. No. 5,466,861 (1995 to SRI Int.; La Jolla Cancer Res. Found.), or by processes analogous thereto.

Posology: Tretinoin is advantageously administered in dosages of 25 mg/m²/day to 45 mg/m²/day by mouth in two divided doses for 30 days after complete remission or up to a maximum of 90 days. Alitretinoin gel 0.1% is advantageously administered initially by application two (2) times a day to cutaneous KS lesions.

Bexarotene is advantageously administered initially as a single daily oral dose of 300 mg/m²/day. The dose may be adjusted to 200 mg/m²/day then to 100 mg/m²/day, or temporarily suspended, if necessitated by toxicity. If there is no tumor response after eight weeks of treatment and if the initial dose of 300 mg/m²/day is well tolerated, the dose may be escalated to 400 mg/m²/day with careful monitoring. Bexarotene gel is advantageously applied initially once every other day for the first week. The application frequency may be increased at weekly intervals to once daily, then twice daily, then three times daily and finally four times daily according to individual lesion tolerance.

### 4. Monoclonal Antibodies.

Any monoclonal antibody e.g. including but not limited to one or more cell surface antigen(s)may be used in the combinations of the invention. Antibody specificity may be assayed or determined using any of a wide variety of techniques well-known to those skilled in the art.

Definition: The term “monoclonal antibody” used herein refers to antibodies from any source, and so includes those that are fully human and also those which contain structural or specificity determining elements derived from other species (and which can be referred to as, for example, chimeric or humanized antibodies).

Technical background: The use of monoclonal antibodies is now widely accepted in anticancer chemotherapy as they are highly specific and can therefore bind and affect disease specific targets, thereby sparing normal cells and causing fewer side-effects than traditional chemotherapies.

One group of cells which have been investigated as targets for antibody chemotherapy for the treatment of various cancers are those bearing the cell-surface antigens comprising the cluster designation (CD) molecules which are over-expressed or aberrantly expressed in tumour cells, for example CD20, CD22, CD33 and CD52 which are over-expressed on the tumour cell surface, most notably in tumours of hematopoietic origin. Antibodies to these CD targets (anti-CD antibodies) include the monoclonal antibodies rituximab (a.k.a. rituxamab), tositumomab and gemtuzumab ozogamicin.

Rituximab/rituxamab is a mouse/human chimeric anti-CD20 monoclonal antibody which has been used extensively for the treatment of B-cell non-Hodgkin's lymphoma including relapsed, refractory low-grade or follicular lymphoma. The product is also being developed for various other indications including chronic lymphocytic leukaemia and rheumatoid arthritis. Side effects of rituximab/rituxamab may include hypoxia, pulmonary infiltrates, acute respiratory distress syndrome, myocardial infarction, ventricular fibrillation or cardiogenic shock. Tositumomab is a cell-specific anti-CD20 antibody labelled with iodine-131, for the treatment of non-Hodgkin's lymphoma and lymphocytic leukaemia. Possible side-effects of tositumomab include thrombocytopenia and neutropenia. Gemtuzumab ozogamicin is a cytotoxic drug (calicheamicin) linked to a human monoclonal antibody specific for CD33. Calicheamicin is a very potent antitumour agent, over 1,000 times more potent than adriamycin. Once released inside the cell, calicheamicin binds in a sequence-specific manner to the minor groove of DNA, undergoes rearrangement, and exposes free radicals, leading to breakage of double-stranded DNA, and resulting in cell apoptosis (programmed cell death). Gemtuzumab ozogamicin is used as a second-line treatment for acute myeloid leukaemia, possible side-effects including severe hypersensitivity reactions such as anaphylaxis, and also hepatotoxicity.

Alemtuzumab (Millennium Pharmaceuticals, also known as Campath) is a humanized monoclonal antibody against CD52 useful for the treatment of chronic lymphocytic leukaemia and Non-Hodgkin lymphoma which induces the secretion of TNF-alpha, IFN-gamma and IL-6.

Preferences: Preferred monoclonal antibodies for use according to the invention include anti-CD antibodies, including alemtuzumab, CD20, CD22 and CD33. Particularly preferred are monoclonal antibody to cell surface antigens, including anti-CD antibodies (for example, CD20, CD22, CD33) as described above. Other preferred monoclonal antibodies include those which target interleukin 6 (IL-6).

Specific embodiments: In one embodiment, the monoclonal antibody is an antibody to the cluster designation CD molecules, for example, CD20, CD22, CD33 and CD52. In another embodiment, the monoclonal antibody to cell surface antigen is selected from rituximab/rituxamab, tositumomab and gemtuzumab ozogamicin. Other monoclonal antibodies that may be used according to the invention include bevacizumab.

Exemplary formulations: Monoclonal antibodies to cell surface antigen(s) for use according to the invention include CD52 antibodies (e.g. alemtuzumab) and other anti-CD antibodies (for example, CD20, CD22 and CD33), as described herein. Preferred are therapeutic combinations comprising a monoclonal antibody to cell surface antigen(s), for example anti-CD antibodies (e.g. CD20, CD22 and CD33) which exhibit an advantageous efficacious effect, for example, against tumour cell growth, in comparison with the respective effects shown by the individual components of the combination.

CD52 selctivity has also been achieved through the combination of a specific ligand with diphtheria toxin which is released intracellularly (denileukin difitox; Ontak). This approach has been licensed for use in the treatment of cutaneous T-cell lymphoma and is under investigation for the treatment of other types of non-hodgkin's lymphoma.

In addition targeting structures other than tumour cells themselves have also been shown to be efficacious in cancer therapy. This approach has been most effective in inhibiting new blood vessel formation using bevacuzimab, a monoclonal antibody directed against circulating Vascular Endothelial Growth Factor. This approach may be useful in the treatment of a wide range of malignancies.

Preferred examples of monoclonal antibodies to cell surface antigens (anti-CD antibodies) include rituximab/rituxamab, tositumomab and gemtuzumab ozogamicin.

Rituximab/rituxamab is commercially available from F Hoffman-La Roche Ltd under the trade name Mabthera, or may be obtained as described in PCT patent specification No. WO 94/11026. Tositumomab is commercially available from GlaxoSmithKline plc under the trade name Bexxar, or may be obtained as described in U.S. Patent specification No 5595721. Gemtuzumab ozogamicin is commercially available from Wyeth Research under the trade name Mylotarg, or may be obtained as described in U.S. Pat. No. 5,877,296.

Biological activity: Monoclonal antibodies (e.g. monoclonal antibodies to one or more cell surface antigen(s)) have been identified as suitable anti-cancer agents. Antibodies are effective through a variety of mechanisms. They can block essential cellular growth factors or receptors, directly induce apoptosis, bind to target cells or deliver cytotoxic payloads such as radioisotopes and toxins.

Posology: The anti-CD antibodies may be administered for example in dosages of 5 to 400 mg per square meter (mg/m²) of body surface; in particular gemtuzumab ozogamicin may be administered for example in a dosage of about 9 mg/m² of body surface; rituximab/rituxamab may be administered for example in a dosage of about 375 mg/m² as an IV infusion once a week for four doses; the dosage for tositumomab must be individually quantified for each patient according to the usual clinical parameters such as age, weight, sex and condition of the patient to ensure appropriate delivery of the radioisotope.

These dosages may be administered for example once, twice or more per course of treatment, which may be repeated for example every 7, 14, 21 or 28 days.

### 5. Camptothecin Compounds

Definition: The term “camptothecin compound” as used herein refers to camptothecin per se or analogues of camptothecin as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: Camptothecin compounds are compounds related to or derived from the parent compound camptothecin which is a water-insoluble alkaloid derived from the Chinese tree Camptothecin acuminata and the Indian tree Nothapodytes foetida. Camptothecin has a potent inhibitory activity against DNA biosynthesis and has shown high activity against tumour cell growth in various experimental systems. Its clinical use in anti-cancer therapy is, however, limited significantly by its high toxicity, and various analogues have been developed in attempts to reduce the toxicity of camptothecin while retaining the potency of its anti-tumour effect. Examples of such analogues include irinotecan and topotecan.

These compounds have been found to be specific inhibitors of DNA topoisomerase I. Topoisomerases are enzymes that are capable of altering DNA topology in eukaryotic cells. They are critical for important cellular functions and cell proliferation. There are two classes of topoisomerases in eukaryotic cells, namely type I and type II. Topoisomerase I is a monomeric enzyme having a molecular weight of approximately 100,000. The enzyme binds to DNA and introduces a transient single-strand break, unwinds the double helix (or allows it to unwind) and subsequently reseals the break before dissociating from the DNA strand.

Irinotecan, namely 7-ethyl-10-(4-(1-piperidino)-1-piperidino)carbonyloxy-(20S)-camptothecin, and its hydrochloride, also known as CPT 11, have been found to have improved potency and reduced toxicity, and superior water-solubility. Irinotecan has been found to have clinical efficacy in the treatment of various cancers especially colorectal cancer. Another important camptothecin compound is topotecan, namely (S)-9-dimethylaminomethyl-10-hydroxy-camptothecin which, in clinical trials, has shown efficacy against several solid tumours, particularly ovarian and cervical cancer and small cell lung cancer or alternatively ovarian cancer and non-small cell lung carcinoma.

Exemplary formulations: A parenteral pharmaceutical formulation for administration by injection and containing a camptothecin compound can be prepared by dissolving 100 mg of a water soluble salt of the camptothecin compound (for example a compound as described in EP 0321122 and in particular the examples therein) in 10 ml of sterile 0.9% saline and then sterilising the solution and filling the solution into a suitable container.

Biological activity: The camptothecin compounds of the combinations of the invention are specific inhibitors of DNA topoisomerase I are described above and have activity against various cancers.

Prior art references: WO 01/64194 (Janssen) discloses combinations of farnesyl transferase inhibitors and camptothecin compounds. EP 137145 (Rhone Poulenc Rorer) discloses camptothecin compounds including irinotecan. EP 321122 (SmithKline Beecham) discloses camptothecin compounds including topotecan.

Problems: Although camptothecin compounds are widely used as chemotherapeutic agents in humans, they are not therapeutically effective in all patients or against all types of tumours. There is therefore a need to increase the inhibitory efficacy of camptothecin compounds against tumour growth and also to provide a means for the use of lower dosages of camptothecin compounds to reduce the potential for adverse toxic side effects to the patient.

Preferences: Preferred camptothecin compounds for use in accordance with the invention include irinotecan and topotecan referred to above. Irinotecan is commercially available for example from Rhone-Poulenc Rorer under the trade name “Campto” and may be prepared for example as described in European patent specification No. 137145 or by processes analogous thereto. Topotecan is commercially available for example from SmithKline Beecham under the trade name “Hycamtin” and may be prepared for example as described in European patent number 321122 or by processes analogous thereto. Other camptothecin compounds may be prepared in conventional manner for example by processes analogous to those described above for irinotecan and topotecan.

Specific embodiments: In one embodiment, the camptothecin compound is irinotecan. In another embodiment, the camptothecin compound is a camptothecin compound other than irinotecan, for example a camptothecin compound such as topotecan.

Posology: The camptothecin compound is advantageously administered in a dosage of 0.1 to 400 mg per square metre (mg/m²) of body surface area, for example 1 to 300 mg/m², particularly for irinotecan in a dosage of about 100 to 350 mg/m² and for topotecan in about 1 to 2 mg/m² per course of treatment. These dosages may be administered for example once, twice or more per course of treatment, which may be repeated daily or every 7, 14, 21 or 28 days in particular every 7, 14, 21 or 28 days.

### 6. Antimetabolites

Definition: The terms “antimetabolic compound” and “antimetabolite” are used as synonyms and define antimetabolic compounds or analogues of antimetabolic compounds as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. Thus, the antimetabolic compounds, otherwise known as antimetabolites, referred to herein consitute a large group of anticancer drugs that interfere with metabolic processes vital to the physiology and proliferation of cancer cells. Such compounds include nucleoside derivatives, either pyrimidine or purine nucleoside analogs, that inhibit DNA synthesis, and inhibitors of thymidylate synthase and/or dihydrofolate reductase enzymes.

Technical background: Antimetabolites (or antimetabolic compounds), constitute a large group of anticancer drugs that interfere with metabolic processes vital to the physiology and proliferation of cancer cells. Such compounds include nucleoside derivatives, either pyrimidine or purine nucleoside analogues, that inhibit DNA synthesis, and inhibitors of thymidylate synthase and/or dihydrofolate reductase enzymes. Anti-tumour nucleoside derivatives have been used for many years for the treatment of various cancers. Among the oldest and most widely used of these derivatives is 5-fluorouracil (5-FU) which has been used to treat a number of cancers such as colorectal, breast, hepatic and head and neck tumours.

In order to enhance the cytotoxic effect of 5-FU, leucovorin has been used to stabilise the resulting thymidylate synthase/5-FU complex thus further increasing its inhibiton. However, various factors limit the use of 5-FU, for example tumour resistance, toxicities, including gastrointestinal and haematological effects, and the need for intravenous administration. Various approaches have been taken to overcome these disadvantages including proposals to overcome the poor bioavailability of 5-FU and also to increase the therapeutic index of 5-FU, either by reducing systemic toxicity or by increasing the amount of active drug reaching the tumour.

One such compound which provides an improved therapeutic advantage over 5-FU is capecitabine, which has the chemical name [1-(5-deoxy-β-D-ribofuranosyl)-5-fluoro-1,2-dihydro-2-oxo-4-pyrimidinyl]-carbamic acid pentyl ester. Capecitabine is a pro-drug of 5-FU which is well absorbed after oral dosing and delivers pharmacologically-active concentrations of 5-FU to tumours. As well as offering potentially superior activity to 5-FU, it can also be used for oral therapy with prolonged administration. Gemcitabine is a nucleoside analogue which has the chemical name 2′-deoxy-2′,2′-difluoro-cytidine, and which has been used in the treatment of various cancers including non-small cell lung cancer, breast, ovarian and pancreatic cancer in particular non-small cell lung cancer and pancreatic cancer. Further anti-tumour nucleosides include cytarabine and fludarabine. Cytarabine, also known as ara-C, which has the chemical name 1-β-D-arabinofuranosylcytosine, has been found useful in the treatment of acute leukemia, chronic myelocytic leukemia and erythroleukemia. Cytarabine, also known as ara-C, which has the chemical name 1-β-D-arabinofuranosylcytosine, has been found useful in the treatment of acute myelocytic leukemia, chronic myelocytic leukemia (blast phase), acute lymphocytic leukemia and erythroleukemia. Fludarabine is a DNA synthesis inhibitor, which has the chemical name 9-β-D-arabinofuranosyl-2-fluoro-adenine, and is used for the treatment of refractory B-cell chronic lymphocytic leukaemia. Other anti-folate antimetabolites used in anticancer chemotherapy include the enzyme inhibitors raltitrexed, pemetrexed, and methotrexate. Raltitrexed is a folate-based thymidylate synthase inhibitor, which has the chemical name N-[5-[N-[(3,4-dihydro-2-methyl-4-oxo-6-quinazolinyl)-methyl-N-methylamino]-2-thenoyl]-L-glutamic acid, and is used in the treatment of advanced colorectal cancer. Pemetrexed is a thymidylate synthase and transferase inhibitor, which has the chemical name N-[4-[2-(2-amino-4,7-dihydro-4-oxo-1H-pyrrolo[2,3-d]pyrimidin-5-yl)ethyl]benzoyl]-L-glutamic acid, disodium salt, and is used for the treatment of mesothelioma and locally advanced or metastatic non-small-cell lung cancer (SCLC) in previously treated patients. Methotrexate is an antimetabolite which interrupts cell division by inhibiting DNA replication through dihydrofolate reductase inhibition, resulting in cell death, and has the chemical name is N-[4-[[(2,4-diamino-6-pteridinyl)methyl]-ethylamino]benzoyl]-L-glutamic acid, and is used for the treatment of acute lymphocytic leukemia, and also in the treatment of breast cancer, epidermoid cancers of the head and neck, and lung cancer, particularly squamous cell and small cell types, and advanced stage non-Hodgkin's lymphomas, in particular in the treatment of breast cancer, epidermoid cancers of the head and neck, and advanced stage non-Hodgkin's lymphomas.

Biological activity: The antimetabolic compounds of the combinations of the invention interfere with metabolic processes vital to the physiology and proliferation of cancer cells as described above and have activity against various cancers.

Problems: These anticancer agents have a number of side-effects especially myelosuppression and in some cases nausea and diarrhoea. There is therefore a need to provide a means for the use of lower dosages to reduce the potential of adverse toxic side effects to the patient.

Preferences: Preferred antimetabolic compounds for use in accordance with the invention include anti-tumour nucleosides such as 5-fluorouracil, gemcitabine, capecitabine, cytarabine and fludarabine and enzyme inhibitors such as ralitrexed, pemetrexed and methotrexate referred to herein. Thus, preferred antimetabolic compounds for use in accordance with the invention are anti-tumour nucleoside derivatives including 5-fluorouracil, gemcitabine, capecitabine, cytarabine and fludarabine referred to herein. Other preferred antimetabolic compounds for use in accordance with the invention are enzyme inhibitors including ralitrexed, pemetrexed and methotrexate.

5-Fluorouracil is widely available commercially, or may be prepared for example as described in U.S. patent specification No. 2802005. Gemcitabine is commercially available for example from Eli Lilly and Company under the trade name Gemzar, or may be prepared for example as described in European patent specification No. 122707, or by processes analogous thereto. Capecitabine is commercially available for example from Hoffman-La Roche Inc under the trade name Xeloda, or may be prepared for example as described in European patent specification No. 698611, or by processes analogous thereto. Cytarabine is commercially available for example from Pharmacia and Upjohn Co under the trade name Cytosar, or may be prepared for example as described in U.S. patent specification No. 3116282, or by processes analogous thereto. Fludarabine is commercially available for example from Schering AG under the trade name Fludara, or may be prepared for example as described in U.S. patent specification No. 4357324, or by processes analogous thereto. Ralitrexed is commercially available for example from AstraZeneca plc under the trade name Tomudex, or may be prepared for example as described in European patent specification No. 239632, or by processes analogous thereto. Pemetrexed is commercially available for example from Eli Lilly and Company under the trade name Alimta, or may be prepared for example as described in European patent specification No. 432677, or by processes analogous thereto. Methotrexate is commercially available for example from Lederle Laboraories under the trade name Methotrexate-Lederle, or may be prepared for example as described in U.S. patent specification No. 2512572, or by processes analogous thereto. Other antimetabolites for use in the combinations of the invention include 6-mercaptopurine, 6-thioguanine, cladribine, 2′-deoxycoformycin and hydroxyurea.

Specific embodiments: In one embodiment, the antimetabolic compound is gemcitabine. In another embodiment, the antimetabolic compound is a antimetabolic compound other than 5-fluorouracil or fludarabine, for example an antimetabolic compound such as gemcitabine, capecitabine, cytarabine, ralitrexed, pemetrexed or methotrexate.

Posology: The antimetabolite compound will be administered in a dosage that will depend on the factors noted above. Examples of dosages for particular preferred antimetabolites are given below by way of example. With regard to anti-tumour nucleosides, these are advantageously administered in a daily dosage of 10 to 2500 mg per square meter (mg/m²) of body surface area, for example 700 to 1500 mg/m², particularly for 5-FU in a dosage of 200 to 500 mg/m², for gemcitabine in a dosage of 800 to 1200 mg/m², for capecitabine in a dosage of 1000 to 1200 mg/m², for cytarabine in a dosage of 100-200 mg/m² and for fludarabine in a dosage of 10 to 50 mg/m².

For the following enzyme inhibitors, examples are given of possible doses. Thus, raltitrexed can be administered in a dosage of about 3 mg/m², pemetrexed in a dosage of 500 mg/m² and methotrexate in a dosage of 30-40 mg/m².

The dosages noted above may generally be administered for example once, twice or more per course of treatment, which may be repeated for example every 7, 14, 21 or 28 days.

### 7. Vinca Alkaloids

Definition: The term “vinca alkaloid” as used herein refers to vinca alkaloid compounds or analogues of vinca alkaloid compounds as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: The vinca alkaloids for use in the combinations of the invention are anti-tumour vinca alkaloids related to or derived from extracts of the periwinkle plant (Vinca rosea). Among these compounds, vinblastine and vincristine are important clinical agents for the treatment of leukaemias, lymphomas and testicular cancer, and vinorelbine has activity against lung cancer and breast cancer.

Biological activity: The vinca alkaloid compounds of the combinations of the invention are tubulin targeting agents and have activity against various cancers.

Problems: Treatment with Vinca alkaloids is accompanied by significant toxicities. For example, vinblastine causes leukopenia which reaches a nadir in 7 to 10 days following drug administration, after which recovery ensues within 7 days, while vincristine demonstrates some neurological toxicity for example numbness and trembling of the extremities, loss of deep tendon reflexes and weakness of distal limb musculature. Vinorelbine has some toxicity in the form of granulocytopenia but with only modest thrombocytopenia and less neurotoxicity than other vinca alkaloids. There is therefore a need to increase the inhibitory efficacy of anti-tumour vinca alkaloids against tumour growth and also to provide a means for the use of lower dosages of anti-tumour vinca alkaloids to reduce the potential of adverse toxic side effects to the patient.

Preferences: Preferred anti-tumour vinca alkaloids for use in accordance with the invention include vindesine, vinvesir, vinblastine, vincristine and vinorelbine. Particularly preferred anti-tumour vinca alkaloids for use in accordance with the invention include vinblastine, vincristine and vinorelbine refererred to above. Vinblastine is commercially available for example as the sulphate salt for injection from Eli Lilly and Co under the trade name Velban, and may be prepared for example as described in German patent specification No. 2124023 or by processes analogous thereto. Vincristine is commercially available for example as the sulphate salt for injection from Eli Lilly and Co under the trade name Oncovin and may be prepared for example as described in the above German patent specification No. 2124023 or by processes analogous thereto. Vincristine is also available as a liposomal formulation under the name Onco-TCS™. Vinorelbine is commercially available for example as the tartrate salt for injection from Glaxo Wellcome under the trade name Navelbine and may be prepared for example as described in U.S. patent specification No. 4307100, or by processes analogous thereto. Other anti-tumour vinca alkaloids may be prepared in conventional manner for example by processes analogous to those described above for vinoblastine, vincristine and vinorelbine.

Another preferred vinca alkaloid is vindesine. Vindesine is a synthetic derivative of the dimeric catharanthus alkaloid vinblastine, is available from Lilly under the tradename Eldisine and from Shionogi under the tradename Fildesin. Details of the synthesis of Vindesine are described in Lilly patent DE2415980 (1974) and by C. J. Burnett et al., J. Med. Chem. 21, 88 (1978).

Specific embodiments: In one embodiment, the vinca alkaloid compound is selected from vinoblastine, vincristine and vinorelbine. In another embodiment, the vinca alkaloid compound is vinoblastine.

Posology: The anti-tumour vinca alkaloid is advantageously administered in a dosage of 2 to 30 mg per square meter (mg/m²) of body surface area, particularly for vinblastine in a dosage of about 3 to 12 mg/m², for vincristine in a dosage of about 1 to 2 mg/m², and for vinorelbine in dosage of about 10 to 30 mg/m² per course of treatment. These dosages may be administered for example once, twice or more per course of treatment, which may be repeated for example every 1, 14, 21 or 28 days.

8. Taxanes (taxoids)

Definition: The term “taxane compound” as used herein refers to taxane compounds or analogues of taxane compounds as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Technical background: The taxanes are a class of compounds having the taxane ring system and related to or derived from extracts from certain species of yew (Taxus) trees. These compounds have been found to have activity against tumour cell growth and certain compounds in this class have been used in the clinic for the treatment of various cancers. Thus, for example, pactitaxel is a diterpene isolated from the bark of the yew tree, Taxus brevifolia, and can be produced by partial synthesis from 10-acetylbacctin, a precursor obtained from yew needles and twigs or by total synthesis, see Holton et al, J. Am. Chem. Soc. 116; 1597-1601 (1994) and Nicholau et al, Nature 367:630 (1994). Paclitaxel has shown anti-neoplastic activity and more recently it has been established that its antitumour activity is due to the promotion of microtubule polymerisation, Kumar N. J., Biol. Chem. 256: 1035-1041 (1981); Rowinsky et al, J. Natl. Cancer Inst. 82: 1247-1259 (1990); and Schiff et al, Nature 277: 655-667 (1979). Paclitaxel has now demonstrated efficacy in several human tumours in clinical trials, McGuire et al, Ann. Int. Med., 111:273-279 (1989); Holmes et al, J. Natl. Cancer Inst. 83: 1797-1805 (1991); Kohn et al J. Natl Cancer Inst. 86: 18-24 (1994); and Kohn et al, American Society for Clinical Oncology, 12 (1993). Paclitaxel is used for the treatment of ovarian, breast and lung cancer, in particular has for example been used for the treatment of ovarian cancer and also breast cancer.

More recently a nanomolar formulation of paclitaxel complexed with albumin has been shown to be at least as efficacious and less myelosuppressive than paclitaxel alone. (APP; Abraxane). Paclitaxel mconjugates with glutamic acid are also in development.

Another taxane compound which has been used in the clinic is docetaxel which has been shown to have particular efficacy in the treatment of advanced breast cancer. Docetaxel has shown a better solubility in excipient systems than paclitaxel, therefore increasing the ease with which it can be handled and used in pharmaceutical compositions.

Biological activity: The taxane compounds of the combinations of the invention are tubulin targeting agents and have activity against various cancers.

Problems: Clinical use of taxanes has demonstrated a narrow therapeutic index with many patients unable to tolerate the side effects associated with its use. There is therefore a need to increase the inhibitory efficacy of taxane compounds against tumour growth and also to provide a means for the use of lower dosages of taxane compounds to reduce the potential of adverse toxic side effects to the patient. The development of taxanes with increased solubility in aqueous solutions would also be desirable.

Preferences: Preferred taxane compounds for use in accordance with the invention include paclitaxel Abraxane or docetaxel referred to herein. Paclitaxel is available commercially for example under the trade name Taxol from Bristol Myers Squibb and docetaxel is available commercially under the trade name Taxotere from Sanofi-Aventis (previously Rhone-Poulenc Rorer). Both compounds and other taxane compounds may be prepared in conventional manner for example as described in EP 253738, EP 253739 and WO 92/09589 or by processes analogous thereto.

Specific embodiments: In one embodiment, the taxane compound is paclitaxel. In another embodiment, the taxane compound is docetaxel.

Posology: The taxane compound is advantageously administered in a dosage of 50 to 400 mg per square metere (mg/m²) of body surface area, for example 75 to 250 mg/m², particularly for paclitaxel in a dosage of about 175 to 250 mg/m² and for docetaxel in about 75 to 150 mg/m² per course of treatment. These dosages may be administered for example once, twice or more per course of treatment, which may be repeated for example every 7,14, 21 or 28 days.

### 9. Epothilones

Definition: As used herein, the term “epothilone” is used to define a class of cytotoxic macrolides with a similar mechanism of action to paclitaxel but with the potential advantage of activity in taxane-resistant settings in preclinical models. The epothilones ixabepilone, patupilone, BMS-310705, KOS-862 and ZK-EPO are in early clinical trials for cancer treatment. Phase I studies have shown that dose-limiting toxicities of epothilones are generally neurotoxicity and neutropenia although initial studies with patupilone indicated that diarrhoea was dose limiting. Neuropathy induced by ixabepilone may be schedule dependent. Response rates in taxane-refractory metastatic breast cancer are relatively modest, but ixabepilone and patupilone have shown promising efficacy in hormone-refractory metastatic prostate cancer and in taxane-refractory ovarian cancer.

Technical Background; Epothilones A and B were originally isolated as anti-fungal fermentation products of the myxobacteria Sorangium cellulosum. Shortly thereafter these agents were demonstrated to stabilize microtubules and induce mitotic arrest. Though their cytotoxic activity relies on the same mechanism as that of the taxanes, the epothilones have a couple of key advantages. Firstly they are not substrates for the multi-drug resistance pump P-gylycoprotein. Secondly they are easier both to produce (because of their bacterial origin) and to manipulate. Chemical syntheses, either total or partial, of these molecules and their analogs allows for modification to enhance their efficacy Mani et al. Anticancer Drugs 2004; 15(6):553-8). Several epothilones or epothilone-derivatives have been shown effective against cell lines and tumor xenografts and are now in clinical trials (Goodin et al. J Clin Oncol 2004; 22(10): 2015-25). An unexpected source for the identification of microtubule stabilizing agents has been marine organisms. Laulimalide and isolaulimalide are natural products of the marine sponge Cacospongia mycofijiensis with strong paclitaxel-like activity, even against P-gp expressing cell lines. Eluetherobin, similar in both respects, is a product of the Eleutherobia species of soft coral.

Biological Activity; Formation of microtubules involves polymerization of heterodimeric α/β-tubulin subunits with multiple isoforms of both α- and β-tubulin present in human cells. Intact microtubule function is required for formation and functioning of the mitotic spindle, and cells treated with agents that bind either tubulin subunits or polymerized microtubules exhibit alterations in spindle formation, as well as arrest at the G2/M phase of the cell cycle, which is associated with induction of apoptosis. Compounds that target microtubules are potent cytotoxic agents, exemplified by the convergent evolution of microtubule-targeting compounds by a variety of plant and marine species. Published studies of three epothilones in current clinical development, epothilone B, aza-epothilone B, and desoxyepothilone B, indicate that these compounds exhibit broad spectrum antitumor activity in cell culture models and in xenografts. Furthermore, epothilones are generally more cytotoxic than paclitaxel in cell culture studies, with IC₅₀ values in the sub- or low nanomolar range in a variety of tumor cell lines (Bollag et al. Cancer Res 55:2325-2333, 1995; Lee et al. Clin Cancer Res 7:1429-1437, 2001; Chou et al. Proc Natl Acad Sci USA 95:9642-9647, 1998; Newman et al. Cancer Chemother Pharmacol 48:319-326, 2001). Preclinical studies also demonstrated important differences with regard to drug resistance mechanisms between epothilones and taxanes. In particular, overexpression of P-glycoprotein minimally affects the cytotoxicity of epothilone B, aza-epothilone B, and desoxyepothilones in cell culture models. Comparison of the cytotoxic effects of epothilone B, aza-epothilone B, and desoxyepothilone B among P-glycoprotein-overexpressing cell lines suggests that desoxyepothilone B is least affected, whereas aza-epothilone B is most affected by P-glycoprotein expression. However, it should be noted that differences among the IC₅₀s of these compounds in P-glycoprotein-overexpressing cell lines are small compared with the differences between these values and IC₅₀s for paclitaxel in these cell lines. Although the significance of P-glycoprotein expression in clinical resistance to taxanes remains uncertain, these results suggest that epothilones may be more active than taxanes in patients with malignancies characterized by high levels of P-glycoprotein expression. In vivo studies indicate that epothilones are active in paclitaxel-sensitive and -resistant tumor models using a variety of schedules. When administered intravenously to mice using intermittent daily or weekly schedules, aza-epothilone B is highly active in ovarian, colon, and breast xenografts and induces cures in an ovarian xenograft model (Pat-7) that is resistant to paclitaxel. Notably, unlike paclitaxel, aza-epothilone B is effective when administered orally in preclinical models. This phenomenon likely relates to the expression of P-glycoprotein in intestinal mucosa, resulting in poor absorption of paclitaxel but not epothilones.

Problems; Sensory neuropathy and myelosuppression has been documented with epothilones

Preferences; Existing structure-activity data provide some insight into the interaction between epothilones and microtubules. Results from several groups indicate that modifications at or near the C12-13 epoxide can affect microtubule-stabilizing activity (Wartmann and Altmann, Curr Med Chem Anti-Canc Agents 2:123-148, 2002). For example, addition of a methyl group to epothilone A at position C12 yields epothilone B, which is approximately twice as potent as epothiloneA or paclitaxel in inducing tubulin polymerization in vitro (Kowalski et al. J Biol Chem 272: 2534-2541, 1997; Nicolaou et al. Nature 387:268-272, 1997, abstr 428). In addition, it is clear that an epoxide at C12-13 is not required for microtubule-binding, because desoxyepothilone B (also known as epothilone D or KOS-862) lacks the C12-13 epoxide and is a more potent microtubule stabilizer in vitrothan epothilone A or B. Less data are available regarding the effects of modifying other regions of epothilone. Despite attempts to improve microtubule binding by altering the C9-C12 region (on the basis of molecular modeling), alterations in this area resulted in loss of cytotoxic activity. By contrast, replacement of the lactone oxygen of epothilone B with a lactam (aza-epothilone B, also known as BMS-247550) does not impair microtubule-polymerizing activity or cytotoxicity. Although a variety of other epothilone analogs have been synthesized, it should be noted that increasing microtubule-stabilizing activity does not always result in increased cytotoxicity, presumably because of the importance of other variables such as cellular accumulation and metabolic stability (Wartmann and Altmann, Curr Med Chem Anti-Canc Agents 2:123-148, 2002). Indeed, replacement of the methyl group at C12 position of desoxyepothilone B with a propanol group results in a compound that is as effective as desoxyepothilone B against the leukemic cell line CCRF-CEM but is significantly less active against a P-glycoprotein-overexpressing subline (IC₅₀ of 17 nmol/L for desoxyepothilone B v 167 nmol/L for the propanol derivative) (Chou et al. Proc Natl Acad Sci USA 95:9642-9647, 1998). Additional modifications of naturally occurring epothilones have been made in an effort to improve solubility, such as BMS-310705, which is a C-21-substituted derivative of epothilone B (Lee et al., Proc Am Assoc Cancer Res 43:a3928, 2002).

Specific embodiments: In one embodiment, the epothilone compound is BMS-247550. In another embodiment, the epothilone compound is Desoxyeopthilone and in another embodiment the epothilone compound is BMS-310705 Posology. BMS-247550 is dosed either 40 mg/m² over 3 hours every 21 days or 6 mg/m² administered over 1 hour daily times 5 days every 3 weeks. Because of the frequency of mucositis and neutropenia in the first 18 patients on the single-dose every-3-week schedule, the dose was reduced to 32 mg/m². EP0906 is dosed either at 2.5 mg/m² weekly for 3 weeks followed by 1 week of rest in one trial, and 6 mg/m² once every 3 weeks. KOS-862 is scheduled at either a single dose every 3 weeks, a daily dose times 3 every 3 weeks, a fixed rate dose every 3 weeks, and a weekly dose for 3 weeks with 1 week rest.

### 10. Platinum Compounds

Definition: The term “platinum compounds” as used herein refers to any tumour cell growth inhibiting platinum compound including platinum coordination compounds, compounds which provide platinum in the form of an ion and analogues of platinum compounds as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Technical background: In the chemotherapeutic treatment of cancers, cisplatin (cis-diaminodichloroplatinum (II)) has been used successfully for many years in the treatment of various human solid malignant tumours for example testicular cancer, ovarian cancer and cancers of the head and neck, bladder, oesophagus and lung.

More recently, other diamino-platinum complexes, for example carboplatin (diamino(1,1-cyclobutane-dicarboxylato)platinum (II)), have also shown efficacy as chemotherapeutic agents in the treatment of various human solid malignant tumours, carboplatin being approved for the treatment of ovarian and small cell lung cancer in particular in the treatment of ovarian cancer. A further antitumour platinum compound is oxaliplatin (L-OHP), a third generation diamino-cyclohexane platinum-based cytotoxic drug, which has the chemical name (1,2-diaminocyclohexane)oxalato-platinum (II). Oxaliplatin is used, for example, for the treatment of metastatic colorectal cancer, based on its lack of renal toxicity and higher efficacy in preclinical models of cancer in comparison to cisplatin. Oxaliplatin is used in combination with 5-FU, for the treatment of metastatic colorectal cancer and is under investigation in the treatment of upper gastrointestinal cancer. An oral platinum derivative is under investigation for the treatment of prostate cancer.

Biological activity: The platinum compounds of the combinations of the invention have activity against various cancers.

Problems: Although cisplatin and other platinum compounds have been widely used as chemotherapeutic agents in humans, they are not therapeutically effective in all patients or against all types of tumours. Moreover, such compounds need to be administered at relatively high dosage levels which can lead to toxicity problems such as kidney damage, myelosuppression and neuropathy. Also, and especially with cisplatin, the compounds cause nausea and vomiting in patients to a varying extent, as well as leucopenia, anemia and thrombocytopenia. There is therefore a need to increase efficacy and also to provide a means for the use of lower dosages to reduce the potential of adverse toxic side effects to the patient.

Preferences: Preferred platinum compounds for use in accordance with the invention include cisplatin, carboplatin and oxaliplatin. Other platinum compounds include chloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane)-(isocitrato)platinum (II); (1,2-diaminocyclohexane)-cis-(pyruvato)platinum (II); onnaplatin; and tetraplatin. Cisplatin is commercially available for example under the trade name Platinol from Bristol-Myers Squibb Corporation as a powder for constitution with water, sterile saline or other suitable vehicle. Cisplatin may also be prepared for example as described by G. B. Kauffman and D. O. Cowan, lnorg. Synth. 7, 239 (1963), or by processes analogous thereto. Carboplatin is commercially available for example from Bristol-Myers Squibb Corporation under the trade name Paraplatin, or may be prepared for example as described in U.S. patent specification No. 4140707, or by processes analogous thereto. Oxaliplatin is commercially available for example from Sanofi-Synthelabo Inc under the trade name Eloxatin, or may be prepared for example as described in U.S. patent specification No. 4169846, or by processes analogous thereto. Other platinum compounds and their pharmaceutical compositions are commercially available and/or can be prepared by conventional techniques.

Specific embodiments: In one embodiment, the platinum compound is selected from chloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane)-(isocitrato)platinum (II); (1,2-diaminocyclohexane)-cis-(pyruvato)platinum (II); onnaplatin; tetraplatin, cisplatin, carboplatin and oxaliplatin. In another embodiment, the platinum compound is a platinum compound other than cisplatin, for example a platinum compound such as chloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane)-(isocitrato)platinum (II); (1,2-diaminocyclohexane)-cis-(pyruvato)platinum (II); onnaplatin; tetraplatin, carboplatin or oxaliplatin, preferably selected from carboplatin and oxaliplatin.

Posology: The platinum coordination compound is advantageously administered in a dosage of 1 to 500 mg per square meter (mg/m²) of body surface area, for example 50 to 400 mg/m² or 500 mg/m² (e.g. 50 to 400 mg/m²) particularly for cisplatin in a dosage of about 75 mg/m², for carboplatin in about 300-500 mg/m² e.g. 300 mg/m², and for oxaliplatin in about 50-100 mg/m². These dosages may be administered for example once, twice or more per course of treatment, which may be repeated for example every 7, 14, 21 or 28 days.

### 11. Topoisomerase 2 Inhibitors

Definition: The term “topoisomerase 2 inhibitor” as used herein refers to topoisomerase 2 inhibitor or analogues of topoisomerase 2 inhibitor as described above, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Technical background: An important class of anticancer drugs are the inhibitors of the enzyme topoisomerase 2 which causes double-strand breaks to release stress build-up during DNA transcription and translation. Compounds that inhibit the function of this enzyme are therefore cytotoxic and useful as anti-cancer agents.

Among the topoisomerase 2 inhibitors which have been developed and used in cancer chemotherapy are the podophyllotoxins. These drugs act by a mechanism of action which involves the induction of DNA strand breaks by an interaction with DNA topoisomerase 2 or the formation of free radicals. Podophyllotoxin, which is extracted from the mandrake plant, is the parent compound from which two glycosides have been developed which show significant therapeutic activity in several human neoplasms, including pediatric leukemia, small cell carcinomas of the lung, testicular tumours, Hodgkin's disease, and non-Hodgkin's lymphomas. Podophyllotoxin has activity in pediatric leukemia, small cell carcinomas of the lung, testicular tumours, Hodgkin's disease, and large cell lymphomas. These derivatives are etoposide (VP-16), which has the chemical name 4′-demethylepipodophyllotoxin 9-[4,6-O—(R)-ethylidene-β-D-glucopyranoside], and teniposide (VM-26), which has the chemical name 4′-demethylepipodophyllotoxin 9-[4,6-O—(R)-2-thenylidene-β-D-glucopyranoside].

Both etoposide and teniposide, however, suffer from certain toxic side-effects especially myelosuppression. Another important class of topoisomerase 2 inhibitors are the anthracycline derivatives which are important anti-tumour agents and comprise antibiotics obtained from the fungus Streptomyces peuticus var. caesius and their derivatives, characterized by having a tetracycline ring structure with an unusual sugar, daunosamine, attached by a glycosidic linkage. Among these compounds, the most widely used include daunorubicin, which has the chemical name 7-(3-amino-2,3,6-trideoxy-L-lyxohexosyloxy)-9-acetyl-7,8,9,10-tetrahydro-6,9,11-trihydroxy-4-methoxy-5,12-naphthacenequinone, doxorubicin, which has the chemical name 10-[(3-amino-2,3,6-trideoxy-α-L-lyxohexopyranosyl)oxy]-7,8,9,10-tetrahydro-6,8,11-trihydroxy-8-(hydroxylacetyl)-1-methoxy-5,12-naphthacenedione, and idarubicin (Zavedos™), which has the chemical name 9-acetyl-[(3-amino-2,3,6-trideoxy-α-L-lyxohexopyranosyl)oxy]-7,8,9,10-tetrahydro-6,9,11-trihydroxy-5,12-naphthacenedione.

Daunorubicin and idarubicin have been used primarily for the treatment of acute leukaemias whereas doxorubicin has been more widely tested against solid tumours particularly breast cancer. Another anthracycline derivative which is useful in cancer chemotherapy is epirubicin. Epirubicin, which has the chemical name (8S-cis)-10-[(3-amino-2,3,6-trideoxy-α-L-arabino-hexopyranosyl)oxy]-7,8,9,10-tetrahydro-6,8,11-trihydroxy-8-(hydroxyacetyl)-1-methoxy-5,12-naphthacenedione, is a doxorubicin analog having a catabolic pathway that involves glucuronidation, by uridine diphosphate-glucuronosyl transferase in the liver (unlike that for doxorubicin), which is believed to account for its shorter half-life and reduced cardiotoxicity. The compound has been used for the treatment of various cancers including cervical cancer, endometrial cancer, advanced breast cancer and carcinoma of the bladder but suffers from the side-effects of myelosuppression and cardiotoxicity. The latter side-effect is typical of anthracycline derivatives which generally display a serious cardiomyopathy at higher cumulative doses. A further type of topoisomerase 2 inhibitor is represented by mitoxantrone, which has the chemical name 1,4-dihydroxy-5,8-bis[[2-[(2-hydroxyethyl)amino]ethyl]amino]-9,10-anthracenedione, and is used for the treatment of multiple sclerosis, non-Hodgkin's lymphoma, acute myelogenous leukaemia, and breast, prostate and liver tumours. Others include losoxantrone and actinomycin D (the latter agent also known as Dactinomycin and Cosmegen Lyovac®).

Side-effects from administration of mitoxantrone include myelosuppression, nausea, vomiting, stomatitis and alopecia

Biological activity: The topoisomerase 2 inhibitors of the combinations of the invention have activity against various cancers as described above.

Problems: This class of cytotoxic compound is associated with side effects, as mentioned above: Thus, there is a need to provide a means for the use of lower dosages to reduce the potential of adverse toxic side effects to the patient.

Preferences: Preferred topoisomerase 2 inhibitor compounds for use in accordance with the invention include anthracycline derivatives, mitoxantrone and podophyllotoxin derivatives as defined to herein.

Preferred anti-tumour anthracycline derivatives for use in accordance with the invention include daunorubicin, doxorubicin, idarubicin and epirubicin referred to above. Daunorubicin is commercially available for example as the hydrochloride salt from Bedford Laboratories under the trade name Cerubidine, or may be prepared for example as described in U.S. patent specification No. 4020270, or by processes analogous thereto. The therapeutic index of daunorubicin in acute myeloid leukemia may be improved by encapsulating the molecule in a liposome (Daunoxome; Gilead/Diatos). Doxorubicin is commercially available for example from Pharmacia and Upjohn Co under the trade name Adriamycin, or may be prepared for example as described in U.S. patent specification No. 3803124, or by processes analogous thereto. Doxorubicin derivatives include pegylated doxorubicin hydrochloride and liposome-encapsulated doxorubicin citrate. Pegylated doxorubicin hydrochloride is commercially available from Schering-Plough Pharmaceuticals under the trade name Caeylx; non-pegylated liposome-encapsulated doxorubicin citrate is commercially available for example from Cephalon Europe under the trade name Myocet. Idarubicin is commercially available for example as the hydrochloride salt from Pharmacia & Upjohn under the trade name Idamycin, or may be prepared for example as described in U.S. patent specification No. 4046878, or by processes analogous thereto. Epirubicin is commercially available for example from Pharmacia and Upjohn Co under the trade name Pharmorubicin, or may be prepared for example as described in U.S. patent specification No 4058519, or by processes analogous thereto. Mitoxantrone is commercially available for example from OSI Pharmaceuticals, under the trade name Novantrone, or may be prepared for example as described in U.S. patent specification No. 4197249, or by processes analogous thereto.

Other anti-tumour anthracycline derivatives may be prepared in conventional manner for example by processes analogous to those described above for the specific anthracycline derivatives.

Preferred anti-tumour podophyllotoxin derivatives for use in accordance with the invention include etoposide and teniposide referred to above. Etoposide is commercially available for example from Bristol-Myers Squibb Co under the trade name VePesid, or may be prepared for example as described in European patent specification Nol 11058, or by processes analogous thereto. Teniposide is commercially available for example from Bristol-Myers Squibb Co under the trade name Vumon, or may be prepared for example as described in PCT patent specification No. WO 93/02094, or by processes analogous thereto. Other anti-tumour podophyllotoxin derivatives may be prepared in conventional manner for example by processes analogous to those described above for etoposide and teniposide.

Specific embodiments: In one embodiment, the topoisomerase 2 inhibitor is an anthracycline derivative, mitoxantrone or a podophyllotoxin derivative. In another embodiment, the topoisomerase 2 inhibitor is selected from daunorubicin, doxorubicin, idarubicin and epirubicin. In a further embodiment, the topoisomerase 2 inhibitor is selected from etoposide and teniposide. Thus, in a preferred embodiment, the topoisomerase 2 inhibitor is etoposide. In another embodiment, the topoisomerase 2 inhibitor is an anthracycline derivative other than doxorubicin, for example a topoisomerase 2 inhibitor such as daunorubicin, idarubicin and epirubicin.

Posology: The anti-tumour anthracycline derivative is advantageously administered in a dosage of 10 to 150 mg per square meter (mg/m²) of body surface area, for example 15 to 60 mg/m², particularly for doxorubicin in a dosage of about 40 to 75 mg/m², for daunorubicin in a dosage of about 25 to 45 mg/m², for idarubicin in a dosage of about 10 to 15 mg/m² and for epirubicin in a dosage of about 100-120 mg/m².

Mitoxantrone is advantageously administered in a dosage of about 12 to 14 mg/m² as a short intravenous infusion about every 21 days.

The anti-tumour podophyllotoxin derivative is advantageously administered in a dosage of 30 to 300 mg/m² of body surface area, for example 50 to 250 mg/m particularly for etoposide in a dosage of about 35 to 100 mg/m, and for teniposide in about 50 to 250 mg/m².

The dosages noted above may generally be administered for example once, twice or more per course of treatment, which may be repeated for example every 7, 14, 21 or 28 days.

### 12. Alkylating Agents

Definition: The term “alkylating agent” or “alkylating agents” as used herein refers to alkylating agents or analogues of alkylating agents as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: Alkylating agents used in cancer chemotherapy encompass a diverse group of chemicals that have the common feature that they have the capacity to contribute, under physiological conditions, alkyl groups to biologically vital macromolecules such as DNA. With most of the more important agents such as the nitrogen mustards and the nitrosoureas, the active alkylating moieties are generated in vivo after complex degradative reactions, some of which are enzymatic. The most important pharmacological actions of the alkylating agents are those that disturb the fundamental mechanisms concerned with cell proliferation, in particular DNA synthesis and cell division. The capacity of alkylating agents to interfere with DNA function and integrity in rapidly proliferating tissues provides the basis for their therapeutic applications and for many of their toxic properties. Alkylating agents as a class have therefore been investigated for their anti-tumour activity and certain of these compounds have been widely used in anti-cancer therapy although they tend to have in common a propensity to cause dose-limiting toxicity to bone marrow elements and to a lesser extent the intestinal mucosa.

Among the alkylating agents, the nitrogen mustards represent an important group of anti-tumour compounds which are characterised by the presence of a bis-(2-chloroethyl) grouping and include cyclophosphamide, which has the chemical name 2-[bis(2-chloroethyl)amino]tetrahydro-2H-1,3,2-oxazaphospholine oxide, and chlorambucil, which has the chemical name 4-[bis(2-chloroethyl)amino]-benzenebutoic acid. Cyclophosphamide has a broad spectrum of clinical activity and is used as a component of many effective drug combinations for non-Hodgkin's lymphoma, Hodgkin's disease, Burkitt's lymphoma and breast cancer. Cyclophosphamide has also been used as a component of combinations for malignant lymphomas.

Ifosfamide (a.k.a. ifosphamide) is a structural analogue of cyclophosphamide and its mechanism of action is presumed to be identical. It has the chemical name 3-(2-chloroethyl)-2-[(2-chloroethyl)amino]tetrahydro-2H-1,3,2-oxazaphosphorin-2-oxide, and is used for the treatment of cervical cancer, sarcoma, and testicular cancer but may have severe urotoxic effects. Chlorambucil has been used for treating chronic lymphocytic leukaemia and non-Hodgkin's lymphoma. Chlorambucil has also been used for treating CLL and malignant lymphomas including lymphosarcoma.

Another important class of alkylating agents are the nitrosoureas which are characterised by the capacity to undergo spontaneous non-enzymatic degradation with the formation of the 2-chloroethyl carbonium ion. Examples of such nitrosourea compounds include carmustine (BiCNU® or BCNU) which has the chemical name 1,3-bis(2-chloroethyl)-1-nitrosourea, and lomustine (CCNU) which has the chemical name 1-(2-chloroethyl)cyclohexyl-1-nitrosourea. Carmustine and lomustine each have an important therapeutic role in the treatment of brain tumours and gastrointestinal neoplasms although these compounds cause profound, cumulative myelosuppression that restricts their therapeutic value.

Another class of alkylating agent is represented by the bifunctional alkylating agents having a bis-alkanesulfonate group and represented by the compound busulfan which has the chemical name 1,4-butanediol dimethanesulfonate, and is used for the treatment of chronic myelogenous (myeloid, myelocytic or granulocytic) leukaemia. However, it can induce severe bone marrow failure resulting in severe pancytopenia. This property has led to its widespread usage as a conditioning agent prior to hematological stem cell transplantation.

Another class of alkylating agent are the aziridine compounds containing a three-membered nitrogen-containing ring which act as anti-tumour agents by binding to DNA, leading to cross-linking and inhibition of DNA synthesis and function. An example of such an agent is mitomycin, an antibiotic isolated from Streptomyces caespitosus, and having the chemical name 7-amino-9α-methoxymitosane.

Mitomycin is used to treat adenocarcinoma of stomach, pancreas, colon and breast, small cell and non-small cell lung cancer, and, in combination with radiation, head and neck cancer, side-effects including myelosuppression, nephrotoxicity, interstitial pneumonitis, nausea and vomiting.

Biological activity: One of the most important pharmacological actions of the alkylating agent in combination with the invention is its ability to disturb the fundamental mechanisms concerned with cell proliferation as herein before defined. This capacity to interfere with DNA function and integrity in rapidly proliferating tissues provides the basis for their therapeutic application against various cancers.

Problems: This class of cytotoxic compound is associated with side effects, as mentioned above. Thus, there is a need to provide a means for the use of lower dosages to reduce the potential of adverse toxic side effects to the patient.

Preferences: Preferred alkylating agents for use in accordance with the invention include the nitrogen mustard compounds cyclophosphamide, ifosfamide/ifosphamide and chlorambucil and the nitrosourea compounds carmustine and lomustine referred to above. Preferred nitrogen mustard compounds for use in accordance with the invention include cyclophosphamide, ifosfamide/ifosphamide and chlorambucil referred to above. Cyclophosphamide is commercially available for example from Bristol-Myers Squibb Corporation under the trade name Cytoxan, or may be prepared for example as described in U.K. patent specification No. 1235022, or by processes analogous thereto. Chlorambucil is commercially available for example from GlaxoSmithKline plc under the trade name Leukeran, or may be prepared for example as described in U.S. patent specification No. 3046301, or by processes analogous thereto. Ifosfamide/ifosphamide is commercially available for example from Baxter Oncology under the trade name Mitoxana, or may be prepared for example as described in U.S. patent specification No. 3732340, or by processes analogous thereto. Preferred nitrosourea compounds for use in accordance with the invention include carmustine and lomustine referred to above. Carmustine is commercially available for example from Bristol-Myers Squibb Corporation under the trade name BiCNU, or may be prepared for example as described in European patent specification No. 902015, or by processes analogous thereto. Lomustine is commercially available for example from Bristol-Myers Squibb Corporation under the trade name CeeNU, or may be prepared for example as described in U.S. patent specification No. 4377687, or by processes analogous thereto. Busulfan is commercially available for example from GlaxoSmithKline pic under the trade name Myleran, or may be prepared for example as described in U.S. patent specification No. 2917432, or by processes analogous thereto. Mitomycin is commercially available for example from Bristol-Myers Squibb Corporation under the trade name Mutamycin. Others include estramustine, mechlorethamine, melphalan, bischloroethylnitrosurea, cyclohexylchloroethylnitrosurea, methylcyclohexylchloroethylnitrosurea, nimustine, procarbazine, dacarbazine, temozolimide and thiotepa.

Specific embodiments: In one embodiment, the alkylating agent is a nitrogen mustard compound selected from cyclophosphamide, ifosfamide/ifosphamide and chlorambucil. In another embodiment, the alkylating agent is a nitrosurea selected from carmustine and lomustine. The alkylating agents further include Busulfan. In one embodiment, the alkylating agents are as herein before defined other than mitomycin C or cyclophosphamide.

Posology: The nitrogen mustard or nitrosourea alkylating agent is advantageously administered in a dosage of 100 to 9000 e.g. 100 to 2500 mg per square meter (mg/m²) of body surface area, for example 100 to 5000, 100 to 2500 or 120 to 500 mg/m², particularly for cyclophosphamide in a dosage of about 100 to 5000 e.g. 100 to 500 mg/m², for ifosfamide/ifosphamide in a dosage of 500-9000 mg/m² e.g. 500-2500 mg/m², for chlorambucil in a dosage of about 0.1 to 0.2 mg/kg, for carmustine in a dosage of about 150 to 200 mg/m² and for lomustine in a dosage of about 100 to 150 mg/m². For bis-alkanesulfonate compounds such as busulphan a typical dose may be 1-2 mg/m², e.g. about 1.8 mg/m².

Aziridine alkylating agents such as mitomycin can be administered for example in a dosage of 15 to 25 mg/m² preferably about 20 mg/m².

The dosages noted above may be administered for example once, twice or more per course of treatment, which may be repeated for example every 7, 14, 21 or 28 days.

### 13. Signalling Inhibitors for Use According to the Invention

Definition: The term “signalling inhibitor” (or “signal transduction inhibitor”) as used herein refers to signalling inhibitors or analogues of signalling inhibitors as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: A malignant tumour is the product of uncontrolled cell proliferation. Cell growth is controlled by a delicate balance between growth-promoting and growth-inhibiting factors. In normal tissue the production and activity of these factors results in differentiated cells growing in a controlled and regulated manner that maintains the normal integrity and functioning of the organ. The malignant cell has evaded this control; the natural balance is disturbed (via a variety of mechanisms) and unregulated, aberrant cell growth occurs.

One driver for growth is the epidermal growth factor (EGF), and the receptor for EGF (EGFR) has been implicated in the development and progression of a number of human solid tumours including those of the lung, breast, prostate, colon, ovary, head and neck. EGFR is a member of a family of four receptors, namely EGFR (HER1 or ErbB1), ErbB2 (HER2/neu), ErbB3 (HER3), and ErbB4 (HER4). These receptors are large proteins that reside in the cell membrane, each having a specific external ligand binding domain, a transmembrane domain and an internal domain which has tyrosine kinase enzyme activity. When EGF attaches to EGFR, it activates the tyrosine kinase, triggering reactions that cause the cells to grow and multiply. EGFR is found at abnormally high levels on the surface of many types of cancer cells, which may divide excessively in the presence of EGF. Inhibition of EGFR activity has therefore been a target for chemotherapeutic research in the treatment of cancer. Such inhibition can be effected by direct interference with the target EGFR on the cell surface, for example by the use of antibodies, or by inhibiting the subsequent tyrosine kinase activity.

Examples of antibodies which target EGFR are the monoclonal antibodies trastuzumab and cetuximab. Amplification of the human epidermal growth factor receptor 2 protein (HER 2) in primary breast carcinomas has been shown to correlate with a poor clinical prognosis for certain patients. Trastuzumab is a highly purified recombinant DNA-derived humanized monoclonal IgG1 kappa antibody that binds with high affinity and specificity to the extracellular domain of the HER2 receptor. In vitro and in vivo preclinical studies have shown that administration of trastuzumab alone or in combination with paclitaxel or carboplatin significantly inhibits the growth of breast tumour-derived cell lines that over-express the HER2 gene product. In clinical studies trastuzumab has been shown to have clinical activity in the treatment of breast cancer. The most common adverse effects of trastuzumab are fever and chills, pain, asthenia, nausea, vomiting, diarrhea, headache, dyspnea, rhinitis, and insomnia. Particularly troublesome is the onset of cardiomyopathy which may be reversible in the majority of patients. Trastuzumab has been approved for the treatment of early and metastatic breast cancer, in particular metastic breast cancer, exhibiting over-expression of the HER2 protein

Cetuximab has been used for the treatment of irotecan-refractory colorectal cancer (CRC) and in combination with radiotherapy in the treatment of head and neck cancer. It is also being evaluated both as a single agent and in combination with other agents for use in the treatment of a variety of other cancers including metastatic pancreatic carcinoma, and non-small-cell lung cancer. The administration of cetuximab can cause serious side effects, which may include difficulty in breathing and low blood pressure.

Another suitable monoclonal antibody for use in the combinations of the invention is panitumumab. Amgen Inc (formerly Immunex and Abgenix Inc) is developing panitumumab (ABX-EGF), a fully human monoclonal antibody against the EGF receptor, for the potential treatment of cancer, such as monotherapy for renal cancer, non-small-cell lung cancer, and CRC in combination with standard chemotherapy as first-line treatment, as third-line monotherapy in advanced CRC, in particular to treat metastatic colorectal cancer (MCC) and in patients who failed standard chemotherapy. Thus ABX-EGF can be administered as a monotherapy or in association with chemotherapy and radiotherapy in order to complement independent approaches for the treatment of cancer.

ABX-EGF is a fully humanized IgG2 monoclonal antibody against the human EGFR. Fully humanized monoclonal antibodies such as ABX-EGF have several advantages over chimeric antibodies, which contain significant amounts of mouse protein. They do not generate human anti-mouse antibodies (HAMA); the risk of inducing hypersensitivity reactions in patients is therefore reduced and the antibodies should demonstrate an increased in vivo lifetime. Such considerations may be important for long-term administration.

It can be prepared as described in WO98/50433 and process analogous thereto.

Panitumumab may be dosed ranging from 0.01 to 5.0 mg/kg once per week, 6.0 mg/kg once every two weeks or 9.0 mg/kg once every three weeks administered by intravenous infusion.

In a Phase 3 pivotal study examining panitumumab as third-line monotherapy in colorectal cancer patients, patients received panitumumab every two weeks.

The farnesyltransferase inhibitor tipifarnib prevents signaling thru ras-mediated pathways and is under investigation for the treatment of myeloid leukemias.

Examples of agents which target EGFR tyrosine kinase activity include the tyrosine kinase inhibitors gefitinib and erlotinib. Gefitinib which has the chemical name 4-(3-chloro-4-fluoroanilino)-7-methoxy-6-(3-morpholinopropoxy)quinazoline, is used for the treatment of non-small-cell lung cancer. It has also been studied for other solid tumours that over-express EGF receptors such as breast and colorectal cancer. It has been found that patients receiving gefitinib may develop interstitial lung disease and eye irritation Erlotinib, which has the chemical name N-(3-ethynyl-phenyl)-6,7-bis(2-methoxyethoxy)-4-quinazoline, has also been used for the treatment of non-small-cell lung cancer, and is being developed for the treatment of various other solid tumours such as pancreatic cancer, the most common side effects being rash, loss of appetite and fatigue; a more serious side effect which has been reported is interstitial lung disease.

Another growth factor which has received attention as a target for anticancer research is the vascular endothelial growth factor (VEGF). VEGF is a key regulator of vasculogenesis during angiogenic processes including wound healing, retinopathy, psoriasis, inflammatory disorders, tumour growth and metastasis. Studies have shown that over-expression of VEGF is strongly associated with invasion and metastasis in human malignant disease.

An example of an antibody that targets the VEGF antigen on the surface of a cell is the monoclonal antibody bevacizumab which is a recombinant humanised monoclonal IgG1 antibody that binds to and inhibits VEGF. Bevacizumab has been used for the treatment of colorectal cancer, for example in combination with chemotherapy e.g. 5-fluorouracil. Bevacizumab also being developed as a potential treatment for other solid tumours such as metastatic breast cancer, metastatic non-small-cell lung cancer and renal cell carcinoma. The most serious adverse events associated with bevacizumab include gastrointestinal perforations, hypertensive crises, nephrotic syndrome and congestive heart failure. Other therapeutic agents in development which target the action of VEGF at alternate points in the signal transduction cascade intiated by this growth factor include sunitinib which is marketed under the trade name Sutent by Sugen/Pfizer and inhibits the kinase activity of the VEGF receptor. Sutent has demonstrated efficacy in Phase III trials in gastrointestinal stromal tumours.

Another growth factor of importance in tumour development is the platelet-derived growth factor (PDGF) that comprises a family of peptide growth factors that signal through cell surface tyrosine kinase receptors (PDGFR) and stimulate various cellular functions including growth, proliferation, and differentiation. PDGF expression has been demonstrated in a number of different solid tumours including glioblastomas and prostate carcinomas. The tyrosine kinase inhibitor imatinib mesylate, which has the chemical name 4-[(4-methyl-1-piperazinyl)methyl]-N-[4-methyl-3-[[4-(3-pyridinyl)-2-ylpyridinyl]amino]-phenyl]benzamide methanesulfonate, blocks activity of the Bcr-Abl oncoprotein and the cell surface tyrosine kinase receptor c-Kit, and as such is approved for the treatment on chronic myeloid leukemia and gastrointestinal stromal tumours. Imatinib mesylate is also a potent inhibitor of PDGFR kinase and is currently being evaluated for the treatment of chronic myelomonocytic leukemia and glioblastoma multiforme, based upon evidence in these diseases of activating mutations in PDGFR. The most frequently reported drug-related adverse events were edema, nausea, vomiting, cramps and musculosketetal pain.

A further growth factor target for cancer chemotherapy is inhibition of Raf which is a key enzyme in the chain reaction of the body's chemistry that triggers cell growth. Abnormal activation of this pathway is a common factor in the development of most cancers, including two-thirds of melanomas. By blocking the action of Raf kinase, it may be possible to reverse the progression of these tumours. One such inhibitor is sorafenib (a.k.a. BAY 43-9006 and Nexavar) which has the chemical name 4-(4-(3-(4-chloro-3-(trifluoromethyl)phenyl)ureido)phenoxy)-N2-methylpyridine-2-carboxamide. Sorafenib targets both the Raf signalling pathway to inhibit cell proliferation and the VEGFR/PDGFR signalling cascades to inhibit tumour angiogenesis. Raf kinase is a specific enzyme in the Ras pathway. Mutations in the Ras gene occur in approximately 20 percent of all human cancers, including 90 percent of pancreatic cancers, 50 percent of colon cancers and 30 percent of non-small cell lung cancers. Sorafenib is being investigated for the treatment of a number of cancers including liver and kidney cancer. The most common side effects of sorafenib are pain, swelling, redness of the hands and/or feet, and also rash, fatigue and diarrhea.

Biological activity: The signalling inhibitors of the combinations of the invention are specific inhibitors of cell signalling proteins as described above and have activity against various cancers. Combinations of compounds of formula I with signalling inhibitors may be beneficial in the treatment and diagnosis of many types of cancer. Combination with a molecularly targeted agent such as a signalling inhibitor (e.g. Iressa, Avastin, herceptin, or Gleevec™) would find particular application in relation to cancers which express or have activated the relevant molecular target such as EGF receptor, VEGF receptor, ErbB2, BCRabl, c-kit, PDGF. Diagnosis of such tumours could be performed using techniques known to a person skilled in the art and as described herein such as RTPCR and FISH.

Problems: There is a need to increase the inhibitory efficacy of signalling inhibitors against tumour growth and also to provide a means for the use of lower dosages of signaling inhibitors to reduce the potential for adverse toxic side effects to the patient.

Preferences: Preferred signalling inhibitors for use in accordance with the invention include antibodies targeting EGFR such as monoclonal antibodies trastuzumab and cetuximab, EGFR tyrosine kinase inhibitors such as gefitinib and erlotinib, VEGF targeting antibody is bevacizumab, PDGFR inhibitor such as imatinib mesylate and Raf inhibitor such as sorafenib referred to herein.

Preferred antibodies targeting EGFR include the monoclonal antibodies trastuzumab and cetuximab. Trastuzumab is commercially available from Genentech Inc under the trade name Herceptin, or may be obtained as described in U.S. patent specification No. 5821337. Cetuximab is commercially available from Bristol-Myers Squibb Corporation under the trade name Erbitux, or may be obtained as described in PCT patent specification No. WO 96/40210.

Preferred EGFR tyrosine kinase inhibitors include gefitinib and erlotinib. Gefitinib is commercially available from AstraZeneca pic under the trade name Iressa, or may be obtained as described in PCT patent specification No. WO 96/33980. Erlotinib is commercially available from Genentech/Roche under the trade name Tarceva, or may be obtained as described in PCT patent specification No. WO 96/30347.

A preferred antibody targeting VEGF is bevacizumab which is commercially available from Genentech Inc under the trade name Avastin, or may be obtained as described in PCT patent specification No. WO 94/10202.

A preferred PDGFR inhibitor is imatinib mesylate which is commercially available from Novartis AG under the trade name Gleevec™ (a.k.a. Glivec®), or may be obtained as described in European patent specification No 564409.

A preferred Raf inhibitor is sorafenib which is available from Bayer AG, or may be obtained as described in PCT patent specification No. WO 00/42012.

Specific embodiments: In one embodiment, the signalling inhibitor is gefitinib (Iressa). In other embodiments the signalling inhibitor is selected from trastuzumab, cetuximab, gefitinib, erlotinib, bevacizumab, imatinib mesylate and sorafenib.

Further combinations of the invention include the following signalling inhibitors: dasatinib, lapatinib, nilotinib, vandetanib, vatalinib and CHIR-258, in particular dasatinib, lapatinib, nilotinib, vandetanib and vatalinib.

BMS is developing dasatinib (Sprycel or BMS-354825) an oral multitargeted kinase inhibitor, for the potential twice-daily treatment of chronic myelogenous leukemia (CML), Philadelphia chromosome-positive (Ph+) acute lymphoblastic leukemia (ALL) and solid tumors. The drug is also under investigation for multiple myeloma (MM) and other hematologic malignancies. Dasatanib has proved effective in Ph+ CML and AML in clinical trials given twice daily at 50-90 mg and also in imatinib resistant patients. Thrombocytopenia and neutropenia were amongst the side effects observed during clinical evaluation of dasatinib.

The structure of dasatinib, a Src/Abl kinase inhibitor is below:

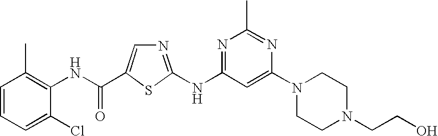

Dasatinib can be prepared by processes described in or analogous to WO 00/062778, WO 2005/076990 and WO 2005/077945.

Novartis is developing nilotinib (AMN-107), an orally available signal transduction inhibitor that targets BCR-ABL, c-kit and PDGF, for the potential treatment of leukemias. The compound is being investigated for chronic myeloid leukemia (CML) and relapsed or refractory acute lymphoblastic leukemia (ALL), systemic mastocytosis or chronic eosinophilic leukemia (hypereosinophilic syndrome), refractory gastrointestinal stromal tumor (GIST). Adverse events included hematological adverse events, headache, fatigue, muscle spasms, and nausea and vomiting. In early clinical studies doses of the order of 400 mg given twice daily have proved effective in treating CML, AML and ALL

The structure of nilotinib is shown below. It can be prepared as described in or analogous to as described in WO 2004/005281 and WO 2005/049032.

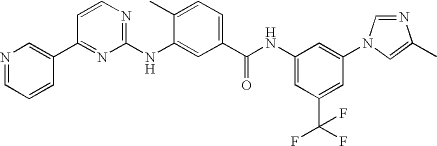

Vatalanib (PTK787/ZK222584) is a VEGF receptor tyrosine kinase angiogenesis inhibitor, under development by Novartis AG (formerly Ciba-Geigy) and Schering AG, for the potential treatment of colorectal cancer. The compound entered trials for colorectal cancer, the first- and second-line treatment of metastatic colorectal cancer (untreated and pretreated metastatic colorectal patients). Schering and Novartis are also investigating vatalanib in other solid tumors e.g. non-small cell lung cancer (NSCLC), as a second-line monotherapy in patients with stage IIIb/IV disease who had relapsed or were refractory to first-line therapy, renal cell cancer and glioblastoma, and potentially prostate, ovarian, breast, pancreas and small cell lung cancers. In addition vatalanib is also investigated for wet age-related macular degeneration (AMD). Vatalanib has been evaluated at doses up to 1,250 mg daily in clinical studies. Adverse events include nausea/vomiting, fatigue, ataxia, lethargy, hypertension, headache, dizziness, diarrhoea, hypertension as well as syncope and neurotoxicity.

Vatalinib (structure shown below) can be prepared as described in or analogues to as described in WO 98/35958

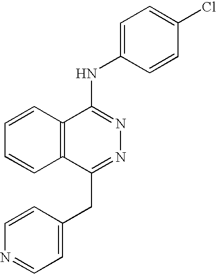

Lapatinib ditosylate (Tykerb or GW2016/572016), an ErbB2 and EGFR dual tyrosine kinase inhibitor, is being developed by GlaxoSmithKline plc (GSK) for the potential treatment of solid tumors.

It is under investigation for various tumors including breast, lung, stomach, bladder and head and neck cancers, in particular for the treatment of patients with refractory advanced or metastatic breast cancer whose tumours express HER-2 and who have failed previous therapies both as a single agent and in combination with other therapies including capecitabine and paclitaxel. The compound had also entered trials for renal cell cancer, advanced and metastatic non-small cell lung cancer (NSCLC) and in the treatment of brain metastases associated with breast cancer. In early clinical evaluation Lapatinib has been evaluated on a twice daily and once daily schedule at doses over the range 500-1500 mg and at doses of 750-1250 mg given twice daily. Side effects include gastrointestinal gaseous symptoms, rash, headache and abnormal liver function tests.

Quinazoline compounds, and ditosylate salts, anhydrate or hydrate forms such as of the structure shown below (lapatinib) can be synthesised using the process described in WO 00/202552 and WO 99/35146 or process analogues thereto.

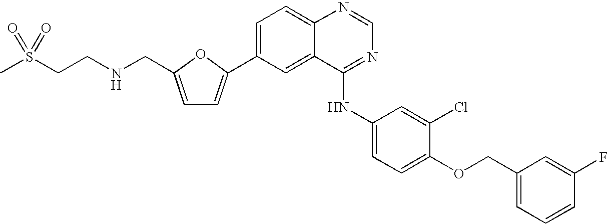

Vandetanib (ZD-6474; Zactima; formerly AZD-6474) is under development by AstraZeneca for the potential once-daily oral treatment of solid and haematological tumors including thyroid, lung, breast, head and neck, brain (i.e. glioma) and multiple myeloma. It is one of a series of inhibitors of vascular endothelial growth factor (VEGF) receptor tyrosine kinase) that also has activity against the EGF and RET receptor tyrosine kinases. Clinical studies have investigated doses of vandetanib in the region of 100-300 mg daily as monotherapy and in combinations. Common adverse effects observed were rash, fatigue, nausea, diarrhea, asymptomatic QTc prolongation

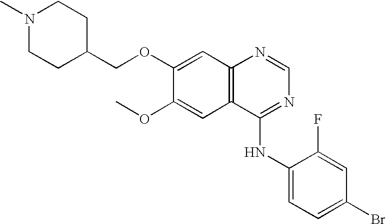

ZD-6474 can be prepared as described in WO 01/32651 and processes analogous therein.

CHIR-258 (GFKI-258; structure shown), is a potent VEGF, FGF and PDGF receptor kinase inhibitor, for the potential oral treatment of various types of cancer. Novartis (formerly Chiron), had initiated a study in acute myelogenous leukemia (AML) patients and multiple myeloma (MM).

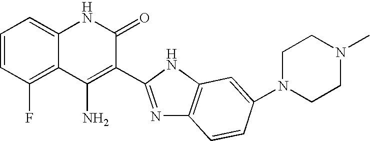

CHIR-258 can be prepared as described in WO 02/22598 and WO 2005/046590 and processes analogous therein.

Another suitable signalling inhibitor for use in the combinations of the invention is axitinib (AG-013736). Pfizer is developing axitinib (AG-13736, AG-013736), an oral inhibitor of the VEGF, PDGF and CSF-1 receptor tyrosine kinases which was discovered by Pfizer's wholly-owned subsidiary Agouron Pharmaceuticals, as an anti-angiogenic agent for the potential treatment of cancer. It is being studied for breast cancer, renal cell carcinoma (RCC), non-small cell lung cancer (NSCLC), melanoma, and carcinomas. The compound has also being investigated for the treatment of acute myeloid leukemia and myelodysplastic syndrome (MDS).

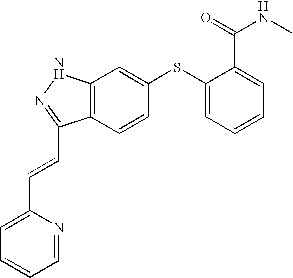

It can be prepared as described in WO 2004/087152, WO 2006/048746 and WO 2006/048745 and process analogous thereto. Axitinib may be dosed at 5 mg PO BID.

Posology: With regard to the EGFR antibodies, these are generally administered in a dosage of 1 to 500 mg per square meter (mg/m²) of body surface area, trastuzumab being advantageously administered in a dosage of 1 to 5 mg/m² of body surface area, particularly 2 to 4 mg/m²; cetuxumab is advantageously administered in a dosage of about 200 to 400 mg/m², preferably about 250 mg/m².

With regard to the EGFR tyrosine kinase inhibitors, these are generally administered in a daily oral dosage of 100 to 500 mg, for example gefitinib in a dosage of about 250 mg and erlotinib in a dosage of about 150 mg.

With regard to the VEGF monoclonal antibody bevacizumab, this is generally administered in a dosage of about 1 to 10 mg/kg for example about 5 mg/kg.

With regard to the PDGF inhibitor imatinib, this is generally administered in a dosage of about 400 to 800 mg per day preferably about 400 mg per day.

With regard to the Raf inhibitor sorafenib, this is administered at a dose of 800 mg daily.

These dosages may be administered for example once, twice or more per course of treatment, which may be repeated for example every 7, 14, 21 or 28 days.

### PKA/B Inhibitors and PKB Pathway Inhibitors

Another preferred class of signaling inhibitor for use in the combinations of the invention are PKA/B inhibitors and PKB pathway inhibitors.

PKB pathway inhibitors are those that inhibit the activation of PKB, the activity of the kinase itself or modulate downstream targets, blocking the proliferative and cell survival effects of the pathway. Target enzymes in the pathway include phosphatidyl inositol-3 kinase (PI3K), PKB itself, mammalian target of rapamycin (MTOR), PDK-1 and p70 S6 kinase and forkhead translocation factor. Several components of the PI 3-kinase/PKB/PTEN pathway are implicated in oncogenesis. In addition to growth factor receptor tyrosine kinases, integrin-dependent cell adhesion and G-protein coupled receptors activate PI 3-kinase both directly and indirectly through adaptor molecules. Functional loss of PTEN (the most commonly mutated tumour-suppressor gene in cancer after p53), oncogenic mutations in PI 3-kinase, amplification of PI 3-kinase and overexpression of PKB have been established in many malignancies. In addition, persistent signaling through the PI 3-kinase/PKB pathway by stimulation of the insulin-like growth factor receptor is a mechanism of resistance to epidermal growth factor receptor inhibitors.

The discovery of non-random, somatic mutations in the gene encoding p110α in a range of human tumours suggests an oncogenic role for the mutated PI 3-kinase enzyme (Samuels, et al., Science, 304 554, April 2004). Mutations in p110α have since been detected in the following human tumours: colon (32%), hepatocellular (36%) and endometroid and clear cell cancer (20%). p110α is now the most commonly mutated gene in breast tumours (25-40%). Forkhead family translocations often occur in acute leukemia.

The PI 3-kinase/PKB/PTEN pathway is thus an attractive target for cancer drug development since such agents would be expected to inhibit proliferation and surmount resistance to cytotoxic agents in cancer cells.

Examples of PKB pathway inhibitors include PI3K Inhibitors such as Semaphore, SF1126 and MTOR inhibitors such as Rapamycin Analogues. RAD 001 (everolimus) from Novartis is an orally available derivative of the compound rapamycin. The compound is a novel macrolide, which is being developed as an antiproliferative drug with applications as an immunosuppressant and anticancer agent. RAD001 exerts its activity on growth-factor dependent proliferation of cells through its high affinity for an intracellular receptor protein, FKBP-12. The resulting FKBP-12/RAD001 complex then binds with mTOR to inhibit downstream signaling events. The compound is currently in clinical development for a wide variety of oncology indications. CCI 779 (temsirolemus) from Wyeth Pharmaceuticals and AP23573 from Ariad Pharmaceuticals are also rapamycin analogues. AP23841 and AP23573 from Ariad Pharmaceutical also target mTOR. Calmodulin inhibitors from Harvard are forkhead translocation inhibitors. (Nature Reviews drug discovery, Exploiting the PI3K/AKT Pathway for Cancer Drug Discovery; Bryan T. Hennessy, Debra L. Smith, Prahlad T. Ram, Yiling Lu and Gordon B. Mills; December 2005, Volume 4; pages 988-1004).

Definitions: The term “PKA/B inhibitor” is used herein to define a compound which has protein kinase B (PKB) and/or protein kinase A (PKA) inhibiting or modulating activity ity, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

The term “PKB pathway inhibitor” is used herein to define a compound which inhibits the activation of PKB, the activity of the kinase itself or modulate downstream targets, blocking the proliferative and cell survival effects of the pathway (including one or more of the target enzymes in the pathway as described herein, including phosphatidyl inositol-3 kinase (PI3K), PKB itself, mammalian target of rapamycin (MTOR), PDK-1 and p70 S6 kinase and forkhead translocation).

Technical background: KRX-0401 (Perifosine/NSC 639966) is a synthetic substituted heterocyclic alkylphosphocholine that acts primarily at the cell membrane targeting signal transduction pathways, including inhibition of PKB phosphorylation. KRX-0401 has been evaluated in phase 1 studies as a potential oral anticancer drug. Dose limiting toxicities included nausea, vomiting and fatigue. Gastrointestinal toxicities increased at higher doses. A phase II trial in refractory sarcoma is planned.

API-2/TCN is a small molecule inhibitor of PKB signaling pathway in tumour cells. Phase I and II clinical trials of API-2/TCN have been conducted on advanced tumours. API-2/TCN exhibited some side effects, which include hepatotoxicity, hypertriglyceridemia, thrombocytopenia, and hyperglycemia.

RX-0201 is being developed as an AKT protein kinase inhibitor for the treatment of solid tumours. In July 2004, a phase I trial was initiated in patients with advanced malignancies. Data from this showed RX-0201 inhibited overexpression of Akt and suppressed cancer growth in brain, breast, cervix, liver, lung, ovary, prostate and stomach tumours, and was well tolerated. By March 2005, US Orphan Drug status had been granted to RX-0201 for several solid tumour types.

Enzastaurin HCI (LY317615) suppresses angiogenesis and was advanced for clinical development based upon anti-angiogenic activity. It is described as a selective PKCβ inhibitor. It also has a direct anti-tumour effect, and suppresses GSK3β phosphorylation. It is currently being investigated for the treatment of glioma and non-Hodgkin's lymphoma.

SR-13668 is claimed to be an orally active specific AKT inhibitor that significantly inhibits phospho-AKT in breast cancer cells both in vitro and in vivo. In vivo assessment in mice showed no adverse effects at doses 10 times more than were needed for antitumour activity.

PX-316 is a D-3-deoxy-phosphatidyl-myo-inositol that binds to the PH domain of PKB, trapping it in the cytoplasm and thus preventing PKB activation. Anti-tumour activity was seen in early xenografts and was well tolerated.

Allosteric, selective inhibitors of PKB based on a 2,3-diphenylquinoxaline core or a 5,6-diphenylpyrazin-2(1H)-one core have been developed (Merck).

KRX-0401: In a Phase I weekly dosing study conducted in Europe, the recommended Phase II dose was 600/mg/week. Subsequent studies conducted in the U.S. have shown that much higher doses are well tolerated when the doses are divided and administered at 4 to 6 hour intervals. In addition, it has been shown that KRX-0401 has a very long half-life in the range of 100 hours. This makes the possibility of a relative non-toxic, intermittent dosing schedule very plausible.

A phase I trial of API-2 was conducted using a 5-day continuous infusion schedule. Dose levels ranged from 10 mg/sq m/day×5 days to 40 mg/sq m/day×5 days. Initially, courses were repeated every 3 to 4 weeks. As cumulative toxicity became manifested, the interval between courses was changed to every 6 weeks. Recommended schedule for Phase II studies is 20 mg/sq m/day for 5 days every 6 weeks. A Phase II trial of TCN-P was conducted in metastatic or recurrent squamous cell carcinoma of the cervix using a 5-day continuous infusion schedule. The starting dose was 35 mg/m²×5 days and courses were repeated every 6 weeks.

Further PKB inhibitors include Perifosine from Keryx Biopharmaceuticals. Perifosine is an oral Akt inhibitor which exerts a marked cytotoxic effect on human tumour cell lines, and is currently being tested in several phase II trials for treatment of major human cancers. KRX-0401 (Perifosine/NSC 639966) has the structure:

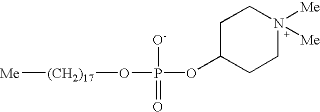

It can be prepared according to Aste Medica patent publication DE4222910 or Xenoport patent publication US2003171303.

API-2/TCN (Triciribine) has the structure:

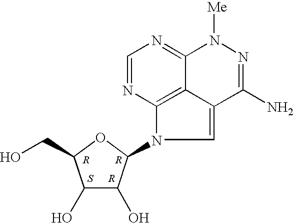

It can be prepared according to Bodor patent publication WO9200988 or Ribapharm patent publication WO2003061385.

Enzastaurin hydrochloride has the structure:

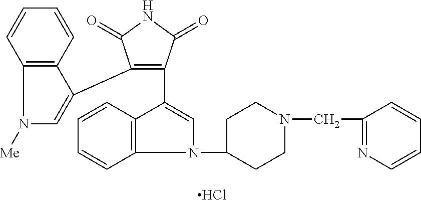

It can be prepared according to Eli Lilly patent publication WO2004006928.

SR 13668 has the structure:

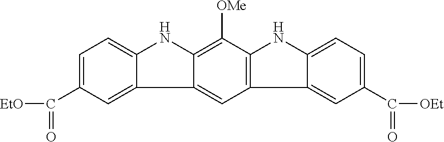

It can be prepared according to SRI International patent publication US2004043965.

NL-71-101 has the structure:

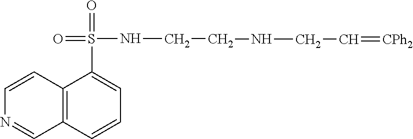

It can be prepared according to Biochemistry (2002), 41(32), 10304-10314 or Peptor patent publication WO2001091754.

DeveloGen (formerly Peptor) is investigating NL-71-101, a protein kinase B (PKB) inhibitor, for the potential treatment of cancer [466579], [539004]. At the beginning of 2003, the compound was undergoing lead optimization [495463]. By February 2004, the company was seeking to outlicense certain development rights to its protein kinase B program [523638].

In 2002, data were published showing that NL-71-101 inhibited the activity of PKB over PKA, PKG and PKC with IC50 values of 3.7, 9, 36 and 104 microM, respectively. NL-71-101 induced apoptosis in OVCAR-3 tumour cells, in which PKB is amplified at concentrations of 50 and 100 microM [466579]. This compound has the structure:

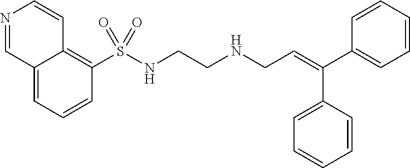

Specific embodiments: Embodiments contemplated include combinations in which the anti-cancer agent is a PKB inhibitor selected from one or more of the specific compounds described above.

### 14. CDK Inhibitors

Preferred CDK inhibitors for use as auxiliary agents in the combinations of the invention are compounds of formula (0) or (I′″) as defined herein. However, CDK inhibitors for use in the combinations of the invention also include the ancillary CDK inhibitors described in more detail below that have cyclin dependent kinase inhibiting or modulating activity and/or glycogen synthase kinase-3 (GSK3) inhibiting or modulating activity. Thus, the combinations of the present invention may comprise (or consist essentially on two or more compounds of formula (I) as defined herein.

In addition to the CDK compounds of formula (0) or (I′″) herein, the combinations of the present invention may include one or more ancillary CDK inhibitors or modulators. Such ancillary CDK inhibitors or modulators may be selected from the various CDK inhibitors described herein and preferred ancillary CDK inhibitors are discussed in more detail below.

Definition: The term “CDK inhibitor” as used herein refers to compounds that inhibit or modulate the activity of cyclin dependent kinases (CDK), including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

The term “ancillary CDK inhibitor” as used herein refers to compounds that inhibit or modulate the activity of cyclin dependent kinases (CDK) and which does not conform to the structure of formula (0) or (I) as defined herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Technical background: CDKs play a role in the regulation of the cell cycle, apoptosis, transcription, differentiation and CNS function. Therefore, CDK inhibitors may find application in the treatment of diseases in which there is a disorder of proliferation, apoptosis or differentiation such as cancer. In particular RB+ve tumours may be particularly sensitive to CDK inhibitors. RB−ve tumours may also be sensitive to CDK inhibitors.

Examples of CDK inhibitors which may be used in combinations according to the invention include seliciclib, alvocidib, 7-hydroxy-staurosporine, JNJ-7706621, BMS-387032 (a.k.a. SNS-032), PHA533533, PD332991, ZK-304709, and AZD-5438.

Seliciclib, which is the R isomer of roscovitine, and otherwise known as CYC 202, has the chemical name (2R)-2-[[9-(1-methylethyl)-6-[(phenylmethyl)-amino]-9H-purin-2-yl]amino]-1-butanol. It is being evaluated in clinical trials for the potential treatment of various cancers including lymphoid leukaemia, non-small-cell lung cancer, glomerulonephritis, mantle cell lymphoma, multiple myeloma, and breast cancer. Observed toxicities in clinical trials include nausea/vomiting and asthenia, skin rash and hypokalemia. Other toxicities included reversible renal impairment and transaminitis, and emesis.

Alvocidib, which is otherwise known as flavopiridol, HMR 1275 or L 86-8275, and which has the chemical name 5,7-dihydroxy-8-(4-N-methyl-2-hydroxypyridyl)-6′-chloroflavone, is being investigated in clinical trials for the potential treatment of various cancers including cancer of the esophagus, stomach, prostate, lung and colon, and also chronic lymphocytic leukaemia, and multiple myeloma, lymphoma; the most common toxicities observed were diarrhea, tumour pain, anemia, dyspnea and fatigue.

7-Hydroxystaurosporine, which is otherwise known as UCN-01 is being evaluated in clinical trials for the potential treatment of various cancers including chronic lymphocytic leukaemia, pancreas tumours and renal tumours; adverse events observed included nausea, headache and hyperglycemia.

JNJ-7706621, which has the chemical name N3-[4-(aminosulfonyl)-phenyl]-1-(2,6-difluorobenzoyl)-1H-1,2,4-triazole-3,5-diamine, is the subject of pre-clinical testing for the potential treatment of melanoma and prostate cancer. BMS-387032 which has the chemical name N-[5-[[[5-(1,1-dimethylethyl)-2-oxazolyl]-methyl]thio]-2-thiazolyl]-4-piperidinecarboxamide, has been evaluated in phase I studies as a potential anticancer drug for patients with metastatic solid tumours such as renal cell carcinomas, non-small-cell lung cancer, head and neck cancers and leiomyosarcoma The drug was well tolerated with transient neutropenia noted as the primary toxicity. Other side-effects included transient liver aminase elevations, gastrointestinal toxicity, nausea, vomiting, diarrhea and anorexia. PHA533533, which has the chemical name (αS)—N-(5-cyclopropyl-1H-pyrazol-3-yl)-α-methyl-4-(2-oxo-1-pyrrolidinyl)-benzene-acetamide, is the subject of pre-clinical testing for the potential treatment of various cancers such as tumours of the prostate, colon and ovary. PD332991, which has the chemical name 8-cyclohexyl-2-[[4-(4-methyl-1-piperazinyl)phenyl]amino]-pyrido[2,3-d]pyrimidin-7(8H)-one, is the subject of pre-clinical testing for the potential treatment of various cancers. Pre-clinical data suggests that it is a highly selective and potent CDK4 inhibitor, demonstrating marked tumour regression in vivo models.

ZK-304709 is an oral dual specificity CDK and VEGFR kinase inhibitor, described in PCT patent specification No. WO 02/096888, and is the subject of pre-clinical testing for the potential treatment of various cancers. AZD-5438 is a selective cyclin-dependent kinase (CDK) inhibitor, which is in pre-clinical development for the treatment of solid cancers. Seliciclib may be prepared for example as described in PCT patent specification No. WO 97/20842, or by processes analogous thereto. Alvocidib, may be prepared for example as described in U.S. patent specification No. 4900727 or by processes analogous thereto. 7-Hydroxystaurosporine may be prepared for example as described in U.S. patent specification No. 4935415, or by processes analogous thereto. JNJ-7706621 may be prepared for example as described in PCT patent specification No. WO 02/057240, or by processes analogous thereto. BMS-387032 may be prepared for example as described in PCT patent specification No. WO 01/44242, or by processes analogous thereto. PHA533533 may be prepared for example as described in U.S. patent specification No. 6455559, or by processes analogous thereto. PD332991, may be prepared for example as described in PCT patent specification No. WO 98/33798, or by processes analogous thereto. ZK-304709 may be prepared for example as described in PCT patent specification No. WO 02/096888, or by processes analogous thereto.

Preferences and specific embodiments: Embodiments contemplated include combinations in which the anti-cancer agent is a CDK inhibitor selected from one or more of the specific compounds described above. Thus, preferred CDK inhibitors for use in combinations according to the invention include seliciclib, alvocidib, 7-hydroxystaurosporine, JNJ-7706621, BMS-387032, PHA533533, PD332991 and ZK-304709. Particular CDK inhibitors for use in combinations according to the invention include seliciclib, alvocidib, 7-hydroxystaurosporine, JNJ-7706621, BMS-387032, PHA533533, PD332991 and ZK-304709.

Posology: The CDK inhibitor may be administered for example in a daily dosage of for example 0.5 to 2500 mg, more preferably 10 to 1000 mg, or alternatively 0.001 to 300 mg/kg, more preferably 0.01 to 100 mg/kg, particularly for seliciclib, in a dosage of 10 to 50 mg; for alvocidib, in a dosage in accordance with the above-mentioned U.S. patent specification No. 4900727; for 7-hydroxystaurosporine in a dosage of 0.01 to 20 mg/kg; for JNJ-7706621 in a dosage of 0.001 to 300 mg/kg; for BMS-387032 in a dosage of 0.001 to 100 mg/kg more preferably 0.01 to 50 mg/kg, and most preferably 0.01 to 20 mg/kg; for PHA533533 in a dosage of 10 to 2500 mg; for PD332991 in a dosage of 1 to 100 mg/kg; and for ZK-304709 in a dosage of 0.5 to 1000 mg preferably 50 to 200 mg.

These dosages may be administered for example once, twice or more per course of treatment, which may be repeated for example every 7, 14, 21 or 28 days.

### 15. COX-2 Inhibitors

Definition: The term “COX-2 inhibitor” is used herein to define compounds which inhibit or modulate the activity of the cyclo-oxygenase-2 (COX-2) enzyme, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Biological activity: The COX-2 inhibitors working via one or more pharmacological actions as described herein have been identified as suitable anti-cancer agents.

Technical background: Recently, research in cancer chemotherapy has focused on the role of the cyclo-oxygenase-2 (COX-2) enzyme in the aetiology of cancer. Epidemiological studies have shown that people who regularly take non-steroidal anti-inflammatory drugs (NSAIDs), for example aspirin and ibuprofen to treat conditions such as arthritis, have lower rates of colorectal polyps, colorectal cancer, and death due to colorectal cancer. NSAIDs block cyclooxygenase enzymes, which are produced by the body in inflammatory processes, and which are also produced by pre-cancerous tissues. For example in colon cancers, a dramatic increase of COX-2 levels is observed. One of the key factors for tumour growth is the supply of blood to support its increased size. Many tumours can harness chemical pathways that prompt the body to create a web of new blood vessels around the cancer, a process called angiogenesis. COX-2 is believed to have a role in this process. It has therefore been concluded that inhibition of COX-2 may be effective for treating cancer, and COX-2 inhibitors have been developed for this purpose. For example celecoxib, which has the chemical name 4-[5-(4-methylphenyl)-3-(trifluoromethyl)-1H-pyrazol-1-yl]benzenesulfonamide, is a selective COX-2 inhibitor that is being investigated for the treatment of various cancers including bladder and esophageal cancer, renal cell carcinoma, cervical cancer, breast cancer, pancreatic cancer non-Hodgkin's lymphoma and non-small cell lung cancer.

Posology: The COX-2 inhibitor (for example celecoxib) can be administered in a dosage such as 100 to 200 mg e.g. daily.

These dosages may also be administered for example once, twice or more per course of treatment, which may be repeated for example every 7, 14, 21 or 28 days.

Problems: The most common adverse effects are headache, abdominal pain, dyspepsia, diarrhea, nausea, flatulence and insomnia. There is a need to provide a means for the use of lower dosages of COX-2 inhibitors to reduce the potential for adverse toxic side effects to the patient.

Preferences and specific embodiments: In one embodiment the COX-2 inhibitor is celecoxib. Celecoxib is commercially available for example from Pfizer Inc under the trade name Celebrex, or may be prepared for example as described in PCT patent specification No. WO 95/15316, or by processes analogous thereto.

Two other commercially available COX-2 inhibitors are Arcoxia (etoricoxib from Merck) and Novartis Cox-2 inhibitor lumiracoxib (Prexige).

### 16. HDAC Inhibitors

Definition: The term “HDAC inhibitor” is used herein to define compounds which inhibit or modulate the activity of histone deacetylases (HDAC), including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Biological activity: The HDAC inhibitors working via one or more pharmacological actions as described herein have been identified as suitable anti-cancer agents.

Technical background: Reversible acetylation of histones is a major regulator of gene expression that acts by altering accessibility of transcription factors to DNA. In normal cells, histone deacetylase (HDAC) and histone acetyltrasferase (HDA) together control the level of acetylation of histones to maintain a balance. Inhibition of HDA results in the accumulation of hyperacetylated histones, which results in a variety of cellular responses. Inhibitors of HDA (HDAI) have been studied for their therapeutic effects on cancer cells. Recent developments in the field of HDAI research have provided active compounds, that are suitable for treating tumours.

Accruing evidence suggests that HDAI are more efficacious when used in combination with other chemotherapeutic agents. There are both synergistic and additive advantages, both for efficacy and safety. Therapeutic effects of combinations of chemotherapeutic agents with HDAI can result in lower safe dosage ranges of each component in the combination. The study of inhibitors of histone deacetylases (HDAC) indicate that these enzymes play an important role in cell proliferation and differentiation. The inhibitor Trichostatin A (TSA) causes cell cycle arrest at both G1 and G2 phases, reverts the transformed phenotype of different cell lines, and induces differentiation of Friend leukaemia cells and others. TSA (and suberoylanilide hydroxamic acid SAHA) have been reported to inhibit cell growth, induce terminal differentiation, and prevent the formation of tumours in mice (Finnin et al., Nature, 401:188-193, 1999).

Trichostatin A has also been reported to be useful in the treatment of fibrosis, e.g. liver fibrosis and liver chirrhosis. (Geerts et al., European Patent Application EPO 827 742, published 11 Mar. 1998).

Preferences and specific embodiments: Preferred HDAC inhibitors for use in accordance with the invention are selected from TSA, SAHA, JNJ-16241199, LAQ-824, MGCD-0103 and PXD-101 (referred to above).

Thus, synthetic inhibitors of histone deacetylases (HDAC) which are suitable for use in the present invention include JNJ-16241199 from Johnson and Johnson Inc, LAQ-824 from Novartis, MGCD-0103 from MethylGene, and PXD-101 from Prolifix.

JNJ-16241199 has the following structure:

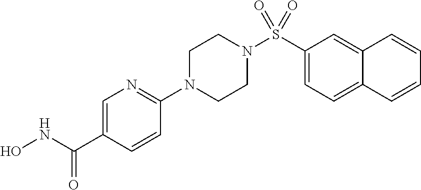

MGCD-0103 has the structure:

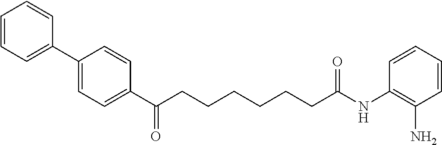

LAQ-824 has the structure:

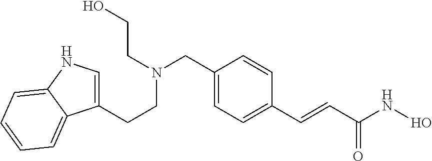

Other inhibitors of histone deacetylases (HDAC) which are suitable for use in the present invention include, but are not limited to, the peptide chlamydocin, and A-173, also from Abbott Laboratories.

A-173 is a succinimide macrocyclic compound with the following structure:

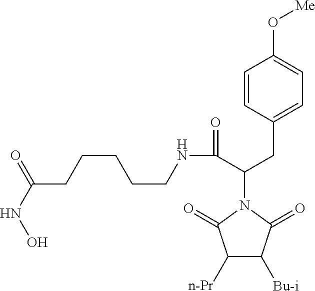

Posology: In general, for HDAC inhibitors it is contemplated that a therapeutically effective amount would be from 0.005 mg/kg to 100 mg/kg body weight, and in particular from 0.005 mg/kg to 10 mg/kg body weight. It may be appropriate to administer the required dose as two, three, four or more sub-doses at appropriate intervals throughout the day. Said sub-doses may be formulated as unit dosage forms, for example, containing 0.5 to 500 mg, and in particular 10 mg to 500 mg of active ingredient per unit dosage form.

### 17. Selective Immunoresponse Modulators

Selective immunoresponse modulators include Lenalidomide and Thalidomide. Lenalidomide (Revlimid) is an oral thalidomide derivative developed by Celgene which is a potent inhibitor of TNF-alpha and interleukin-1 beta which is being developed for the treatment of 5q-myelodysplastic syndrome multiple myeloma, chronic lymphocytic leukaemia gliomas, cutaneous T-cell lymphoma and epithelial ovarian cancer.

Lenalidomide (3-(4-amino-1-oxoisoindolin-2-yl)piperidine-2,6-dione) has the following structure:

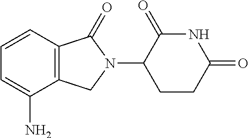

Thalidomide is a sedative and anti-emetic that became widely recognized as a result of reports of its teratogenic effects, most notably limb deformities in up to 12,000 children born to women who had received thalidomide in Europe and Canada during the 1960s. Celgene has developed and launched thalidomide as an oral TNF-alpha inhibitor (Sold to Pharmion). Extensive clinical evidence has accumulated with regard to the potential antitumor activity of thalidomide in several types of neoplasias, with notable activity in relapsed/refractory multiple myeloma, Waldenstrom's macroglobulinemia (WM) and myelodysplastic syndromes (MDS). There is also evidence of biological activity in acute myeloid leukemia, myelofibrosis with myeloid metaplasia, renal cell carcinoma, malignant gliomas, prostate cancer, Kaposi's sarcoma and colorectal carcinoma.

Thalidomide (1,3-dioxo-2-(2,6-dioxopiperidin-3-yl)isoindoline) has the following structure:

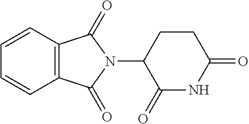

Posology: Thalidomide may be advantageously administered in dosages of 100 to 800 mg/day continuously as tolerated. Lenalidomide may be advantageously administered in 5- to 40-mg doses continuously as tolerated.

### 18. DNA Methylase Inhibitors

Definition: The term “DNA methylase inhibitor” or “DNA methyltransferase inhibitor” as used herein refers to a compound which directly or indirectly perturbs, disrupts, blocks, modulates or inhibits the methylation of DNA, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. They are also referred to as “hypomethylating agents”.

Biological activity: The DNA methylase inhibitors working via one or more pharmacological actions as described herein have been identified as suitable anti-cancer agents.

Technical background: One target for cancer chemotherapy is DNA synthesis, which may depend on appropriate methylation of tumour DNA. Compounds which directly or indirectly perturb, disrupt, block, modulate or inhibit the methylation of DNA may therefore be useful anticancer drugs.

The DNA methylase inhibitor temozolomide is used for the treatment of glioblastoma multiforme, and first-line treatment of patients with advanced metastatic malignant melanoma (such as first-line treatment of patients with advanced metastatic malignant melanoma) and has also being investigated and used for the treatment of malignant glioma at first relapse. This compound undergoes rapid chemical conversion at physiological pH to the active compound, monomethyl triazeno imidazole carboxamide (MTIC) which is responsible for the methylation of DNA at the 06 position of guanine residues (which appears to lead to a suppression in expression of DNA methyltransferase and so produce hypomethylation).

Problems: The most common side effects associated with temozolomide therapy are nausea, vomiting, headache, fatigue, thrombocytopenia and constipation. There is a need to increase the inhibitory efficacy of DNA\methylase inhibitors and to provide a means for the use of lower dosages of signaling inhibitors to reduce the potential for adverse toxic side effects to the patient.

Preferences and specific embodiments: In one embodiment, the DNA methylase inhibitor is temozolomide (3,4-dihydro-3-methyl-4-oxoimidazo[5,1-d]-as-tetrazine-8-carboxamide). Temozolomide is commercially available for example from Schering Corporation under the trade name Temodar, or may be prepared for example as described in German patent specification No. 3231255, or by processes analogous thereto.

A further DNA methyltransferase inhibitor for use in the combinations of the invention is Decitabine (a.k.a. Dacogen) having the structure shown below:

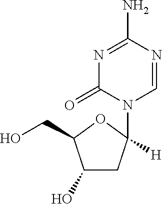

SuperGen Inc and MGI Pharma Inc have developed decitabine (Dacogen), an inhibitor of DNA methyltransferase, preventing methylation of cytosine residues on DNA and leading to hypomethylation of gene promoters, thereby reactivating silenced genes. Decitabine/Dacogen is cytotoxic to a broad range of malignant cells in vitro. It shows significant activity against acute myeloid leukemia (AML), chronic myeloid leukemia (CML) and myelodysplastic syndromes (MDS). Decitabine/Dacogen is indicated for the treatment of myelodysplastic syndromes (MDS) and secondary MDS (including chronic myelomonocytic leukemia, refractory anemia, refractory anemia with ringed sideroblasts, refractory anemia with excess blasts and refractory anemia with excess blasts in transformation).

Decitabine/Dacogen is an analog of deoxycytidine (beta-D-anomer of 2′-deoxy-5-azacytidine). It differs from deoxycytidine by substitution at position 5 of the pyrimidine ring with nitrogen. Decitabine contains deoxyribose, in contrast to the related analog, Pharmion Corp's 5-azacytidine (Vidaza), which contains a ribose sugar. Decitabine is, therefore, a deoxynucleoside and is incorporated into DNA, but not RNA, in contrast to 5-azacytidine which is incorporated into RNA. Decitabine and 5-azacytidine differ from other pyrimidine analogs, such as cytosine arabinoside and gemcitabine, by modification at position 5 of the pyrimidine ring. This distinctive feature, which is not present in these latter drugs, is responsible for inhibition of DNA methyltransferase. Pseudoisocytidine and 5-fluoro-2′-deoxycytidine, further analogs with modifications of the 5 position of the pyrimidine ring, also inhibit demethylation.

Decitabine/Dacogen is dosed at 15 mg/m2 over a three hour period every 8 hours for 3 days every 6 weeks as a cycle of therapy or on a daily dosing schedule with a one hour infusion usually delivered at 20 mg/m2 per day either for one week or two weeks every 6 weeks as a cycle

At toxic doses decitabine/Dacogen produces leukopenia, thrombocytopenia and weight loss. The major toxicity of decitabine is myelosuppression, which is proportional to dose and duration of therapy. The effects are pronounced at high doses (>200 mg/m2/day), and myelosuppression is enhanced by concomitant administration of other cytotoxic drugs. Neutropenic infection and other complications of myelosuppression have proved fatal. Non-hematological side effects include nausea, vomiting, mucositis and alopecia.

Decitabine/Dacogen and other analogues thereof can be made as outlined in U.S. Pat. No. 0,343,2549 and further discussed on WO 006/017278 and WO 2006/037024 to SuperGen Inc.

A further DNA methyltransferase inhibitor for use in the combinations of the invention is azacytidine (a.k.a. 5-azacitidine, 5-azacytidine or Vidaza) a sc administered hypomethylating agent and DNA methyltransferase inhibitor. It is indicated for the treatment of all myelodysplastic syndrome (MDS) subtypes, including refractory anemia (RA) or RA with ringed sideroblasts, RA with excess blasts, RA with excess blasts in transformation and chronic myelomonocytic leukemia.

5-azacitidine (Vidaza) can be administered twice-daily subcutaneously or via the iv route administration for MDS treatment.

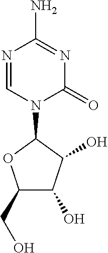

It can be prepared as described in DE 1922702, GB 1227691 and FR 2008048 from Ceskoslovenska Akademie Ved and WO 2004082618, WO 2004082619 and WO 2004082822 from Pharmion and process analogous thereto.

Posology: The DNA methylating agent (for example temozolomide) can be administered in a dosage such as 0.5 to 2.5 mg per square meter (mg/m²) of body surface area, particularly about 1.3 mg/m². These dosages may be administered for example once, twice or more per course of treatment, which may be repeated for example every 7, 14, 21 or 28 days.

### 19. Proteasome Inhibitors

Definition: The term “proteasome inhibitor” as used herein refers to compounds which directly or indirectly perturb, disrupt, block, modulate or inhibit the half-life of many short-lived biological processes, such as those involved in the cell cycle. The term therefore embraces compounds which block the action of proteasomes (large protein complexes that are involved in the turnover of other cellular proteins). The term also embraces the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above.

Biological activity: The proteasome inhibitors working via one or more pharmacological actions as described herein have been identified as suitable anti-cancer agents.

Technical background: Another class of anticancer agents are the proteasome inhibitors. Proteasomes control the half-life of many short-lived biological processes, such as those involved in the cell cycle. Therefore, proteasome malfunction can lead to abnormal regulation of the cell cycle and uncontrolled cell growth.

The cell cycle is controlled by both positive and negative signals. In a normal cell, proteasomes break down proteins that inhibit the cell cycle, such as cyclin-dependent kinase inhibitors. Inhibition of proteasome function causes cell cycle arrest and cell death. Tumour cells are more susceptible to these effects than normal cells, in part because they divide more rapidly and in part because many of their normal regulatory pathways are disrupted. The mechanism for the differential response of normal and cancer cells to proteasome inhibition is not fully understood. Overall, cancer cells are more susceptible to proteasome inhibitors and, as a result, these inhibitors may be an effective treatment for certain cancers.

One such proteasome inhibitor is bortezimib, which has the chemical name [(1R)-3-methyl-1-[[(2S)-1-oxo-3-phenyl-2-[(pyrazinylcarbonyl)amino]propyl]amino]butyl]-boronic acid. Bortezimib specifically interacts with a key amino acid, namely threonine, within the catalytic site of the proteasome. Bortezimib is being used for the treatment of multiple myeloma and also for a number of other cancers, including leukemia and lymphoma, and prostate, pancreatic and colorectal carcinoma. In addition Velcade is useful for the treatment of non-Hodgkin's lymphoma.

Problems: The most common side effects with bortezimib are nausea, tiredness, diarrhea, constipation, decreased platelet blood count, fever, vomiting, and decreased appetite. Bortezimib can also cause peripheral neuropathy.

Thus, there is a need to provide a means for the use of lower dosages to reduce the potential of adverse toxic side effects to the patient.

Preferences and specific embodiments: Preferred proteasome inhibitors for use in accordance with the invention include bortezimib. Bortezimib is commercially available for example from Millennium Pharmaceuticals Inc under the trade name Velcade, or may be prepared for example as described in PCT patent specification No. WO 96/13266, or by processes analogous thereto.

Posology: The proteasome inhibitor (such as bortezimib) can be administered in a dosage such as 100 to 200 mg/m². These dosages may be administered for example once, twice or more per course of treatment, which may be repeated for example every 7, 14, 21 or 28 days.

The antibiotic bleomycin may also be used as a cytotoxic agent as an anti-cancer agent according to the invention.

### 20. Aurora Inhibitors

In one embodiment of the invention, the auxiliary compound is an inhibitor of Aurora kinase(s). Particular Aurora inhibitors for use in the combinations of the invention are described in more detail below.

Definition: The term “Aurora kinase inhibitor” (or simply “Aurora inhibitor”) as used herein refers to compounds that inhibit or modulate the activity of any of the Aurora kinase isoforms A, B and/or C as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof, as described above.

Technical Background Aurora kinases play a role in regulating the cell cycle and in particular in the process of cellular mitosis (they have an important role in the mitotic phase of the cell cycle). Therefore, Aurora kinase inhibitors may find application in the treatment of diseases in which there is a disorder of proliferation, cell division, differentiation such as cancer. In particular tumours with mitotic and or spindle defects may be particularly sensitive to CDK inhibitors.

Examples of Aurora kinase inhibitors include AZD1152, MK0457 (VX680), PHA-739358, MLN-8054, MP-235 in particular MK0457 (VX680), PHA-739358, MLN-8054, MP-235. AZD1152 is undergoing clinical evaluation. AZD1152 is a pro-drug which is converted rapidly to the active moiety AZD1152-HQPA in the plasma (AZD-1152 hydroxy-QPA, structure shown below). In early studies in patients with advanced solid malignancies, AZD1152 given in a 2 hr infusion weekly, induces p53 independent cellular multinucleation and polyploidy, resulting in apoptosis. These early studies indicate neutropenia is the dose-limiting toxicology (ASCO 2006).

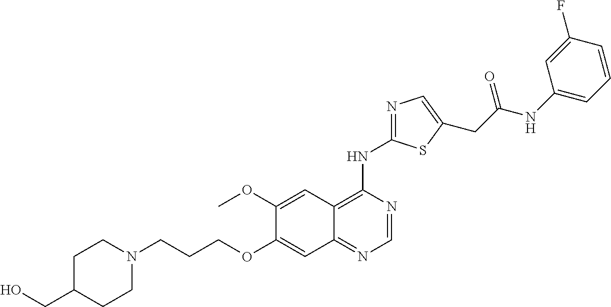

AZD1152 and AZDl152-HQPA can be synthesized as described in WO 02/00649 or by processes analogous thereto.

MK0457 (VX-680) is undergoing clinical evaluation. MK0457 has been given to patients with refractory malignancies in a continuous 5 day infusion every 28 days. These early studies indicate neutropenia is the dose-limiting toxicology (ASCO 2006).

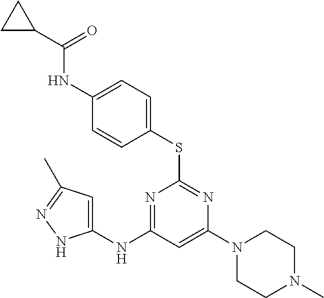

MK0457 can be synthesised as described in Harrington et al, Nat. Med. 2004 March; 10(3):262-7 and WO 02/057259, WO 02/059111, WO 02/059112, WO 02/062789, WO 02/068415, WO 02/066461, WO 02/050065, WO 02/050066 and in particular WO 2004/000833, and by processes analogous thereto.

PHA-739358, the structure of which is shown below, is currently being evaluated by Nerviano Medical Sciences SrI in a multicenter phase 1 dose escalation clinical trials.

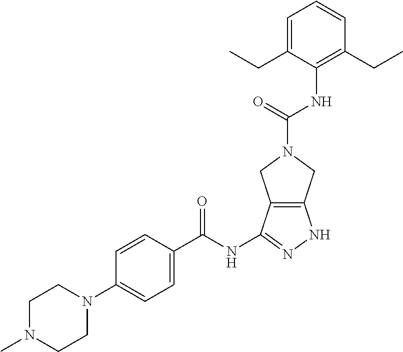

PHA-739358 can be synthesised as described in Fancelli et al, Journal of Medicinal Chemistry (2005), 48(8), 3080-3084 and WO02/12242 and by processes analogous thereto.

MLN-8054 the chemical name of which is 4-[9-Chloro-7-(2,6-difluoro-phenyl)-5H-benzo[c]pyrimido[4,5-e]azepin-2-ylamino]-benzoic acid (structure shown below) is currently being evaluated in multicenter phase 1 dose escalation clinical trials in patients with refractory solid tumours including lymphomas.

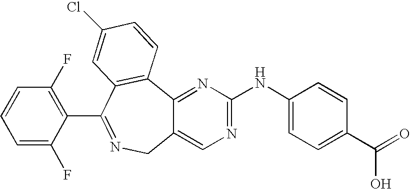

MLN-8054 can be synthesised as described in WO 2005/111039, and by processes analogous thereto.

SuperGen, following the acquisition of Montigen in April 2006, is investigating a series of small molecule Aurora-2 kinase inhibitors that induce apoptosis and inhibit cell division, including MP-235 (HPK-62) (4-(6,7-Dimethoxy-9H-1,3,9-triaza-fluoren-4-yl)-piperazine-1-carbothioic acid [4-(pyrimidin-2-ylsulfamoyl)-phenyl]-amide, structure shown), for the potential treatment of various cancers, including pancreatic cancer. MP-235 can be synthesised as described in WO 2005/037825 and by processes analogous thereto

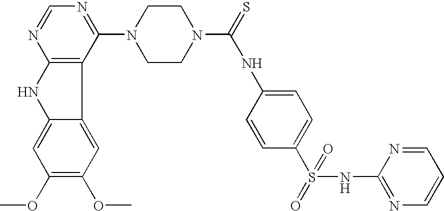

### 21. Hsp90 Inhibitors

Definition: The term Hsp90 inhibitor as used herein refers to compounds that inhibit or modulate the activity of Heat Shock Protein 90 as described herein.

Technical Background: In response to cellular stresses including heat, toxins, radiation, infection, inflammation, and oxidants, all cells produce a common set of heat shock proteins (Hsps) (Macario & de Macario 2000). Most heat shock proteins act as molecular chaperones. Chaperones bind and stabilize proteins at intermediate stages of folding and allow proteins to fold to their functional states. Hsp90 is the most abundant cytosolic Hsp under normal conditions. There are two human isoforms of Hsp90, a major form Hsp90α and minor form Hsp90β. Hsp90 binds proteins at a late stage of folding and is distinguished from other Hsps in that most of its protein substrates are involved in signal transduction. It has been shown that ATP bound at the N-terminal pocket of Hsp90 is hydrolysed. This ATPase activity results in a conformational change in Hsp90 that is required to enable conformational changes in the client protein.

Activation of Hsp90 is further regulated through interactions with a variety of other chaperone proteins and can be isolated in complex with other chaperones including Hsp70, Hip, Hop, p23, and p50cdc37. Many other co-chaperone proteins have also been demonstrated to bind Hsp90. There is some evidence that Hsp90 is found primarily within “activated” multichaperone complexes in the tumour cells as opposed to “latent” complexes in normal cells.

Increased genetic instability associated with the cancer phenotype leads to an increase in the production of non-native or mutant proteins. The ubiquitin pathway also serves to protect the cell from non-native or misfolded proteins, by targeting these proteins for proteasomal degradation. Mutant proteins are by their nature not native and therefore have the potential to show structural instability and an increased requirement for the chaperone system. (Giannini et al., Mol Cell Biol. 2004; 24(13):5667-76).

The number of reported Hsp90 client proteins now exceeds 100. Since many of its client proteins are involved in cell signalling proliferation and survival, Hsp90 has received major interest as an oncology target. Hsp90 protein kinase client proteins implicated in cell proliferation and survival include the following; Cellular Src (c-Src), ErbB2 (Her2/neu), Polo-like kinases (Plks), Akt (PKB), c-Raf, B-RAF, Mek, epidermal growth factor receptor (EGFR), FMS-like tyrosine kinase 3 (FLT3), c-met, Cdk1, Cdk2, Cdk4, and Cdk6, Wee-1, Mutant p53, Hypoxia inducible factor-1a (HIF-1a)

Examples of Hsp90 inhibitors include herbimycin, geldanamycin (GA), 17-AAG e.g. Kos-953 and CNF-1010, 17-DMAG (Kos-1022), CNF-2024 (an oral purine), and IPI-504, in particular 17-MG e.g. Kos-953 and CNF-1010, 17-DMAG (Kos-1022), CNF-2024, and IPI-504. Preferred compounds are geldanamycin analogs such as 17-MG e.g. Kos-953 and CNF-1010, 17-DMAG (Kos-1022), and IPI-504.

Ansamycin antibiotics herbimycin, geldanamycin (GA) and 17-allylamino-17-desmethoxygeldanamycin (17-AAG) are ATP binding site inhibitors that block the binding of ATP and prevent conversion to the mature complex (Grenert et. al., 1997. J Biol. Chem., 272:23834-23850). Despite Hsp90 being ubiquitously expressed, GA and its analogues have a higher binding affinity for Hsp90 derived from tumour vs. normal cell lines (Kamal et. al., Nature 2003; 425: 407-410). GA also shows more potent cytotoxic activity in tumour cells and is sequestered at higher concentrations within tumours in xenograft mouse models (Brazidec J. Med. Chem. 2004, 47, 3865-3873). Furthermore the ATP-ase activity of Hsp90 is elevated in cancer cells and is an indication of the increased level of stress in these cells. Hsp90 gene amplification has also been reported to occur in the later stages of cancer (Jolly and Morimoto JNCI Vol. 92, No. 19, 1564-1572, 2000).

17-AAG (NSC-330507, 17-allylaminogeldanamycin) is an injectable semisynthetic derivative of geldanamycin and a polyketide inhibitor of Hsp90 identified at the University of Maryland under development by Kosan Biosciences, in collaboration with the National Cancer Institute (NCl) and the UK Institute of Cancer Research, for the potential treatment of cancer. Studies of 17-AAG have been initiated in melanoma, multiple myeloma, non-Hodgkin's lymphoma (NHL) and Hodgkin's lymphoma (HL) and as a combination therapy with imatinib (qv) for chronic myelogenous leukemia (CML).

The structure of 17-AAG is outlined below. It can be prepared as described in WO 02/36574 and processes analogous those described therein.

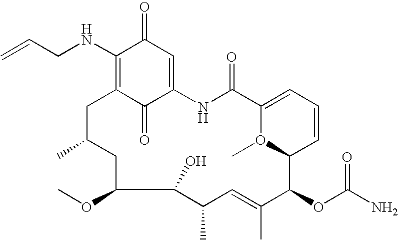

KOS-953 is a 17-MG formulation developed by Kosan that replaces the DMSO-egg lecithin vehicle used in the original formulation, with the aim of improving patient tolerability and providing greater stability. This can be prepared as described in WO 2005/110398 and processes analogous those described therein.

Conforma is developing CNF-1010, an organic solvent-free lipid-based formulation of 17-MG (qv) for the potential iv treatment of cancer. This can be prepared as described in WO 03/026571, WO 02/069900 and WO 2006/050333 and processes analogous those described therein. An oral formulation of 17-AAG is described by Conforma in US 2006/0067953.

17-DMAG (17-dimethylaminoethylamino-17-demethoxygeldanamycin hydrochloride, NSC-707545; structure shown) is an analog of 17-AAG (qv). It is a water-soluble geldanamycin derivative and it is being investigated for advanced solid tumors. Kosan, under license from the National Cancer Institute (NCl), is developing an iv formulation of KOS-1022 (17-DMAG), for the potential treatment of solid tumors. Kosan is also developing an oral formulation of KOS-1022 (qv) for the same indication.

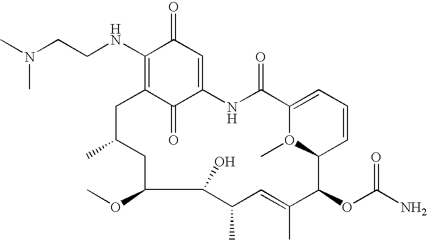

It can be prepared as described in WO 03/013430 and processes analogous to those described therein.

Infinity is developing the Hsp90 inhibitor IPI-504, a further analog of 17-AAG (qv) that is soluble in aqueous formulations for iv administration, for the potential treatment of cancer. Infinity started studies of IPI-504 in multiple myeloma (MM), and gastrointestinal stromal tumors (GIST), and the compound has potential for other haematological cancers and solid tumors.

The structure of IPI-504, a reduced form of 17-AAG called 18,21-didehydro-17-demethoxy-18,21-dideoxo-18,21-dihydroxy-17-(2-propenylamino)-geldanamycin monohydrochloride, is shown below. It can be prepared as described in WO 2005/063714 and processes analogous those described therein.

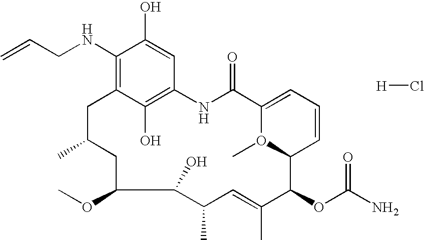

Conforma Therapeutics is developing CNF-2024, a synthetic oral Hsp 90 inhibitor, for the potential treatment of cancer. CNF-2024 is an oral purine analogue.

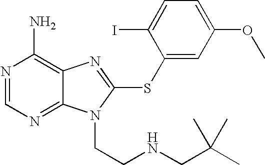

It can be prepared as described in J Med Chem (2006) 49: 817-828.

### 22. Checkpoint Targeting Agents

The cell proliferation cycle is a complex process during which the cell first replicates its chromosomes and then undergoes cell division or cytokinesis. At various stages of the cycle, mechanisms exist to prevent further progression through the cycle until all appropriate events have occurred. This ensures the integrity of the DNA of the cell as it progresses through the cycle in the required sequential manner. One such checkpoint is known to occur in mitosis. This is variously referred to as the mitotic or spindle checkpoint. Cells are held at this checkpoint until all chromosomes are appropriately attached to the mitotic spindle via their centrosomes. Defects in this checkpoint lead to either aneuploid phenotypes, typical of cancer cells or an imbalance of chromosomes in daughter cells. Some cancer therapies are known to act by disruption of this checkpoint causing chromosome mis-alignment or premature cytokinesis leading to activation of a checkpoint that results in preferential death of the tumour cell. For example the taxanes and epothilones are classes of agents which cause stabilisation of spindle microtubules preventing the normal spindle contraction process. The vinca alkaloids are another class of agents which act to prevent spindle formation via an action on tubulin the principal protein in the microtubules. Agents which cause DNA damage or disrupt DNA replication including platinum compounds and nucleoside analogues such as 5-FU lead to cell arrest at checkpoints and subsequent cell death. They thus require a functional checkpoint for their therapeutic action.

Further checkpoint targeting agents are those that cause DNA damage or disrupt DNA replication including platinum compounds such as cisplatin and nucleoside analogues such as 5-FU leading to cell arrest at checkpoints and subsequent cell death. In this context a combination of Aurora kinase inhibitors with the platinum compounds and nucleoside analogues would be expected to be beneficial as they could sensitise cells to the cytotoxic effects. Particular platinum compounds and nucleoside analogues are described herein.

Further checkpoint targeting agents that activate, interfere with or modulate the cell cycle checkpoints which would also be expected to be particularly beneficial for use in combinations of the invention include polo-like kinase inhibitors (Plks), CHK kinase inhibitors, inhibitors of the BUB kinase family and kinesin inhibitors. Polo-like kinases are important regulators of cell cycle progression during M-phase. Plks are involved in the assembly of the mitotic spindle apparatus and in the activation of CDK/cyclin complexes. Plk1 regulates tyrosine dephosphorylation of CDKs through phosphorylation and activation of Cdc25C. CDK1 activation in turn leads to spindle formation and entry into M phase. The importance of Checkpoint kinases such as Chk1 and Chk2 is described herein.

Thus other agents in development which act to disrupt the mitotic checkpoint and therefore could be combined beneficially with the compounds of the invention include polo-like kinase inhibitors (e.g. BI-2536), CHK kinase inhibitors (e.g. Irofulven (a CHK2 inhibitor), 7-hydroxystaurosporine (UCN-01, an inhibitor of both CHK1 and PKC) and PD-321852), inhibitors of the BUB kinase family, and kinesin inhibitors (also known as mitotic kinesin spindle protein (KSP) inhibitors) such as CK0106023, CK-0060339 and SB-743921 (structures shown below).

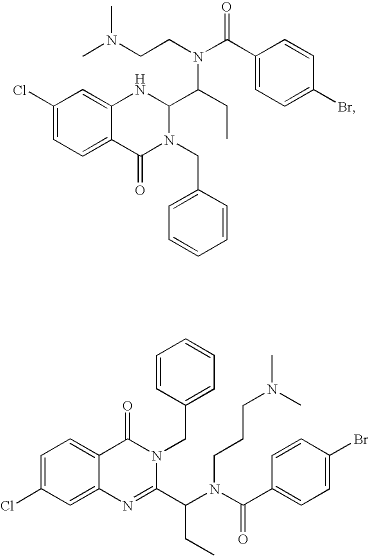

CK0106023, CK-0060339 and SB-743921 can be prepared and used as described in WO 01/30768 and WO 01/98278 and processes analogous thereto.

CHK kinase inhibitors include irofulven, UCN-01 and PD-321852. Irofulven (structure shown) is a semisynthetic compound derived from illudin S, a toxin from the Omphalotus illudens mushroom, for the potential treatment of refractory and relapsed tumors, including ovarian, prostate, hepatocellular, breast, lung and colon cancers, and gliomas. This can be synthesised as described in WO 98/05669 or processes analogous thereto.

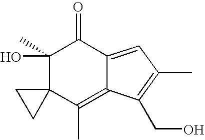

PD-321852, a checkpoint kinase Chk I inhibitor, (structure shown), is being investigated by Pfizer for the potential treatment of cancer.

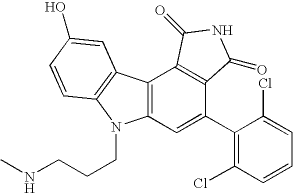

It can be prepared and used as described in WO 01/53274, WO 01/53268 and in particular WO 03/091255 or processes analogous thereto.

BI-2536 (structure shown below) an inhibitor of the serine-threonine kinase polo-like kinase-1 (PLK-1), for the potential treatment of solid tumors. It can be prepared and used as described in WO2004/076454, WO 2006/018220, WO 2006/018221 and WO 2006/018222 or processes analogous thereto.

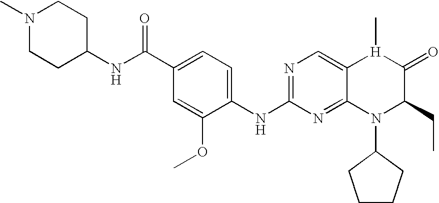

In addition, checkpoint targeting agents that arrest cells in G2/M phase could also be combined with the combinations of the invention to have a similar beneficial effect. Therefore Platinum compounds and CDK inhibitors would be therefore be expected to be beneficial in combination with the combinations of the invention and are thus further Checkpoint Targeting Agents. Particular Platinum compounds and CDK inhibitors are described herein.

Thus, examples of Checkpoint Targeting Agents for use according to the invention include Platinum compounds, nucleoside analogues, CDK inhibitors, Taxanes, Vinca alkaloids, polo-like kinase inhibitors, CHK kinase inhibitors, inhibitors of the BUB kinase family and kinesin inhibitors, in particular Platinum compounds, nucleoside analogues, Taxanes and Vinca alkaloids more particularly checkpoint targeting agents which target the mititoic checkpoint such as Taxanes and Vinca alkaloids. Particular combinations of the invention include cisplatin or vinblastine or taxol or 5FU, in particular taxol.

Particular examples of Checkpoint Targeting Agents for use according to the invention include polo-like kinase inhibitors, CHK kinase inhibitors, inhibitors of the BUB kinase family and kinesin inhibitors. In one embodiment the optional auxiliary agents for use in the combinations of the invention are selected from BI-2536, Irofulven, 7-hydroxystaurosporine, PD-321852, CK0106023, CK-0060339 and SB-743921.

### 23. DNA Repair Inhibitors

DNA repair inhibitors include PARP inhibitors.

Definition: The term “PARP inhibitor” is used herein to define compounds which inhibit or modulate the activity of the family of Poly adenosine diphosphate rbose (poly(ADP-Ribose)) nuclear enzymes, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described above. They may also be referred to as “DNA repair inhibitors”.

Biological activity: PARP inhibitors have a role as chemosensitizing agents (for example by preventing DNA repair after anticancer therapy) and may have a role in enhancing overall patient response to anti-cancer treatments. PARP inhibitors may also act in isolation as anti cancer agents in patients whose tumours have intrinsic deficiencies in DNA repair.

Technical background: The PARP enzyme synthesizes poly(ADP-ribose), a branched polymer that can consists of over 200 ADP-ribose units. The protein acceptors of poly(ADP-ribose) are directly or indirectly involved in maintaining DNA integrity. They include histones, topoisomerases, DNA and RNA polymerases, DNA ligases, and Ca 2′- and Mg 2, -dependent endonucleases. PARP protein is expressed at a high level in many tissues, most notably in the immune system, heart, brain and germ-line cells. Under normal physiological conditions, there is minimal PARP activity. However, DNA damage causes an immediate activation of PARP by up to 500-fold.

PARP is activated by damaged DNA fragments and, once activated, catalyzes the attachment of up to 100 ADP-ribose units to a variety of nuclear proteins, including histones and PARP itself. It is also known that PARP inhibitors, such as 3-amino benzamide, affect overall DNA repair in response, for example, to hydrogen peroxide or ionizing radiation. The pivotal role of PARP in the repair of DNA strand breaks is well established, especially when caused directly by ionizing radiation or, indirectly after enzymatic repair of DNA lesions induced by methylating agents, especially temozolamide, topoisomerases I inhibitors and other chemotherapeutic agents as cisplatin and bleomycin. A variety of studies using knockout mice, trans-dominant inhibition models (over-expression of the DNA-binding domain), antisense and small molecular weight inhibitors have demonstrated the role of PARP in repair and cell survival after induction of DNA damage. The inhibition of PARP enzymatic activity should lead to an enhanced sensitivity of tumor cells towards DNA damaging treatments.

PARP inhibitors have been reported to be effective in radiosensitizing (hypoxic) tumor cells and effective in preventing tumor cells from recovering from potentially lethal and sublethal damage of DNA after radiation therapy, presumably by their ability to prevent DNA strand break rejoining and by affecting several DNA damage signaling pathways. PARP inhibitors have been used to treat cancer. A recent comprehensive review of the state of the art has been published by Li and Zhang in IDrugs 2001, 4(7): 804.

Preferences and specific embodiments: Preferred PARP inhibitors for use in accordance with the invention are selected from Bendamustine (5-[Bis(2-chloroethyl)amino]-1-methyl-2-benzimidazolebutyric acid or α-[1-Methyl-5-[bis(.beta.-chloroethyl)amino]-2-benzimidazolyl]butyric acid), available from Bayer, INO-1001 (Pardex) from Inotek Pharmaceuticals, BSI-201 from BiPar Sciences, AG-014699 from Pfizer, and ONO-2231 (N-[3-(3,4-dihydro-4-oxo-1-phthalazinyl)phenyl]-4-morpholinebutanamide methanesulfonate) from Ono Pharmaceutical.

Posology: The PARP inhibitors are advantageously administered in daily dosages of 20-100 mg, for example 80-120 mg/m2 iv over a 30 to 60 min infusion over a 21 day cycle for Bendamustine.—The key PARP inhibitor is a Pfizer product which is in phase III combination trials in metastatic melanoma. It is administered intravenously on days one thru five of a twenty-one day cycle dose?

### 24. Inhibitors of G-protein Coupled Receptors (GPCR)

A preferred GPCR is Atrasentan (3-Pyrrolidinecarboxylic acid, 4-(1,3-benzodioxol-5-yl)-1-[2-(dibutylamino)-2-oxoethyl]-2-(4-methoxyphenyl)-, [2R-(2.alpha., 3. beta., 4.alpha.)]-). Atrasentan, from Abbott Laboratories, is a potent and selective endothelin A receptor antagonist for the treatment of prostate tumors. There is also evidence of biological activity in other cancer types such as glioma, breast tumor, lung tumor, brain tumor, ovary tumor, colorectal tumor and renal tumor.

Posology: Atrasentan may be advantageously administered orally in dosages of e.g. 10 mg daily.

### Anti-cancer Agent Combinations

The combinations of the invention may comprise one, two or more auxiliary compounds. In such embodiments, the auxiliary compounds may be anti-cancer agents. In such embodiments, the anticancer agents may be independently selected from carboplatin, cisplatin, taxol, taxotere, gemcitabine, and vinorelbine. Preferably the anti-cancer agents are carboplatin, taxol and vinorelbine, or carboplatin and taxol.

Combinations of compounds of formula (I) or (0) and compounds of formula (I′″) with carboplatin, taxol and vinorelbine or combinations of compounds of formula (I) or (0) and compounds of formula (I′″) with carboplatin and taxol, are particularly suitable for treating Non-Small cell lung cancer.

Alternatively, combinations of compounds of formula (I) or (0) and compounds of formula (I′″) with platinum agents, taxol, taxotere, gemcitabine, pemetrexed, mitomycin, ifosfamide, vinorelbine, erlotinib and bevacizumab or combinations with carboplatin and taxol or cisplatin and gemcitabine are particularly suitable for treating Non-Small cell lung cancer.

In one embodiment, the anti-cancer agents are independently selected from 5-FU, leucovorin, oxaliplatin, CPT 11, and bevacizumab. Preferably, the anti-cancer agents are 5-FU, leucovorin and CPT 11 or 5-FU, leucovorin and oxaliplatin.

Combinations of compounds of formula (I) or (0) and compounds of formula (I′″) with 5-FU, leucovorin and CPT 11 or a combination of compounds of formula (I) or (0) and compounds of formula (I′″) with 5-FU, leucovorin and oxaliplatin, are particularly suitable for treating colon cancer. In addition, combinations of compounds of formula (I) or (0) and compounds of formula (I′″) with 5-FU, leucovorin and CPT 11 or a combination with 5-FU, leucovorin and oxaliplatin, each with bevacizumab, are particularly suitable for treating colon cancer.

In one embodiment, the anti-cancer agents are independently selected from methotrexate, taxanes, anthracyclines e.g. doxorubicin, herceptin, lapatinib, bevacizumab, mitozantrone, epothilones, 5-FU, and cyclophosphamide. In another embodiment, the anti-cancer agents are independently selected from methotrexate, taxanes, anthracyclines e.g. doxorubicin, herceptin, 5-FU, and cyclophosphamide. In one embodiment, the anti-cancer agents are independently selected from taxanes, anthracyclines e.g. doxorubicin, herceptin, 5-FU, and cyclophosphamide. In one embodiment, the anti-cancer agents are independently selected from 5-FU, methotrexate, cyclophosphamide and doxorubicin. Preferably the anti-cancer agents are 5-FU, methotrexate and cyclophosphamide or 5-FU, doxorubicin and cyclophosphamide or doxorubicin and cyclophosphamide.

Combinations of compounds of formula (I) or (0) and compounds of formula (I′″) with 5-FU, methotrexate and cyclophosphamide, or a combination of compounds of formula (I) or (0) and compounds of formula (I′″) with 5-FU, doxorubicin and cyclophosphamide, or combinations of compounds of formula (I) or (0) and compounds of formula (I′″) with doxorubicin and cyclophosphamide, are particularly suitable for treating breast cancer.

In one embodiment, the anti-cancer agents are independently selected from cyclophosphamide, doxorubicin (hydroxydaunorubicin), vincristine, and prednisone. In another embodiment, the anti-cancer agents are independently selected from cyclophosphamide, doxorubicin (hydroxydaunorubicin), vincristine, bortezomib, rituximab and prednisone. Preferably the anti-cancer agents are cyclophosphamide, doxorubicin (hydroxydaunorubicin), vincristine and prednisone, or cyclophosphamide, vincristine and prednisone, with or without rituximab. Preferably the anti-cancer agents are cyclophosphamide, doxorubicin (hydroxydaunorubicin), vincristine and prednisone, or cyclophosphamide, vincristine and prednisone.

Combinations of compounds of formula (I) or (0) and compounds of formula (I′″) with cyclophosphamide, doxorubicin (hydroxydaunorubicin), vincristine and prednisone are particularly suitable for treating non Hodgkin's lymphoma (and in particular high grade non Hodgkin's lymphoma). Combinations of compounds of formula (I) or (0) and compounds of formula (I′″) with cyclophosphamide, doxorubicin (hydroxydaunorubicin), vincristine, rituximab, and prednisone are also particularly suitable for treating non Hodgkin's lymphoma (and in particular high grade non Hodgkin's lymphoma).

Combinations of compounds of formula (I) or (0) and compounds of formula (I′″) with cyclophosphamide, vincristine and prednisone are particularly suitable for treating non Hodgkin's lymphoma (and in particular low grade non Hodgkin's lymphoma). Combinations of compounds of formula (I) or (0) and compounds of formula (I′″) with cyclophosphamide, vincristine, rituximab, and prednisone are also particularly suitable for treating non Hodgkin's lymphoma (and in particular low grade non Hodgkin's lymphoma).

In one embodiment, the anti-cancer agents may be independently selected from vincristine, doxorubicin, and dexamethasone. In another embodiment, the anti-cancer agents are independently selected from vincristine, thalidomide, doxorubicin, bortezomib and dexamethasone. Preferably the anti-cancer agents are vincristine, doxorubicin and dexamethasone.

Combinations of compounds of formula (I) or (0) and compounds of formula (I′″) with vincristine, doxorubicin, thalidomide and dexamethasone are particularly suitable for treating multiple myeloma. In addition, combinations with vincristine, doxorubicin and dexamethasone are particularly suitable for treating multiple myeloma.

In one embodiment, the anti-cancer agents are independently selected from: (a) fludarabine and rituxamab or (b) fludarabine, almentuzamab and rituxamab. Preferably the anti-cancer agents are fludarabine and rituxamab.

Combinations of compounds of formula (I) or (0) and compounds of formula (I′″) with fludarabine and rituxamab are particularly suitable for treating chronic lymphocytic leukemia.

In one embodiment the combination of the invention optionally excludes combination of two or more of the following anti-cancer agents selected from a topoisomerase inhibitor, an alkylating agent, a antimetabolite, DNA binders, monoclonal antibodies, signal transduction inhibitors and microtubule inhibitors (tubulin targeting agents), such as cisplatin, cyclophosphamide, doxorubicin, irinotecan, fludarabine, 5FU, taxanes and mitomycin C.

In one embodiment the combination of the invention includes at least one anti-cancer agent selected from an antiandrogen, a histone deacetylase inhibitor (HDAC), cylcooxygenase-2 (COX-2) inhibitor, proteasome inhibitor, DNA methylation inhibitor and a CDK inhibitor.

### Disease-specific Anti-cancer Agent Combinations

### Multiple Myeloma

Combinations of compounds of formula (I) or (0) and compounds of formula (I′″) with vincristine, doxorubicin, thalidomide and dexamethasone are particularly suitable for treating multiple myeloma. In addition, combinations with vincristine, doxorubicin and dexamethasone are particularly suitable for treating multiple myeloma.

Particularly suitable for treating multiple myeloma are combinations of compounds of formula (I) or (0) and compounds of formula (I′″) with: (a) monoclonal antibodies (e.g. those targeting Interleukin 6); (b) proteasome inhibitors (e.g. bortezomib); (c) proteasome inhibitors and corticosteroids (e.g. velcade and dexamethasone); and (d) corticosteroids, alkylating agents and lenolidamide/thalidomide (e.g. prednisolone, melphalan and thalidomide).

### Melanoma

Particularly suitable for treating melanoma are combinations of compounds of formula (I) or (0) and compounds of formula (I′″) with: (a) DNA methylase inhibitors/hypomethylating agents (e.g. temozolamide); (b) alkylating agents (e.g. dacarbazine or fotemustine); and (c) DNA methylase inhibitors/hypomethylating agents (e.g. temozolamide) and DNA repair inhibitors/PARP inhibitors.

### Breast Cancer

Particularly suitable for treating breast cancer are combinations of compounds of formula (I) or (0) and compounds of formula (I′″) with: (a) monoclonal antibodies (e.g. trastuzumab and bevicizamab); (b) monoclonal antibodies (e.g. trastuzumab and bevicizamab) and taxanes; and (c) antimetabolites (e.g. capecitabine) and signalling inhibitors (e.g. lapatinib).

### Prostate Cancer

Particularly suitable for treating prostate cancer are combinations of compounds of formula (I) or (0) and compounds of formula (I′″) with hormones and G-protein coupled receptor inhibitors.

### Non Small Cell Lung Cancer (NSCLC)

Particularly suitable for treating NSCLC are combinations of compounds of formula (I) or (0) and compounds of formula (I′″) with: (a) platinum compounds and taxanes; and (b) platinum compounds and antimetabolites.

### Specific Combinations of the Invention

Particular combinations according to the invention include compounds of formula (I) or (0), and compound of formula (I′″) and subgroups thereof as defined herein with the following two or more anti-cancer agents:

For cancer (and in particular acute myeloid leukemia) treatment, two or more anti-cancer agents independently selected from two or more of anthracycline, Ara C (a.k.a. Cytarabine), 6-mercaptopurine, thiopurine, methotrexate, mitoxantrone, daunorubicin, idarubicin, gemtuzumab ozogamicin and granulocyte colony stimulating factors. In addition, for cancer (and in particular acute myeloid leukemia) treatment, two or more anti-cancer agents independently selected from two or more of anthracycline, Ara C (a.k.a. Cytarabine), 6-mercaptopurine, methotrexate, mitoxantrone, daunorubicin, idarubicin, gemtuzumab ozogamicin and granulocyte colony stimulating factors. Alternatively, the two or more anti-cancer agents may be independently selected from two or more of anthracycline, Ara C (a.k.a. Cytarabine), daunorubicin, idarubicin, gemtuzumab ozogamicin and granulocyte colony stimulating factors.

For cancer (and in particular breast cancer) treatment, two or more anti-cancer agents independently selected from bevacizumab, taxanes, methotrexate, paclitaxel, docetaxel, gemcitabine, anastrozole, exemestane, letrozole, tamoxifen, doxorubicin, herceptin, 5-fluorouracil, cyclophosphamide, epirubicin and capecitabine, particularly 5-FU, methotrexate and cyclophosphamide; 5FU, doxorubicin and cyclophosphamide; or doxorubicin and cyclophosphamide. Preferably, for cancer (and in particular breast cancer) treatment, the two or more anti-cancer agents may also be independently selected from taxanes, methotrexate, paclitaxel, docetaxel, gemcitabine, anastrozole, exemestane, letrozole, tamoxifen, doxorubicin, herceptin, 5-fluorouracil, cyclophosphamide, epirubicin and capecitabine, particularly 5-FU, methotrexate and cyclophosphamide; 5FU, doxorubicin and cyclophosphamide; or doxorubicin and cyclophosphamide.

Typical dosing regimens include:
Cyclophosphamide at 100 mg/m² PO Daily×14 days, Doxorubicin at 30 mg/m² IV Day 1 & day 8 and fluorouracil at 500 mg/m² IV Day 1 & day 8, repeated every 28 days for up to 6 cycles
Cyclophosphamide at 600 mg/m² IV Day 1 and Doxorubicin at 60 mg/m² IV Day 1, repeated every 21 days for up to 4 cycles

For cancer (and in particular chronic lymphocytic leukemia (CLL)) treatment, two or more anti-cancer agents independently selected from alemtuzumab, chlorambucil, cyclophosphamide, vincristine, predinisolone, fludarabine, mitoxantrone and rituximab/rituxamab, particularly fludarabine and rituxamab. Preferably, for cancer (and in particular chronic lymphocytic leukemia (CLL)) treatment, the two or more anti-cancer agents are independently selected from chlorambucil, cyclophosphamide, vincristine, predinisolone, fludarabine, mitoxantrone and rituximab/rituxamab, particularly fludarabine and rituxamab.

For cancer (and in particular chronic myeloid leukemia (CML)) treatment, two or more anti-cancer agents independently selected from hydroxyurea, cytarabine, and imatinib. In addition, for cancer (and in particular chronic myeloid leukemia (CML)) treatment, the two or more anti-cancer agents are independently selected from hydroxyurea, cytarabine, Interferon-alpha and imatinib. Alternatively for cancer (and in particular chronic myeloid leukemia (CML)) treatment, two or more anti-cancer agents independently selected from hydroxyurea, cytarabine, dasatinib, nilotinib and imatinib.

For cancer (and in particular Colon Cancer treatment), two or more anti-cancer agents independently selected from cetuximab, 5-Fluorouracil, pantuzumab, leucovorin, irinotecan, oxaliplatin, raltirexed, capecitabine, bevacizumab, oxaliplatin, CPT 11. Alternatively for cancer (and in particular Colon Cancer treatment), two or more anti-cancer agents independently selected from cetuximab, 5-Fluorouracil, leucovorin, irinotecan, oxaliplatin, raltirexed, capecitabine, bevacizumab, oxaliplatin, CPT 11, particularly 5-Fluorouracil, Leucovorin and CPT 11 or Fluorouracil, Leucovorin and Oxaliplatin.

Alternatively, for cancer (and in particular Colon Cancer treatment), two or more anti-cancer agents independently selected from 5-Fluorouracil, leucovorin, irinotecan, oxaliplatin, raltirexed, capecitabine, bevacizumab, oxaliplatin, CPT 11 and Avastin, particularly 5-Fluorouracil, Leucovorin and CPT 11 or Fluorouracil, Leucovorin and Oxaliplatin.

Typical dosing regimens include:
Fluorouracil at 400-425 mg/m² IV Days 1 to 5 and Leucovorin at 20 mg/m² IV Days 1 to 5, repeated every 28 days for 6 cycles
Irinotecan at 100-125 mg/m² IV over 90 minutes Days 1, 8, 15 & 22, Folinic acid at 20 mg/m2 IV Days 1, 8, 15 & 22, and Fluorouracil at 400-500 mg/m2 IV Days 1, 8, 15 & 22, repeated every 42 days until disease progression
Oxaliplatin at 85 mg/m2 IV in 500 mL of D5W over 120 minutes Day 1, Folinic acid at 200 mg/m2 IV over 120 minutes Days 1 & 2, Fluorouracil at 400 mg/m2 IV bolus, after Folinic Acid, Days 1 & 2, then Fluorouracil at 600 mg/m2 CIV over 22 hours Days 1 & 2, repeated every 12 days for up to 12 cycles

For cancer (and in particular multiple myeloma treatment), two or more anti-cancer agents independently selected from vincristine, doxorubicin, thalidomide, dexamethasone, melphalan, prednisone, cyclophosphaimde, etoposide, pamidronate, zoledronate and bortezomib, particulary vincristine, doxorubicin and, dexamethasone. Alternatively, for cancer (and in particular multiple myeloma treatment), two or more anti-cancer agents independently selected from vincristine, doxorubicin, dexamethasone, melphalan, prednisone, cyclophosphaimde, etoposide, pamidronate, zoledronate and bortezomib, particulary vincristine, doxorubicin and dexamethasone.

For cancer (and in particular Non-Hodgkin's lymphoma treatment), two or more anti-cancer agents independently selected from cyclophosphamide, doxorubicin/hydroxydaunorubicin, vincristine/Onco-TCS (V/O), prednisolone, methotrexate, cytarabine, bleomycin, etoposide, rituximab/rituxamab, fludarabine, cisplatin, and ifosphamide, particularly cyclophosphamide, doxorubicin (hydroxydaunorubicin), vincristine and prednisone for high grade NHL or cyclophosphamide, vincristine and prednisone for low grade NHL.

For cancer (and in particular Non Small Cell Lung Cancer (NSCLC)) treatment, two or more anti-cancer agents may be independently selected from bevacizumab, gefitinib, erlotinib, cisplatin, carboplatin, etoposide, mitomycin, vinblastine, paclitaxel, docetaxel, gemcitabine and vinorelbine, especially taxol, vinorelbine and carboplatin or taxol and carboplatin. Alternatively for cancer (and in particular Non Small Cell Lung Cancer (NSCLC)) treatment, two or more anti-cancer agents may be independently selected from bevacizumab, gefitinib, erlotinib, cisplatin, carboplatin, mitomycin, vinblastine, paclitaxel, docetaxel, gemcitabine and vinorelbine. Particulalrly preferred for cancer (and in particular Non Small Cell Lung Cancer (NSCLC)) treatment, two or more anti-cancer agents are independently selected from cisplatin, carboplatin, etoposide, mitomycin, vinblastine, paclitaxel, docetaxel, gemcitabine and vinorelbine, especially taxol, vinorelbine and carboplatin or taxol and carboplatin. In particular the two or more anti-cancer agents are independently selected from gemcitabine and cisplatin.

Typical dosing regimens include:
Gemcitabine at 1000 mg/ml IV Days 1, 8 & 15, and Cisplatin at 75-100 m g/m² IV Day 1, repeated every 28 days for 4-6 cycles
Paclitaxel at 135-225 mg/m² IV over 3 hrs Day 1 and Carboplatin at AUC 6.0 IV Day 1, repeated every 21 days for 4-6 cycles Docetaxel at 75 mg/m² IV Day 1, and Carboplatin at AUC 5 or 6 IV Day 1, repeated every 21 days for 4-6 cycles
Docetaxel at 75 mg/m² IV Day 1, and Cisplatin at 75 mg/m² IV Day 1, repeated every 21 days for 4-6 cycles

For cancer (and in particular ovarian cancer) treatment, two or more anti-cancer agents independently selected from platinum compounds (for example Cisplatin, Carboplatin), taxol, doxorubicin, liposomal doxorubicin, paclitaxel, docetaxel, gemcitabine, melphalan and mitoxantrone.

For cancer (and in in particular prostate cancer) treatment, two or more anti-cancer agents independently selected from mitoxantrone, prednisone, buserelin, goserelin, bicalutamide, nilutamide, flutamide, cyproterone acetate, megestrol/megestrel, diethylstilboestrol, docetaxel, paclitaxel, zoledronic acid and taxotere. Alternatively, for cancer (and in particular prostate cancer) treatment, two or more anti-cancer agents independently selected from mitoxantrone, prednisone, buserelin, goserelin, bicalutamide, nilutamide, flutamide, cyproterone acetate, megestrol/megestrel, diethylstilboestrol, docetaxel, paclitaxel, zoledronic acid, prednisolone and taxotere.

### Pharmaceutical Formulations

While it is possible for the active compounds in the combinations of the invention to be administered without any accompanying pharmaceutical excipients, it is preferable to present them as a pharmaceutical composition (e.g. formulation) comprising at least one active compound together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound of formula (0), as defined above, together with an atleast one compound of formula (I′″) and optionally with one or more auxilliary compound and one or more pharmaceutically acceptable carriers, excipients, buffers, adjuvants, stabilizers, or other materials, as described herein.

The term “pharmaceutically acceptable” as used herein pertains to combinations, compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides combinations comprising (or consisting essentially of) a compound of formula (I′″) and compounds of the formula (I) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, interalia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

Pharmaceutical compositions for parenteral injection can also comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If a compound or combination is not stable in aqueous media or has low solubility in aqueous media, it can be formulated as a concentrate in organic solvents. The concentrate can then be diluted to a lower concentration in an aqueous system, and can be sufficiently stable for the short period of time during dosing. Therefore in another aspect, there is provided a pharmaceutical composition comprising a non aqueous solution composed entirely of one or more organic solvents, which can be dosed as is or more commonly diluted with a suitable IV excipient (saline, dextrose; buffered or not buffered) before administration (Solubilizing excipients in oral and injectable formulations, Pharmaceutical Research, 21(2), 2004, p201-230). Examples of solvents and surfactants are propylene glycol, PEG300, PEG400, ethanol, dimethylacetamide (DMA), N-methyl-2-pyrrolidone (NMP, Pharmasolve), Glycerin, Cremophor EL, Cremophor RH 60 and polysorbate. Particular non aqueous solutions are composed of 70-80% propylene glycol, and 20-30% ethanol. One particular non aqueous solution is composed of 70% propylene glycol, and 30% ethanol. Another is 80% propylene glycol and 20% ethanol. Normally these solvents are used in combination and usually diluted at least 2-fold before IV bolus or IV infusion. The typical amounts for bolus IV formulations are ˜50% for Glycerin, propylene glycol, PEG300, PEG400, and ˜20% for ethanol. The typical amounts for IV infusion formulations are ˜15% for Glycerin, 3% for DMA, and ˜10% for propylene glycol, PEG300, PEG400 and ethanol.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds for use in the combinations of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

Solid dispersions of drugs are generally produced by melt or solvent evaporation methods. For melt processing, the materials (excipients) which are usually semisolid and waxy in nature, are heated to cause melting and dissolution of the drug substance, followed by hardening by cooling to very low temperatures. The solid dispersion can then be pulverized, sieved, mixed with excipients, and encapsulated into hard gelatin capsules or compressed into tablets. Alternatively the use of surface-active and self-emulsifying carriers allows the encapsulation of solid dispersions directly into hard gelatin capsules as melts. Solid plugs are formed inside the capsules when the melts are cooled to room temperature.

Solid solutions can also be manufactured by dissolving the drug and the required excipient in either an aqueous solution or a pharmaceutically acceptable organic solvent, followed by removal of the solvent, using a pharmaceutically acceptable method, such as spray drying. The resulting solid can be particle sized if required, optionally mixed with exipients and either made into tablets or filled into capsules.

A particularly suitable polymeric auxiliary for producing such solid dispersions or solid solutions is polyvinylpyrrolidone (PVP).

This combination could contain solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Accordingly, in a further embodiment, the invention provides a package containing separate dosage units, one or more of which contain a compound of the formula (0), (I⁰), (I), (Ia), (Ib), (II), (III), (IV), (IVa), (Va), (Vb), (VIa), (VIb), (VII) or (VIII) and sub-groups thereof as defined herein, and one or more of which contain a compound of the formula (I′″) and optionally one or more of which contain an auxiliary compound. Dosage units containing a compound of the formula (0), (I⁰), (I), (Ia), (Ib), (II), (III), (IV), (IVa), (Va), (Vb), (VIa), (VIb), (VII) or (VIII) and sub-groups thereof as defined herein, a compound of Formula (I′″) as defined herein and optionally an auxiliary inhibitor have suitable amounts of active ingredient as defined herein. A package contains enough tablets, capsules or the like to treat a patient for a pre-determined period of time, for instance for 2 weeks, 1 month or 3 months.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The combinations or their constituent components (e.g. the compounds of the formula (I) or (0) and formula (I′″)) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The combination will be administered to a patient in need thereof (for example a human or animal patient) in an amount sufficient to achieve the desired therapeutic effect.

### Methods of Treatment

The combinations of the invention will be useful in the prophylaxis or treatment of a range of disease states or conditions mediated by cyclin dependent kinases and glycogen synthase kinase-3. Examples of such disease states and conditions are set out above.

The combination is generally administered to a subject in need of such administration, for example a human or animal patient, preferably a human.

The combination will typically be administered in amounts that are therapeutically or prophylactically useful and which generally are non-toxic. However, in certain situations (for example in the case of life threatening diseases), the benefits of administering a combination of the invention may outweigh the disadvantages of any toxic effects or side effects, in which case it may be considered desirable to administer the combination in amounts that are associated with a degree of toxicity.

The constituent compounds of the combinations of the invention may be administered over a prolonged term to maintain beneficial therapeutic effects or may be administered for a short period only. Alternatively they may be administered in a pulsatile or continuous manner.

A typical daily dose of the constituent compounds of the combination can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compounds can be administered on a daily basis or on a repeat basis every 2, or 3, or 4, or 5, or 6, or 7, or 10 or 14, or 21, or 28 days for example.

The constituent compounds of the combinations of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg.

The compounds may be administered once or more than once each day. The compounds can be administered continuously (i.e. taken every day without a break for the duration of the treatment regimen). Alternatively, the compounds can be administered intermittently, i.e. taken continuously for a given period such as a week, then discontinued for a period such as a week and then taken continuously for another period such as a week and so on throughout the duration of the treatment regimen. Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

Ultimately, however, the quantity of compound administered and the type of composition used will be commensurate with the nature of the disease or physiological condition being treated and will be at the discretion of the physician.

Accordingly, a person skilled in the art would know through their common general knowledge the dosing regimes and combination therapies to use. It will be appreciated that the preferred method and order of administration and the respective dosage amounts and regimes for each component of the combination will depend on the particular compounds of formula (0) and of Formula (I′″), and the optional further auxiliary compound(s) being administered, their route of administration, the particular tumour being treated and the particular host being treated. The optimum method and order of administration and dosage amounts and regime can be readily determined by those skilled in the art using conventional methods and in view of the information set out herein.

The combinations of the invention as defined herein can be further combined and/or administered with one of more other compounds for treatment of a particular disease state, for example a neoplastic disease such as a cancer as hereinbefore defined. Examples of other therapeutic agents or treatments that may be administered together (whether concurrently or at different time intervals) with the combinations of the invention include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferases
Cytokines and retinoids
Chromatin targeted therapies
Radiotherapy, and,
Other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF). Also included are agents that inhibit bone resorption such as bisphosphonate agents e.g. zoledronate, pamidronate and ibandronate, agents that suppress inflammatory responses (such as dexamethazone, prednisone, and prednisolone) and agents used to reduce blood levels of growth hormone and IGF-I in acromegaly patients such as synthetic forms of the brain hormone somatostatin, which includes octreotide acetate which is a long-acting octapeptide with pharmacologic properties mimicking those of the natural hormone somatostatin. Further included are agents such as leucovorin, which is used as an antidote to drugs that decrease levels of folic acid, or folinic acid it self and agents such as megestrol acetate which can be used for the treatment of side-effects including oedema and thromoembolic episodes.

Each of the compounds present in the combinations of the invention may be given in individually varying dose schedules and via different routes.

The compound of the formula (0) and compound of formula (I′″) may be simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The combinations of the invention may also be administered in conjunction with non-chemotherapeutic treatments such as radiotherapy, photodynamic therapy, gene therapy; surgery and controlled diets.

For use in combination therapy, the compound of the formula (I) and the compound of formula (I′″) and one, two, three, four or more optional auxiliary compounds can be, for example, formulated together in a dosage form containing two, three, four or more auxiliary compounds. In an alternative, the constituent compounds of the combination of the invention may be formulated separately and presented together in the form of a kit, optionally with instructions for their use.

A person skilled in the art would know through his or her common general knowledge the dosing regimes and combination therapies to use.

### Methods of Diagnosis

The combinations of the invention comprise pyrazole compounds that inhibit or modulate the activity of Cyclin Dependent Kinases (CDK) and/or Glycogen Synthase Kinases (GSK, e.g. GSK-3) and optionally one or more auxiliary compounds. The auxiliary compounds may themselves exhibit protein kinase modulatory or inhibitory activity and such activity may be quite distinct from that of the pyrazole component of the combinations (as described infra). Thus, depending on the identity of the auxiliary compound(s) present, the combination as a whole may inhibit or modulate the activity of one or more of a range of different protein kinases, including those described below.

Prior to administration of a combination comprising a compound of the formula (0) and a compound of formula (I′″) and optionally an auxiliary compound as defined herein, a patient may be screened to determine whether a disease or condition from which the patient is or may be suffering is one which would be susceptible to treatment with a combination having activity against Aurora and/or cyclin dependent kinases.

For example, a biological sample taken from a patient may be analysed to determine whether a condition or disease, such as cancer, that the patient is or may be suffering from is one which is characterised by a genetic abnormality or abnormal protein expression which leads to over-activation of CDKs or to sensitisation of a pathway to normal CDK activity. Examples of such abnormalities that result in activation or sensitisation of the CDK2 signal include up-regulation of cyclin E, (Harwell R M, Mull B B, Porter D C, Keyomarsi K.; J Biol. Chem. 2004 Mar. 26; 279(13):12695-705) or loss of p21 or p27, or presence of CDC4 variants (Rajagopalan H, Jallepalli P V, Rago C, Velculescu V E, Kinzler K W, Vogelstein B, Lengauer C.; Nature. 2004 Mar. 4; 428(6978):77-81). Tumours with mutants of CDC4 or up-regulation, in particular over-expression, of cyclin E or loss of p21 or p27 may be particularly sensitive to CDK inhibitors. Alternatively or in addition, a biological sample taken from a patient may be analysed to determine whether a condition or disease, such as cancer, that the patient is or may be suffering from is one which is characterised by upregulation of Aurora kinase and thus may be particularly sensitive to Aurora inhibitors. The term up-regulation includes elevated expression or over-expression, including gene amplification (i.e. multiple gene copies) and increased expression by a transcriptional effect, and hyperactivity and activation, including activation by mutations.

Thus, the patient may be subjected to a diagnostic test to detect a marker characteristic of over-expression, up-regulation or activation of Aurora kinase or the patient may be subjected to a diagnostic test to detect a marker characteristic of up-regulation of cyclin E, or loss of p21 or p27, or presence of CDC4 variants. The term diagnosis includes screening. By marker we include genetic markers including, for example, the measurement of DNA composition to identify mutations of Aurora or CDC4. The term marker also includes markers which are characteristic of up regulation of Aurora or cyclin E, including enzyme activity, enzyme levels, enzyme state (e.g. phosphorylated or not) and mRNA levels of the aforementioned proteins. Tumours with upregulation of cyclin E, or loss of p21 or p27 may be particularly sensitive to CDK inhibitors. Tumours may preferentially be screened for upregulation of cyclin E, or loss of p21 or p27 prior to treatment. Thus, the patient may be subjected to a diagnostic test to detect a marker characteristic of up-regulation of cyclin E, or loss of p21 or p27.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, see Ewart-Toland et al., (Nat. Genet. 2003 August; 34(4):403-12), that individuals forming part of the sub-population possessing the Ile31 variant of the STK gene (the gene for Aurora kinase A) may have an increased susceptibility to certain forms of cancer. Therefore that such individuals suffering from cancer will benefit from the administration of combinations having Aurora kinase inhibiting activity. A patient suffering from, or suspected of suffering from, a cancer may therefore be screened to determine whether he or she forms part of the Ile31 variant sub-population. In addition, it has been found, Rajagopalan et al (Nature. 2004 Mar. 4; 428(6978):77-81), that there were mutations present in CDC4 (also known as Fbw7 or Archipelago) in human colorectal cancers and endometrial cancers (Spruck et al, Cancer Res. 2002 Aug. 15; 62(16):4535-9). Identification of individual carrying a mutation in CDC4 may mean that the patient would be particularly suitable for treatment with a CDK inhibitor. Tumours may preferentially be screened for presence of a CDC4 variant prior to treatment. The screening process will typically involve direct sequencing, oligonucleotide microarray analysis, or a mutant specific antibody.

Tumours with activating mutants of Aurora or up-regulation of Aurora including any of the isoforms thereof, may be particularly sensitive to Aurora inhibitors. Tumours may preferentially be screened for up-regulation of Aurora or for Aurora possessing the Ile31 variant prior to treatment (Ewart-Toland et al., Nat. Genet. 2003 August; 34(4):403-12). Ewart-Toland et al identified a common genetic variant in STK15 (resulting in the amino acid substitution F311) that is preferentially amplified and associated with the degree of aneuploidy in human colon tumors. These results are consistent with an important role for the Ile31 variant of STK15 in human cancer susceptibility. In particular, this polymorphism in Aurora A has been suggested to be a genetic modifier fir developing breast carcinoma (Sun et al., Carcinogenesis, 2004, 25(11), 2225-2230).

The Aurora A gene maps to the chromosome 20q13 region that is frequently amplified in many cancers e.g. breast, bladder, colon, ovarian, pancreatic. Patients with a tumour that has this gene amplification might be particularly sensitive to treatments targeting Aurora kinase inhibition

Methods of identification and analysis of mutations and up-regulation of protein e.g. Aurora isoforms and chromosome 20q13 amplification are known to a person skilled in the art. Screening methods could include, but are not limited to, standard methods such as reverse-transcriptase polymerase chain reaction (RT-PCR) or in-situ hybridisation.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of CDC4 variants, Aurora up-regulation and mutants of Aurora could be applicable in the present case.

Therefore, all of these techniques could also be used to identify tumours particularly suitable for treatment with the combinations of the invention. Tumours with mutants of CDC4 or up-regulation, in particular over-expression, of cyclin E or loss of p21 or p27 may be particularly sensitive to CDK inhibitors as components of the combinations. Tumours may preferentially be screened for up-regulation, in particular over-expression, of cyclin E (Harwell R M, Mull B B, Porter D C, Keyomarsi K.; J Biol. Chem. 2004 Mar. 26; 279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4; 428(6978):77-81).

Patients with mantle cell lymphoma (MCL) could be selected for treatment with a combination of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. 2000 Apr. 1; 95(7):2253-61) proposed that cyclin D1-positivity should be included as one of the standard criteria for MCL, and that innovative therapies for this incurable disease should be explored on the basis of the new criteria. Jones et al (J Mol Diagn. 2004 May; 6(2):84-9) developed a real-time, quantitative, reverse transcription PCR assay for cyclin D1 (CCNDI) expression to aid in the diagnosis of mantle cell lymphoma (MCL). Howe et al (Clin Chem. 2004 January;50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a combination of the invention.

Prior to administration of a combination of the invention, a patient may be screened to determine whether a disease or condition from which the patient is or may be suffering is one which would be susceptible to treatment with a combination having activity against Flt3, C-abl, PDK1. These techniques may also be used for screening for diseases or conditions caused by the up-regulation or mutants of Flt3, C-abl, PDK1, kinases.

These techniques may also be used for screening for diseases or conditions caused by the up-regulation or mutants of VEGFR kinases, include, but are not limited to, standard methods such as reverse-transcriptase polymerase chain reaction (RT-PCR) or in-situ hybridisation.

In addition, mutant forms of, for example, VEGFR2 can be identified by direct sequencing of, for example, tumour biopsies using PCR and methods to sequence PCR products directly as hereinbefore described. The skilled artisan will recognize that all such well-known techniques for detection of the over expression, activation or mutations of the aforementioned proteins could be applicable in the present case.

Abnormal levels of proteins such as VEGFR can be measured using standard enzyme assays, for example, those assays described herein. Activation or overexpression could also be detected in a tissue sample, for example, a tumour tissue, by measuring the tyrosine kinase activity with an assay such as that available from Chemicon International. The tyrosine kinase of interest would be immunoprecipitated from the sample lysate and its activity measured.

Alternative methods for the measurement of the over expression or activation of VEGFR including the isoforms thereof, include the measurement of microvessel density. This can for example be measured using methods described by Orre and Rogers (Int J Cancer 1999 84(2) 101-8). Assay methods also include the use of markers, for example, in the case of VEGFR these include CD31, CD34 and CD105 (Mineo et al. J Clin Pathol. 2004 57(6) 591-7).

Activating mutations of FLT3 are frequently observed in acute myeloid leukaemia, myelodysplastic syndromes (MDS) and some cases with acute lymphoblastic leukemia (ALL). Cancer patients with activating mutants of FLT3 can be screened for presence of the length mutations or internal tandem duplication mutations as an indication of those most sensitive to inhibitors of FLT3.

Patients with tumours harbouring cells expressing the resisance mutants of BCR-abl e.g. T315I can be identified using the methods described herein.

Therefore in addition the methods described herein could be used to diagnose activating mutations of FLT3, mutants of C-Abl e.g. T315I.

### Antifungal Use

In a further aspect, the invention provides the use of the combinations comprising (or consisting essentially of) a compound of formula (I′″) and a compound of the formula (0) and sub-groups thereof as defined herein as antifungal agents.

The combinations of the invention may be used in animal medicine (for example in the treatment of mammals such as humans), or in the treatment of plants (e.g. in agriculture and horticulture), or as general antifungal agents, for example as preservatives and disinfectants.

In one embodiment, the invention provides a combination as defined herein for use in the prophylaxis or treatment of a fungal infection in a mammal such as a human.

Also provided is the use of a combination as defined herein for the manufacture of a medicament for use in the prophylaxis or treatment of a fungal infection in a mammal such as a human.

For example, combination of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The combinations of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of formula (I′″) and a compound of the formula (0) and sub-groups thereof as defined herein together with an auxilliary agent and an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a combination as defined herein.

The invention also provides a method of treating a fungal infection in a plant or seed which comprises treating the plant or seed with an antifungally effective amount of a fungicidal composition containing a combination as defined herein.

Differential screening assays may be used to select for those compounds with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocardiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus arrhizus, Rhizopus oryzae, Absidia corymbifera, Absidia ramosa, or Mucor pusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted. Alternatively, a turbidity assay in liquid cultures can be performed and a protocol outlining an example of this assay can be found in the Examples below.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the combinations as defined herein can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level can be from 0.01 to 10 mg/kg (in divided doses), depending on inter alia the potency of the combination when administered by either the oral or parenteral route. Tablets or capsules of the combination or its constituent compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal combinations can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulation of the combinations of the invention in insecticides, such as for use in management of insects like the fruit fly.

In yet another embodiment, certain of the subject CDK inhibitors can be selected for use in the combinations of the invention on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the combinations of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the combinations of the invention in the control of wood decaying fungi and in the treatment of soil where plants grow, paddy fields for seedlings, or water for perfusion. Also contemplated by the invention is the use of the combinations as defined herein to protect stored grain and other non-plant loci from fungal infestation.

### EXAMPLES

Illustrative examples are set out below and are organized into two separate sets, each numbered for internal consistency and each corresponding to a distinct class of constituent compounds used in the combinations of the invention. In the case of set B, it should be noted that the compounds illustrated therein are a subset of the compounds exemplified in set A. In each set, examples are set out relating to combinations with the optional auxiliiary compounds as described and defined above. The techniques described in these examples are of general applicability and may be used to assess the efficacy of combinations of the compounds of formula (0) with compounds of formula (I′″) with any of the optional auxiliary compounds described herein.

### Set A: Examples Relating to Compounds of the Formula (0)

The invention will now be illustrated, but not limited, by reference to the specific embodiments described in the following examples.

In the examples, the compounds prepared were characterised by liquid chromatography and mass spectroscopy (LC-MS) using the system and operating conditions set out in WO2005/012256 at pages 116 et seq.

The starting materials for each of the Examples are commercially available unless otherwise specified.

Examples 1 to 254 of WO2005/012256 at pages 121 to 222 are incorporated herein by reference, so that examples of the preparation of the following compounds are specifically described herein:
4-Amino-1H-pyrazole-3-carboxylic acid phenylamide
4-Acetylamino-1H-pyrazole-3-carboxylic acid (4-fluoro-phenyl)-amide
4-(2,2,2-Trifluoro-acetylamino)-1H-pyrazole-3-carboxylic acid (4-fluoro-phenyl)-amide
4-[(5-Oxo-pyrrolidine-2-carbonyl)-amino]-1H-pyrazole-3-carboxylic acid (4-fluoro-phenyl)-amide
4-Phenylacetylamino-1H-pyrazole-3-carboxylic acid (4-fluoro-phenyl)-amide
4-(2-1H-Indol-3-yl-acetylamino)-1H-pyrazole-3-carboxylic acid (4-fluoro-phenyl)-amide
4-(2-Benzenesulphonyl-acetylamino)-1H-pyrazole-3-carboxylic acid (4-fluoro-phenyl)-amide
4-[2-(5-Amino-tetrazol-1-yl)-acetylamino]-1H-pyrazole-3-carboxylic acid (4-fluoro-phenyl)-amide
N-[3-(4-Fluoro-phenylcarbamoyl)-1H-pyrazol-4-yl]-6-hydroxy-nicotinamide
4-[3-(4-Chloro-phenyl)-propionylamino]-1H-pyrazole-3-carboxylic acid (4-fluoro-phenyl)-amide
4-(3-4H-[1,2,4]Triazol-3-yl-propionylamino)-1H-pyrazole-3-carboxylic acid (4-fluoro-phenyl)-amide
4-[2-(1-Methyl-1H-indol-3-yl)-acetylamino]-1H-pyrazole-3-carboxylic acid (4-fluoro-phenyl)-amide
4-[(1-Hydroxy-cyclopropanecarbonyl)-amino]-1H-pyrazole-3-carboxylic acid (4-fluoro-phenyl)-amide
1-Acetyl-piperidine-4-carboxylic acid [3-(4-fluoro-phenylcarbamoyl)-1H-pyrazol-4-yl]-amide
4-[3-(4-Methyl-piperazin-1-yl)-propionylamino]-1H-pyrazole-3-carboxylic acid (4-fluoro-phenyl)-amide
4-(2-1H-Imidazol-4-yl-acetylamino)-1H-pyrazole-3-carboxylic acid (4-fluorophenyl)-amide
4-(3-Morpholin-4-yl-propionylamino)-1H-pyrazole-3-carboxylic acid (4-fluorophenyl)-amide
4-(3-Piperidin-1-yl-propionylamino)-1H-pyrazole-3-carboxylic acid (4-fluoro-phenyl)-amide
4-Cyclohexylamino-1H-pyrazole-3-carboxylic acid (4-fluoro-phenyl)-amide
4-Isopropylamino-1H-pyrazole-3-carboxylic acid (4-fluoro-phenyl)-amide
4-(2-Hydroxy-1-methyl-ethylamino)-1H-pyrazole-3-carboxylic acid (4-fluorophenyl)-amide
4-(1-Ethyl-propylamino)-1H-pyrazole-3-carboxylic acid (4-fluoro-phenyl)-amide
4-(3-Chloro-pyrazin-2-ylamino)-1H-pyrazole-3-carboxylic acid (4-fluoro-phenyl)-amide
4-(Pyrazin-2-ylamino)-1H-pyrazole-3-carboxylic acid (4-fluoro-phenyl)-amide
4-(2-Methoxy-benzoylamino)-1H-pyrazole-3-carboxylic acid (4-fluoro-phenyl)-amide
4-Benzoylamino-1H-pyrazole-3-carboxylic acid (4-fluoro-phenyl)-amide
4-(Cyclohexanecarbonyl-amino)-1H-pyrazole-3-carboxylic acid (4-fluoro-phenyl)-amide
4-[(1-Methyl-cyclopropanecarbonyl)-amino]-1H-pyrazole-3-carboxylic acid (4-fluoro-phenyl)-amide
4-(2-Hydroxy-acetylamino)-1H-pyrazole-3-carboxylic acid (4-fluoro-phenyl)-amide
4-(2,2-Dimethyl-propionylamino)-1H-pyrazole-3-carboxylic acid (4-fluoro-phenyl)-amide
4-(3-Hydroxy-propionylamino)-1H-pyrazole-3-carboxylic acid (4-fluoro-phenyl)-amide
4-(2-Fluoro-benzoylamino)-1H-pyrazole-3-carboxylic acid (4-fluoro-phenyl)-amide
4-(3-Fluoro-benzoylamino)-1H-pyrazole-3-carboxylic acid (4-fluoro-phenyl)-amide
4-(3-Methoxy-benzoylamino)-1H-pyrazole-3-carboxylic acid (4-fluoro-phenyl)-amide
4-(4-Nitro-benzoylamino)-1H-pyrazole-3-carboxylic acid (4-fluoro-phenyl)-amide
4-[(3-Methyl-furan-2-carbonyl)-amino]-1H-pyrazole-3-carboxylic acid (4-fluoro-phenyl)-amide
4-[(Furan-2-carbonyl)-amino]-1H-pyrazole-3-carboxylic acid (4-fluoro-phenyl)-amide
4-[(3H-Imidazole-4-carbonyl)-amino]-1H-pyrazole-3-carboxylic acid (4-fluoro-phenyl)-amide
4-(4-Fluoro-benzoylamino)-1H-pyrazole-3-carboxylic acid (4-fluoro-phenyl)-amide
4-(2,6-Difluoro-benzoylamino)-1H-pyrazole-3-carboxylic acid (4-fluoro-phenyl)-amide
4-(3-Nitro-benzoylamino)-1H-pyrazole-3-carboxylic acid (4-fluoro-phenyl)-amide
1H-Indole-3-carboxylic acid [3-(4-fluoro-phenylcarbamoyl)-1H-pyrazol-4-yl]-amide
4-(4-Hydroxymethyl-benzoylamino)-1H-pyrazole-3-carboxylic acid (4-fluoro-phenyl)-amide
4-(3-Methyl-benzoylamino)-1H-pyrazole-3-carboxylic acid (4-fluoro-phenyl)-amide
4-(2-Methyl-benzoylamino)-1H-pyrazole-3-carboxylic acid (4-fluoro-phenyl)-amide
4-(4-Methyl-benzoylamino)-1H-pyrazole-3-carboxylic acid (4-fluoro-phenyl)-amide
4-[(2-Methyl-thiophene-3-carbonyl)-amino]-1H-pyrazole-3-carboxylic acid (4-fluoro-phenyl)-amide
Quinoline-2-carboxylic acid [3-(4-fluoro-phenylcarbamoyl)-1H-pyrazol-4-yl]-amide
4-[(Thiophene-3-carbonyl)-amino]-1H-pyrazole-3-carboxylic acid (4-fluoro-phenyl)-amide
4-(2-fluoro-3-methoxy-benzoylamino)-1H-pyrazole-3-carboxylic acid (4-fluoro-phenyl)-amide
4-[2-(2-Pyrrolidin-1-yl-ethoxy)-benzoylamino]-1H-pyrazole-3-carboxylic acid 4-fluorophenylamide
4-(2,6-Difluoro-benzoylamino)-1H-pyrazole-3-carboxylic acid (1-methyl-piperidin-4-yl)-amide
4-(Cyclohexyl-methyl-amino)-1H-pyrazole-3-carboxylic acid (4-fluoro-phenyl)-amide
4-(Pyridin-2-ylamino)-1H-pyrazole-3-carboxylic acid (4-fluoro-phenyl)-amide
4-[(4-Amino-1-methyl-1H-imidazole-2-carbonyl)-amino]-1H-pyrazole-3-carboxylic acid (4-fluoro-phenyl)-amide
4-{[4-(2,6-Difluoro-benzoylamino)-1H-pyrazole-3-carbonyl]-amino}-cyclohexanecarboxylic acid
4-(2,6-difluoro-benzoylamino)-1H-pyrazole-3-carboxylic acid [5-fluoro-2-(1-methyl-piperidin-4-yloxy)-phenyl]-amide
4-(2,6-Difluoro-benzoylamino)-1H-pyrazole-3-carboxylic acid [5-fluoro-2-(2-pyrrolidin-1-yl-ethoxy)-phenyl]-amide
4-(4-Methyl-piperazin-1-yl)-1H-pyrazole-3-carboxylic acid (4-fluoro-phenyl)-amide
4-Morpholin-4-yl-1H-pyrazole-3-carboxylic acid (4-fluoro-phenyl)-amide
4-(2,4-Dichloro-phenyl)-1H-pyrazole-3-carboxylic acid 4-(4-methyl-piperazin-1-yl)-benzylamide
4-(2,4-Dichloro-phenyl)-1H-pyrazole-3-carboxylic acid 4-methylsulphamoylmethyl-benzylamide
4-Phenyl-1H-pyrazole-3-carboxylic acid amide
4-phenyl-1H-pyrazole-3-carboxylic acid phenylamide
4-Phenyl-1H-pyrazole-3-carboxylic acid 4-(4-methyl-piperazin-1-yl)-benzylamide
4-Phenyl-1H-pyrazole-3-carboxylic acid (6-methoxy-pyridin-3-yl) amide
4-(3-Benzyloxy-phenyl)-1H-pyrazole-3-carboxylic acid 4-(4-methyl-piperazin-1-yl)-benzylamide
4-(3-Hydroxy-phenyl)-1H-pyrazole-3-carboxylic acid 4-(4-methyl-piperazin-1-yl)-benzylamide
4-(5-Methyl-3H-imidazol-4-yl)-1H-pyrazole-3-carboxylic acid (4-fluoro-phenyl)-amide
4-(2,5-Dimethyl-pyrrol-1-yl)-1H-pyrazole-3-carboxylic acid (4-fluoro-phenyl)-amide
4-(3-Hydroxymethyl-phenyl)-1H-pyrazole-3-carboxylic acid phenylamide
4-(2,6-dichloro-benzoylamino)-1H-pyrazole-3-carboxylic acid piperidin-4-ylamide hydrochloride
4-Methanesulfonylamino-1H-pyrazole-3-carboxylic acid (4-fluoro-phenyl)-amide
4-(2,6-Difluoro-benzoylamino)-1H-pyrazole-3-carboxylic acid [1-(2-fluoro-ethyl)-piperidin-4-yl]-amide
4-(2,6-Dichloro-benzoylamino)-1H-pyrazole-3-carboxylic acid (6-chloro-pyridin-3-yl)-amide
4-(2,6-Dichloro-benzoylamino)-1H-pyrazole-3-carboxylic acid (6-amino-pyridin-3-yl)-amide
4-(2,6-Dichloro-benzoylamino)-1H-pyrazole-3-carboxylic acid (6-methoxy-pyridin-3-yl)-amide
4-[3-Chloro-5-(4-methyl-piperazin-1-yl)-benzoylamino]-1H-pyrazole-3-carboxylic acid cyclohexylamide
4-(2,6-Difluoro-benzoylamino)-1H-pyrazole-3-carboxylic acid [1-(2,2-difluoro-ethyl)-piperidin-4-yl]-amide
4-[3-(4-Methyl-piperazin-1-yl)-benzoylamino]-1H-pyrazole-3-carboxylic acid cyclohexylamide
4-(2,6-dichloro-benzoylamino)-1H-pyrazole-3-carboxylic acid piperidin-4-ylamide acetic acid salt
Methanesulphonic acid salt of 4-(2,6-dichloro-benzoylamino)-1H-pyrazole-3-carboxylic acid piperidin-4-ylamide

The compounds set out in the Tables below:

| Structure | LCMS |
|---|---|
| 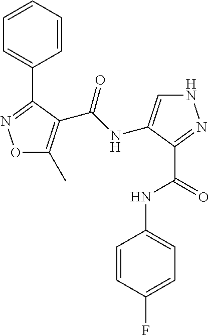 | Rₜ 3.20 min [M + H]⁺ 406.07 |
| 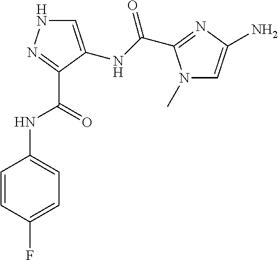 | Rₜ 2.35 min m/z 343.72 |
| 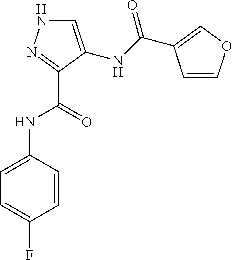 | Rₜ 3.51 min m/z 314.62 |
| 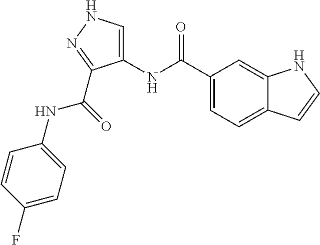 | Rₜ 3.79 min m/z 363.67 |
| 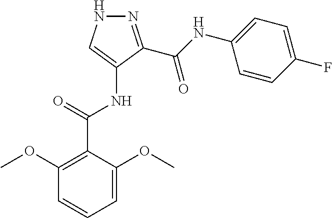 | Rₜ 3.68 min m/z 384.69 |
| 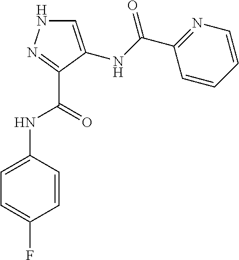 | Rₜ 3.61 min m/z 326.10 |
| 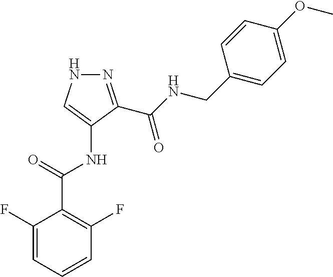 | Rₜ 3.51 min m/z 387.11 |
| 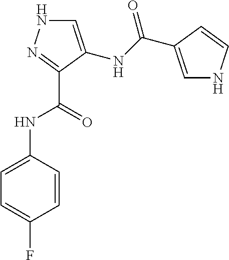 | Rₜ 3.11 min m/z 313.65 |
| 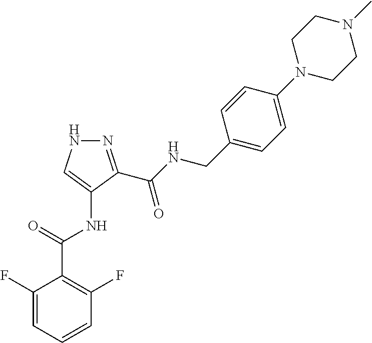 | Rₜ 2.20 min m/z 455.19 |
| 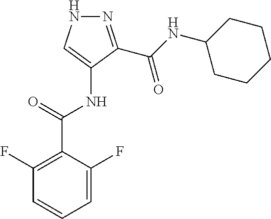 | Rₜ 3.95 min m/z 349.09 |
| 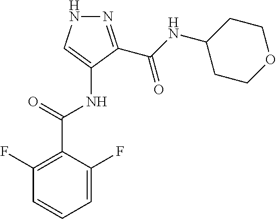 | Rₜ 2.39 min m/z 351.07 |
| 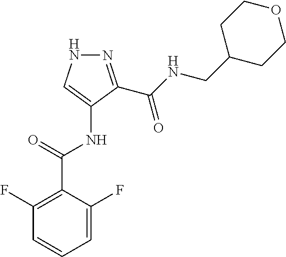 | Rₜ 2.83 min m/z 365.13 |
| 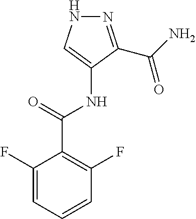 | Rₜ 2.10 min m/z 266.97 |
| 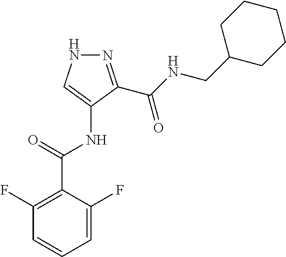 | Rₜ 3.22 min m/z 363.10 |
| 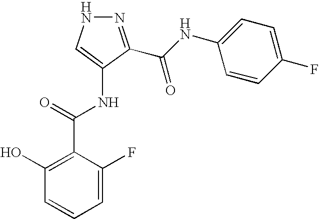 | Rₜ 4.48 min m/z 358.96 |
| 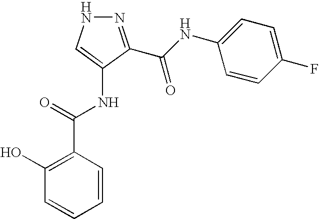 | Rₜ 3.93 min m/z 340.96 |
| 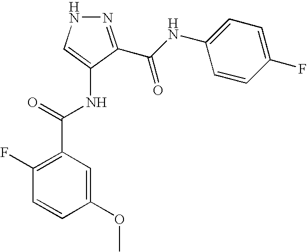 | Rₜ 4.11 min m/z 373.01 |
| 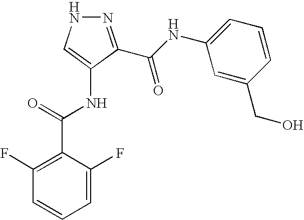 | Rₜ 2.56 min mlz 373.05 |
| 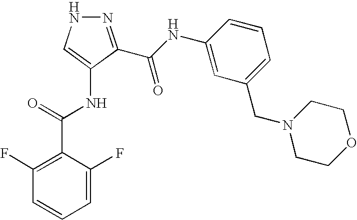 | Rₜ 1.99 min m/z 442.09 |
| 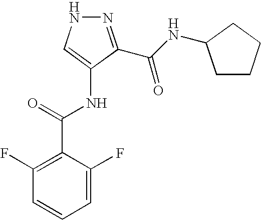 | Rₜ 3.65 min m/z 335.03 |
| 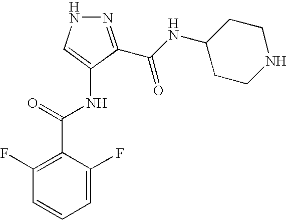 | Rₜ 1.57 min m/z 350.10 |
| 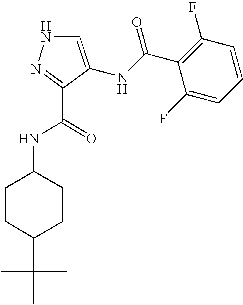 | Rₜ 5.05 min m/z 405.14 |
| 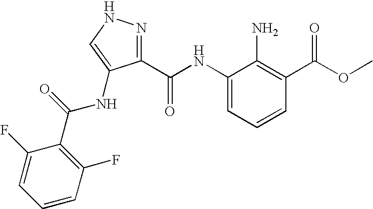 | Rₜ 2.87 min m/z 416.07 |
| 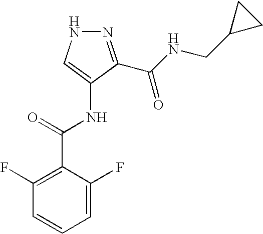 | Rₜ 3.41 min m/z 321.03 |
| 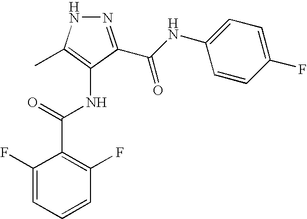 | Rₜ 3.42 min m/z 375.05 |
| 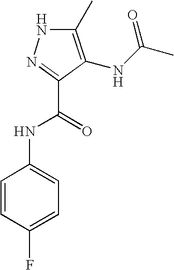 | Rₜ 2.37 min m/z 277.04 |
| 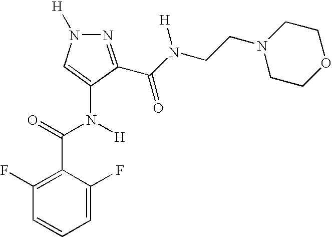 | [M + H]⁺ 380 Rₜ 1.42 |
| 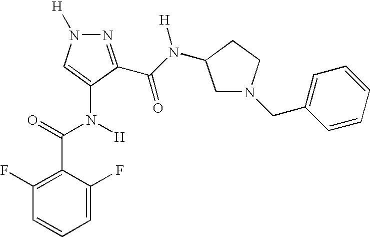 | [M + H]⁺ 426 Rₜ 1.93 |
| 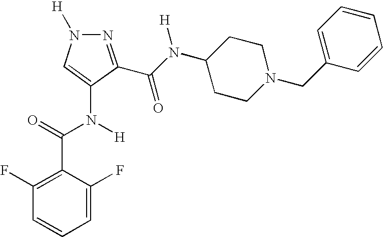 | [M + H]⁺ 440 Rₜ 1.87 |
| 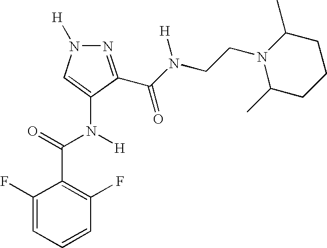 | [M + H]⁺ 406 Rₜ 2.78 |
| 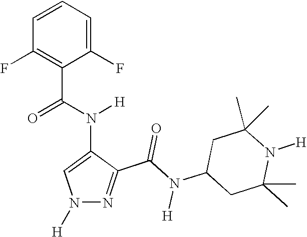 | [M + H]⁺ 406 Rₜ 2.55 |
| 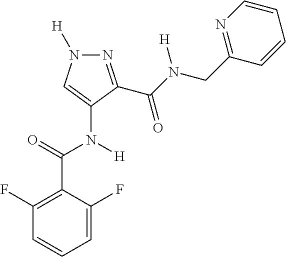 | [M + H]⁺ 358 Rₜ 1.98 |
| 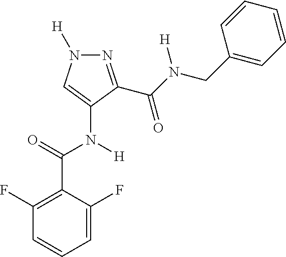 | [M + H]⁺ 357 Rₜ 3.37 |
| 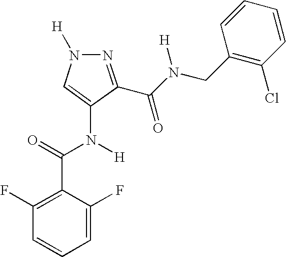 | [M + H]⁺ 391 Rₜ 3.16 |
| 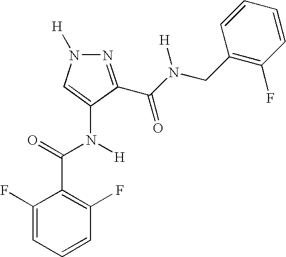 | [M + H]⁺ 375 Rₜ 3.02 |
| 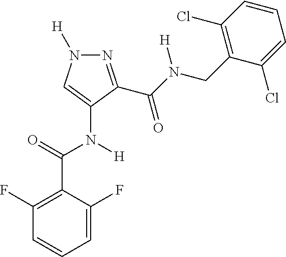 | [M + H]⁺ 425 Rₜ 3.27 |
| 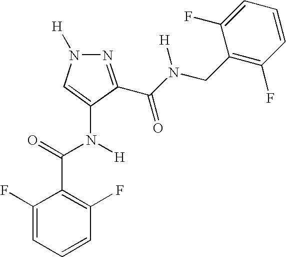 | [M + H]⁺ 393 Rₜ 3.01 |
| 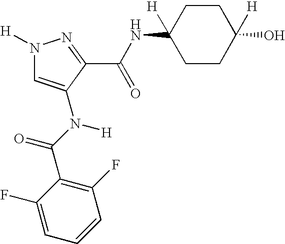 | [M + H]⁺ 365 Rₜ 2.22 |
| 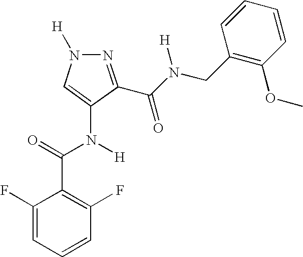 | [M + H]⁺ 387 Rₜ 3.05 |
| 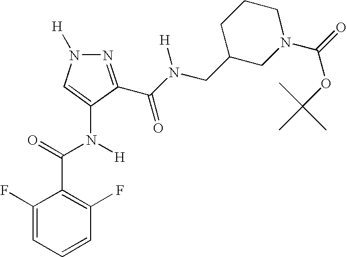 | [M + H]⁺ 464 Rₜ 3.17 |
| 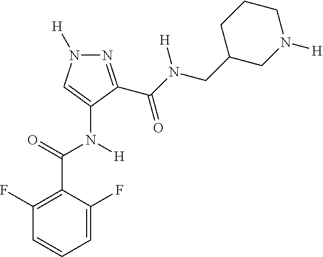 | [M + H]⁺ 364 Rₜ 1.76 |
| 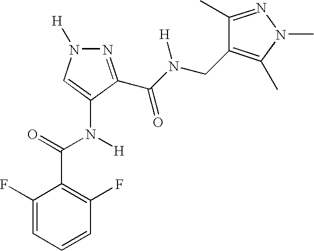 | [M + H]⁺ 389 Rₜ 2.36 |
| 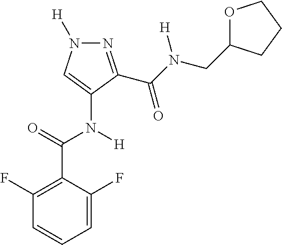 | [M + H]⁺ 351 Rₜ 2.55 |
| 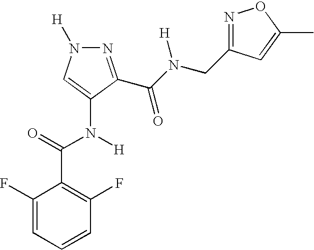 | [M + H]⁺ 362 Rₜ 2.63 |
| 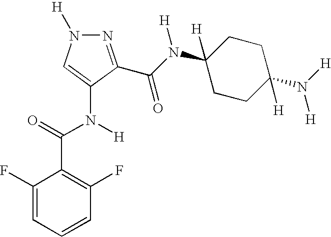 | [M + H]⁺ 364 Rₜ 1.75 |
| 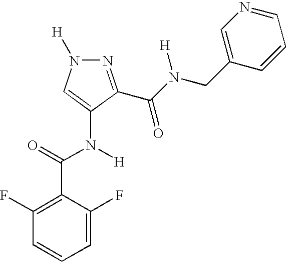 | [M + H]⁺ 358 Rₜ 3.2 |
| 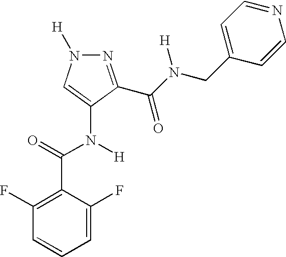 | [M + H]⁺ 358 Rₜ 1.77 |
| 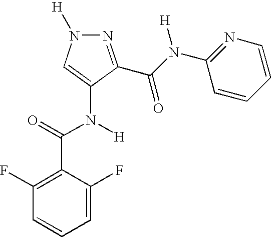 | [M + H]⁺ 344 Rₜ 2.71 |
| 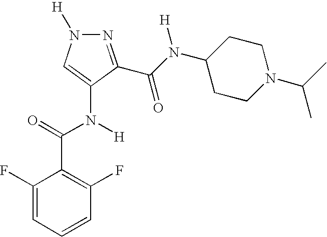 | [M + H]⁺ 392 Rₜ 2.57 |
| 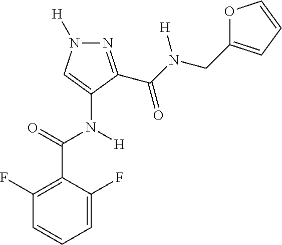 | [M + H]⁺ 347 Rₜ 2.8 |
| 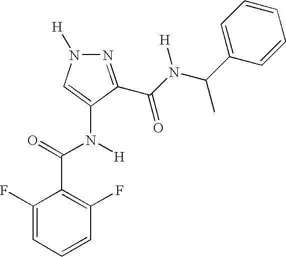 | [M + H]⁺ 371 Rₜ 3.1 |
| 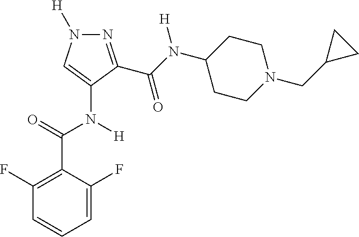 | [M + H]⁺ 404 Rₜ 2.7 |
| 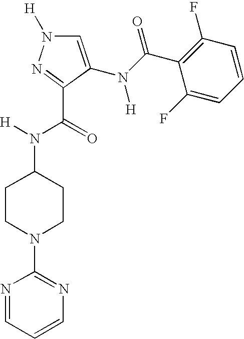 | [M + H]⁺ 428 Rₜ 2.63 |
| 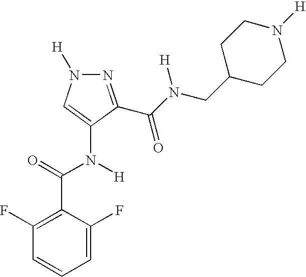 | [M + H]⁺ 364 Rₜ 1.75 |
| 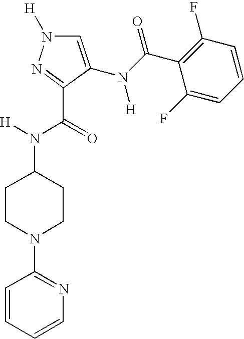 | [M + H]⁺ 427 Rₜ 2.71 |
| 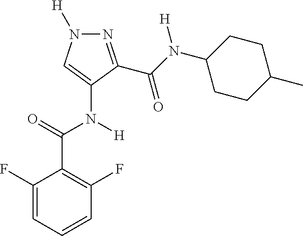 | [M + H]⁺ 363 Rₜ 3.34 |
| 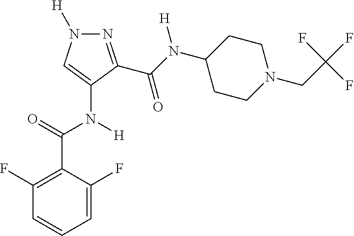 | [M + H]⁺ 432 Rₜ 2.63 |
| 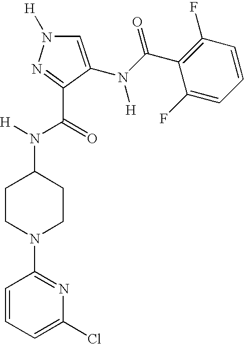 | [M + H]⁺ 461 Rₜ 3.3 |
| 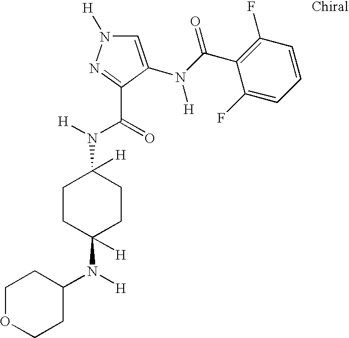 | [M + H]⁺ 448 Rₜ 1.87 |
| 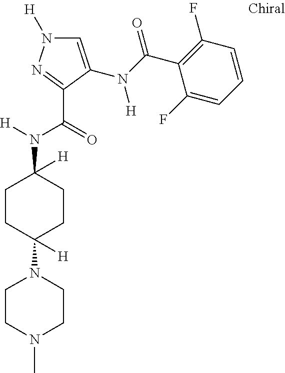 | [M + H]⁺ 447 Rₜ 1.65 |
| 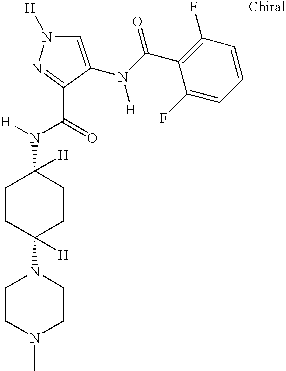 | [M + H]⁺ 447 Rₜ 1.72 |
| 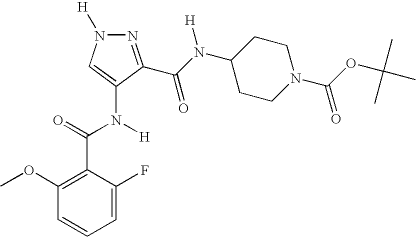 | [M + H]⁺ 462 Rₜ 2.97 |
| 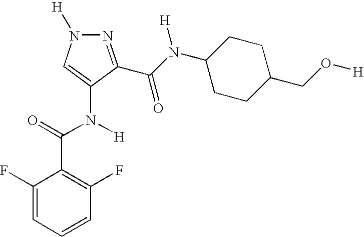 | [M + H]⁺ 379 Rₜ 2.45 |
| 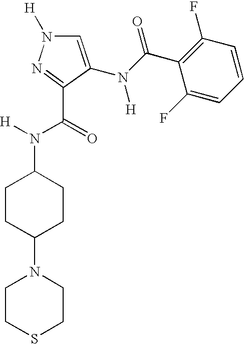 | [M + H]⁺ 450 Rₜ 1.97 |
| 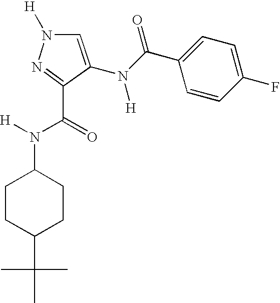 | [M + H]⁺ 387 Rₜ 3.83 |
| 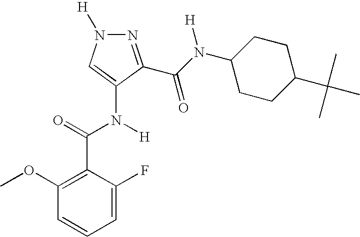 | [M + H]⁺ 417 Rₜ 3.65 |
| 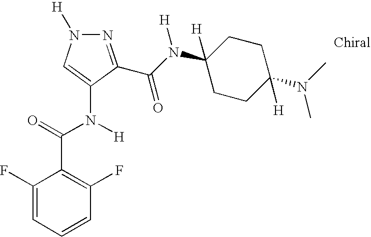 | [M + H]⁺ 392 Rₜ 1.85 |
| 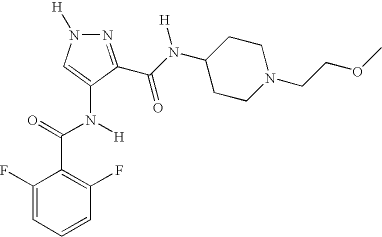 | [M + H]⁺ 408 Rₜ 1.82 |
| 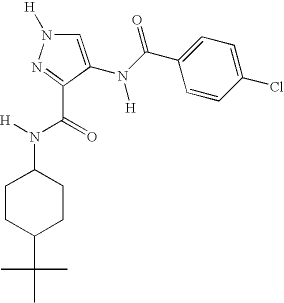 | [M + H]⁺ 403 Rₜ 4.02 |
| 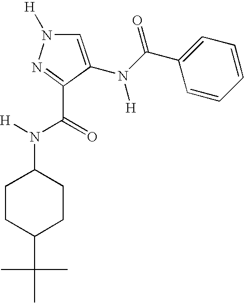 | [M + H]⁺ 369 Rₜ 3.78 |
| 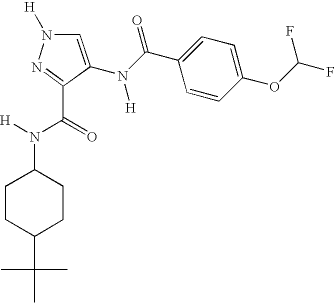 | [M + H]⁺ 435 Rₜ 3.83 |
| 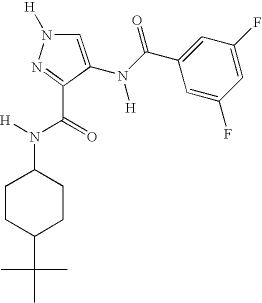 | [M + H]⁺ 405 Rₜ 3.96 |
| 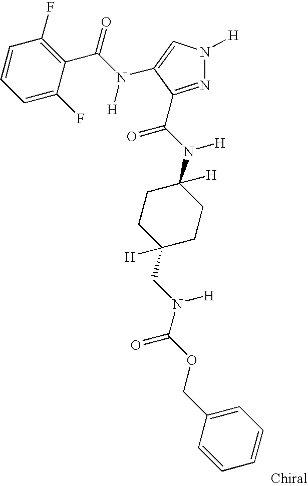 | [M + H]⁺ 512 Rₜ 3.1 |
| 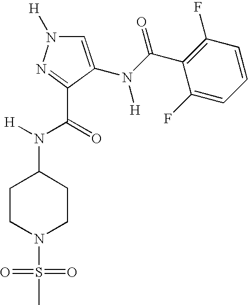 | [M + H]⁺ 428 Rₜ 2.45 |
| 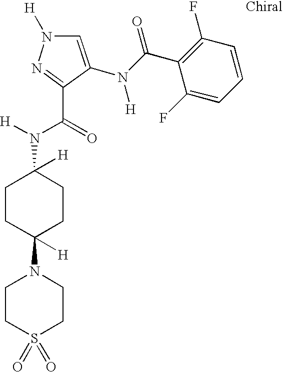 | [M + H]⁺ 482 Rₜ 1.96 |
| 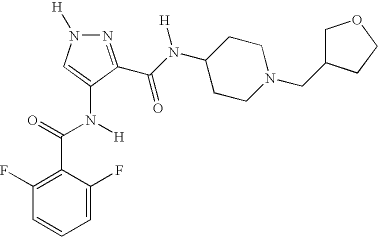 | [M + H]⁺ 434 Rₜ 2.3 |
| 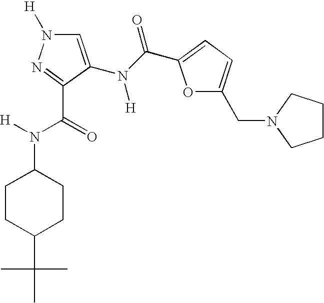 | [M + H]⁺ 442 Rₜ 2.39 |
| 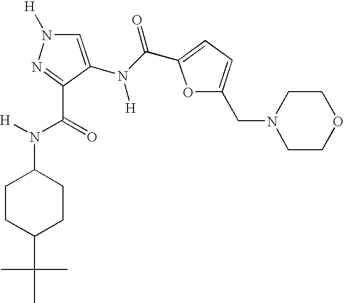 | [M + H]⁺ 458 Rₜ 2.26 |
| 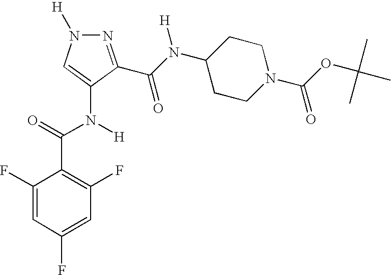 | [M + H]⁺ 468 Rₜ 3.07 |
| 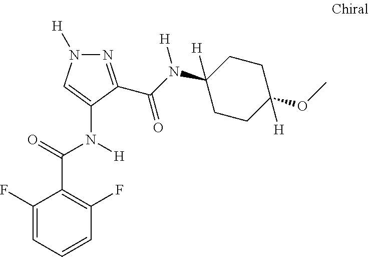 | [M + H]⁺ 379 Rₜ 2.6 |
| 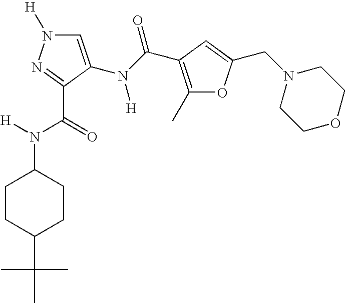 | [M + H]⁺ 472 Rₜ 2.40 |
| 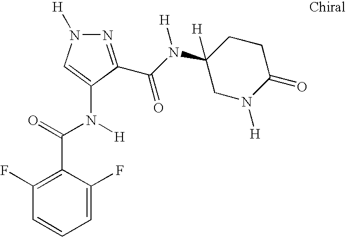 | [M + H]⁺ 364 Rₜ 2.1 |
| 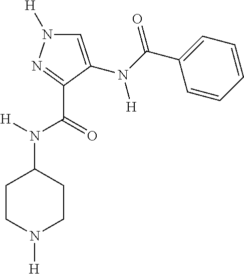 | [M + H]⁺ 314 Rₜ 1.78 |
| 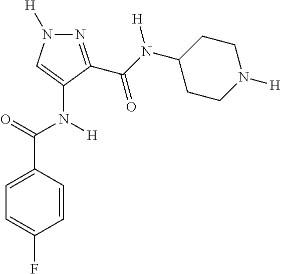 | [M + H]⁺ 332 Rₜ 1.89 |
| 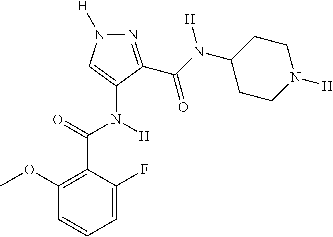 | [M + H]⁺ 362 Rₜ 1.78 |
| 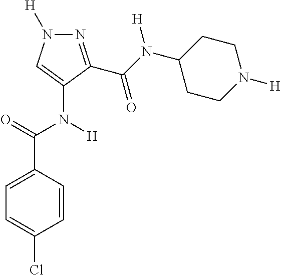 | [M + H]⁺ 348 Rₜ 2.01 |
| 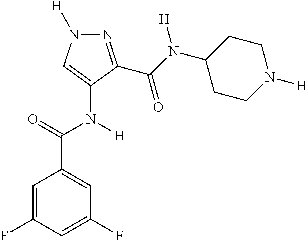 | [M + H]⁺ 350 Rₜ 1.97 |
| 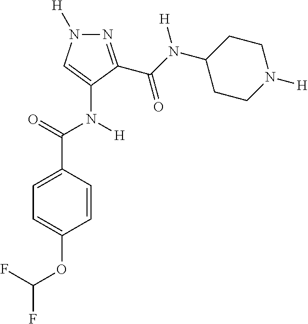 | [M + H]⁺ 380 Rₜ 2.01 |
| 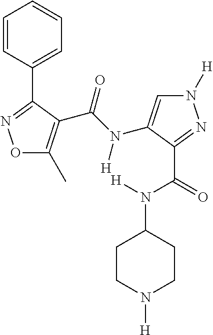 | [M + H]⁺ 395 Rₜ 1.94 |
| 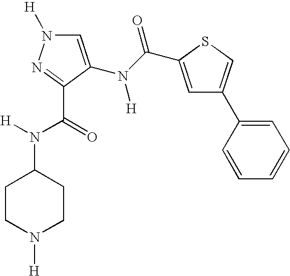 | [M + H]⁺ 396 Rₜ 2.11 |
| 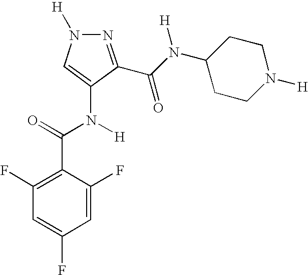 | [M + H]⁺ 368 Rₜ 1.76 |
| 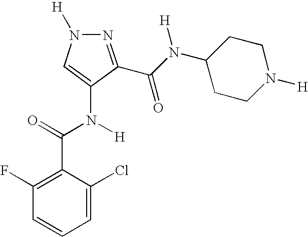 | [M + H]⁺ 366 Rₜ 1.78 |
| 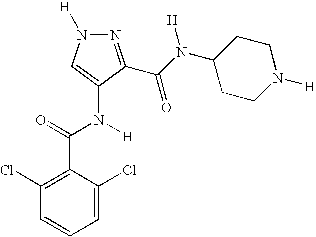 | [M + H]⁺ 383 Rₜ 1.87 |
| 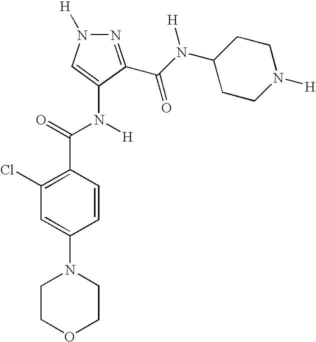 | [M + H]⁺ 433 Rₜ 1.89 |
| 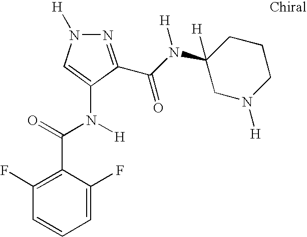 | [M + H]⁺ 350 Rₜ 1.76 |
| 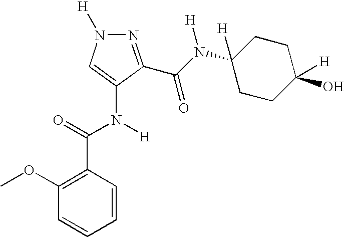 | [M + H]⁺ 359 Rₜ 2.29 |
| 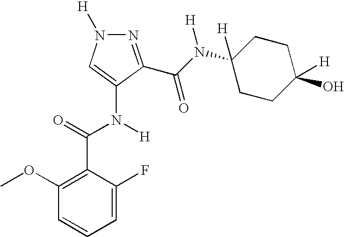 | [M + H]⁺ 377 Rₜ 2.22 |
| 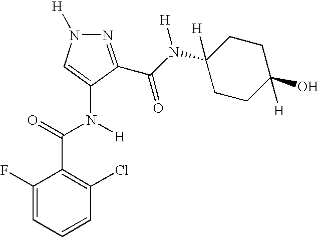 | [M + H]⁺ 381 Rₜ 2.34 |
| 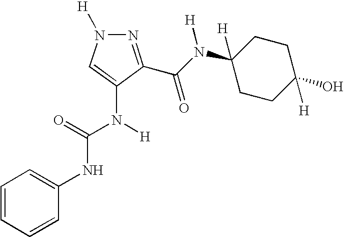 | [M + H]⁺ 344 Rₜ 2.28 |
| 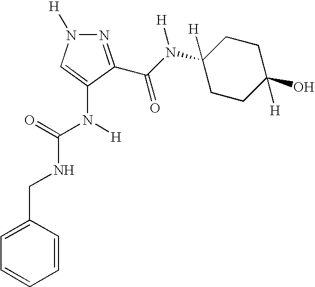 | [M + H]⁺ 358 Rₜ 2.22 |
| 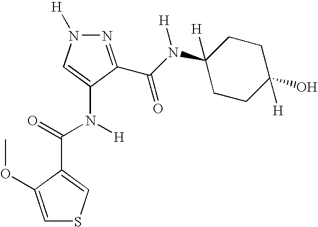 | [M + H]⁺ 365 Rₜ 2.21 |
| 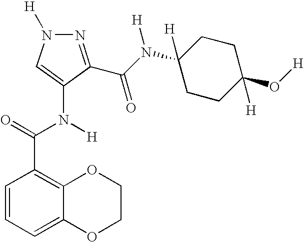 | [M + H]⁺ 387 Rₜ 2.29 |
| 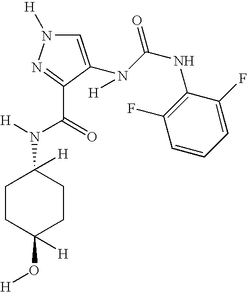 | [M + H]⁺ 380 Rₜ 2.17 |
| 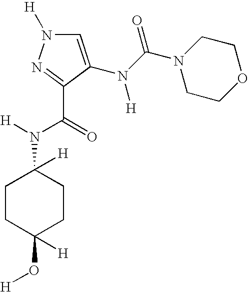 | [M + H]⁺ 338 Rₜ 1.68 |
| 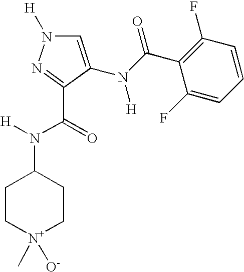 | [M + H]⁺ 380 Rₜ 1.83 |
| 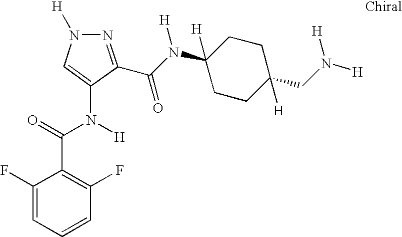 | [M + H]⁺ 378 Rₜ 1.78 |
| 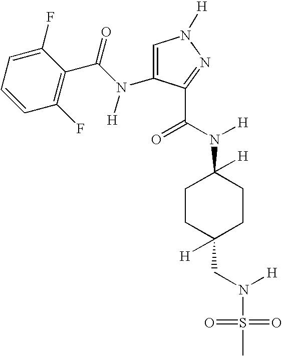 | [M + H]⁺ 456 Rₜ 2.54 |
| 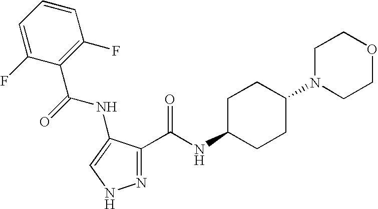 | [M + H]⁺ 434 Rₜ 1.97 |
| 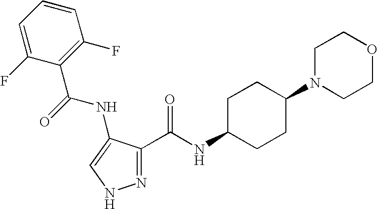 | [M + H]⁺ 434 Rₜ 2.03 |
| 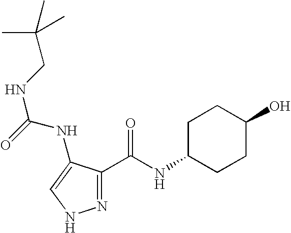 | [M + H]⁺ 338 Rₜ 2.28 |
| 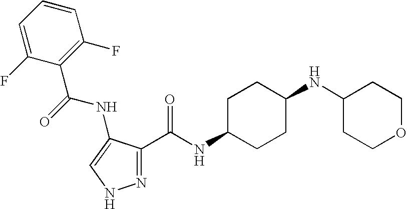 | [M + H]⁺ 448 Rₜ 1.97 |
| 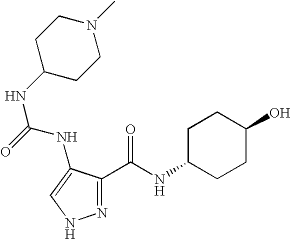 | [M + H]⁺ 365 Rₜ 0.34 |
| 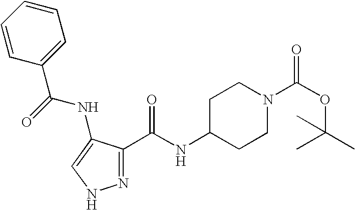 | [M + H]⁺ 414.13 Rₜ 3.05 |
| 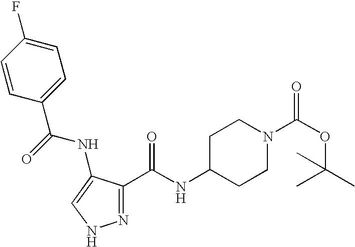 | [M + H]⁺ 432.12 Rₜ 3.12 |
| 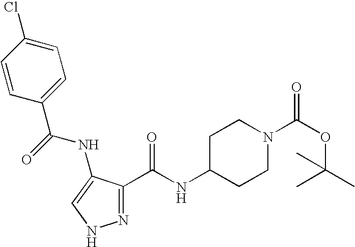 | [M + H]⁺ 448.06 Rₜ 3.33 |
| 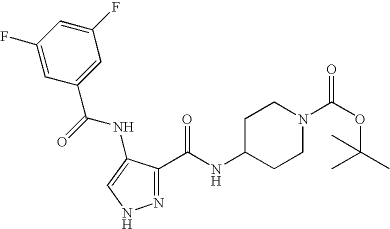 | [M + H]⁺ 450.08 Rₜ 3.29 |
| 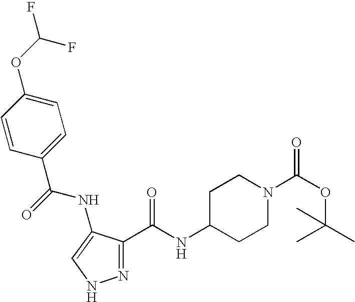 | [M + H]⁺ 480.05 Rₜ 3.18 |
| 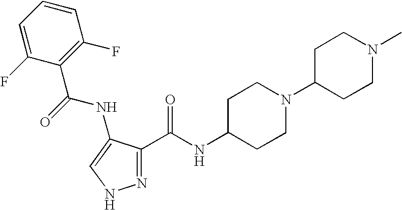 | [M + H]⁺ 447 Rₜ 2.01 |
| 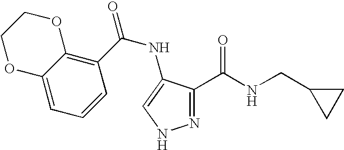 | [M + H]⁺ 343.05 Rₜ 3.38 (polar method) |
| 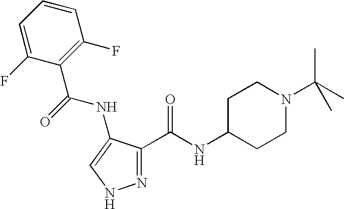 | [M + H]⁺ 406 Rₜ 1.85 |
| 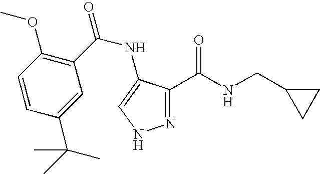 | [M + H]⁺ 371.09 Rₜ 3.27 (polar method) |
| 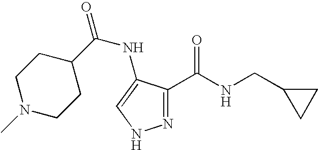 | [M + H]⁺ 306.06 Rₜ 1.53 |
| 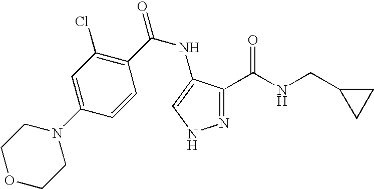 | [M + H]⁺ 403.98 Rₜ 2.78 |
| 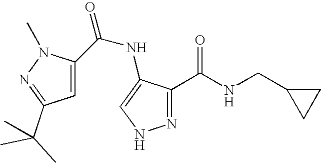 | [M + H]⁺ 345.05 Rₜ 3.03 |
| 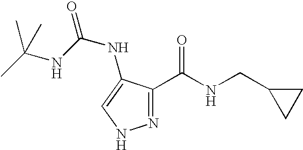 | [M + H]⁺ 280.05 Rₜ 3.75 (basic method) |
| 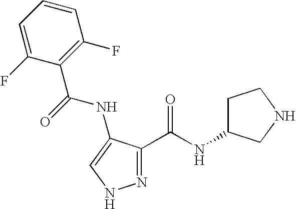 | [M + H]⁺ 336 Rₜ 1.67 |
| 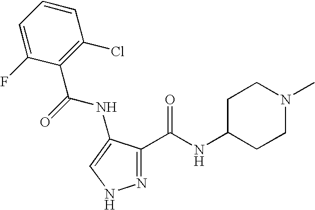 | [M + H]⁺ 380.05 Rₜ 1.78 |
| 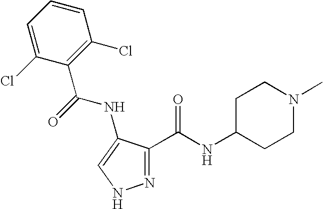 | [M + H]⁺ 396.02 Rₜ 1.86 |
| 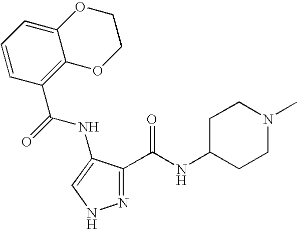 | [M + H]⁺ 386.10 Rₜ 1.88 |
| 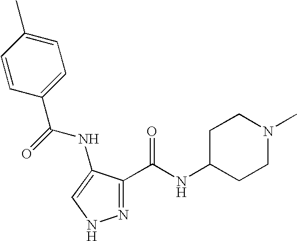 | [M + H]⁺ 342.10 Rₜ 1.95 |
| 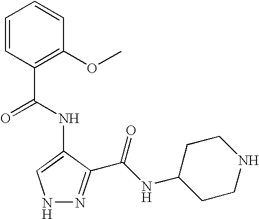 | [M + H]⁺ = 344 Rₜ = 1.87 |
| 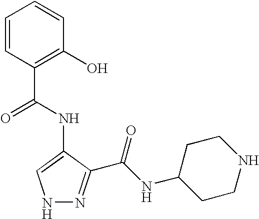 | [M + H]⁺ = 330 Rₜ = 1.80 |
| 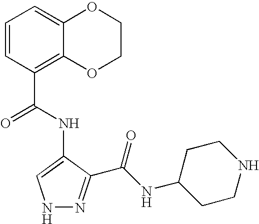 | [M + H]⁺ = 372 Rₜ = 1.87 |
| 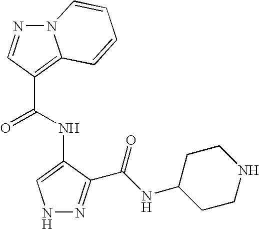 | [M + H]⁺ = 354 Rₜ = 1.77 |
| 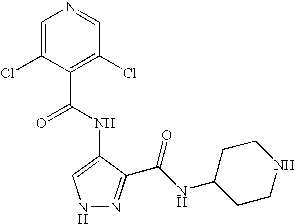 | [M + H]⁺ = 383/385 Rₜ = 1.72 |
| 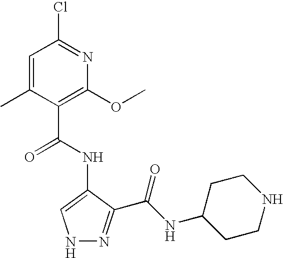 | [M + H]⁺ = 393/395 Rₜ = 1.86 |
| 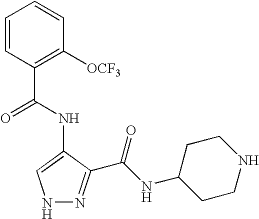 | [M + H]⁺ = 398 Rₜ = 1.94 |
| 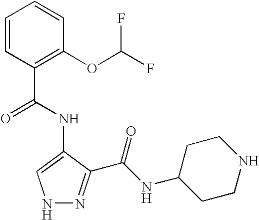 | [M + H]⁺ = 330 Rₜ = 1.80 |
| 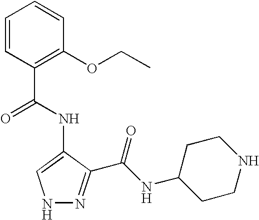 | [M + H]⁺ = 358 Rₜ = 1.89 |
| 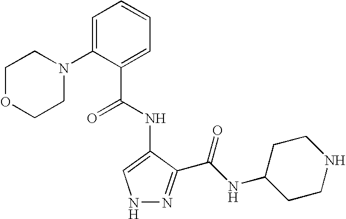 | [M + H]⁺ = 399 Rₜ = 1.88 |
| 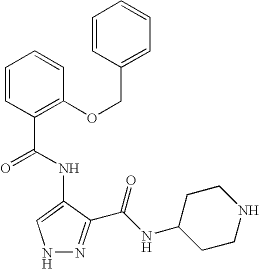 | [M + H]⁺ = 420 Rₜ = 2.13 |
| 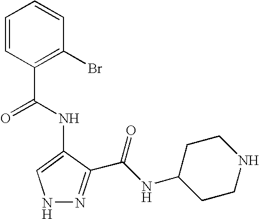 | [M + H]⁺ = 392/394 Rₜ = 1.84 |
| 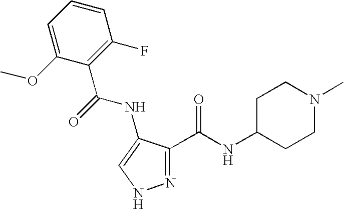 | [M + H]⁺ 376.14 Rₜ 1.78 |
| 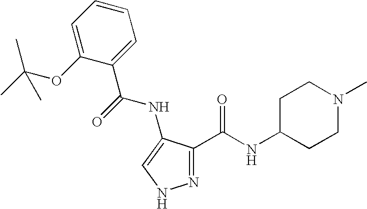 | [M + H]⁺ 400.17 Rₜ 2.08 |
| 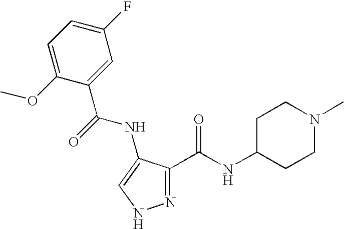 | [M + H]⁺ 376.15 Rₜ 1.92 |
| 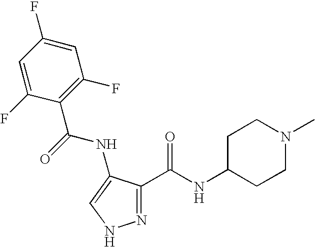 | [M + H]⁺ 382.12 Rₜ 1.77 |
| 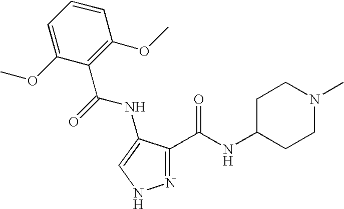 | [M + H]⁺ 388.18 Rₜ 1.73 |
| 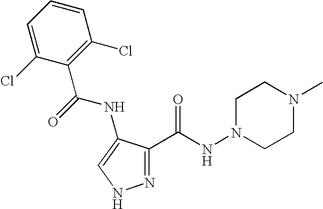 | [M = H]⁺ = 397/399 Rₜ = 1.83 |
| 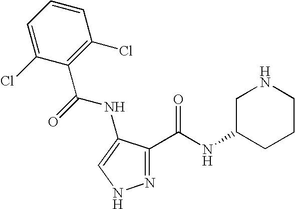 | [M + H]⁺ 382.02 Rₜ 1.82 |
| 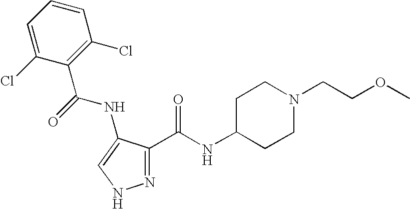 | [M + H]⁺ 440.22 Rₜ 1.92 |
| 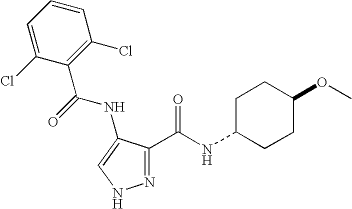 | [M + H]⁺ 411.20 Rₜ 2.97 |
| 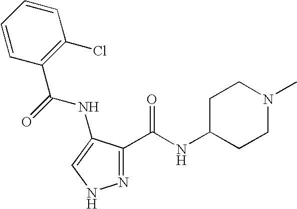 | [M + H]⁺ 362.11 Rₜ 1.91 |
| 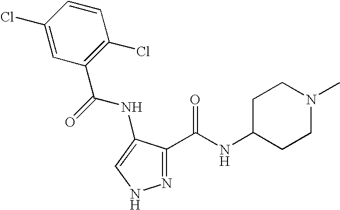 | [M + H]⁺ 396.08 Rₜ 2.06 |
| 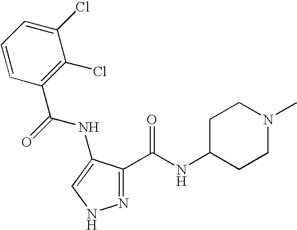 | [M + H]⁺ 396.06 Rₜ 2.04 |
| 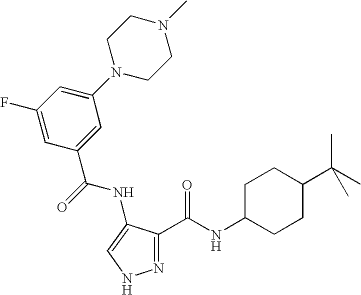 | [M + H]⁺ 485 Rₜ 2.59 |
| 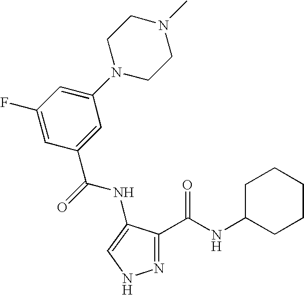 | [M + H]⁺ 429 Rₜ 2.25 |
| 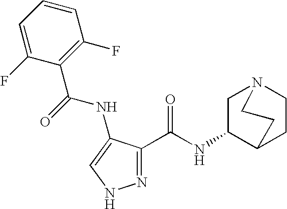 | [M + H]⁺ = 376 Rₜ = 1.85 |
| 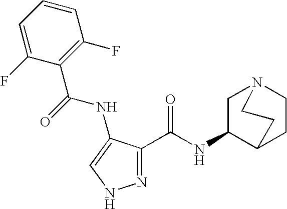 | [M + H]⁺ = 376 Rₜ = 1.87 |
| 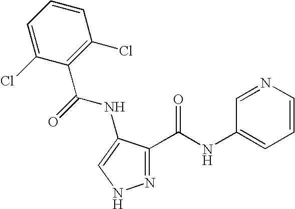 | [M + H]⁺ = 376/378 Rₜ = 2.23 |
| 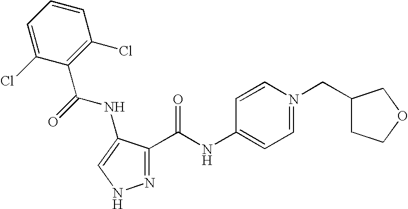 | [M + H]⁺ = 466/468 Rₜ = 1.98 |
| 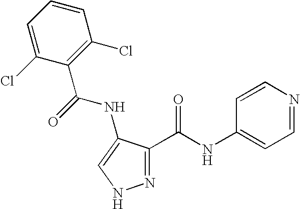 | [M + H]⁺ = 376/378 Rₜ = 2.09 |
| 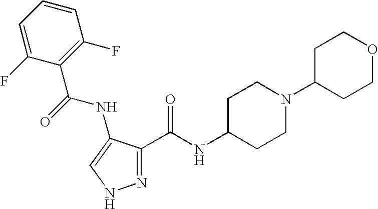 | [M + H]⁺ = 434 Rₜ = 1.82 |
| 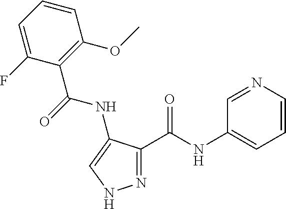 | [M + H]⁺ = 356 Rₜ = 2.11 |
| 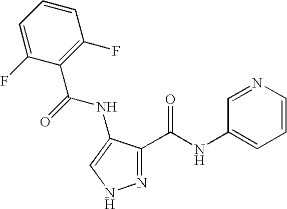 | [M + H]⁺ = 344 Rₜ = 2.09 |

### Example 1

### General Procedure A: Preparation of Amide from Amino-Pyrazole

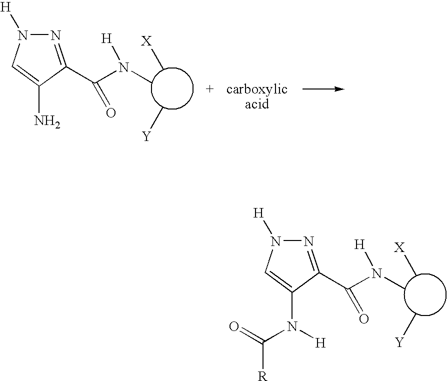

To a stirred solution of the appropriate 4-amino-1H-pyrazole-3-carboxylic acid amide (0.23 mmol), EDAC (52 mg; 0.27 mmol) and HOBt (37 mg; 0.27 mmol) in 5 ml of N,N-dimethylformamide was added the corresponding carboxylic acid (0.25 mmol), and the mixture was then left at room temperature overnight. The reaction mixture was evaporated and the residue purified by preparative LC/MS, to give the product.

### General Procedure B: Deprotection of Piperidine Ring Nitrogen by Removal of tert-Butoxycarbonyl Group

A product of Procedure B containing a piperidine group bearing an N-tert-butoxycarbonyl (t-Boc) protecting group (40 mg) was treated with saturated ethyl acetate/HCl, and stirred at room temperature for 1 hour. A solid precipitated out of the reaction mixture, which was filtered off, washed with ether, and then dried to give 25 mg product (LC/MS: [M+H]⁺ 364).

| | | | |
|---|---|---|---|
| Example 1 | 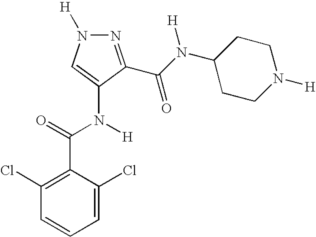 | Procedure A followed by Procedure B | [M + H]⁺ 383 Rₜ 1.87 |

### Example 2

### Preparation of 4-(2,6-dichloro-benzoylamino)-1H-pyrazole-3-carboxylic acid piperidin-4-ylamide hydrochloride

### 2A. 4-(2,6-dichloro-benzoylamino)-1H-pyrazole-3-carboxylic acid

2,6-dichlorobenzoyl chloride (8.2 g; 39.05 mmol) was added cautiously to a solution of 4-amino-1H-pyrazole-3-carboxylic acid methyl ester (prepared in a manner analogous to 165B) (5 g; 35.5 mmol) and triethylamine (5.95 ml; 42.6 mmol) in dioxan (50 ml) then stirred at room temperature for 5 hours. The reaction mixture was filtered and the filtrate treated with methanol (50 ml) and 2M sodium hydroxide solution (100 ml), heated at 50° C. for 4 hours, and then evaporated. 100 ml of water was added to the residue then acidified with concentrated hydrochloric acid. The solid was collected by filtration, washed with water (100 ml) and sucked dry to give 10.05 g of 4-(2,6-dichloro-benzoylamino)-1H-pyrazole-3-carboxylic acid as a pale violet solid.

### 2B. 4-{[4-(2,6-dichloro-benzoylamino)-1H-pyrazole-3-carbonyl]-amino}-piperidine-1-carboxylic acid tert-butyl ester

A mixture of 4-(2,6-dichloro-benzoylamino)-1H-pyrazole-3-carboxylic acid (6.5 g, 21.6 mmol), 4-amino-1-BOC-piperidine (4.76 g, 23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken up in 5% MeOH-DCM (˜30 ml). The insoluble material was collected by filtration and, washed with DCM and dried in vacuo to give 4-{[4-(2,6-dichloro-benzoylamino)-1H-pyrazole-3-carbonyl]-amino}-piperidine-1-carboxylic acid tert-butyl ester (5.38 g) as a white solid. The filtrate was reduced in vacuo and the residue purified by column chromatography using gradient elution 1:2 EtOAc/hexane to EtOAc to give further 4-{[4-(2,6-dichloro-benzoylamino)-1H-pyrazole-3-carbonyl]-amino}-piperidine-1-carboxylic acid tert-butyl ester (2.54 g) as a white solid.

### 2C. 4-(2,6-dichloro-benzoylamino)-1H-pyrazole-3-carboxylic acid Piperidin-4-ylamide

A solution of 4-{[4-(2,6-dichloro-benzoylamino)-1H-pyrazole-3-carbonyl]-amino}-piperidine-1-carboxylic acid tert-butyl ester (7.9 g) in MeOH (50 mL) and EtOAc (50 ml) was treated with sat. HCl-EtOAc (40 mL) then stirred at r.t. overnight. The product did not crystallise due to the presence of methanol, and therefore the reaction mixture was evaporated and the residue triturated with EtOAc. The resulting off white solid was collected by filtration, washed with EtOAc and sucked dry on the sinter to give 6.3 g of 4-(2,6-dichloro-benzoylamino)-1H-pyrazole-3-carboxylic acid piperidin-4-ylamide as the hydrochloride salt. (LC/MS: Rₜ 5.89, [M+H]⁺ 382/384).

### Example 3

### Preparation of 4-(2,6-dichloro-benzoylamino)-1H-pyrazole-3-carboxylic acid piperidin-4-ylamide acetic acid salt

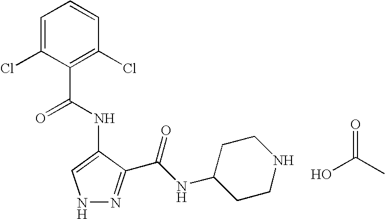

To a solution of 4-(2,6-dichloro-benzoylamino)-1H-pyrazole-3-carboxylic acid piperidin-4-ylamide hydrochloride salt (Example 2C) 20.6 g, 50 mmol) in water (500 ml) stirring at ambient temperature was added sodium bicarbonate (4.5 g, 53.5 mmol). The mixture was stirred for 1 hour and the solid formed collected by filtration and dried in vacuo azeotroping with toluene (×3) to give the corresponding free base of 4-(2,6-dichloro-benzoylamino)-1H-pyrazole-3-carboxylic acid piperidin-4-ylamide.

¹H NMR (400 MHz, DMSO-d₆) δ 10.20 (s, 1H), 8.30 (s, 1H), 8.25 (d, 1H), 7.60-7.50 (m, 3H), 3.70 (m, 1H), 3.00 (d, 2H), 2.50 (m, 2H), 1.70 (d, 2H), 1.50 (m, 2H).

To a stirred suspension of 4-(2,6-dichloro-benzoylamino)-1H-pyrazole-3-carboxylic acid piperidin-4-ylamide (10.0 g, 26.2 mmol) in methanol (150 ml) was added glacial acetic acid (15 ml, 262 mmol) at ambient temperature. After 1 h, a clear solution was obtained which was reduced in vacuo azeotroping with toluene (×2). The residue was then triturated with acetonitrile (2×100 ml) and the solid dried in vacuo to give 4-(2,6-dichloro-benzoylamino)-1H-pyrazole-3-carboxylic acid piperidin-4-ylamide acetic acid salt (10.3 g) as a white solid.

¹H NMR (400 MHz, DMSO-d₆) δ 10.20 (s, 1H), 8.40 (d, 1H), 8.35 (s, 1H), 7.60-7.50 (m, 3H), 3.85 (m, 1H), 3.00 (d, 2H), 2.60 (t, 2H), 1.85 (s, 3H), 1.70 (d, 2H), 1.55 (m, 2H)

### Example 4

### Synthesis of the methanesulphonic acid salt of 4-(2,6-dichloro-benzoylamino)-1H-pyrazole-3-carboxylic acid piperidin-4-ylamide

The methane sulphonic acid salt of 4-(2,6-dichloro-benzoylamino)-1H-pyrazole-3-carboxylic acid piperidin-4-ylamide may be prepared by the synthetic route shown in the Scheme below.

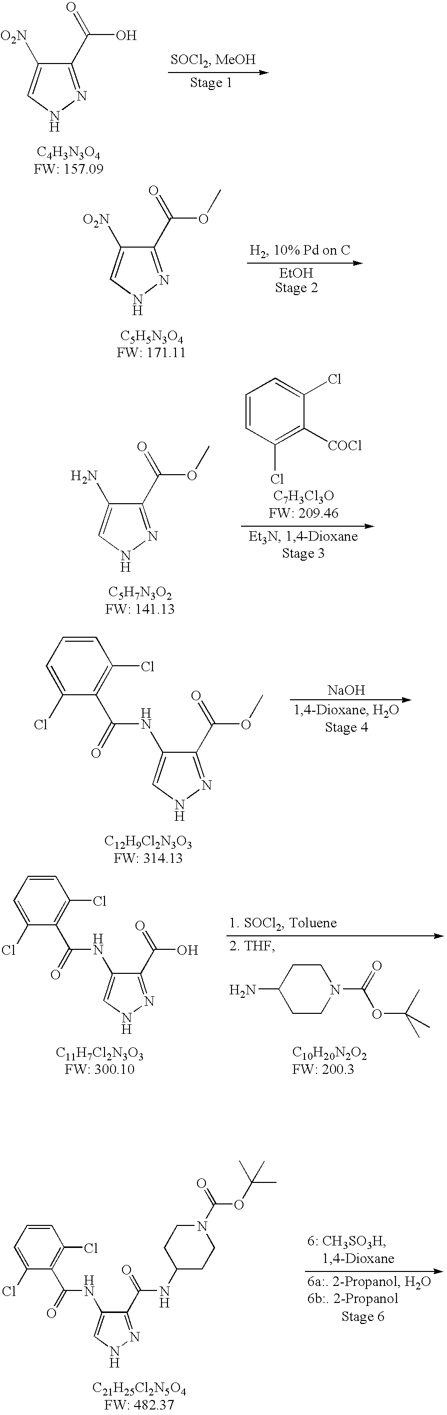

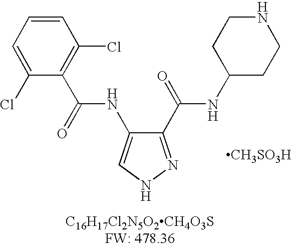

### Stage 1: Preparation of 4-nitro-1H-Pyrazole-3-carboxylic acid methyl ester

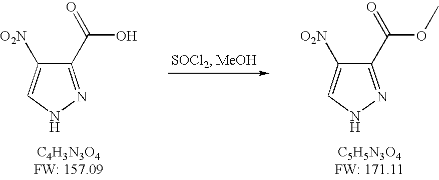

A 20 L reaction vessel equipped with a digital thermometer and stirrer was charged with 4-nitro-1H-pyrazole-3-carboxylic acid (1.117 Kg, 7.11 mol, 1 wt) and methanol (8.950 L, 8 vol). The reaction mixture was stirred under nitrogen, cooled to 0 to 5° C., thionyl chloride (0.581 L, 8.0 mol, 0.52 vol) added over 180 minutes and the resultant mixture allowed to warm to and stir at 18 to 22° C. overnight, after which time ¹H NMR analysis (d₆-DMSO) indicated reaction completion. The reaction mixture was concentrated under reduced pressure at 40 to 45° C., the residue treated with toluene and re-concentrated (3×2.250 L, 3×2 vol) under reduced pressure at 40 to 45° C. to give 4-nitro-1H-pyrazole-3-carboxylic acid methyl ester as an off-white solid (1.210 Kg, 99.5%).

### Stage 2: Preparation of 4-amino-1H-pyrazole-3-carboxylic acid methyl ester

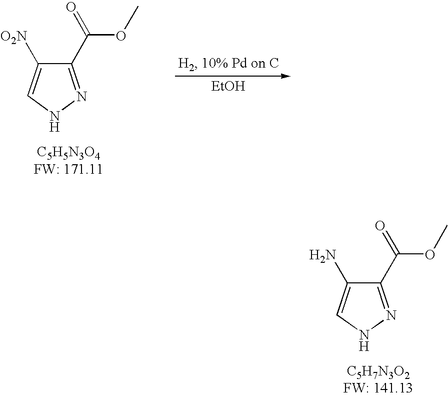

A 20 L reaction vessel equipped with a digital thermometer and stirrer was charged with palladium on carbon (10% wet paste, 0.170 Kg, 0.14 wt) under nitrogen. In a separate vessel a slurry of 4-nitro-1H-pyrazole-3-carboxylic acid methyl ester (1.210 Kg, 7.07 mol, 1 wt) in ethanol (12.10 L, 10 vol) was warmed to 30 to 35° C. to effect dissolution and the solution added to the catalyst under nitrogen. Following a nitrogen-hydrogen purge sequence an atmosphere of hydrogen was introduced and the reaction mixture maintained at 28 to 30° C. until reaction completion (5 to 10 hours) was noted by ¹H NMR analysis (d₆-DMSO). Following a purge cycle, the reaction mixture under nitrogen was filtered and the liquors concentrated under reduced pressure to give 4-amino-1H-pyrazole-3-carboxylic acid methyl ester (0.987 Kg, 98.9%).

### Stage 3: Preparation of 4-(2,6-dichlorobenzoylamino)-1H-pyrazole-3-carboxylic acid methyl ester

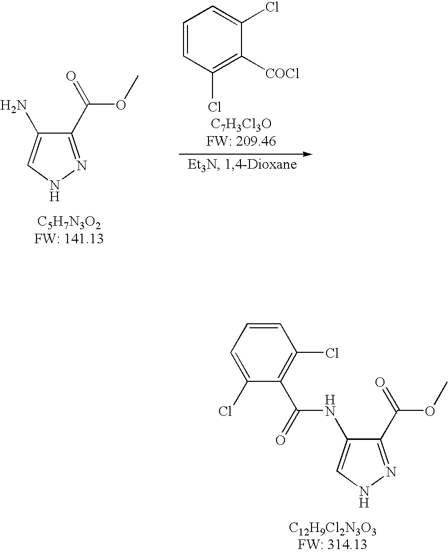

A solution of 4-amino-1H-pyrazole-3-carboxylic acid methyl ester (0.634 Kg, 4.49 mol, 1 wt) in 1,4-dioxane (8.90 L, 9 vol) under nitrogen was treated with triethylamine (0.761 L, 5.46 mol, 1.2 vol) followed by 2,6-dichlorobenzoyl chloride (0.710 L, 4.96 mol, 0.72 vol) such that the internal temperature was maintained in the range 20 to 25° C. Residual 2,6-dichlorobenzoyl chloride was washed in with a line rinse of 1,4-dioxane (0.990 L, 1 vol) and the reaction mixture stirred at 18 to 250 C until complete (16 hours) by TLC analysis (eluent: ethyl acetate: heptanes 3:1; R_{f amine} 0.25, R_{f product} 0.65). The reaction mixture was filtered, the filter-cake washed with 1,4-dioxane (2×0.990 L, 2×1 vol) and the combined filtrates (red) progressed to Stage 4 without further isolation.

### Stage 4: Preparation of 4-(2,6-dichlorobenzoylamino)-1H-pyrazole-3-carboxylic acid

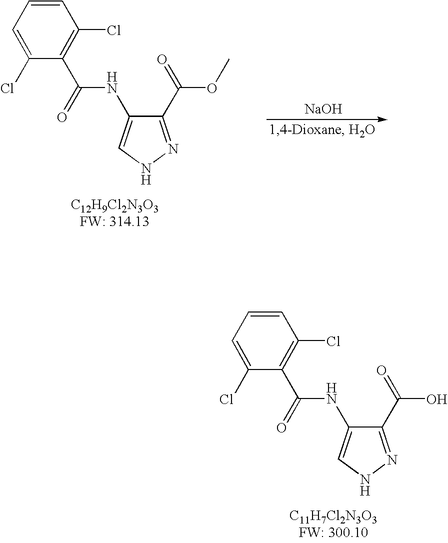

To a solution of sodium hydroxide (0.484 Kg, 12.1 mol) in water (6.05 L) was charged a solution of the Stage 3 ester in one portion: (1.099 Kg, 3.50 mol in 6.00 L). The reaction mixture was stirred to completion at 20 to 25° C. as determined by TLC analysis (eluent: ethyl acetate:heptanes 3:1; R_{f ester} 0.65, R_{f stage 4} baseline). The reaction mixture was concentrated under reduced pressure at 45 to 50° C., the oily residue diluted with water (9.90 L) and acidified to pH 1 with concentrated hydrochloric acid such that the temperature was maintained below 30° C. The resulting precipitate was collected by filtration, washed with water (5.00 L), pulled dry on the filter and subsequently washed with heptanes (5.00 L). The filter-cake was charged to a 20 L rotary evaporator flask and drying completed azeotropically with toluene (2×4.50 L) to afford 4-(2,6-dichlorobenzoylamino)-1H-pyrazole-3-carboxylic acid as a yellow solid (1.044 Kg, approx. 99.5%).

### Stage 5: Preparation of 4-{[4-(2,6-dichlorobenzoylamino)-1H-pyrazole-3-carbonyl]amino}piperidine-1-carboxylic acid tert-butyl ester

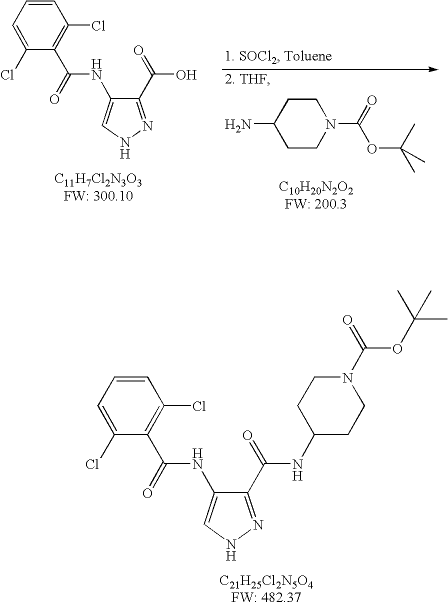

Stage 4 product (1.0 wt) and toluene (10.0 vol) were charged to a suitably sized flange flask equipped with a mechanical stirrer, dropping funnel and thermometer. The contents were stirred under nitrogen at 16 to 25° C. and thionyl chloride (0.3 vol) was added slowly. The contents were then heated to 80 to 100° C. and stirred at this temperature until the reaction was judged complete by ¹H NMR. Further toluene (up to 10 vol) could be added at this stage if the contents were to become too thick to stir. Once complete, the mixture was cooled to between 40 and 50° C. and then concentrated under vacuum at 45 to 50° C. to dryness. The residue was then azeo-dried with toluene (3×2.0 vol).

The isolated solid was transferred to a suitably sized flask and tetrahydrofuran (5.0 vol) was charged. The contents were stirred under nitrogen at 16 to 25° C. and triethylamine (0.512 vol) was added. To a separate flask was charged 4-amino-piperidine-1-carboxylic acid tert-butyl ester (0.704 wt) and tetrahydrofuran (5.0 vol). The contents were agitated until complete dissolution was achieved and the solution was then charged to the reaction flask, maintaining the temperature between 16 and 30° C. The reaction mixture was then heated to between 45 and 50° C. and the contents stirred until judged complete by ¹H NMR. The contents were then cooled to between 16 and 25° C. and water (5.0 vol) was charged. Mixed heptanes (0.5 vol) were added, the contents were stirred for up to 10 minutes and the layers were separated. The aqueous phase was then extracted with tetrahydrofuran:mixed heptanes [(9:1), 3×5.0 vol]. The organic phases were combined, washed with water (2.5 vol) and then concentrated under vacuum at 40 to 45° C. The residue was azeotroped with toluene (3×5.0 vol) and concentrated to dryness to yield the crude Stage 5 product.

The solid was then transferred to a suitably sized flask, methanol: toluene [(2.5:97.5), 5.0 vol] was added and the slurry was stirred under nitrogen for 3 to 18 hours. The contents were filtered, the filter-cake was washed with toluene (2×0.7 vol) and the solid was then dried under vacuum at 40 to 50° C. to yield 4-{[4-(2,6-dichlorobenzoylamino)-1H-pyrazole-3-carbonyl]amino}piperidine-1-carboxylic acid tert-butyl ester as an off-white solid.

Two batches of Stage 4 product (0.831 kg per batch) were processed in this way to give a total of 2.366 kg (88.6% yield) of 4-{[4-(2,6-dichlorobenzoylamino)-1H-pyrazole-3-carbonyl]amino}piperidine-1-carboxylic acid tert-butyl ester.

### Stage 6: Preparation of 4-(2,6-dichlorobenzoylamino)-1H-pyrazole-3-carboxylic acid piperidin-4-ylamide methanesulphonate

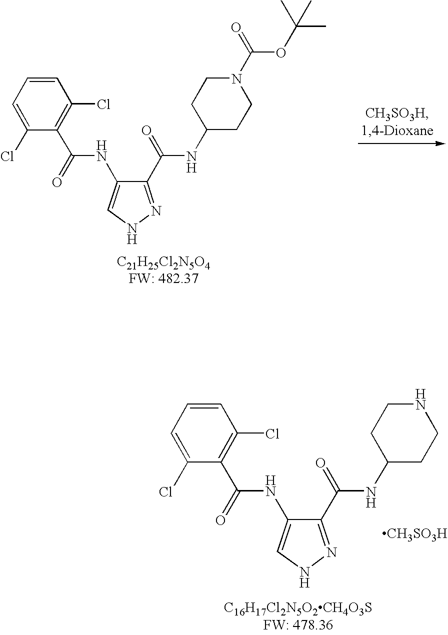

Stage 5 product (1.0 wt) and 1,4-dioxane (30.0 vol) were charged to a suitably sized flange flask equipped with a mechanical stirrer, dropping funnel and thermometer. The contents were stirred under nitrogen and heated to between 80 and 90° C. Methanesulphonic acid (0.54 vol) was added over 30 to 60 minutes and the contents were then heated to 95 to 105° C. and stirred in this temperature range until the reaction was judged complete by ¹H NMR. Once complete, the contents were cooled to between 20 and 30° C. and the resultant precipitate collected by filtration. The filter-cake was washed with 2-propanol (2×2.0 vol) and pulled dry on the filter for 3 to 24 hours to give crude 4-(2,6-dichlorobenzoylamino)-1H-pyrazole-3-carboxylic acid piperidin-4-ylamide methanesulphonate as a free-flowing off-white solid (80.0 to 120.0% w/w, uncorrected for impurities or solutes).

Several batches of Stage 5 product were processed in this way and the details of the quantities of starting material and product for each batch are set out in Table 1 below.

TABLE 1
Yields from the deprotection step - Stage 6

| | Input (g) of (4-{[4-(2,6- | | |
|---|---|---|---|
| | Dichloro-benzoylamino)- | Output (g) of [4-(2,6- | |
| | 1H-pyrazole-3- | Dichlorobenzoyl-amino)- | Chemical |
| | carbonyl]amino}- | 1H-pyrazole-3-carboxylic | purity |
| | piperidine-1-carboxylic | acid piperidin-4-ylamide | (HPLC % |
| Batch | acid tert-butyl ester) | methanesulphonate] | area) |
| 1 | 590.0 | 579.6 | 97.88 |
| | | 99.1% th, 98.2% w/w | |
| 2 | 521.0 | 532.7 | 98.09 |
| | | 103.1% th, 102.2% w/w | |
| 3 | 523.8 | 511.7 | 98.17 |
| | | 98.5% th, 97.7% w/w | |
| 4 | 518.4 | 596.3 | 98.24 |
| | | 116.0% th, 115.0% w/w | |
| 5 | 563.2 | 600.1 | 98.16 |
| | | 107.4% th, 106.6% w/w | |
| 6 | 563.1 | 565.2 | 98.49 |
| | | 101.2% th, 100.4% w/w | |
| 7 | 560.4 | 553.9 | 98.70 |
| | | 99.7% th, 98.8% w/w | |
| 8 | 569.7 | 560.6 | 98.41 |
| | | 99.2% th, 98.4% w/w | |

### Stage 6a: Recrystallisation of 4-(2,6-dichlorobenzoylamino)-1H-pyrazole-3-carboxylic acid piperidin-4-ylamide methanesulphonate

The product of Stage 6 was recrystallised to ensure that any residual levels of Boc-protected product of Stage 5 were no greater than 0.25%. Four batches of Stage 6 product were recrystallised using the following protocol.

Crude Stage 6 product and 2-propanol (10.0 vol) were charged to a suitably sized flask equipped with a mechanical stirrer, dropping funnel and thermometer. The contents were stirred under nitrogen and heated to between 75 and 85° C. Water (up to 2.5 vol) was then charged to the contents until a clear solution was obtained. The contents were then cooled to between 40 and 60° C. and concentrated under vacuum at 40 to 50° C. until the reaction volume was reduced by approximately 50%. 2-Propanol (3.0 vol) was charged to the flask and the contents were concentrated at 40 to 50° C. until approximately 3.0 vol of solvent was removed. This process was then repeated twice more with 2-propanol (2×3.0 vol) and the water content was checked. The resultant slurry was then cooled to between 0 and 5° C. and stirred at this temperature for 1 to 2 hours. The contents were filtered, the filter-cake was washed with 2-propanol (2×1.0 vol) and then pulled dry on the filter for up to 24 hours. The solid was transferred to drying trays and dried under vacuum at 45 to 50° C. to constant weight to give 4-(2,6-dichlorobenzoylamino)-1H-pyrazole-3-carboxylic acid piperidin-4-ylamide methanesulphonate as an off-white solid (60.0 to 100.0% w/w).

The recrystallisation yields for the four batches ranged between 85.6% and 90.4% and the purities of the recrystallised product ranged from 99.29% to 99.39%. A second recrystallisation increased the purity still further.

The 4-(2,6-dichlorobenzoylamino)-1H-pyrazole-3-carboxylic acid piperidin-4-ylamide methanesulphonate produced by this route had a melting point (by DSC) of 379.8° C.

### Removal of Residual Boc-protected Product of Stage 5

In some cases, when the methanesulphonate salt was dissolved in acetate buffer, a fine precipitate consisting of residual traces of the Boc-protected free base was observed. Several techniques may be used for removing or preventing the formation of the precipitate, as set out below.

### (a) Filtration

A mixture of the methanesulphonate salt in 200 mM acetate buffer was drawn from a vial into a 20 mL single-use syringe using a sterile needle, and a clinical grade 0.2 μm filter (a Sartorius Minisart sterile single use filter unit) was then attached to the syringe. The plunger of the syringe was slowly depressed and the filtrate collected in a clean, clear glass vial. The content of the vial was a clear, colourless solution of the methanesulphonate salt free of particulate matter.

### (b) Heating in Aqueous Acid

A mixture of the methanesulphonate salt and methanesulphonic acid (0.4 eq.) in water (10 vol) was heated at 100° C. for 4 hours, and then cooled to 60° C. Analysis by TLC indicates that the methanesulphonate salt was present as a single component. 2-Propanol (10 vol) was added and the mixture cooled to 40° C. The mixture was reduced in vacuo to approximately 10 volumes, then a further portion of 2-propanol added (10 vol) and the mixture again reduced to 10 volumes. This cycle was repeated a further three times. The mixture was cooled in an ice-bath and the solid formed collected by filtration, washed with 2-propanol (5 vol) and dried in vacuo to give the methanesulphonate salt as a white to off-white solid.

### (c) Organic-aqueous Extractions

A mixture of the methanesulphonate salt and methanesulphonic acid (0.4 eq.) in water (10 vol) was heated at 100° C. for 3 hours, and then cooled to ambient temperature. To this mixture was added THF-heptane (9:1, 10 vol) and the resultant mixture stirred vigorously to give a solution. The layers were separated and the aqueous phase washed with THF-heptane (9:1, 2×10 vol) then ethyl acetate (2×10 vol). To the aqueous phase was added 2-propanol (10 vol) and the solution was reduced in vacuo to approximately 5 volumes, then a further portion of 2-propanol added (10 vol) and the mixture again reduced to 5 volumes. This cycle was repeated a further three times. The solid formed was collected by filtration, washed with 2-propanol (5 vol) and dried in vacuo to give the methanesulphonate salt as a white to off-white solid.

### (d) Chromatography

The use of chromatographic techniques may provide a route for removing non-polar impurities from the methanesulphonate salt. The use of reverse-phase methods will be particularly useful.

### Biological Activity

The biological activities of the compounds of (0), (I⁰), (I), (Ia), (Ib), (II), (III), (IV), (IVa), (Va), (Vb), (VIa), (VIb), (VII) or (VIII) and sub-groups thereof as defined in WO 2005/012256 (PCT/GB2004/003179) (and therefore herein also by dint of the incorporation of the relevant subject matter of WO 2005/012256 (PCT/GB2004/003179) by reference, see infra) as inhibitors of CDK kinases, GSK-3 kinase and as inhibitors of cell growth are demonstrated by the examples set out below.

### Example 5

### Measurement of CDK2 Kinase Inhibitory Activity (IC₅₀)

See Example 246 of WO 2005/012256 at pages 213-214 (which is incorporated herein by reference).

### Example 6

### CDK Selectivity Assays

See Example 247 of WO 2005/012256 at pages 214-215 (which is incorporated herein by reference).

### Example 7A

### Measurement of Activated CDK2/CyclinA Kinase Inhibitory Activity Assay (IC₅₀)

See Example 12 of WO 2006/077426 at pages 98-99 (which is incorporated herein by reference).

### 7B. CDK1/CyclinB Assay

See Example 248B of WO 2005/012256 at pages 216-217 (which is incorporated herein by reference).

### Example 8

### Assay Procedure for CDK4

See Example 249 of WO 2005/012256 at pages 217 (which is incorporated herein by reference).

### Example 9

### Measurement of Inhibitory Activity against Glycogen Synthase Kinase-3 (GSK-3)

See Example 251 of WO 2005/012256 at pages 218-219 (which is incorporated herein by reference).

### Example 10

### Anti-proliferative Activity

See Example 15 of WO 2006/077426 at pages 100-101 (which is incorporated herein by reference).

### HCT-116 Cell Line

In assays against the human colon carcinoma cell line HCT 116 (ECACC No. 91091005), the compound of Example 1 has an IC₅₀ value of less than 20 μM.

### Example 11

The effect of the compound 4-(2,6-dichloro-benzoylamino)-1H-pyrazole-3-carboxylic acid piperidin-4-ylamide (“Compound I”) in combination with the auxiliary compounds 5FU, Gemcitibine, Paclitaxel, and Iressa (Compound II) were assessed using the following technique:

### IC₅₀ Shift Assay

Human colon carcinoma cell line HT29 (ECACC No. 91072201) cells were seeded onto 96-well tissue culture plates at a concentration of 5×10³ cells/well. Cells were allowed to recover overnight prior to addition of compound(s) or vehicle control (0.2% DMSO) as follows;

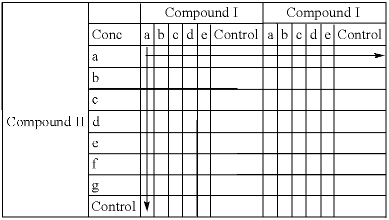

Compounds were added according to one of the following schedules;
a) Concurrent for 72 hours.
b) Compound I for 24 hours followed by Compound II for 48 hours.
c) Compound II for 24 hours followed by Compound I for 48 hours.

Following a total of 72 hours compound incubation, Alamar Blue™ was added to a final concentration of 10% (v/v) and incubated at 37° C. for 6 hours. Fluorescent product was quantified by reading at d535/25× (excitation) and d590/20 m (emission) on a Fusion Reader (Perkin Elmer). The IC₅₀ for Compound II in the presence of varying doses of Compound I was determined. Synergy was determined when the IC₅₀ shifted down in the presence of sub-effective doses of Compound I. Additivity was determined when the response to Compound II and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

1. Gemcitibine

Combinations of Compound I and Gemcitibine were shown to be additive in the IC₅₀ shift assay performed in A2780 cells. This effect was observed when compounds were added concurrently for 72 h or when Gemcitibine was added for 24 h followed by Compound I for a further 48 h. The data obtained are summarised in FIGS. 1 and 2 of WO2006/077424 using the example of the IC₅₀ response curves to Gemcitibine in the presence and absence of 0.3 μM Compound I added concurrently.

2. Paclitaxel

Combinations of Compound I and Paclitaxel were shown to be additive or synergistic in the IC₅₀ shift assay dependent upon schedule. The studies were performed in A2780 cells. Additive effects were observed when compounds were added concurrently for 72 h and synergy when Paclitaxel was added for 24 h followed by Compound I for a further 48 h. The data obtained are summarised in FIGS. 3, 4, 5 and 6 of WO2006/077424 using examples of the IC₅₀ response curves to Paclitaxel in the presence and absence of 0.3 μM Compound I.

3. 5FU

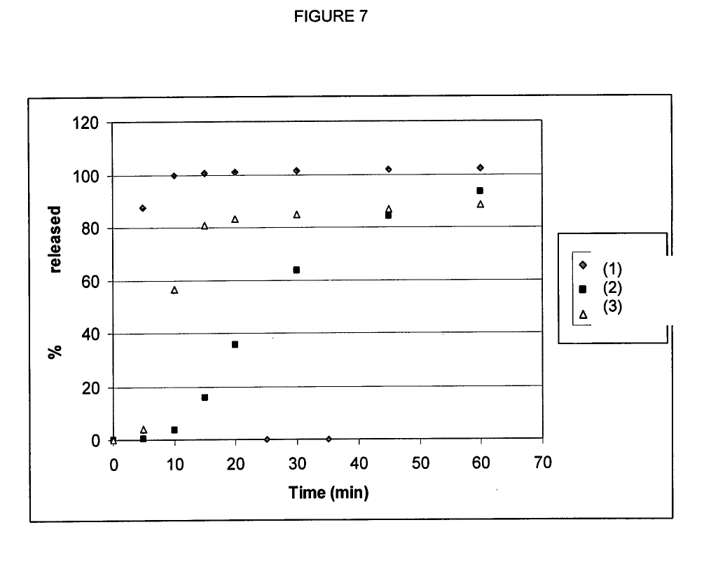

Combinations of Compound I and 5-FU were shown to be mildly synergistic in the IC₅₀ shift assay performed in A2780 cells. This effect was observed when compounds were added concurrently for 72 h. The data obtained are summarised in FIGS. 7 and 8 of WO2006/077424 using the example of the IC₅₀ response curves to 5-FU in the presence and absence of 0.15 μM Compound I when added concurrently.

4. Iressa

Combinations of Compound I and Iressa were shown to be synergistic in the IC₅₀ shift assay performed in A2780 cells. This effect was observed when compounds were added concurrently for 72 h. The data obtained are summarised in FIGS. 9 and 10 of WO2006/077424 using examples of the IC₅₀ response curves to Iressa in the presence and absence of 0.2 μM Compound I. These data were confirmed in the HCT116 and SkBR3 cell lines.

### Example 12

The effect of the compound 4-(2,6-dichloro-benzoylamino)-1H-pyrazole-3-carboxylic acid piperidin-4-ylamide (“Compound I”) in combination with camptothecin was assessed using the following technique:

### 1. IC₅₀ Shift Assay

Human colon carcinoma cell line HT29 (ECACC No. 91072201) cells were seeded onto 96-well tissue culture plates at a concentration of 5×10³ cells/well. Cells were allowed to recover overnight prior to addition of compound(s) or vehicle control (0.2% DMSO) as follows;

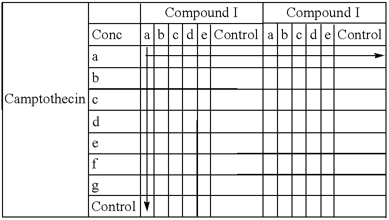

Compounds were added according to one of the following schedules;
a) Concurrent for 72 hours.
b) Compound I for 24 hours followed by camptothecin for 48 hours.
c) Camptothecin for 24 hours followed by Compound I for 48 hours.

Following a total of 72 hours compound incubation, Alamar Blue™ was added to a final concentration of 10% (v/v) and incubated at 37° C. for 6 hours. Fluorescent product was quantified by reading at d535/25× (excitation) and d590/20 m (emission) on a Fusion Reader (Perkin Elmer). The IC₅₀ for camptothecin in the presence of varying doses of Compound I was determined. Synergy was determined when the IC₅₀ shifted down in the presence of sub-effective doses of Compound I. Additivity was determined when the response to camptothecin and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

Combinations of Compound I and camptothecin were shown to be additive in the IC₅₀ shift assay performed in HT29 cells. This effect was observed when compounds were added concurrently for 72 hours or when camptothecin was added for 24 hours followed by Compound I for a further 48 hours. Similar additivity was observed when Compound I was added prior to camptothecin. The data obtained are summarised in FIGS. 11 and 12 of WO2006/077424 using the example of the IC₅₀ response curves to camptothecin in the presence and absence of 0.1 μM Compound I in Schedule (b) (Compound I added followed by camptothecin).

### Example 13

The effect of the compound 4-(2,6-dichloro-benzoylamino)-1H-pyrazole-3-carboxylic acid piperidin-4-ylamide (“Compound I”) in combination with vinblastine was assessed using the following technique:

### 1. IC₅₀ Shift Assay

Human colon carcinoma cell line HT29 (ECACC No. 91072201) cells were seeded onto 96-well tissue culture plates at a concentration of 5×10³ cells/well. Cells were allowed to recover overnight prior to addition of compound(s) or vehicle control (0.2% DMSO) as follows;

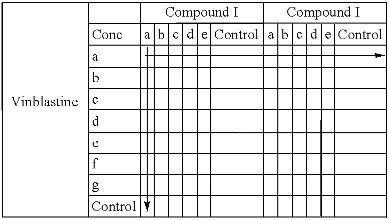

Compounds were added according to one of the following schedules;
d) Concurrent for 72 hours.
e) Compound I for 24 hours followed by vinblastine for 48 hours.
f) Vinblastine for 24 hours followed by Compound I for 48 hours.

Following a total of 72 hours compound incubation, Alamar Blue™ was added to a final concentration of 10% (v/v) and incubated at 37° C. for 6 hours. Fluorescent product was quantified by reading at d535/25× (excitation) and d590/20 m (emission) on a Fusion Reader (Perkin Elmer). The IC₅₀ for vinblastine in the presence of varying doses of Compound I was determined. Synergy was determined when the IC₅₀ shifted down in the presence of sub-effective doses of Compound I. Additivity was determined when the response to vinblastine and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

Combinations of Compound I and Vinblastine were shown to be additive in the IC₅₀ shift assay performed in A2780 cells. This effect was observed when compounds were added concurrently for 72 h or when Vinblastine was added for 24 h followed by compound I for a further 48 h. The data obtained are summarised below in FIGS. 13 and 14 of WO2006/077424 using the example of the IC₅₀ response curves to Vinblastine in the presence and absence of 0.3 μM Compound I when added concurrently.

### Example 14

The effect of the compound 4-(2,6-dichloro-benzoylamino)-1H-pyrazole-3-carboxylic acid piperidin-4-ylamide (“Compound I”) in combination with cisplatin was assessed using the following technique:

### 1. IC₅₀ Shift Assay

Human colon carcinoma cell line HT29 (ECACC No. 91072201) cells were seeded onto 96-well tissue culture plates at a concentration of 5×10³ cells/well. Cells were allowed to recover overnight prior to addition of compound(s) or vehicle control (0.2% DMSO) as follows;

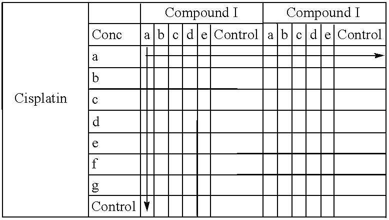

Compounds were added according to one of the following schedules;
g) Concurrent for 72 hours.
h) Compound I for 24 hours followed by cisplatin for 48 hours.
i) Cisplatin for 24 hours followed by Compound I for 48 hours.

Following a total of 72 hours compound incubation, Alamar Blue™ was added to a final concentration of 10% (v/v) and incubated at 37° C. for 6 hours. Fluorescent product was quantified by reading at d535/25× (excitation) and d590/20 m (emission) on a Fusion Reader (Perkin Elmer). The IC₅₀ for cisplatin in the presence of varying doses of Compound I was determined. Synergy was determined when the IC₅₀ shifted down in the presence of sub-effective doses of Compound I. Additivity was determined when the response to cisplatin and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

Combinations of compound I and Cisplatin were shown to be additive in the IC₅₀ shift assay performed in A2780 cells. This effect was observed when compounds were added concurrently for 72 h or when Cisplatin was added for 24 h followed by compound I for a further 48 h. The data obtained are summarised below in FIGS. 15 and 16 of WO2006/077424 using the example of the IC₅₀ response curves to Cisplatin in the presence and absence of 0.3 μM compound I when added concurrently.

### Example 15

The effect of the compound 4-(2,6-dichloro-benzoylamino)-1H-pyrazole-3-carboxylic acid piperidin-4-ylamide (“Compound I”) in combination with etoposide was assessed using the following technique:

### 1. IC₅₀ Shift Assay

Human colon carcinoma cell line HT29 (ECACC No. 91072201) cells were seeded onto 96-well tissue culture plates at a concentration of 5×10³ cells/well. Cells were allowed to recover overnight prior to addition of compound(s) or vehicle control (0.2% DMSO) as follows;

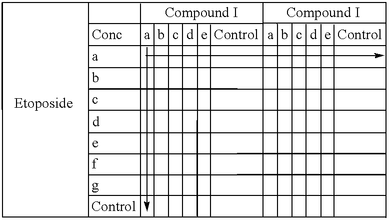

Compounds were added according to one of the following schedules;
j) Concurrent for 72 hours.
k) Compound I for 24 hours followed by etoposide for 48 hours.
l) Etoposide for 24 hours followed by Compound I for 48 hours.

Following a total of 72 hours compound incubation, Alamar Blue™ was added to a final concentration of 10% (v/v) and incubated at 37° C. for 6 hours. Fluorescent product was quantified by reading at d535/25× (excitation) and d590/20 m (emission) on a Fusion Reader (Perkin Elmer). The IC₅₀ for etoposide in the presence of varying doses of Compound I was determined. Synergy was determined when the IC₅₀ shifted down in the presence of sub-effective doses of Compound I. Additivity was determined when the response to etoposide and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

Combinations of Compound I and Etoposide were shown to be additive in the IC₅₀ shift assay performed in A2780 cells. This effect was observed when compounds were added concurrently for 72 h or when Etoposide was added for 24 h followed by compound I for a further 48 h. The data obtained are summarised below in FIGS. 17 and 18 of WO2006/077424 using the example of the IC₅₀ response curves to Etoposide in the presence and absence of 0.075 μM Compound I when added concurrently.

### Example 16

### Pharmaceutical Formulations

(i) Lyophilised formulation I

Aliquots of formulated compound of formula (0), (I⁰), (I), (Ia), (Ib), (II), (III), (IV), (IVa), (Va), (Vb), (VIa), (VIb), (VII) or (VIII) and sub-groups thereof as defined in WO 2005/012256 (PCT/GB2004/003179) (and therefore herein also by dint of the incorporation of the relevant subject matter of WO 2005/012256 (PCT/GB2004/003179) by reference, see infra) are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

### (ii) Injectable Formulation II

A formulation for i.v. delivery by injection or infusion can be prepared by dissolving the compound of formula (0), (I⁰), (I), (Ia), (Ib), (II), (III), (IV), (IVa), (Va), (Vb), (VIa), (VIb), (VII) or (VIII) and sub-groups thereof as defined herein (e.g. in a salt form) in water at 20 mg/ml. The vial is then sealed and sterilised by autoclaving.

iii) Injectable Formulation III

A formulation for i.v. delivery by injection or infusion can be prepared by dissolving the compound of formula (0), (I⁰), (I), (Ia), (Ib), (II), (III), (IV), (IVa), (Va), (Vb), (VIa), (VIb), (VII) or (VIII) and sub-groups thereof as defined herein (e.g. in a salt form) in water containing a buffer (e.g. 0.2 M acetate pH 4.6) at 20 mg/ml. The vial is then sealed and sterilised by autoclaving.

### iv) Injectable Formulation IV

A parenteral composition for administration by injection can be prepared by dissolving a compound (e.g. in a salt form) in water containing 10% propylene glycol to give a concentration of active compound of 1.5% by weight. The solution is then sterilised by filtration, filled into an ampoule and sealed.

### (v) Injectable Formulation V

A parenteral composition for injection is prepared by dissolving in water a compound (e.g. in salt form) (2 mg/ml) and mannitol (50 mg/ml), sterile filtering the solution and filling into sealable 1 ml vials or ampoules.

### (vi) Subcutaneous Injection Formulation VI

A composition for sub-cutaneous administration is prepared by mixing a compound with pharmaceutical grade corn oil to give a concentration of 5 mg/ml. The composition is sterilised and filled into a suitable container.

(vii) Tablet Formulation

A tablet composition containing a compound or an acid addition salt thereof as defined herein is prepared by mixing 50 mg of the compound or its salt with 197 mg of lactose (BP) as diluent, and 3 mg magnesium stearate as a lubricant and compressing to form a tablet in known manner.

(viii) Capsule Formulation

A capsule formulation is prepared by mixing 100 mg of a compound or an acid addition salt thereof as defined herein with 100 mg lactose and filling the resulting mixture into standard opaque hard gelatin capsules.

### (ix) Lyophilised Formulation

Aliquots of formulated compound or an acid addition salt thereof as defined herein are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

(x) Concentrate for Use in i.v. Administration

An aqueous buffered solution is prepared by dissolving 4-(2,6-dichlorobenzoylamino)-1H-pyrazole-3-carboxylic acid piperidin-4-ylamide methanesulphonate at a concentration of 20 mg/ml in a 0.2M sodium acetate/acetic acid buffer at a pH of 4.6.

The buffered solution is filled, with filtration to remove particulate matter, into a container (such as class 1 glass vials) which is then sealed (e.g. by means of a Florotec stopper) and secured (e.g. with an aluminium crimp). If the compound and formulation are sufficiently stable, the formulation is sterilised by autoclaving at 121° C. for a suitable period of time. If the formulation is not stable to autoclaving, it can be sterilised using a suitable filter and filled under sterile conditions into sterile vials. For intravenous administration, the solution can be dosed as is, or can be injected into an infusion bag (containing a pharmaceutically acceptable excipient, such as 0.9% saline or 5% dextrose), before administration.

### (xi) Injectable Formulation of a Camptothecin Compound

A parenteral pharmaceutical formulation for administration by injection and containing a camptothecin compound can be prepared by dissolving 100 mg of a water soluble salt of the camptothecin compound (for example a compound as described in EP 0321122 and in particular the examples therein) in 10 ml of sterile 0.9% saline and then sterilising the solution and filling the solution into a suitable container

### Example 17

### Determination of the crystal structure of 4-(2,6-dichlorobenzoylamino)-1H-pyrazole-3-carboxylic acid piperidin-4-ylamide methanesulphonate by X-ray diffraction

See Example 2 and FIG. 1 of WO 2006/077426 at pages 81-85 (which is incorporated herein by reference).

### Example 18

### Preparation of 4-(2,6-dichloro-benzoylamino)-1H-pyrazole-3-carboxylic acid piperidin-4-ylamide acetic acid salt

See Example 8 and FIG. 2 of WO 2006/077426 at pages 94-95 (which is incorporated herein by reference).

### Set B: Examples Relating to Compounds of the Formula (I′″)

The invention will now be illustrated, but not limited, by reference to the specific embodiments described in the following examples.

In the examples, the following abbreviations are used.
AcOH acetic acid
BOC tert-butyloxycarbonyl
CDI 1,1-carbonyldiimidazole
DMAW90 Solvent mixture: DCM: MeOH, AcOH, H₂O (90:18:3:2)
DMAW120 Solvent mixture: DCM: MeOH, AcOH, H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
P.E. petroleum ether
SiO₂ silica
TBTU N,N,N′,N′-tetramethyl-O-(benzotriazol-1-yl)uronium tetrafluoroborate
THF tetrahydrofuran

### Analytical LC-MS System and Method Description

In the examples, the compounds prepared were characterised by liquid chromatography and mass spectroscopy using the systems and operating conditions set out below. Where atoms with different isotopes are present, and a single mass quoted, the mass quoted for the compound is the monoisotopic mass (i.e. ³⁵Cl; ⁷⁹Br etc.). Several systems were used, as described below, and these were equipped with, and were set up to run under, closely similar operating conditions. The operating conditions used are also described below.

### Waters Platform LC-MS System:

| | |
|---|---|
| HPLC System: | Waters 2795 |
| Mass Spec Detector: | Micromass Platform LC |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ |
| | MAX-RP 80A, 2.0 × 50 mm |

### Analytical Long Acidic Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 80A, 2.0 × 150 mm |

### Platform MS Conditions:

| | |
|---|---|
| Capillary voltage: | 3.6 kV (3.40 kV on ES negative) |
| Cone voltage: | 25 V |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Waters Fractionlynx LC-MS System:

| | |
|---|---|
| HPLC System: | 2767 autosampler-2525 binary gradient pump |
| Mass Spec Detector: | Waters ZQ |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 4 minutes |
| Flow: | 2.0 ml/min |
| Column: | Phenomenex Synergi 4μ MAX-RP 80A, 4.6 × 50 mm |

### Fractionlynx MS Conditions:

| | |
|---|---|
| Capillary voltage: | 3.5 kV (3.2 kV on ES negative) |
| Cone voltage: | 25 V (30 V on ES negative) |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Preparative LC-MS Systems:

### Waters Fractionlynx System:

Hardware:
2767 Dual Loop Autosampler/Fraction Collector
2525 preparative pump
CFO (column fluidic organiser) for column selection
RMA (Waters reagent manager) as make up pump
Waters ZQ Mass Spectrometer
Waters 2996 Photo Diode Array detector
Waters ZQ Mass Spectrometer

Software:
Masslynx 4.0

Waters MS Running Conditions:

| | |
|---|---|
| Capillary voltage: | 3.5 kV (3.2 kV on ES Negative) |
| Cone voltage: | 25 V |
| Source Temperature: | 120° C. |
| Multiplier: | 500 V |
| Scan Range: | 125-800 amu |
| Ionisation Mode: | ElectroSpray Positive or |
| | ElectroSpray Negative |

### Agilent 1100 LC-MS Preparative System:

Hardware:
Autosampler: 1100 series “prepALS”
Pump: 1100 series “PrepPump” for preparative flow gradient and 1100 series “QuatPump” for pumping modifier in prep flow
UV detector: 1100 series “MWD” Multi Wavelength Detector
MS detector: 1100 series “LC-MSD VL”
Fraction Collector: 2×“Prep-FC”
Make Up pump: “Waters RMA”
Agilent Active Splitter

Software:
Chemstation: Chem32

Agilent MS Running Conditions:

| | |
|---|---|
| Capillary voltage: | 4000 V (3500 V on ES Negative) |
| Fragmentor/Gain: | 150/1 |
| Drying gas flow: | 13.0 L/min |
| Gas Temperature: | 350° C. |
| Nebuliser Pressure: | 50 psig |
| Scan Range: | 125-800 amu |
| Ionisation Mode: | ElectroSpray Positive or |
| | ElectroSpray Negative |

### Chromatographic Conditions:

Columns:
1. Low pH chromatography:
Phenomenex Synergy MAX-RP, 10μ, 100×21.2 mm
(alternatively used Thermo Hypersil-Keystone HyPurity Aquastar, 5μ, 100×21.2 mm for more polar compounds)
2. High pH chromatography:
Phenomenex Luna C18 (2), 10□, 100×21.2 mm
(alternatively used Phenomenex Gemini, 5p, 100×21.2 mm)

Eluents:
1. Low pH chromatography:
Solvent A: H₂O+0.1% Formic Acid, pH˜1.5
Solvent B: CH₃CN+0.1% Formic Acid
2. High pH chromatography:
Solvent A: H₂O+10 mM NH₄HCO₃+NH₄OH, pH=9.2
Solvent B: CH₃CN
3. Make up solvent:
MeOH+0.2% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min
Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)
Wash: 1.2 minute wash step was performed at the end of the gradient
Re-equilibration: 2.1 minutes re-equilibration step was ran to prepare the system for the next run
Make UP flow rate: 1 ml/min

Solvent:

All compounds were usually dissolved in 100% MeOH or 100% DMSO

From the information provided someone skilled in the art could purify the compounds described herein by preparative LC-MS.

The starting materials for each of the Examples are commercially available unless otherwise specified.

### Example 1

### 1A.4-Nitro-1H-pyrazole-3-carboxylic acid methyl ester

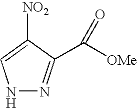

Thionyl chloride (2.90 ml, 39.8 mmol) was slowly added to a mixture of 4-nitro-3-pyrazolecarboxylic acid (5.68 g, 36.2 mmol) in MeOH (100 ml) at ambient temperature and the mixture stirred for 48 hours. The mixture was reduced in vacuo and dried through azeotrope with toluene to afford 4-nitro-1H-pyrazole-3-carboxylic acid methyl ester as a white solid.

¹H NMR (400 MHz, DMSO-d₆) δ 14.4 (s, 1H), 8.9 (s, 1H), 3.9 (s, 3H)

### 1B. 4-Amino-1H-pyrazole-3-carboxylic acid methyl ester

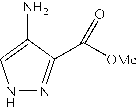

A mixture of 4-nitro-1H-pyrazole-3-carboxylic acid methyl ester and 10% Pd/C in EtOH was stirred under an atmosphere of hydrogen for 20 hours. The mixture was filtered through a plug of Celite, reduced in vacuo and dried through azeotrope with toluene to afford 4-amino-1H-pyrazole-3-carboxylic acid methyl ester.

¹H NMR (400 MHz, MeOD) δ 7.2 (s, 1H), 3.9 (s, 3H)

### 1C. 4-(2,6-dichloro-benzoylamino)-1H-pyrazole-3-carboxylic acid

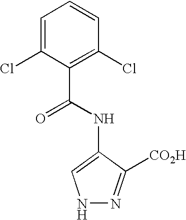

2,6-dichlorobenzoyl chloride (8.2 g; 39.05 mmol) was added cautiously to a solution of 4-amino-1H-pyrazole-3-carboxylic acid methyl ester (5 g; 35.5 mmol) and triethylamine (5.95 ml; 42.6 mmol) in dioxane (50 ml) then stirred at room temperature for 5 hours. The reaction mixture was filtered and the filtrate treated with methanol (50 ml) and 2M sodium hydroxide solution (100 ml), heated at 50° C. for 4 hours, and then evaporated. 100 ml of water was added to the residue then acidified with concentrated hydrochloric acid. The solid was collected by filtration, washed with water (100 ml) and sucked dry to give 10.05 g of 4-(2,6-dichloro-benzoylamino)-1H-pyrazole-3-carboxylic acid as a pale violet solid. (LC/MS: Rₜ 2.26, [M+H]⁺ 300/302).

### 1D. 4-{[4-(2,6-dichloro-benzoylamino)-1H-pyrazole-3-carbonyl]-amino}-piperidine-1-carboxylic acid tert-butyl ester

A mixture of 4-(2,6-dichloro-benzoylamino)-1H-pyrazole-3-carboxylic acid (6.5 g, 21.6 mmol), 4-amino-1-BOC-piperidine (4.76 g, 23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken up in 5% MeOH-DCM (˜30 ml). The insoluble material was collected by filtration and, washed with DCM and dried in vacuo to give 4-{[4-(2,6-dichloro-benzoylamino)-1H-pyrazole-3-carbonyl]-amino}-piperidine-1-carboxylic acid tert-butyl ester (5.38 g) as a white solid. The filtrate was reduced in vacuo and the residue purified by column chromatography using gradient elution 1:2 EtOAc/hexane to EtOAc to give further 4-{[4-(2,6-dichloro-benzoylamino)-1H-pyrazole-3-carbonyl]-amino}-piperidine-1-carboxylic acid tert-butyl ester (2.54 g) as a white solid.

### 1E. 4-(2,6-dichloro-benzoylamino)-1H-pyrazole-3-carboxylic acid piperidin-4-ylamide hydrochloride

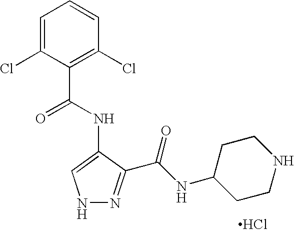

A solution of 4-{[4-(2,6-dichloro-benzoylamino)-1H-pyrazole-3-carbonyl]-amino}-piperidine-1-carboxylic acid tert-butyl ester (7.9 g) in MeOH (50 mL) and EtOAc (50 ml) was treated with sat. HCl-EtOAc (40 mL) then stirred at r.t. overnight. The product did not crystallise due to the presence of methanol, and therefore the reaction mixture was evaporated and the residue triturated with EtOAc. The resulting off white solid was collected by filtration, washed with EtOAc and sucked dry on the sinter to give 6.3 g of 4-(2,6-dichloro-benzoylamino)-1H-pyrazole-3-carboxylic acid piperidin-4-ylamide as the hydrochloride salt. (LC/MS: R. 5.89, [M+H]⁺ 382/384).

### 1F. 4-(2,6-dichloro-benzoylamino)-1H-pyrazole-3-carboxylic acid (1-methanesulphonyl-piperidin-4-yl)-amide

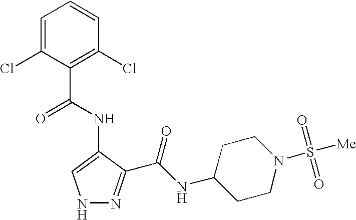

To a mixture of 4-(2,6-dichloro-benzoylamino)-1H-pyrazole-3-carboxylic acid piperidin-4-ylamide hydrochloride (1 mmol) in acetonitrile (10 ml) was added diisopropylethylamine (2.2 mmol) followed by the methanesulphonyl chloride (1 mmol). The mixture was stirred at ambient temperature for 16 hours then reduced in vacuo. The residue was partitioned between ethyl acetate and water, the layers separated and the organic portion washed with brine, dried (MgSO₄) and reduced in vacuo to give the title compound. [M+H]⁺ 460 Rₜ 2.67. LC/MS. r.t. 2.67 min; m/z 460.11

¹H NMR: (400 MHz, DMSO-d₆) δ 13.51 (s, 1H), 10.20 (s, 1H), 8.50 (d, J=8.0 Hz, 1H), 8.41 (s, 1H), 7.66-7.56 (m, 3H), 3.95-3.89 (m, 1H), 3.61 (d, J=12.0 Hz, 2H), 2.92 (s, 3H), 2.84 (t, J=12.0 Hz, 2H), 1.89-1.86 (m, 2H), 1.79-1.70 (m, 2H)

### Example 2

### 4-(2,6-dichloro-benzoylamino)-1H-pyrazole-3-carboxylic acid (1-isopropyl-sulphonyl-Piperidin-4-yl)-amide

See Example 2 of WO 2006/077416 at page 71 (which is incorporated herein by reference).

### Example 3

### 4-(2,6-dichloro-benzoylamino)-1H-pyrazole-3-carboxylic acid (1-ethyl-sulphonyl-piperidin-4-yl)-amide

See Example 3 of WO 2006/077416 at page 71 (which is incorporated herein by reference).

### Example 4

### 4-(2,6-dichloro-benzoylamino)-1H-pyrazole-3-carboxylic acid (1-propyl-sulphonyl-piperidin-4-yl)-amide

See Example 4 of WO 2006/077416 at page 72 (which is incorporated herein by reference).

### Example 5

### Synthesis of 4-(2,6-dichloro-benzoylamino)-1H-pyrazole-3-carboxylic acid (1-methanesulphonyl-piperidin-4-yl)-amide and crystals thereof

The compound 4-(2,6-dichloro-benzoylamino)-1H-pyrazole-3-carboxylic acid (1-methanesulphonyl-piperidin-4-yl)-amide can be prepared by the synthetic sequence illustrated in Scheme 1 above and described in more detail below.

### Stage 1: Preparation of 4-nitro-1H-pyrazole-3-carboxylic acid methyl ester

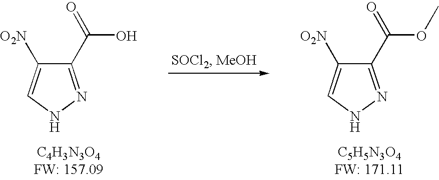

4-Nitro-1H-pyrazole-3-carboxylic acid (1.350 Kg, 8.59 Mol, 1.0 wt) and methanol (10.80 L, 8.0 vol) were charged to a flange flask equipped with a mechanical stirrer, condenser and thermometer. The suspension was cooled to 0 to 5° C. under nitrogen and thionyl chloride (0.702 L, 9.62 Mol, 0.52 vol) added at this temperature. The mixture was warmed to 15 to 25° C. over 16 to 24 hours. Reaction completion was determined by ¹H NMR analysis (d₆-DMSO). The mixture was concentrated under vacuum at 35 to 45° C. and toluene (2.70 L, 2.0 vol) charged to the residue and removed under vacuum at 35 to 45° C. The toluene azeotrope was repeated twice using toluene (2.70 L, 2.0 vol) to give 4-nitro-1H-pyrazole-3-carboxylic acid methyl ester [1.467 Kg, 99.8% th, 108.7% w/w, ¹H NMR (d₆-DMSO) concordant with structure, no entrained solvent] as an off-white solid.

### Stage 2: Preparation of 4-amino-1H-pyrazole-3-carboxylic acid methyl ester

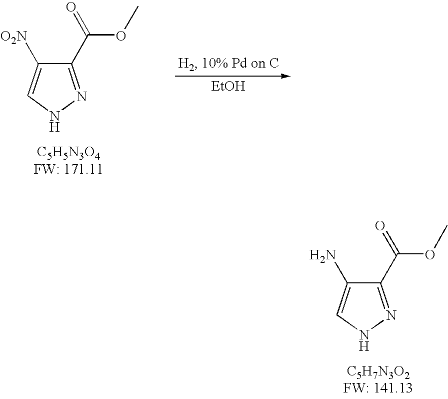

A suspension of 4-nitro-1H-pyrazole-3-carboxylic acid methyl ester (1.467 Kg, 8.57 Mol, 1.0 wt) and ethanol (14.70 L, 10.0 vol) was heated to and maintained at 30 to 35° C. until complete dissolution occurred. 10% Palladium on carbon (10% Pd/C wet paste, 0.205 Kg, 0.14 wt) was charged to a separate flask under nitrogen and a vacuum/nitrogen purge cycle performed (×3). The solution of 4-nitro-1H-pyrazole-3-carboxylic acid methyl ester in ethanol was charged to the catalyst and the vacuum/nitrogen purge cycle repeated (×3). A vacuum/hydrogen purge cycle was performed (×3) and the reaction placed under an atmosphere of hydrogen. The reaction mixture was stirred at 28 to 30° C. until deemed complete by ¹H NMR analysis (d₆-DMSO). The mixture was filtered under nitrogen and concentrated under vacuum at 35 to 45° C. to give 4-amino-1H-pyrazole-3-carboxylic acid methyl ester [1.184 Kg, 97.9% th, 80.7% w/w, ¹H NMR (d₆-DMSO) concordant with structure, corrected for 0.27% w/w entrained ethanol] as an off-white solid.

### Stage 3: Preparation of 4-(2,6-dichlorobenzoylamino)-1H-pyrazole-3-carboxylic acid methyl ester

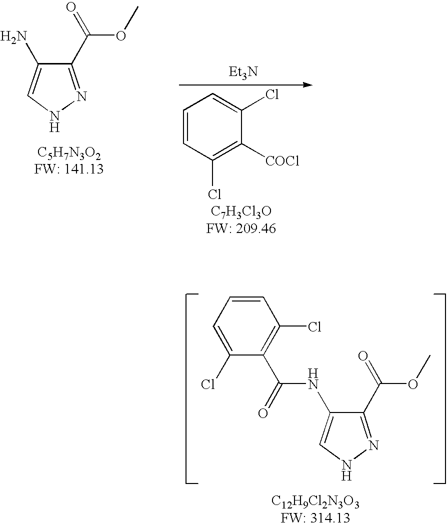

Triethylamine (1.42 L, 10.20 Mol, 1.2 vol) was added to solution of 4-amino-1H-pyrazole-3-carboxylic acid methyl ester (1.184 Kg, 8.39 Mol, 1.0 wt) in 1,4-dioxane (10.66 L, 9.0 vol) at 15 to 25° C. under nitrogen. 2,6-Dichlorobenzoyl chloride (1.33 L, 9.28 Mol, 1.12 vol) was charged at 15 to 25° C. followed by a line rinse of 1,4-dioxane (1.18 L, 1.0 vol) and the reaction mixture stirred at 15 to 25° C. for 14 to 24 hours. Reaction completion was determined by ¹H NMR analysis¹. The reaction mixture was filtered, the filter-cake washed with 1,4-dioxane (2×1.18 L, 2×1.0 vol) and the combined filtrates progressed to Stage 4 without further isolation. ¹ A sample of the reaction mixture was filtered, the filtrates dissolved in d₆-DMSO and a ¹H NMR spectrum obtained

### Stage 4: Preparation of 4-(2,6-dichlorobenzoylamino)-1H-pyrazole-3-carboxylic acid

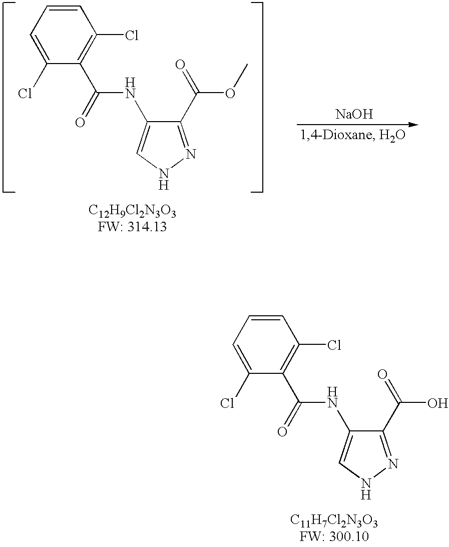

A solution of 4-(2,6-dichlorobenzoylamino)-1H-pyrazole-3-carboxylic acid methyl ester (1.308 Kg, 4.16 Mol, 1.0 wt) in 1,4-dioxane (6.47 L, 5.0 vol) was charged, in one portion, to 2M aq. sodium hydroxide solution (7.19 L, 14.38 Mol, 5.5 vol) at 35 to 45° C. The reaction mixture was cooled to 15 to 25° C. over 14 to 24 hours. Reaction completion was determined by TLC analysis². The reaction mixture was concentrated under vacuum at 45 to 50° C. The resultant oily residue was diluted with water (11.77 L, 9.0 vol) and acidified to pH1 with conc. aq. hydrochloric acid at 15 to 30° C. The precipitate was collected by filtration, washed with water (5.88 L, 4.5 vol), pulled dry on the filter and a displacement wash with heptanes (5.88 L, 4.5 vol) added. The filter-cake was charged to a 20 L rotary evaporator flask and azeo-dried with toluene (2×5.23 L, 2×4.0 vol) to afford 4-(2,6-dichlorobenzoylamino)-1H-pyrazole-3-carboxylic acid [1.207 Kg, 96.6% th, 92.3% w/w, ¹H NMR (d₆-DMSO) concordant with structure, 98.31% by HPLC area] as a yellow solid. ² Eluant: Ethyl acetate. UV visualisation. R_{f ester} 0.5, R_{f Stage 4} 0.0

### Stage 5: Preparation of 4-{[4-(2,6-dichlorobenzoylamino)-1H-pyrazole-3-carbonyl]amino}-piperidine-1-carboxylic acid tert-butyl ester

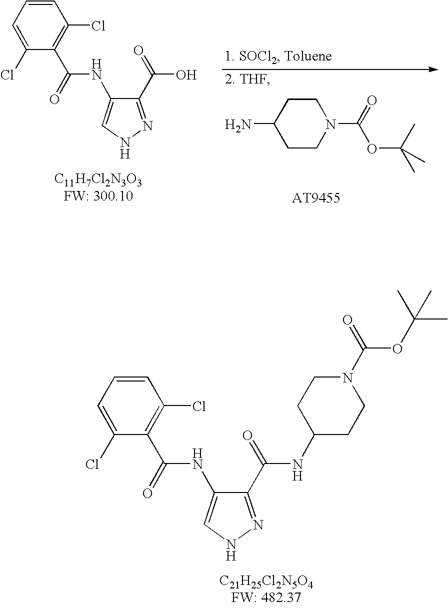

Thionyl chloride (0.25 L, 3.43 Mol, 0.3 vol) was added to a stirred suspension of 4-(2,6-dichlorobenzoylamino)-1H-pyrazole-3-carboxylic acid (0.806 Kg, 2.69 Mol, 1.0 wt) in toluene (8.00 L, 10.0 vol) under nitrogen at 16 to 25° C. The contents were then heated to and stirred at 80 to 100° C. for 16 to 24 hours. Reaction completion was determined by ¹H NMR analysis. The reaction mixture was cooled to 40 to 50° C., concentrated to dryness under vacuum at 45 to 50° C. and the residue azeo-dried with toluene (3×1.60 L, 3×2.0 vol) under vacuum at 45 to 50° C. to afford a white solid. The solid was transferred to a suitable vessel, tetrahydrofuran (4.00 L, 5.0 vol) charged, the contents stirred under nitrogen and triethylamine (0.42 L, 3.01 Mol, 0.512 vol) added at 16 to 25° C. A solution of 4-aminopiperidine-1-carboxylic acid tert-butyl ester (0.569 Kg, 2.84 Mol, 0.704 wt) in tetrahydrofuran (4.00 L, 5.0 vol) was then added to the reaction flask at 16 to 30° C. and the reaction mixture heated to and stirred at 45 to 50° C. for 2 to 16 hours. Reaction completion was determined by ¹H NMR analysis. The reaction mixture was cooled to 16 to 25° C. and quenched with water (4.00 L, 5.0 vol) and mixed heptanes (0.40 L, 0.5 vol). The contents were stirred for up to 10 minutes, the layers separated and the aqueous phase extracted with tetrahydrofuran:mixed heptanes [(9:1), 3×4.00 L, 3×5.0 vol]. The combined organic phases were washed with water (1.81 L, 2.5 vol) and concentrated under vacuum at 40 to 45° C. The residue was azeo-dried with toluene (3×4.00 L, 3×5.0 vol) to yield crude 4-{[4-(2,6-dichlorobenzoylamino)-1H-pyrazole-3-carbonyl]amino}-piperidine-1-carboxylic acid tert-butyl ester (1.257 Kg, 97.1% th, 156.0% w/w, corrected for 0.90% w/w entrained solvent). Several batches of compound were prepared in this way and the batches were combined for purification.

Crude 4-{[4-(2,6-dichlorobenzoylamino)-1H-pyrazole-3-carbonyl]amino}-piperidine-1-carboxylic acid tert-butyl ester (5.22 Mol, 1.0 wt), toluene (12.00 L, 4.87 vol) and methanol (0.30 L, 0.13 vol) were stirred under nitrogen for 3 to 18 hours at 16 to 25° C. The solid was isolated by filtration, the filter-cake washed with toluene (2×1.60 L, 2×0.7 vol) and dried under vacuum at 40 to 50° C. to yield 4-{[4-(2,6-dichlorobenzoylamino)-1H-pyrazole-3-carbonyl]amino}-piperidine-1-carboxylic acid tert-butyl ester [2.242 Kg, 86.6% th, 139.2% w/w, ¹H NMR (d₆-DMSO) concordant, 99.41% by HPLC area] as an off-white solid.

### Stage 6: Preparation of 4-(2,6-dichlorobenzoylamino)-1H-pyrazole-3-carboxylic acid piperidin-4-ylamide methanesuldhonate

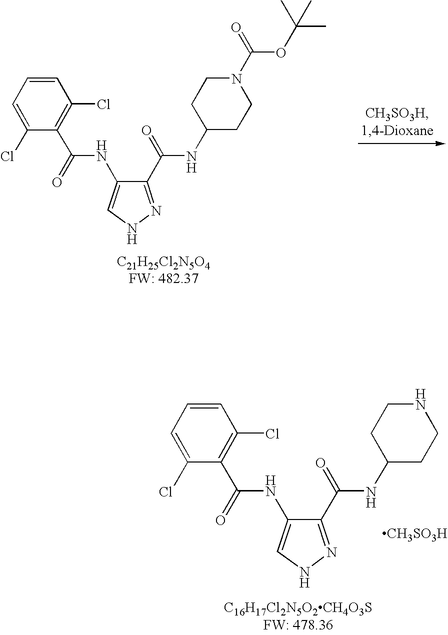

4-{[4-(2,6-Dichlorobenzoylamino)-1H-pyrazole-3-carbonyl]amino}-piperidine-1-carboxylic acid tert-butyl ester (0.561 Kg, 1.16 Mol, 1.0 wt) and 1,4-dioxane (14.00 L, 26.0 vol) were stirred under nitrogen and heated to 80 to 90° C. Methanesulphonic acid (0.30 L, 4.62 Mol, 0.54 vol) was added over 30 to 60 minutes at 80 to 90° C. and the contents heated to and maintained at 95 to 105° C. for 1 to 24 hours. Reaction completion was determined by ¹H NMR analysis. The reaction mixture was cooled to 20 to 30° C. and the resulting precipitate collected by filtration. The filter-cake was washed with propan-2-ol (2×1.10 L, 2×2.0 vol) and pulled dry on the filter for 3 to 24 hours to give 4-(2,6-dichlorobenzoylamino)-1H-pyrazole-3-carboxylic acid piperidin-4-ylamide methanesulphonate [0.558 Kg, 100.2% th, 99.4% w/w, ¹H NMR (d₆-DMSO) concordant with structure, 98.13% by HPLC area] as an off-white solid.

### Stage 7: Preparation of 4-(2,6-dichlorobenzoylamino)-1H-pyrazole-3-carboxylic acid (1-methanesulphonyl-piperidin-4-yl)-amide

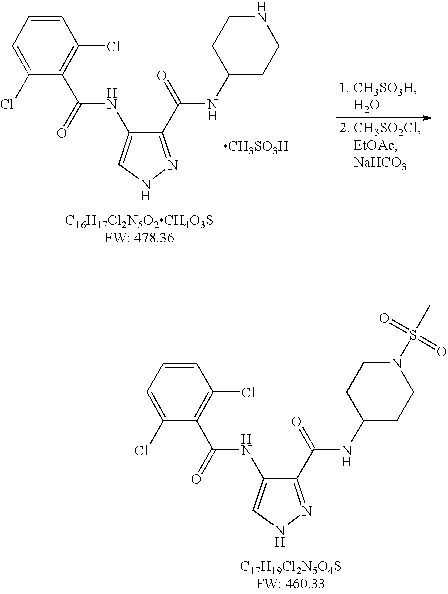

Methanesulphonic acid (0.055 L, 0.85 Mol, 0.1 vol) was added to a stirred suspension of 4-(2,6-dichlorobenzoylamino)-1H-pyrazole-3-carboxylic acid piperidin-4-ylamide methanesulphonate (0.562 Kg, 1.17 Mol, 1.0 wt) in water (5.60 L, 10.0 vol) at 15 to 40° C. The reaction mixture was heated to and stirred at 95 to 105° C. for 80 to 100 minutes. Reaction completion was determined by HPLC analysis. The mixture was cooled to 15 to 20° C., sodium hydrogen carbonate (1.224 Kg, 14.57 Mol, 2.18 wt) charged at 15 to 25° C. followed by ethyl acetate (4.20 L, 7.5 vol) and the temperature adjusted to 15 to 25° C. as necessary. Methanesulphonyl chloride (0.455 L, 5.88 Mol, 0.81 vol) was added in five aliquots over 120 to 180 minutes at 15 to 25° C. and the reaction mixture stirred for a further 30 to 45 minutes. Reaction completion was determined by HPLC analysis. The ethyl acetate was removed under vacuum at 35 to 45° C., the resulting slurry filtered, the filter-cake washed with water (0.56 L, 1.0 vol) and transferred to a suitably sized flask. Water (2.81 L, 5.0 vol) was charged and the mixture stirred for 30 to 40 minutes at 15 to 25° C. then filtered, the filter-cake washed with water (0.56 L, 1.0 vol) and pulled dry on the pad for 1 to 24 hours. The collected solids were dried under vacuum at 40 to 50° C. to give crude 4-(2,6-dichlorobenzoylamino)-1H-pyrazole-3-carboxylic acid (1-methanesulphonyl-piperidin-4-yl)-amide [0.490 Kg, 90.7% th, 87.2% w/w, ¹H NMR (d₆-DMSO) concordant with structure, 98.05% by HPLC area] as an off-white solid.

### Stage 8: Recrystallisation of 4-(2,6-dichlorobenzoylamino)-1H-pyrazole-3-carboxylic acid (1-methanesulphonyl-piperidin-4-yl)-amide

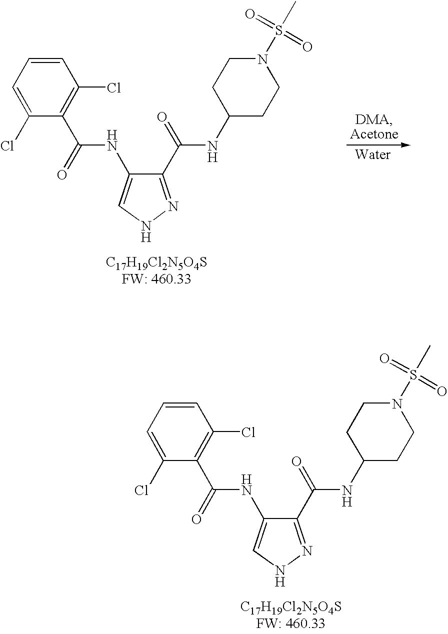

Crude 4-(2,6-dichlorobenzoylamino)-1H-pyrazole-3-carboxylic acid (1-methanesulphonyl-piperidin-4-yl)-amide (5.506 Kg, 11.96 Mol, 1.0 wt), N,N-dimethylacetamide (8.00 L, 1.5 vol) and acetone (11.00 L, 2.0 vol) were stirred under nitrogen and heated to 40 to 50° C. The resulting solution was clarified by filtration through glass microfibre paper and the filtrates heated to 60 to 80° C. Water (10.50 L, 2.0 vol) was added at 60 to 80° C. such that reflux was maintained throughout. The mixture was cooled to and aged at 15 to 25° C. for 14 to 24 hours, the crystallised solid isolated by filtration, the filter-cake washed with water (6.00 L, 1.0 vol) and transferred to a suitable vessel. Water (11.00 L, 2.0 vol) was charged, the mixture stirred for 30 to 40 minutes at 15 to 25° C. and then filtered. The filter-cake was washed with water (6.00 L, 1.0 vol) and pulled dry on the filter for at least 30 minutes. The solid was dried under vacuum at 40 to 50° C. to yield 4-(2,6-dichlorobenzoylamino)-1H-pyrazole-3-carboxylic acid (1-methanesulphonyl-piperidin-4-yl)-amide [4.530 Kg, 82.3% th, 82.3% w/w, ¹H NMR (d₆-DMSO) concordant with structure, 99.29% by HPLC area] as a white solid.

### Example 6

### Alternative Synthesis of 4-(2,6-dichloro-benzoylamino)-1H-pyrazole-3-carboxylic acid (1-methanesulphonyl-piperidin-4-yl)-amide

### Step 1: Synthesis of 4-[(4-nitro-1H-pyrazole-3-carbonyl)-amino]-piperidine-1-carboxylic acid tert-butyl ester

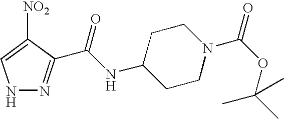

4-Nitropyrazole-3-carboxylic acid (20.0 g, 127.4 mmol) was suspended in CH₂Cl₂/DMF (99:1, 400 mL), treated cautiously with oxalyl chloride (11.6 mL, 134 mmol) and then stirred at room temperature for 16 h. The reaction mixture was evaporated then re-evaporated with toluene (×3) to give a yellow solid. The resultant acid chloride was suspended in dioxane (400 mL), treated with triethylamine (26.4 mL, 190 mmol) followed by 4-amino-1-BOC-piperidine (25.0 g, 125 mmol) and stirred at room temperature for 6 h. The reaction mixture was filtered and the solid collected stirred in water (500 mL) and then re-filtered. The solid collected was dried in vacuo, azeotroping with toluene, to give the title compound (37.6 g).

### Step 2: Synthesis of 4-nitro-1H-pyrazole-3-carboxylic acid piperidin-4-ylamide

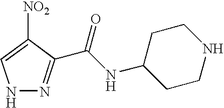

4-[(4-Nitro-1H-pyrazole-3-carbonyl)-amino]-piperidine-1-carboxylic acid tert-butyl ester (20.0 g, 59.0 mmol) was suspended in dioxane-CH₂Cl₂ (1:1, 400 ml) and treated with 4M HCl in dioxane (100 mL). The mixture was stirred at room temperature for 16 h and the solid formed collected by filtration, and dried in vacuo to give the title compound as a white solid (13.8 g).

### Step 3: Synthesis of 4-nitro-1H-pyrazole-3-carboxylic acid (1-methanesulphonyl-piperidin-4-yl)-amide

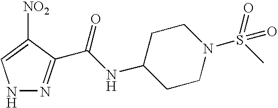

To a suspension of 4-nitro-1H-pyrazole-3-carboxylic acid piperidin-4-ylamide (13.7 g, 50.0 mmol) in dioxane-acetonitrile (1:1, 250 mL) was added triethylamine (17.4 mL, 125 mmol) followed by methanesulphonyl chloride (4.26 mL, 55.0 mmol). The mixture was stirred at 45° C. for 5 h then reduced in vacuo. To the residue was added water (500 mL), the mixture stirred for 20 min and the solid collected by filtration and dried in vacuo, azeotroping with toluene (×3), to give the title compound as an off-white solid (12.8 g)

### Step 4: Synthesis of 4-amino-1H-pyrazole-3-carboxylic acid (1-methanesulphonyl-piperidin-4-yl)-amide

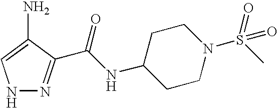

4-Nitro-1H-pyrazole-3-carboxylic acid (1-methanesulphonyl-piperidin-4-yl)-amide (5.0 g) was dissolved in DMF (30 mL), treated with 10% palladium on carbon (0.5 g) then hydrogenated at room temperature and 45 psi until the reaction was complete. The reaction mixture was filtered through Celite and reduced in vacuo. The residue was triturated with water (200 mL) and the resultant solid collected by filtration and dried in vacuo, azeotroping with toluene (×3) to give the title compound as the major product (3.5 g)

### Step 5: Synthesis of 4-(2,6-dichloro-benzoylamino)-1H-pyrazole-3-carboxylic acid (1-methanesulphonyl-piperidin-4-yl)-amide

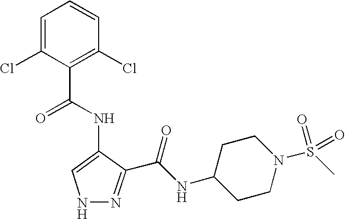

To a mixture of 4-amino-1H-pyrazole-3-carboxylic acid (1-methanesulphonyl-piperidin-4-yl)-amide (3.4 g, ˜10 mmol) and triethylamine (1.53 mL, 11 mmol) in dioxane (50 mL) at 45° C. was slowly added 2,6-dichlorobenzoyl chloride (1.4 mL, 10 mmol). The mixture was heated at 45° C. for 2 h, poured into water (250 mL) and then extracted with EtOAc (2×200 mL). The combined organic extracts were reduced in vacuo and purified by column chromatography on silica gel eluting with P.E-EtOAc (1:0-0:1). The product containing fractions were reduced in vacuo and the residue taken up in 2M aqueous NaOH-MeOH (1:1, 50 mL) and stirred at ambient temperature for 2 h. The MeOH was removed in vacuo and the mixture extracted with EtOAc. The organic portion was washed with brine, dried over MgSO₄ and reduced in vacuo. The residue was purified by hot slurry with EtOH to give the title compound as an off-white solid (2.52 g).

### Example 7

### Determination of the crystal structure of 4-(2,6-dichlorobenzoylamino)-1H-pyrazole-3-carboxylic acid (1-methanesulphonyl-piperidin-4-yl)-amide by X-ray diffraction

A crystal was obtained by evaporation of a CHCl₃ solution of the compound 4-(2,6-dichlorobenzoylamino)-1H-pyrazole-3-carboxylic acid (1-methanesulphonyl-piperidin-4-yl)-amide prepared as described in Example 6.

The crystal used for the diffraction experiment was colourless and of irregular shape with dimensions 0.15×015×0.04 mm³. Crystallographic data were collected at 104 K using CuKα radiation (λ=1.5418 Å) from a Rigaku rotating anode RU3HR, Osmic blue confocal optics, AFC9 ¼χ goniometer and a Rigaku Jupiter CCD detector. Images were collected in three w scans at 2θ=15° and four scans at 2θ=90° with a detector to crystal distance of 67 mm. Data collection was controlled by CrystalClear software and images were processed and scaled by Dtrek. Due to a high absorption coefficient (μ=4.04 mm⁻¹) data had to be corrected using 4^{th} order Fourier absorption correction. It was found that the crystals belong to a monoclinic space group C2/c (# 15) with crystal lattice parameters a=9.15, b=31.32, c=7.93 Å, β=113.3°, α=γ=90°. One short room temperature scan was taken to check crystal lattice parameters and symmetry. It was found that symmetry is the same as at 104 K and crystal lattice parameters are similar (room temperature a=9.19, b=31.31, c=8.09 Å, β=115.2°). The unit cell dimensions a, b & c have a deviation (s.u., standard uncertainty) of 5%.

The crystal structure was solved using direct methods implemented in SHELXS-97. Intensity data for a total of 2682 unique reflections in a resolution range from 15.67-0.84 Å (2.82<θ<66.54) were used in the refinement of 263 crystallographic parameters by SHELXL-97. Final statistical parameters were: wR2=0.1749 (all data), R_{F}=0.0663 (data with I>2σ(I)) and goodness of fit S=1.035.

Only one molecule of free base was found in the asymmetric unit. The elemental composition of the asymmetric unit was C₁₇H₁₉Cl₂N₅O₄S and the calculated density of the crystals is 1.47 Mg/m³. Hydrogen atoms were generated on geometrical grounds while the location of heteroatom bound hydrogen atoms was confirmed by inspection of Fo-Fc difference maps. The positional and thermal parameters of hydrogen atoms were constricted to ride on corresponding non-hydrogen atoms. The thermal motion of non-hydrogen atoms was modelled by anisotropic thermal factors (see FIG. 1).

The crystal structure contains one intramolecular (N6-H . . . O14 2.812 Å) and one intermolecular hydrogen bond (see FIG. 2). The molecules are linked together into chains by intermolecular H-bond N1-H . . . O22 2.845 Å. Dichlorophenyl moieties from different chains stack together forming compact 3D packing.

A thermal ellipsoid representation of the structure generated by the X-ray diffraction study is provided in FIG. 1 and packing diagram is in FIG. 2.

The coordinates for the atoms making up the structure of the free base of 4-(2,6-dichlorobenzoylamino)-1H-pyrazole-3-carboxylic acid (1-methanesulphonyl-piperidin-4-yl)-amide are as set out in cif format in Table 1 below.

TABLE 1
space group: C2/c (# 15)
unit cell at 104K with a, b & c having 5% s.u.:
a = 9.150
b = 31.320
c = 7.930
alpha = gamma = 90.00
beta = 113.30
loop_{—}
_atom_site_label
_atom_site_type_symbol
_atom_site_fract_x
_atom_site_fract_y
_atom_site_fract_z
_atom_site_U_iso_or_equiv
_atom_site_adp_type
_atom_site_occupancy
_atom_site_symmetry_multiplicity
_atom_site_calc_flag
_atom_site_refinement_flags
_atom_site_disorder_assembly
_atom_site_disorder_group
C11 C1 1.55055(16) 0.20997(4) 1.6202(2) 0.0376(4) Uani 1 1 d . . .
C12 C1 0.97743(17) 0.20548(4) 1.6837(3) 0.0447(5) Uani 1 1 d . . .
S1 S 0.57041(12) 0.07771(3) 0.25572(15) 0.0212(3) Uani 1 1 d . . .
O7 O 1.3597(5) 0.14890(12) 1.8380(5) 0.0376(10) Uani 1 1 d . . .
O14 O 1.0227(4) 0.12633(10) 1.1610(5) 0.0266(8) Uani 1 1 d . . .
O22 O 0.4600(4) 0.04232(10) 0.1911(5) 0.0285(9) Uani 1 1 d . . .
O23 O 0.6695(4) 0.08741(13) 0.1578(5) 0.0282(9) Uani 1 1 d . . .
N1 N 1.2370(5) 0.02604(12) 1.5929(6) 0.0215(9) Uani 1 1 d . . .
H1 H 1.2665 0.0019 1.6538 0.026 Uiso 1 1 calc . . .
N2 N 1.1481(5) 0.02788(12) 1.4095(6) 0.0241(10) Uani 1 1 d . . .
N6 N 1.2053(5) 0.13987(12) 1.5365(6) 0.0226(9) Uani 1 1 d . . .
H6 H 1.1513 0.1533 1.4330 0.027 Uiso 1 1 calc . . .
N15 N 0.9606(5) 0.05870(11) 1.0508(6) 0.0192(9) Uani 1 1 d . . .
H15 H 0.9804 0.0313 1.0720 0.023 Uiso 1 1 calc . . .
N19 N 0.6881(4) 0.06785(12) 0.4705(5) 0.0185(9) Uani 1 1 d . . .
C3 C 1.1279(5) 0.06988(14) 1.3718(7) 0.0196(10) Uani 1 1 d . . .
C4 C 1.2051(5) 0.09437(14) 1.5332(7) 0.0210(10) Uani 1 1 d . . .
C5 C 1.2765(6) 0.06537(16) 1.6738(8) 0.0240(11) Uani 1 1 d . . .
H5 H 1.3393 0.0714 1.7992 0.029 Uiso 1 1 calc . . .
C7 C 1.2811(6) 0.16340(14) 1.6846(7) 0.0243(11) Uani 1 1 d . . .
C8 C 1.2638(7) 0.21135(14) 1.6550(8) 0.0239(11) Uani 1 1 d . . .
C9 C 1.3834(6) 0.23627(16) 1.6278(7) 0.0260(11) Uani 1 1 d . . .
C10 C 1.3723(7) 0.27967(18) 1.6094(8) 0.0331(13) Uani 1 1 d . . .
H10 H 1.4564 0.2955 1.5978 0.040 Uiso 1 1 calc . . .
C11 C 1.2352(7) 0.30098(16) 1.6076(8) 0.0333(14) Uani 1 1 d . . .
H11 H 1.2266 0.3311 1.5928 0.040 Uiso 1 1 calc . . .
C12 C 1.1136(7) 0.27794(18) 1.6273(8) 0.0354(14) Uani 1 1 d . . .
H12 H 1.0207 0.2921 1.6242 0.043 Uiso 1 1 calc . . .
C13 C 1.1291(6) 0.23383(16) 1.6518(8) 0.0321(14) Uani 1 1 d . . .
C14 C 1.0327(5) 0.08684(14) 1.1863(7) 0.0218(11) Uani 1 1 d . . .
C16 C 0.8492(5) 0.07270(14) 0.8678(7) 0.0184(10) Uani 1 1 d . . .
H16 H 0.7916 0.0985 0.8838 0.022 Uiso 1 1 calc . . .
C17 C 0.9342(5) 0.08479(14) 0.7426(7) 0.0211(11) Uani 1 1 d . . .
H17A H 0.9903 0.0595 0.7223 0.025 Uiso 1 1 calc . . .
H17B H 1.0142 0.1073 0.8019 0.025 Uiso 1 1 calc . . .
C18 C 0.8119(5) 0.10120(15) 0.5567(7) 0.0225(10) Uani 1 1 d . . .
H18A H 0.7612 0.1276 0.5760 0.027 Uiso 1 1 calc . . .
H18B H 0.8665 0.1080 0.4743 0.027 Uiso 1 1 calc . . .
C20 C 0.6048(5) 0.05454(15) 0.5920(7) 0.0242(11) Uani 1 1 d . . .
H20A H 0.5265 0.0319 0.5305 0.029 Uiso 1 1 calc . . .
H20B H 0.5466 0.0792 0.6132 0.029 Uiso 1 1 calc . . .
C21 C 0.7264(6) 0.03785(14) 0.7776(7) 0.0234(11) Uani 1 1 d . . .
H21A H 0.6712 0.0302 0.8584 0.028 Uiso 1 1 calc . . .
H21B H 0.7798 0.0120 0.7578 0.028 Uiso 1 1 calc . . .
C24 C 0.4560(6) 0.12321(16) 0.2544(8) 0.0279(12) Uani 1 1 d . . .
H24A H 0.5263 0.1479 0.2999 0.042 Uiso 1 1 calc . . .
H24B H 0.3984 0.1181 0.3338 0.042 Uiso 1 1 calc . . .
H24C H 0.3796 0.1288 0.1288 0.042 Uiso 1 1 calc . . .

### Example 8

### X-Ray Powder Diffraction (XRPD) Studies of Crystals of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide were prepared using the recrystallisation method described in Example 5 Step 8.

The crystal samples for X-ray powder diffraction (XRPD) data collection were gently ground by marble mortar and loaded into a crystallographic capillary (from Hampton Research, Quartz or Glass Type 10, 0.4 or 0.7 mm diameter). Diffraction patterns were collected at room temperature using CuKα radiation (λ=1.5418 Å) from a Rigaku rotating anode RU3HR, Osmic blue confocal optics, ¼χ goniometer and a Rigaku HTC image plate detector. 2D Images were collected while spinning φ axis with a detector to crystal distance of 250 mm. Data collection was controlled by CrystalClear software and 2D images were converted to 1D plot (20 vs. Intensity) by Datasqueeze (intensity averaged over the azimuthal angle 0<X<360° for 2θ range 3-30° in 0.01° or 0.02° steps). An in house program AstexXRPD was used for manipulation and visualisation of 1D XRPD patterns (FIG. 3).

TABLE 2
2θ, d-spacing and relative intensity of main peaks.

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 16.57 | 5.35 | 59 |
| 16.95 | 5.23 | 62 |
| 19.53 | 4.55 | 37 |
| 20.42 | 4.35 | 76 |
| 20.88 | 4.25 | 23 |
| 22.66 | 3.92 | 100 |
| 24.33 | 3.66 | 40 |
| 24.99 | 3.56 | 16 |

### Example 9

### Physicochemical Studies on 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 5 Step 8 were subjected to differential scanning calorimetry studies and thermogravimetric analysis.

### Differential Scanning Calorimetry Study

Approximately 1-3 mg of sample (accurately weighed) were placed into an aluminium DSC pan and crimped using an aluminium lid to ensure a tight seal. The sample was then placed into a Pyris Diamond DSC (Perkin-Elmer) equipped with a liquid nitrogen cooling unit and allowed to equilibrate at 25° C. until a stable heat flow response was seen. A dry helium purge gas at a flow rate of 20 ml/min was used to produce an inert atmosphere and prevent oxidation of the sample during heating. The sample was then scanned from 25-400° C. at a scan rate of 200° C./min and the resulting heat flow response (mW) measured against temperature. Prior to experimental analysis the instrument was temperature and heat-flow calibrated using an indium reference standard.

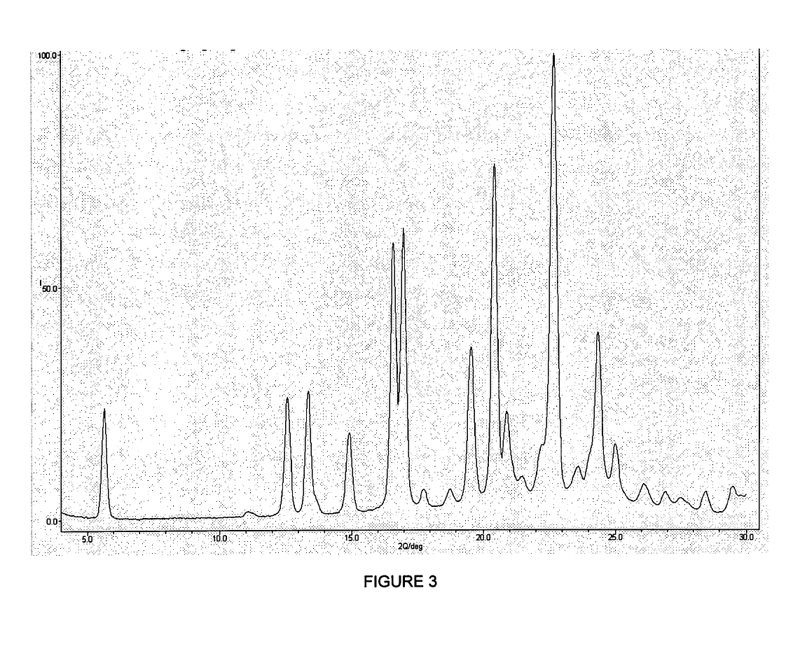

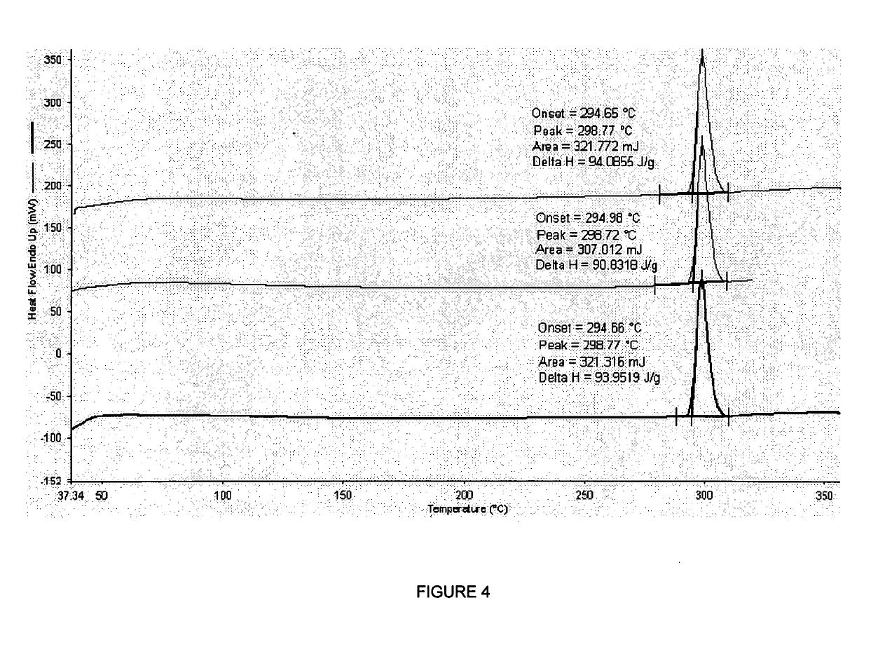

A DSC scan of the compound is shown in FIG. 4.

### Thermogravimetric Analysis

Approximately 5 mg of sample (accurately weighed) was placed into a platinum TGA pan and loaded into a TGA 7 gravimetric analyser. The sample under study was then heated at a rate of 10° C./min (from ambient to 300° C.) and the resulting change in weight monitored. A dry nitrogen purge gas at a flow rate of 20 ml/min was used to produce an inert atmosphere and prevent oxidation of the sample during heating. Prior to analysis the instrument was weight calibrated using a 100 mg reference standard and temperature calibrated using an Alumel reference standard (using the Curie point transition temperature).

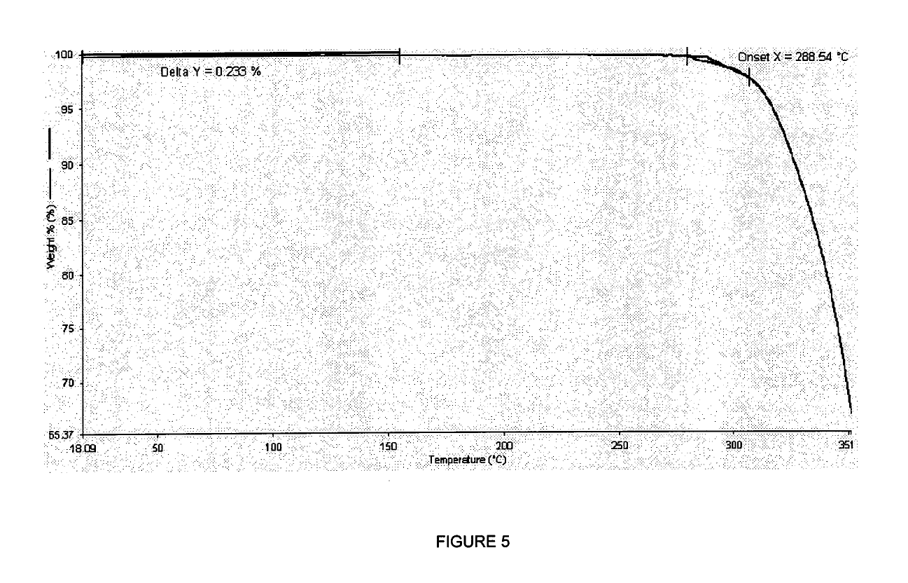

The weight loss profile of the compound is shown in FIG. 5.

### Results and Conclusions

From the resulting DSC thermograms obtained, a single defined and co-operative endothermic transition was seen onset ca. 294.5-295° C., indicative of the thermally induced melting of the crystalline lattice. No significant transitions were apparent prior to the main melting endotherm, indicating little/no loss of chemisorbed (bound) volatiles from the sample (as a result of dehydration/desolvation) as well as no detectable presence of amorphous content. This lack of a hydrated or solvated state was confirmed using TGA (FIG. 5) which showed a mass loss of approximately 0.2% up to 150° C. This suggests the existence of this drug form in the solely anhydrous crystalline state with no detectable polymorphic impurities or polymorphic transformations occurring.

The TGA plot (FIG. 5), shows a significant event at about 288° C. which occurred with an onset prior to the main melt transition, suggesting a small degree of thermally induced partial degradation of the sample prior to and during the melt. This degradation process was accelerated at temperatures greater than 300° C.

### Example 10

### Vapour Sorption/Desorption Analysis of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 5 Step 8 were subjected to vapour sorption/desorption analysis in order to test for the propensity of this sample to form a hydrated state.

Approximately 20 mg of sample was placed into a wire-mesh vapour sorption balance pan and loaded into an ‘IgaSorp’ vapour sorption balance (Hiden Analytical Instruments) held at 25+/−0.1° C. The sample was then dried by maintaining a 0% humidity environment (using mass flow control apparatus) until no further weight change was recorded. Subsequently, the sample was then subjected to a ramping profile from 0-90% relative humidity (% RH) at 10% RH increments, maintaining the sample at each step until equilibration had been attained (99.5% step completion).

Upon reaching equilibration, the % RH within the apparatus was ramped to the next step and the equilibration procedure repeated. After completion of the sorption cycle, the sample was then dried using the same procedure. The weight change during the sorption/desorption cycles was then monitored, allowing for the hygroscopic nature of the sample to be determined.

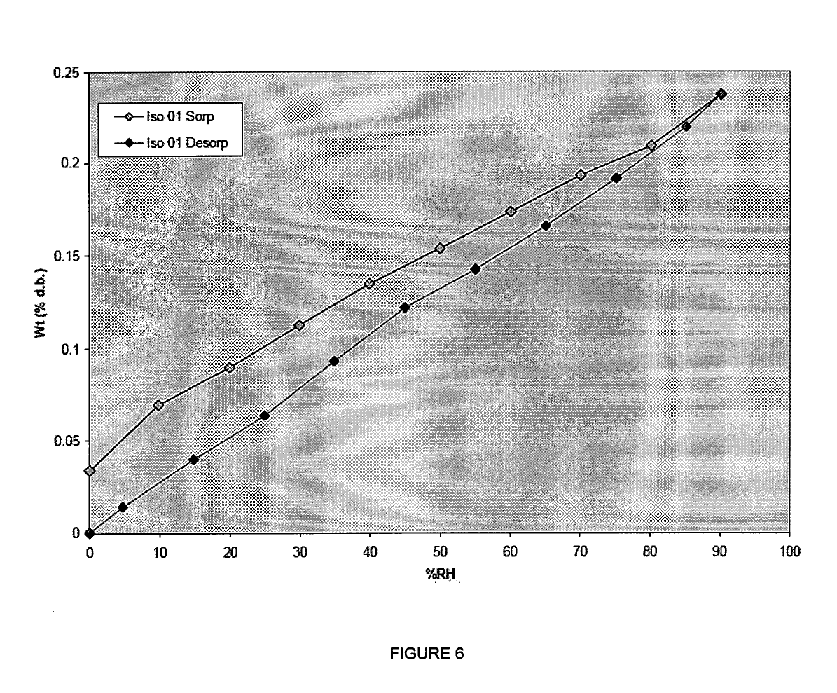

A vapour sorption/desorption profile of the compound is shown in FIG. 6.

During initial drying of the sample (at 0% RH), a weight loss of approximately 0.01% was seen, corresponding to the removal of loosely bound physi-sorbed or unbound surface adsorbed water present on the particles prior to analysis. Subsequently, increasing the relative humidity stepwise to 90% RH resulted in corresponding small incremental weight increases, totalling 0.24% upon equilibration at 90% RH. These small degrees of mass uptake seen upon storage at the varying humidities was the result of simple surface adsorption of a monolayer of water onto the particle surfaces with no true crystalline hydrate formation evident. This suggests that the compound is physically stable with regard to hygroscopicity and does not convert to the hydrated state upon storage in elevated humidity conditions.

### Biological Activity

### Example 11

### Measurement of Activated CDK2/CyclinA Kinase Inhibitory Activity Assay (IC₅₀)

See Example 5 of WO 2006/077416 at pages 72-73 (which is incorporated herein by reference).

### Example 12

### Measurement of Activated CDK1/CyclinB Kinase Inhibitory Activity Assay (IC₅₀)

See Example 6 of WO 2006/077416 at page 73 (which is incorporated herein by reference).

### Example 13

### GSK3-β Kinase Inhibitory Activity Assay

See Example 7 of WO 2006/077416 at pages 73-74 (which is incorporated herein by reference).

### Example 14

### Anti-proliferative Activity

See Example 8 of WO 2006/077416 at page 74 (which is incorporated herein by reference).

### Example 15

### Determination of Oral Bioavailability

See Example 9 of WO 2006/077416 at pages 74-75 (which is incorporated herein by reference).

### Example 16

### Xenograph Studies

See Example 10 of WO 2006/077416 at pages 75-76 (which is incorporated herein by reference).

### Example 17

### Comparative Example

See Example 11 of WO 2006/077416 at pages 76-77 (which is incorporated herein by reference).

### Pharmaceutical Formulations

### Example 18

See Example 12 of WO 2006/077416 at pages 77-79 (which is incorporated herein by reference).

### Example 19

### Pharmaceutical Formulations Containing a Solid Dispersion of 4-(2,6-dichloro-benzoylamino)-1H-pyrazole-3-carboxylic acid (1-methanesulphonyl-piperidin-4-yl)-amide in Polyvinylpyrrolidone (PVP)

This example describes the preparation of granule compositions containing a spray dried solid dispersion of 4-(2,6-dichloro-benzoylamino)-1H-pyrazole-3-carboxylic acid (1-methanesulphonyl-piperidin-4-yl)-amide and the K30 grade of polyvinylpyrrolidone (Kollidon K30) available from BASF ChemTrade GmbH of Burgbernheim, Germany). The molecular weight of the PVP is in the range 44,000-54,000.

The solid dispersion was prepared by dissolving 4-(2,6-dichloro-benzoylamino)-1H-pyrazole-3-carboxylic acid (1-methanesulphonyl-piperidin-4-yl)-amide in a 1:1 (v/v) mixture of ethanol and dichloromethane to give a concentration of the compound of 50 mg/mL, and then adding PVP K30 in a ratio of compound to PVP of 1:3.

The solute was then spray dried in a Niro Mobile Minor 2000 spray dryer. The powder collected from the spray dryer was dried under vacuum.

The spray drying conditions were as follows:

| Nozzle internal diameter (ID): | 1 mm |
|---|---|
| Tubing ID: | 3 mm |
| Inlet temperature: | 180° C. |
| Exhaust temperature: | 85° C. |
| Atomisation pressure: | 1.0 bar |
| Process gas flow: | 3.2 mbar (83 kg/h of nitrogen) |
| Process gas: | nitrogen |
| Solution dry weight (compound + PVP): | 1980 g |
| Flow rate: | 123 g/min |
| Yield: | 84.85% |

The particle size distribution of the spray dried solid dispersion, following drying, was measured using a laser diffraction apparatus and gave D10, D50 and D90 figures as follows:

| | |
|---|---|
| D10/μm | 17.53 |
| D50/μm | 49.08 |
| D90/μm | 93.26 |

In the following example, the solid dispersion of 4-(2,6-dichloro-benzoylamino)-1H-pyrazole-3-carboxylic acid (1-methanesulphonyl-piperidin-4-yl)-amide in PVP is referred to as “Compound of formula (I)/PVP”.

The following materials were blended for 30 seconds in a high shear mixer:—

| | |
|---|---|
| Dicalcium phosphate (Emcompress ™) | 32.8 g |
| Silicified microcrystalline cellulose (ProSolv HD90 ™) | 10.9 g |
| Compound of formula (I)/PVP | 35.2 g |
| Croscarmellose sodium (Ac-Di-Sol ™) | 11.1 g |

The powder blend was then compressed using a Freund roller compactor. The following settings were required to produce a ribbon:—

| | |
|---|---|
| Feed speed: | 60 rpm |
| Roller speed: | 2 rpm |
| Roller pressure: | 180 kgf/cm2 |

The ribbon of compressed powder was ground through a 710 μm sieve and the resulting granules were collected in a suitable container. An aliquot of the granule mass (9.0 g) was mixed with a further aliquot of Ac-Di-Sol (1.0 g). The quantity of the granule mass that could be filled into size 0 capsules was determined (both flush-filled and tightly packed). Results are summarised below.

Capsule fill weight

| Flush-filled | Tightly packed |
|---|---|
| 282 mg (24.8 mg compound) | 431 mg (37.9 mg) |

### Disintegration Tests

For rapid release oral formulations, it is desirable that disintegration of the dosage form and release of the active ingredient should occur within 15 minutes. The capsule formulation described was therefore subjected to disintegration testing using a standard tablet/capsule disintegration apparatus (European Pharmacopoeia, 4^{th} Edition). Distilled water was used as the disintegration medium. The volume of the disintegration medium was 800 mL and the temperature was maintained at 37° C. (+/−1° C.). The assessment of dispersion/dissolution behaviour of the formulation was made by observation alone. The disintegration times are set out in Table below.

| Quantity of Compound of | |
|---|---|
| formula (I) per capsule | |
| (mg) | Disintegration time (min) |
| 24.8 (flush-filled) | 4 |
| 37.9 (tightly packed) | 5 |

### Dissolution Testing

The rate of dissolution of the capsule formulation was compared with the rate of dissolution of (1) the non-encapsulated solid dispersion of PVP and the compound of formula (I) containing no further excipients and (2) the solid dispersion (1) packed tightly into a size 0 capsule, and (3) the formulated sample.

The dissolution testing was conducted using the paddle apparatus as described in the European Pharmacopoeia, 4^{th} Edition.

The results of the dissolution studies are shown in FIG. 7. FIG. 7 is a graph of solubility against time for several formulations containing a solid dispersion of 4-(2,6-dichloro-benzoylamino)-1H-pyrazole-3-carboxylic acid (1-methanesulphonyl-piperidin-4-yl)-amide and PVP, where (1) indicates the non-encapsulated solid dispersion of PVP and the compound of formula (I) containing no further excipients; (2) indicates the solid dispersion (1) packed tightly into a size 0 capsule and (3) indicates the formulated sample.

The results show that dissolution of the non-encapsulated solid dispersion was quicker than the dissolution of the capsule sample. In the tightly packed encapsulated sample, the PVP is probably binding the particles together, thus retarding the release of the compound of formula (I). Interestingly, the formulated sample exhibited a much more rapid compound release profile compared with the non-formulated, encapsulated sample, which indicates that the high proportion of disintegrant in the formulation is effective in countering the binding capacity of the PVP.

### Example 20

### Determination of Antifungal Activity

See Example 13 of WO 2006/077416 at pages 79-80 (which is incorporated herein by reference). The antifungal activity of the compounds of the formula (I) can be determined using the following protocol.

### Example 21

### Protocol for the Biological Evaluation of Control of In Vivo Whole Plant Fungal Infection

See Example 14 of WO 2006/077416 at page 81 (which is incorporated herein by reference).

### Example 22

### Assay for Therapeutic Efficacy

The effect of a compound of formula (0) (e.g. Compound I) in combination with a compound of formula (I′″) can be assessed using the following technique:

### IC₅₀ Shift Assay

Cells from human cells lines (e.g. HCT116, U87MG, A549) were seeded onto 96-well tissue culture plates at a concentration of 2.5×10³, 6.0×10³, or 4.0×10³ cells/well respectively. Cells were allowed to recover for 48 hours prior to addition of compound(s) or vehicle control (0.35% DMSO) as follows:

Compounds were added concurrent for 96 hours.

Following a total of 96 hours compound incubation, cells were fixed with ice-cold 10% (w/v) trichloroacetic acid for 1 hour on ice and then washed four times with dH₂O using a plate washer (Labsystems Wellwash Ascent) and air-dried. Cells were then stained with 0.4% (w/v) Sulforhodamine B (Sigma) in 1% acetic acid for 20 min at room temperature and then washed four times with 1% (v/v) acetic acid and air-dried before the addition of 10 mM Tris buffer to solubilise the dye. Colourmetric product was quantified by reading at Abs490 nm on a Wallac Victor2 plate reader (1420 multilabel counter, Perkin Elmer Life Sciences). The IC₅₀ for Compound II in the presence of varying doses of Compound I was determined. Synergy was determined when the IC₅₀ shifted down in the presence of sub-effective doses of Compound I. Additivity was determined when the response to Compound II and Compound I together resulted in an effect equivalent to the sum of the two compounds individually. Antagonistic effects were defined as those causing the IC₅₀ to shift upwards, i.e. those where the response to the two compounds was less than the sum of the effect of the two compounds individually.

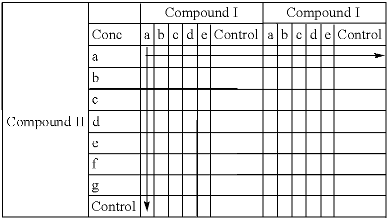

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-154. (canceled)

155. A combination comprising:
(a) a compound of formula (Ia): 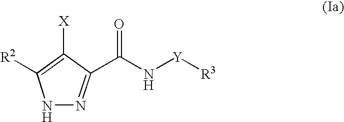
or salts or tautomers or N-oxides thereof,
wherein
X is a group R¹-A-NR⁴—;
A is a bond, C═O, NR⁹(C═O) or O(C═O) wherein R⁹ is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is a carbocyclic or heterocyclic group having from 3 to 12 ring members; or a C₁₋₈ hydrocarbyl group optionally substituted by one or more substituents selected from fluorine, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₄ hydrocarbyl group optionally substituted by halogen, hydroxyl or C₁₋₄ alkoxy;
R³ is selected from hydrogen and carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy; and
(b) a compound of formula (I′″): 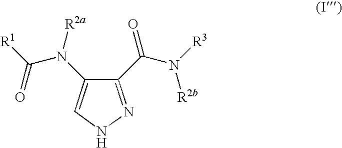
or salts or tautomers, or N-oxides thereof;
wherein:
R¹ is 2,6-dichlorophenyl;
R^{2a} and R^{2b} are both hydrogen;
and R³ is a group: 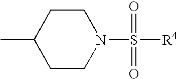
where R⁴ is C₁₋₄ alkyl.

156. The combination of claim 155 wherein the compound of formula (Ia) has the formula (Ib): 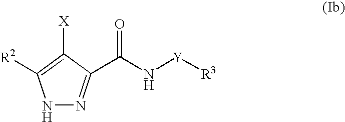
or salts or tautomers or N-oxides thereof;
wherein
X is a group R¹-A-NR⁴—;
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is a carbocyclic or heterocyclic group having from 3 to 12 ring members; or a C₁₋₈ hydrocarbyl group optionally substituted by one or more substituents selected from fluorine, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

157. The combination of claim 155 wherein the compound of formula (Ia) has the formula (II′): 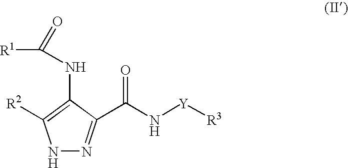
wherein R¹, R², R³ and Y are as defined in claim 155.

158. The combination of claim 155 wherein the compound of formula (Ia) has the formula (IV): 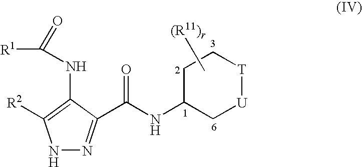
or salts or tautomers or N-oxides thereof;
wherein R¹ and R² are as defined in claim 155;
an optional second bond may be present between carbon atoms numbered 1 and 2;
one of U and T is selected from CH₂, CHR¹³, CR¹¹R¹³, NR¹⁴, N(O)R¹⁵, O and S(O)ₜ; and the other of U and T is selected from, NR¹⁴, O, CH₂, CHR¹¹, C(R¹¹)₂, and C═O; r is 0, 1, 2, 3 or 4; t is 0, 1 or 2;
R¹¹ is selected from hydrogen, halogen, C₁₋₃ alkyl and C₁₋₃ alkoxy;
R¹³ is selected from hydrogen, NHR¹⁴, NOH, NOR¹⁴ and R^{a}-R^{b};
R^{a} is a bond, O, CO, XIC(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NRC, X¹C(X²), C(X²)X¹ or X¹C(X²)X¹;
R^{c} is selected from hydrogen and C₁₋₄ hydrocarbyl;
X¹ is O, S or NRC and X² is ═O, ═S or ═NR^{c};
R¹⁴ is selected from hydrogen and R^{d} —R^{b};
R^{d} is selected from a bond, CO, C(X²)X¹, SO₂ and SO₂NR^{c}; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group, provided that U and T cannot be 0 simultaneously.

159. The combination of claim 158 wherein the compound of formula (Ia) has the formula (IVa): 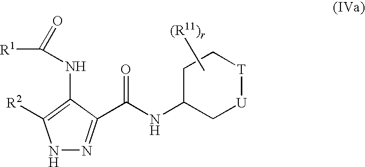
or salts or tautomers or N-oxides thereof;
wherein one of U and T is selected from CH₂, CHR¹³, CR¹¹R¹³, NR¹⁴, N(O)R¹⁵, O and S(O)ₜ; and the other of U and T is selected from CH₂, CHR¹¹, C(R¹¹)₂, and C═O; r is 0, 1 or 2; t is 0, 1 or 2;
R¹¹ is selected from hydrogen and C₁₋₃ alkyl;
R¹³ is selected from hydrogen and R^{a}-R^{b};
R¹⁴ is selected from hydrogen and R^{d}-R^{b};
R^{d} is selected from a bond, CO, C(X²)X¹, SO₂ and SO₂NR^{c}; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group.

160. The combination of claim 159 wherein the compound of formula (Ia) has the formula (VIa): 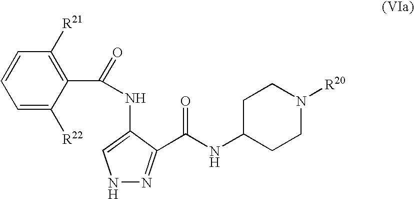
or salts or tautomers or N-oxides thereof;
wherein R²⁰ is selected from hydrogen and methyl;
R²¹ is selected from fluorine and chlorine; and
R²² is selected from fluorine, chlorine and methoxy; or
one of R²¹ and R²² is hydrogen and the other is selected from chlorine, methoxy, ethoxy, difluoromethoxy, trifluoromethoxy and benzyloxy.

161. The combination of claim 160 wherein the compound of formula (Ia) has the formula (VIb): 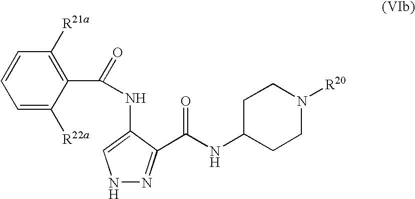
or salts or tautomers or N-oxides thereof;
wherein R²⁰ is selected from hydrogen and methyl;
R^{21a} is selected from fluorine and chlorine; and
R^{22a} is selected from fluorine, chlorine and methoxy.

162. The combination of claim 161 wherein the compound of the formula (VIb) is selected from:
4-(2,6-difluoro-benzoylamino)-1H-pyrazole-3-carboxylic acid piperidin-4-ylamide;
4-(2,6-difluoro-benzoylamino)-1H-pyrazole-3-carboxylic acid (1-methyl-piperidin-4-yl)-amide;
4-(2,6-dichloro-benzoylamino)-1H-pyrazole-3-carboxylic acid piperidin-4-ylamide; and
4-(2-fluoro-6-methoxy-benzoylamino)-1H-pyrazole-3-carboxylic acid piperidin-4-ylamide;
and salts thereof.

163. The combination of claim 162 wherein the compound of the formula (VIb) is 4-(2,6-dichloro-benzoylamino)-1H-pyrazole-3-carboxylic acid piperidin-4-ylamide or a salt thereof.

164. The combination of claim 155 wherein the compound of formula (I′″) is 4-(2,6-dichloro-benzoylamino)-1H-pyrazole-3-carboxylic acid (1-methanesulphonyl-piperidin-4-yl)-amide.

165. The combination of claim 163 wherein the compound of formula (I′″) is 4-(2,6-dichloro-benzoylamino)-1H-pyrazole-3-carboxylic acid (1-methanesulphonyl-piperidin-4-yl)-amide.

166. The combination of claim 155 further comprising one or more auxiliary compound(s).

167. The combination of claim 166 wherein the auxiliary compound is selected from:
(i) a taxane compound
(ii) an antimetabolic compound selected from gemcitabine, capecitabine, cytarabine, ralitrexed, pemetrexed, and methotrexate;
(iii) a signalling inhibitor selected from trastuzumab, cetuximab, gefitinib, erlotinib, bevacizumab, imatinib mesylate, sorafenib, dasatinib, lapatinib, nilotinib, vandetanib, vatalinib, axitinib and CHIR-258;
(iv) a cytokine, cytokine activating agent or retinoid;
(v) a camptothecin compound;
(v) a vinca alkaloid compound;
(vi) a platinum compound;
(vii) a topoisomerase 2 inhibitor;
(viii) an antiandrogen or an antiestrogen;
(ix) a GnRH analog;
(x) a monoclonal antibody to cell surface antigens (or an anti-CD antibody);
(xi) an alkylating agent;
(xii) a HDAC inhibitor;
(xiii) a COX-2 inhibitor;
(xiv) a DNA methylation inhibitor;
(xv) a proteasome inhibitor; and
(xvi) a CDK inhibitor;

168. The combination of claim 167 wherein the auxiliary compound is selected from
(a) a taxane compound selected from paclitaxel and docetaxel;
(b) an antimetabolic compound selected from gemcitabine, capecitabine, cytarabine, ralitrexed, pemetrexed, and methotrexate;
(c) a signalling inhibitor selected from trastuzumab, cetuximab, gefitinib, erlotinib, bevacizumab, imatinib mesylate, sorafenib, dasatinib, lapatinib, nilotinib, vandetanib, vatalinib, axitinib and CHIR-258.
(d) a cytokine, cytokine activating agent or retinoid selected from an interferon, an interleukin, tretinoin, alitretinoin and bexarotene;
(e) a camptothecin compound selected from camptothecin, irinotecan and topotecan;
(f) a vinca alkaloid selected from vinorelbine, vinblastine and vincristine;
(g) a platinum compound selected from chloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane)-(isocitrato)platinum (II); (1,2-diaminocyclohexane)-cis-(pyruvato)platinum (II); onnaplatin; tetraplatin, cisplatin, carboplatin and oxaliplatin;
(h) a topoisomerase 2 inhibitor selected from anthracyclines derivatives, mitoxantrone, and podophyllotoxin derivatives.
(i) an antiandrogen or an antiestrogen selected from the aromatase inhibitors letrozole, anastrozole, exemestane and aminoglutethimide; and the antiandrogens tamoxifen, fulvestrant, raloxifene, toremifene, droloxifene, letrazole, anastrazole, exemestane, bicalutamide, luprolide, megestrol acetate, aminoglutethimide and bexarotene;
(j) a GnRH analog selected from goserelin and leuprolide;
(k) a monoclonal antibody to cell surface antigens (or an anti-CD antibody) selected from CD20, CD22, CD33, CD52, rituximab, tositumomab and gemtuzumab;
(l) an alkylating agent selected from a nitrogen mustard compound, nitrosourea compound and busulfan;
(m) a HDAC inhibitor selected from TSA, SAHA, JNJ-16241199, LAQ-824, MGCD-0103 and PXD-101;
(n) a COX-2 inhibitor which is celecoxib;
(o) a DNA methylation inhibitor selected from temozolomide, decitabine and 5-azacitidine;
(p) a proteasome inhibitor which is bortezimib; and
(q) a CDK inhibitor selected from seliciclib, alvocidib, 7-hydroxystaurosparine, JNJ-7706621, BMS-387032, Pha533533, PD332991, ZK-304709 and AZD-5438.

169. The combination of claim 155 comprising two or more auxiliary compounds independently selected from: an antimetabolic compound, a taxane compound, an epothilone, an Hsp90 inhibitor, a signalling inhibitor, a camptothecin compound, a vinca alkaloid compound, a platinum compound, a topoisomerase 2 inhibitor, an antiandrogen, a monoclonal antibody, an alkylating agent, a histone deacetylase inhibitor (HDAC), a cylcooxygenase-2 (COX-2) inhibitor, a proteasome inhibitor, DNA methylation inhibitor, a CDK inhibitor and an Aurora inhibitor.

170. The combination of claim 155 in the form of a pharmaceutical pack, kit or patient pack.

171. A method for treating, alleviating or reducing the incidence of a disease or condition comprising or arising from abnormal cell growth in a mammal, which method comprises administering to the mammal a combination according to claim 155 in an amount effective in inhibiting abnormal cell growth.

172. A method for the treatment of a cancer in a warm-blooded animal, which comprises administering to said animal an effective amount of an compound of formula (I′″) sequentially or simultaneously with an effective amount of a compound of formula (Ia) wherein the compounds of formula (I′″) and formula (Ia) are as defined in claim 155.

173. A method of enhancing or potentiating the response rate in a patient suffering from a cancer where the patient is being treated with an compound of formula (I′″), which method comprises administering to the patient, in combination with the compound of formula (I′″), a compound of formula (Ia) as defined in claim 155.

174. The combination of claim 165 further comprising one or more auxiliary compound(s) wherein the auxiliary compound is selected from:
(a) a taxane compound selected from paclitaxel and docetaxel;
(b) an antimetabolic compound selected from gemcitabine, capecitabine, cytarabine, ralitrexed, pemetrexed, and methotrexate;
(c) a signalling inhibitor selected from trastuzumab, cetuximab, gefitinib, erlotinib, bevacizumab, imatinib mesylate, sorafenib, dasatinib, lapatinib, nilotinib, vandetanib, vatalinib, axitinib and CHIR-258.
(d) a cytokine, cytokine activating agent or retinoid selected from an interferon, an interleukin, tretinoin, alitretinoin and bexarotene;
(e) a camptothecin compound selected from camptothecin, irinotecan and topotecan;
(f) a vinca alkaloid selected from vinorelbine, vinblastine and vincristine;
(g) a platinum compound selected from chloro(diethylenediamino)-platinum (II) chloride; dichloro(ethylenediamino)-platinum (II); spiroplatin; iproplatin; diamino(2-ethylmalonato)platinum (II); (1,2-diaminocyclohexane)malonatoplatinum (II); (4-carboxyphthalo)-(1,2-diaminocyclohexane)platinum (II); (1,2-diaminocyclohexane)-(isocitrato)platinum (II); (1,2-diaminocyclohexane)-cis-(pyruvato)platinum (II); onnaplatin; tetraplatin, cisplatin, carboplatin and oxaliplatin;
(h) a topoisomerase 2 inhibitor selected from anthracyclines derivatives, mitoxantrone, and podophyllotoxin derivatives.
(i) an antiandrogen or an antiestrogen selected from the aromatase inhibitors letrozole, anastrozole, exemestane and aminoglutethimide; and the antiandrogens tamoxifen, fulvestrant, raloxifene, toremifene, droloxifene, letrazole, anastrazole, exemestane, bicalutamide, luprolide, megestrol acetate, aminoglutethimide and bexarotene;
(j) a GnRH analog selected from goserelin and leuprolide;
(k) a monoclonal antibody to cell surface antigens (or an anti-CD antibody) selected from CD20, CD22, CD33, CD52, rituximab, tositumomab and gemtuzumab;
(l) an alkylating agent selected from a nitrogen mustard compound, nitrosourea compound and busulfan;
(m) a HDAC inhibitor selected from TSA, SAHA, JNJ-16241199, LAQ-824, MGCD-0103 and PXD-101;
(n) a COX-2 inhibitor which is celecoxib;
(o) a DNA methylation inhibitor selected from temozolomide, decitabine and 5-azacitidine;
(p) a proteasome inhibitor which is bortezimib; and
(q) a CDK inhibitor selected from seliciclib, alvocidib, 7-hydroxystaurosparine, JNJ-7706621, BMS-387032, Pha533533, PD332991, ZK-304709 and AZD-5438.
